SEQUENCE LISTING (1 sequence)

SEQ ID NO: 1 - protein, 9 aa, artificial
  synthetic
RPPGFSPFR

(19) United States
(12) Patent Application Publication
(10) Pub. No.: US 2005/0089473 A1
(43) Pub. Date: Apr. 28, 2005

(54) Potassium channel mediated delivery of agents through the blood-brain barrier

(75) Inventors: Keith L. Black, Los Angeles, CA (US); Nagendra S. Ningaraj, Brentwood, TN (US)

Correspondence Address: KING & SPALDING LLP, 191 PEACHTREE STREET, N.E., ATLANTA, GA, 30303-1763, US

(73) Assignee: Cedars-Sinai Medical Center

(21) Appl. No.: 10/938,674

(22) Filed: Sep. 10, 2004

Related U.S. Application Data
(60) Provisional application No. 60/548,636, filed on Feb. 27, 2004.
(60) Provisional application No. 60/528,440, filed on Dec. 10, 2003.
(60) Provisional application No. 60/502,159, filed on Sep. 10, 2003.

(51) Int. Cl. A61K 49/00; A61K 31/445; A61K 31/513

(52) U.S. Cl. 424/9.1; 514/269; 514/317

(57) ABSTRACT

This invention includes pharmaceutical compositions, methods and kits for the treatment or diagnosis of a malignant tumors, including brain tumors, and diseases or disorders characterized by abnormal brain tissue.

### CROSS REFERENCE TO RELATED APPLICATIONS

The present application claims priority to U.S. Provisional Patent Application Ser. No. 60/548,636 filed Feb. 27, 2004, U.S. Provisional Patent Application Ser. No. 60/528,440 filed Dec. 10, 2003, and U.S. Provisional Patent Application Ser. No. 60/502,159 filed Sep. 10, 2003, the contents of all of which are incorporated herein by reference.

### FIELD OF THE INVENTION

This invention includes pharmaceutical compositions, methods and kits for the treatment or diagnosis of a malignant tumors, including brain tumors, and diseases or disorders characterized by abnormal brain tissue.

### BACKGROUND OF THE INVENTION

The blood-brain barrier (BBB) is a transvascular permeability barrier that tightly controls entry of substances into the brain. Unlike capillaries that serve other areas of the body, the capillaries that perfuse the brain are lined with special endothelial cells that lack fenestrations and are sealed by endothelial tight junctions. This tight endothelium provides a physical barrier that together with metabolic barriers is thought to form the basis of the BBB. Maintenance of the blood-brain barrier may involve endogenous nitric oxide production and a cyclic GMP-dependent mechanism (Liu, S. M. and Sundqvist, T. “Nitric oxide and cGMP regulate endothelial permeability and F-actin distribution in hydrogen peroxide-treated endothelial cells” Exp. Cell. Res. 1997, 235(1), 238-44).

The BBB protects the brain against pathogens (e.g., viruses) and other dangers of the circulatory system, including changes in the composition of the systemic blood supply (e.g., electrolyte levels). The barrier is not complete, however, and permits entry of certain substances, such as small fat-soluble (lipophilic) molecules that can freely diffuse through the barrier. The BBB also permits entry of essential nutrients, such as glucose and amino acids, which are vital to brain function. These nutrients are generally water soluble (hydrophilic), and require more complex mechanisms for crossing the BBB, such as carrier-mediated transport, receptor-mediated transcytosis and absorptive-mediated transcytosis.

While protective under normal circumstances, the BBB frustrates delivery of drugs and other therapeutic molecules to the brain. It has been reported that the BBB blocks delivery of more than 98% of central nervous system (CNS) drugs (Pardridge, W J. “Nature Rev.: Drug Discovery 2002 1:131-139). The drug delivery challenge posed by the BBB is compelling, particularly as the population ages and the incidence of neurodegenerative diseases such as stroke, Alzheimer's disease, and Parkinson's disease increase in prevalence. The problem is particularly acute for patients with malignant brain tumors, who cannot benefit from anticancer drugs effective in treating tumors elsewhere in the body. A solution to drug delivery across the BBB would produce an exponential increase in the number of drugs available for the treatment and prevention of brain-based disorders.

An ideal strategy for delivery of therapeutic molecules to abnormal brain regions would involve selective opening of only that portion of the BBB that serves the abnormal region. The portion of the BBB that serves a brain tumor is known as the blood-brain tumor barrier (BTB). Several strategies have been explored to permit enhanced delivery of drugs to abnormal brain tissue (Misra A, Ganesh S, Shahiwala A. “Drug delivery to the central nervous system” J Pharm Pharmaceuti Sci. 2003 6(2):252-273). Some of these efforts have focused on changing the therapeutic molecule, rather than altering the barrier. Rational drug design or drug delivery strategies have attempted to “lipidize” otherwise poorly-lipid soluble compounds, either by developing lipophilic analogs or packing hydrophilic drugs in liposomes. This approach has been limited by the relative instability of lipophilic analogs in the blood, and the rapid removal of these analogs from the blood as a direct result of their increased lipid solubility.

Other strategies have focused on circumventing the barrier, for example by directly injecting drugs into the brain or through the use of implantable drug delivery devices (i.e., Gliadel Wafers™, Guildford Pharmaceutical). Not only are these strategies highly invasive, they do not provide specific delivery to the abnormal brain region. Intracerebral implants, moreover, are ineffective against larger tumors (i.e., greater than 500 μm) because drug diffusion from these devices is limited. Intracerebral implants are generally ineffective against tumors that are highly diffused, such as gliomas (e.g., glioblastoma multiforme (GBM)).

Disruption of the BBB provides another strategy for enhanced drug delivery. A variety of disruption techniques have been studied, but most have produced disappointing results or unacceptable toxicity (Misra A, Ganesh S, Shahiwala A. “Drug delivery to the central nervous system” J Pharm Pharmaceuti Sci. 2003 6(2):252-273). These include, for example, the infusion of solvents such as dimethyl sulfoxide or ethanol (Broadwell R D, Salcman M, Kaplan R S. “Morphologic effect of dimethyl sulfoxide on the blood-brain barrier” Science. 1982 9:217(4555):164-6; Greig N H, Sweeney D J, Rapoport S I. “Inability of dimethyl sulfoxide to increase brain uptake of water-soluble compounds: implications to chemotherapy for brain tumors” Cancer Treat Rep. 1985 69(3):305-12), metals such as aluminum (Favarato M, Zatta P, Perazzolo M, Fontana L, Nicolini M. “Aluminum(III) influences the permeability of the blood-brain barrier to [14C]sucrose in rats” Brain Res. 1992 569(2):330-5), as well as X-irradiation (Qin D, Ma J, Xiao J, Tang Z. “Effect of brain irradiation on blood-CSF barrier permeability of chemotherapeutic agents” Am J Clin Oncol. 1997 20(3):263-5) and the induction of pathological conditions associated with BBB disruption (i.e., hypertension, ischemia) (Suzuki N, Sako K, Yonemasu Y. “Effects of induced hypertension on blood flow and capillary permeability in rats with experimental brain tumors” J Neurooncol. 1991 10(3):213-8). Other experimental strategies have included co-administration of convulsants and anti-convulsants, and the systemic administration of anti-neoplastic agents (Neuwelt E A, Barnett P, Barranger J, McCormick C, Pagel M, Frenkel E. “Inability of dimethyl sulfoxide and 5-fluorouracil to open the blood-brain barrier” Neurosurgery. 1983 12(1):29-34). The mechanisms responsible for many of these techniques are poorly understood.

Osmotic disruption has shown some clinical promise but suffers from limitations that prevent its widespread use. Osmotic disruption works by initiating endothelial cell shrinkage and causes the tight junctions to open, thereby increasing permeability. Hypertonic mannitol, for example, has been shown to disrupt the BBB in rats (Bhattacharjee A K, Nagashima T, Kondoh T, Tamaki. “Quantification of early blood-brain barrier disruption by in situ brain perfusion technique” Brain Res Brain Res Protoc. 2001, 8, 126-31) and humans (Siegal T, Rubinstein R, Bokstein F, Schwartz A, Lossos A, Shalom E, Chisin R, Gomori J M. “In vivo assessment of the window of barrier opening after osmotic blood-brain barrier disruption in humans” J Neurosurg. 2000, 92, 599-605). Osmotic disruption has been shown to enhance uptake of relatively large therapeutics, such as viral vectors (Neuwelt E A et al., “Delivery of ultraviolet-inactivated herpes virus across an osmotically modified blood-brain barrier” J. Neurosurg. 1991, 74(3), 475-479; Doran S E et al., “Gene expression from recombinant viral vectors in the central nervous system after blood-brain barrier disruption” Neurosurgery 1995, 36(5), 965-70; Nilayer G et al., “Delivery of herpesvirus and adenovirus to nude rat intracerebral tumors after osmotic blood-brain barrier disruption” Proc. Natl. Acad. Sci. USA 1995, 92(21), 9829-9833).

Osmotic disruption, however, produces a general opening of the BBB that is characterized by an unfavorable toxic/therapeutic ratio. This method produces a 10-fold increase in the permeability of the normal brain endothelium, but only a 25% increase in the permeability of the tumor microvasculature ((Misra A, Ganesh S, Shahiwala A. “Drug delivery to the central nervous system” J Pharm Pharmaceuti Sci. 2003 6(2):252-273; Pardridge, W M. Blood-brain barrier genomics. In: W. M. Pardridge (ed.), Brain Drug Targeting—The Future of Brain Drug Development, United Kingdom: Cambridge University Press, 2001, 275-3000). Other undesirable side effects include physiological stress, transient increase in intra-cranial pressure, and unwanted delivery of anti-cancer agents to normal brain tissue (Cucullo L, Marchi N, Marroni M, Fazio V, Namura S, Janigro D. “Blood-Brain Barrier Damage Induces Release of {alpha}2-Macroglobulin” Mol Cell Proteomics 2003 2: 234-241).

Biochemical disruption of the BBB has also been explored. Biochemical disruption is based on the observation that the permeability of tumor capillaries is enhanced relative to that of normal brain capillaries by administration of certain vasoactive molecules. Endothelium-dependent regulation of cerebral blood vessel function is impaired in brain tumors (Morimoto, T., Aoyag, M., Tamaki, M., Yshino, Y., Hori, H., Duan, L., Yano, T., Shibsta, M., Ohno, K., Hirakawa, K., and Yamaguchi, N. “Increased levels of tissue endostatin in human malignant gliomas” Clin. Cancer Res. 2002, 8(9), 2933-2938; Cobbs, C. S., Brenman, J. E., Aldape, K. D., Bredt, D. S., and Isreal, M. A. “Expression of nitric oxide synthase in human central nervous system tumors” Cancer Res. 1995, 55, 727-730), which might affect tumor capillary permeability in response to vasomodulators. Thus, biochemical disruption promises a selectively that osmotic disruption cannot provide.

Certain vasoactive compounds, such as leukotriene (LTC₄), bradykinin (BK) and histamine, have been shown to selectively disrupt the BBB (i.e., to disrupt the BBB). (Black K L, Hoff Y R, McGillicuddy J E, and Gebarski S S. “Increased leukotriene C4 and vasogenic edema surrounding brain tumors in humans” Ann. Neurol., 1986, 19, 592-595; Inamura T, and Black, K L. “Bradykinin selectively opens blood-tumor barrier in experimental brain tumors” J. Cereb. Blood. Flow. Metab., 1994 14: 862-870; Inamura T, Nomura T, Ikezaki K, Fukui M, Pollinger G, and Black K L. “Intracarotid histamine infusion increases blood tumor permeability in RG2 glioma” Neurol. Res. 1994 16: 125-128; Black, K L, Cloughesy T, Huang S C, Gobin, Y P, Zhou Y, Grous J, Nelson G, Farahani K, Hoh C K, and Phelps M. “Intracarotid infusion of RMP-7, a bradykinin analog, and transport of gallium-68 ethylenediamine tetraacetic acid into human gliomas” J. Neurosurg. 1997 86: 603-609; Sugita M and Black K L. “Cyclic GMP-specific phosphodiesterase inhibition and intracarotid bradykinin infusion enhances permeability into brain tumors” Cancer Res., 1998 58: 914-920; Hashizume K and Black K L. “Increased endothelial vesicular transport correlates with increased blood-tumor barrier permeability induced by bradykinin and leukotriene C4” J. Neuropathol. Exp. Neurol. 2002 61: 725-735).

Leukotriene. LTC₄ is a biologically active molecule formed from the unsaturated fatty acid, arachidonic acid (AA), via the 5-lipooxygenase pathway. The conversion of AA to leukotriene (LTC₄) via the epoxide intermediate LTA₄ is Ca²⁺ dependent. The levels of LTC₄ in the brain are elevated in brain tumors. Intracarotid infusion of LTC₄ selectively increases capillary permeability in brain tumors but not in normal brain (Black K L, Hoff Y R, McGillicuddy J E, and Gebarski S S. “Increased leukotriene C₄ and vasogenic edema surrounding brain tumors in humans” Ann. Neurol. 1986 19: 592-595; Chio C C, Baba T, Black K L. “Selective blood-tumor pro-barrier disruption by leukotrienes” J Neurosurg 1992 77:407-410; Hashizume K and Black K L. “Increased endothelial vesicular transport correlates with increased blood-tumor barrier permeability induced by bradykinin and leukotriene C4” J. Neuropathol. Exp. Neurol. 2002 61: 725-735). Unfortunately, the effects of leukotrienes are slight and limited to limited to small molecules (Black K L and Chio C C. “Increased opening of blood-tumour barrier by leukotriene C4 is dependent on size of molecules” Neurol Res. 1992 14(5):402-4). Accordingly, LTC₄ does not significantly increase the delivery of larger water soluble molecules to brain tumors.

The mechanism responsible for the selective effects of certain vasoactive compounds on brain tumor capillary permeability has been examined. The selective effect of leukotriene is now understood to an enzymatic barrier present in normal brain capillaries, but absent from their brain tumor counterparts. Normal brain capillaries are rich in the BBB enzyme γ-glutamyl transpeptidase (γ-GTP), which acts as an enzyme barrier, rapidly degrading LTC₄ and preventing it from acting on capillary permeability. Abnormal brain capillaries and tumor capillaries lack γ-GTP and its enzymatic barrier effect (Black, K L., Hoff Y R., McGillicuddy J E, and Gebarski S S. “Increased leukotriene C₄ and vasogenic edema surrounding brain tumors in humans” Ann. Neurol. 198619: 592-595). The selective effects of histamine on the permeability of brain tumor capillaries are believed to be mediated by H2 receptors (Inamura T, Nomura T, Ikezaki K, Fukui M, Pollinger G, Black K L. “Intracarotid histamine infusion increases blood tumour permeability in RG2 glioma” Neurol Res. 1994 16(2):125-8; Nomura T, Ikezaki K, Natori Y, Fukui M. “Altered response to histamine in brain tumor vessels: the selective increase of regional cerebral blood flow in transplanted rat brain tumor” J Neurosurg. 1993 79(5):722-8). These mechanisms are specific to these two particular compounds, and do not provide an explanation for the effects of other vasoactive compounds such as bradykinin or bradykinin analogs.

Bradykinin. B K, a nonapeptide (Arg-Pro-Pro-Gly-Phe-Ser-Pro-Phe-Arg), is formed from kinogen (Hall J M “Bradykinin receptors” Gen Pharmacol. 1997 28: 1-6). The vasoactive nanopeptide bradykinin and agonists or polypeptide analogs thereof (e.g., receptor-mediated permeabilizers [RMPs] such as RMP-7) have been administered intravenously to increase BBB permeability to co-administered neuropharmaceutical or diagnostic agents. (U.S. Pat. No. 5,112,596 entitled “Method for increasing blood-brain barrier permeability by administering a bradykinin agonist of blood-brain barrier permeability” to B. Malfroy-Camine; U.S. Pat. No. 5,268,164 entitled “Increasing blood brain barrier permeability with permeabilizer peptides” to J. W. Kozarich et al.). Unfortunately, intravenous infusion causes undesirable hypotensive effects and has proven disappointing in the clinic (Prados M D, Schold S C JR S C, Fine H A, Jaeckle K, Hochberg F, Mechtler L, Fetell M R, Phuphanich S, Feun L, Janus T J, Ford K, Graney W. “A randomized, double-blind, placebo-controlled, phase 2 study of RMP-7 in combination with carboplatin administered intravenously for the treatment of recurrent malignant glioma” Neuro-oncol. 2003, 5, 96-103).

Intracarotid infusion of lower doses of bradykinin was found to produce selective opening of the BTB (Inamura T and Black K L. “Bradykinin selectively opens blood-tumor barrier in experimental brain tumors” J. Cereb. Blood. Flow. Metab. 1994 14: 862-870). Bradykinin does not increase permeability in the normal BBB except at very high doses (Hall J M “Bradykinin receptors” Gen Pharmacol. 1997 28: 1-6). Intracarotid infusion of bradykinin increases permeability 2- to 12-fold in brain tumor capillaries, which allows transport across the BTB of molecules ranging in molecular weight from 100 to 70,000. A method for selectively delivering to abnormal brain tissue a neuropharmaceutical agent (e.g., 5-fluorouracil, cisplatin, methotrexate, or monoclonal antibodies) or a diagnostic agent (e.g., technicium-99 glucoheptonate, gallium-EDTA, and ferrous magnetic or iodinated contrasting agents) employed intracarotid infusion of bradykinin, or a bradykinin analog, such as RMP the bradykinin or bradykinin analog was administered approximately contemporaneously with the agent (U.S. Pat. Nos. 5,527,778 and 5,434,137 entitled “Method for selective opening of abnormal brain tissue capillaries” to K. L. Black). Disruption by bradykinin or RMP has been shown to enhance transvascular delivery of viral particles to malignant cells in the brains of rats (N. G. Rainov “Selective uptake of viral and monocrystalline particles delivered intra-arterially to experimental brain neoplasms” Hum. Gene. Ther. 1995, 6(12), 1543-1552; N. G. Rainov et al. “Long-term survival in a rodent brain tumor model by bradykinin-enhanced intra-arterial delivery of a therapeutic herpes simplex virus vector” Cancer Gene Ther. 1998, 5(3), 158-162; F. H. Barnett et al. “Selective delivery of herpes virus vectors to experimental brain tumors using RMP-7” Cancer Gene Ther. 1998, 6(1):14-20).

Disruption of the BBB by bradykinin, while advantageously selective, suffers several major limitations. The effect of BK and RMP-7 is transient, lasting only 20 minutes, and tumor capillaries become refractory to BK's effect for up to 60-min after administration (Ningaraj, N. S., Rao, M., Hashizume, K., Asotra, K., and Black, K, L. “Regulation of blood-brain tumor barrier permeability by calcium-activated potassium channels” J. Pharmacol. Exp. Ther. 2002, 301, 838-851; Sugita, M. and Black, K. L. “Cyclic GMP-specific phosphodiesterase inhibition and intracarotid bradykinin infusion enhances permeability into brain tumors” Cancer Res. 1998, 58, 914-920). Moreover, bradykinin may not be effective for enhancing permeability in many brain tumor patients. The differential effect of bradykinin has been shown to be mediated by BK type 2 (B2) receptors, which are variably expressed among tumors ((Pizard A, Blaukat A, Muller-Esterl W, Alhenc-Gelas F, Rajerison R M: “Bradykinin-induced internalization of the human B2 receptor requires phosphorylation of three serine and two threonine residues at its carboxyl tail” J Biol Chem 1999 274:12738-12747); Liu Y, Hashizume K, Samoto K, Sugita M, Ningaraj N Asotra K, and Black K L. “Correlation between bradykinin-induced blood tumor barrier permeability and B2 receptor expression in experimental brain tumors” Neurological Res. 2001 23: 379-387). Variable clinical results using the bradykinin analog RMP-7 to increase delivery of chemotherapeutic agents has been attributed to differential B2 expression among brain tumors (Black K L, Cloughesy T, Huang S C, Gobin Y P, Zhou Y, Grous J, Nelson G, Farahani K, Hoh C K, and Phelps M. “Intracarotid infusion of RMP-7, a bradykinin analog, and transport of gallium-68 ethylenediamine tetraacetic acid into human gliomas” J. Neurosurg 1997 86: 603-609). In human gliomas, intracarotid infusion of bradykinin results in a wide range of Ki changes from 2% to 58% (FIG. 17). Differences in B2 receptor density (FIG. 18), as well as differences in the ability of tumor microvessels to degrade cGMP and/or other unexplored factors may provide an explanation.

Bradykinin's activity as a powerful vasodilator further limits use of the drug and its analogs to enhance BBB permeability. BK may adversely lower blood pressure, reduce cerebral blood flow, or contribute to brain edema in some patients. (A. M. Butt “Effect of inflammatory agents on electrical resistance across the blood-brain barrier in pial microvessels of anesthetized rats”. Brain Res. 1995, 696(1-2), 145-150). In addition, bradykinin constricts smooth muscle and stimulates pain receptors.

Bradykinin's ability to selectively deliver agents across the blood-brain barrier has been shown to involve nitric oxide and ultimately, cGMP (Nakano, S., Matsukao, K., and Black, K. L., “Increased brain tumor microvessel permeability after intracarotid bradykinin infusion is mediated by nitric oxide” Cancer Res 1996, 56:4027-4031; Sugita, M. et al. “Nitric oxide and cyclic GMP attenuate sensitivity of the tumor barrier to bradykinin” Neurological Research 1998, 20, 559-563: Sugita, M. and Black, K. L., “CGMP-specific phosphodiesterase inhibition and intracarotid bradykinin infusion enhances permeability into brain tumors” Cancer Res 1998, 58:914-920).

Using immunoblot and immunolocalization studies, Black et al. established that K_{Ca} channels were over-expressed in rat brain tumor capillary endothelium and tumor cells, and demonstrated the functional presence of K_{Ca} channels in isolated rat brain tumor capillary endothelial and tumor cells (Ningaraj N S, Rao M, Hashizume K, Asotra K and Black K L. “Regulation of blood-brain tumor barrier permeability by calcium-activated potassium channels” J. Pharmacol. Exp. Ther. 2002, 301, 838-851). The K_{Ca} channel has been shown to be the convergence point of a bradykinin signaling pathway involving nitric oxide, soluble guanylyl cyclase and cGMP. While bradykinin has been shown to activate K_{Ca} channels, other known activators of K_{CA} do not act as vasodilators, for example, 1,3-dihydro[2-hydroxy(trifluoromethyl)phenyl](trifluoromethyl)-2H-benzimidazolone (NS-1619) (M. Holland et al. “Effects of the BK_{Ca} channel activator, NS1619, on rat cerebral artery smooth muscle” Br. J. Pharmacol. 1996, 117(1): 119-129).

K_{ATP} channels have also been shown to be overexpressed in hypoxic and ischemic conditions (Kitazono T, Faraci F M, Taguchi H, Heistad D D. “Role of potassium channels in cerebral blood vessels” Stroke 1995 26:1713-1723; Brian J E. “Recent insights into the regulation of cerebral circulation” Clin Exp Pharmacol Physiol 1996 23(6-7) 449-57) and in and around brain tumors (Ruoshlati, E. “Specialization of tumor vasculature” Nat. Rev. Cancer 2002 2:83-90; Ningaraj N S, Rao M K, Black K L. “Adenosine 5′-triphosphate-sensitive potassium channel-mediated blood-brain tumor barrier permeability increase in a rat brain tumor model” Cancer Research 2003 63: 8899-8911).

WO 01/54680 discloses methods of delivering a medicament to an abnormal brain region by administering to the subject a potassium channel activator (i.e., activator of calcium-activated or ATP-dependent potassium channels [K_{Ca} or K_{ATP}]). Potassium channel activators include direct agonists (other than bradykinin or bradykinin analogs), such as NS-1619 (a direct agonist of K_{Ca}) or minoxidil (a direct agonist of K_{ATP}). Potassium channel activators are also said to include compounds that indirectly activate potassium channels, such as indirect activators (e.g., nitric oxide, nitric oxide donors and other activators of soluble guanylyl cyclase).

US 20030072748 discloses methods of inducing apoptosis of a malignant cell, which employs a calcium-activated potassium channel (K_{Ca}) activators and is useful for treating a malignant tumor in a human subject. Activators are said to include, for example, NS-1619, activators of soluble guanylyl cyclase, YC-1 and guanylyl cyclcase activating proteins.

Despite advances in the art, improved biochemical approaches are needed to increase permeability and to enhance delivery of hydrophilic therapeutic drugs or small-to large-sized molecules, including contrast-enhancing agents, cytotoxic chemotherapeutic agents, therapeutic proteins monoclonal antibodies, cytokines, effecter cells, and viral particles and viral vectors in vivo to abnormal brain tissue selectively with little or no drug delivery to normal brain.

Therefore, it is an object of the invention to provide improved methods and compositions to selectively increase the permeability of the blood brain barrier, and in particular, the blood tumor barrier.

### SUMMARY OF THE INVENTION

The present invention discloses compositions and methods to selectively increase the permeability of the blood brain barrier to administer therapeutic, prophylactic and diagnostic agents.

In one embodiment, a method is disclosed for delivering a therapeutic, prophylactic or diagnostic agent to an abnormal brain region in a mammalian subject by administering one or more activators of an ATP-sensitive potassium channel in combination with one or more activators of a calcium-activated potassium channel, under conditions, and in an amount sufficient to increase the permeability to the therapeutic, prophylactic or diagnostic agent of a capillary or arteriole delivering blood to cells of the abnormal brain region; and also administering to the subject the therapeutic, prophylactic or diagnostic agent, so that the therapeutic or diagnostic agent is delivered selectively to the cells of the abnormal brain region.

In one embodiment of the method, the activator of the ATP-sensitive potassium channel is a direct agonist. In a particular embodiment, the direct agonist of the ATP-sensitive potassium channel is minoxidil, cromakalim, levcromakalim, emakalim, bimakalim, celikalim, rimakalim, pinacidil, aprikalim, picartamide, diazoxide or nicorandil.

In another embodiment of the method, the activator of the ATP-sensitive potassium channel is an indirect activator. In one particular embodiment, the indirect activator is an adenylyl cyclase activator. In another embodiment, the indirect activator is an activator of cyclic AMP (cAMP) dependent protein kinases. In a further embodiment, the indirect activator is an agent that increases the formation of cAMP or prevents the breakdown of cAMP.

In one embodiment of the invention, the activator of the calcium-activated potassium channel is a direct agonist. In a particular embodiment, the direct agonist of the calcium-activated potassium channel is NS1619, NS-1608, NS-04, NS-08 or EBIO.

In another embodiment, the activator of the calcium-activated potassium channel is an indirect activator. In one embodiment, the indirect activator is an activator of soluble guanylyl cyclase. In a particular embodiment, the activator of soluble guanylyl cyclase is nitric oxide (NO)-independent. In one embodiment, the NO-independent activator is cyclase is carbon monoxide, a porphyrin, a metallopophyrin, YC-1, BAY-2272, BAY 41-2272, BAY 41-8543.

In another embodiment, a method is disclosed for delivering a therapeutic, prophylactic or diagnostic agent to an abnormal brain region in a mammalian subject by administering one or more direct agonists of an ATP-sensitive potassium channel (e.g., minoxidil sulfate) in combination with one or more direct agonists of a calcium-activated potassium channel (e.g., NS1619), under conditions, and in an amount sufficient to increase the permeability to the therapeutic, prophylactic or diagnostic agent of a capillary or arteriole delivering blood to cells of the abnormal brain region; and administering to the subject the therapeutic, prophylactic or diagnostic agent, so that the therapeutic or diagnostic agent is delivered selectively to the cells of the abnormal brain region.

The methods of the present invention are useful in treating abnormal brain regions. In one embodiment of the invention, the abnormal brain region is a region of tumor tissue. In a particular embodiment, the tumor tissue is malignant. In another embodiment of the present invention, the abnormal brain region is a region of tissue physiologically affected by a stroke, ischemia, neurodegeneration, injury, trauma or infection.

Therapeutic, prophylactic and diagnostic agents that can be delivered to abnormal regions of the brain according to the present invention include a wide variety of agents. In one embodiment of the invention, the therapeutic agent is an anti-proliferative agent such as a chemotherapeutic agent. In another embodiment, the therapeutic agent is an agent used to treat stroke or ischemia. In a further embodiment, the therapeutic agent is an anti-neurodegenerative agent. In still a further embodiment, the therapeutic agent is a cytokine or therapeutic protein. In yet another embodiment, the therapeutic agent is a DNA expression vector, viral vector or therapeutic oligonucleotide.

According to another aspect of the invention, a method is disclosed for delivering a therapeutic, prophylactic or diagnostic agent to a malignant tumor in a mammalian subject by administering one or more activators of an ATP-sensitive potassium channel in combination with one or more activators of a calcium-activated potassium channel, under conditions, and in an amount sufficient to increase the permeability to the therapeutic, prophylactic or diagnostic agent of a capillary or arteriole delivering blood to cells of the malignant tumor; and administering to the subject the therapeutic or diagnostic agent, so that the therapeutic, prophylactic or diagnostic agent is delivered selectively to the cells of the abnormal brain region.

In one embodiment of the method, the malignant tumor is a located in the brain. In another embodiment of the method, the malignant tumor is located in the breast, bone, prostate, liver, lung, larynx, gall bladder, head, neck, stomach, kidney, skin cervix, connective tissue, adrenal gland, pancreas, spine, thorax, peritoneum, bowel, colon, rectum, or lymphatic system of the mammalian subject.

Modes of administration suitable for delivery of the activators and therapeutic, prophylactic and diagnostic agents of the present invention include parenteral, intravenous, intra-synovial, intrathecal, intra-arterial, intra-carotid, intraspinal, intrasternal, peritoneal, percutaneous, surgical implant, internal surgical paint, infusion pump, or via catheter. In one embodiment, the activators are delivered by intravenous or intra-arterial infusion or injection. In another embodiment, the activators are delivered by intra-carotid infusion or injection.

In one embodiment of the present invention, the therapeutic or diagnostic agent is administered to the mammalian subject simultaneously or substantially simultaneously with the activators. In another embodiment, the therapeutic or diagnostic agent is administered to the mammalian subject prior the activators. In a further embodiment, the therapeutic or diagnostic agent is administered to the mammalian subject after the activators.

### BRIEF DESCRIPTION OF THE FIGURES

FIG. 1 is a schematic representation of the differences between the BBB and BTB with regard to their morphology and biochemical response to K_{ATP} channel agonists. BTB capillaries and surrounding tumor cells over-express K_{ATP} channels and, therefore, may readily respond to activation by K_{ATP} channel agonists resulting in enhanced formation of transport vehicles in tumor capillary endothelial and tumor cells. Confocal microscopic immunolocalization of K_{ATP} channels (green) and vWF (red) human normal brain and tumor capillary endothelial cells and tumor cells was performed using Kᵢᵣ6.2 and vWF antibodies. Yellow indicates the co-localization of K_{ATP} channels in capillary endothelial cells. Brain tumor capillaries over-express K_{ATP} channels and were co-localized with vWF on endothelial cells. In contrast, K_{ATP} channels are hardly detected in normal brain microvessel endothelial cells, and may be non-responsive to K_{ATP} channel agonist. The strategy involves biochemical modulation of K_{ATP} channels for selective and enhanced drug delivery to tumors with little or no drug delivery to normal brain tissue. EC: endothelial cell, TJ: tight junction. FIG. 1 and FIGS. 2-9 are published in Ningaraj N S, Rao M K, Black K L. “Adenosine 5′-triphosphate-sensitive potassium channel-mediated blood brain tumor barrier permeability increase in a rat brain tumor model” Cancer Research 2003 63(24):8899-911).

FIG. 2 is a graphical representation of the quantitative increases in BTB permeability. (A) Autoradiographs of coronal brain sections showing little [¹⁴C]-AIB delivery in a rat with an intracranial RG2 tumor following i.c. vehicle (PBS+0.5% DMSO) infusion, but significantly enhanced delivery in an MS-treated rat. The MS-induced increase in [¹⁴C]-AIB delivery was significantly diminished when Gib was co-infused. For comparison, the scale at left shows pseudocolor intensities of tissue calibrated [¹⁴C] standards from 40-450 nCi/g specific activities. (B) the mean Ki for [¹⁴C]-AIB significantly increased after i.c. infusion of MS (30 μg/kg/min for 15 min) compared to a vehicle-treated group. The Ki increase was significantly attenuated by co-administration of Gib (N=4). Co-infusion of a K_{Ca} channel inhibitor, IBTX, with MS failed to block the MS-induced Ki increases. (C) In addition, MS (30 μg/kg/min for 15 min) enhanced BTB permeability in GBM-xenograft model compared in a PBS-treated group, and was significantly attenuated by co-administration of Gib. Data are presented as mean±S.D (N=4). ***P<0.001 versus vehicle group. **P<0.01 versus MS-treated group.

FIG. 3 depicts the results of a time-course study. (A) MS and NS-1619 elicited sustained increases in mean Ki values for up to 30 and 60 min, respectively. To contrast, BK-induced Ki increase was transient and issued for 15-20 min. Prolonged BK infusion (up to 60 min) failed to sustain the Ki increase. Data in A, B. and C are resented as mean±S.D. ***P<0.001 versus vehicle group (N=4). (B) Ki values within RG2 tumors comparing BTB permeability to radiotracers of different sizes including AIB, carboplatin, (CPN) and dextran (Dex) with and without MS infusion. The Ki values in the MS-treated groups were significantly (***P<0.001) higher than in the vehicle-treated group. (C) A synergistic effect of MS with NS-1619 (i.c.) in RG2 tumor-bearing rats was observed. The Ki value for [¹⁴C]-AIB delivery following combination treatment was significantly (**P<0.01) higher compared to groups treated with MS or NS-1619 alone. Veh-1 (PBS+0.5% DMSO), Veh-2 (PBS+0.5% ethanol), Veh-3 (PBS-0.5% DMSO and ethanol),

FIG. 4 illustrates ErbB-2 and GFAP expression. (A) over-expression of erbB-2 receptors was demonstrated using Her-2 and Neo antibodies in vitro human (a) and rat (b) cells, and in vivo GBM (d) and RG2 tumor (e), respectively. Low erbB-2 expressing MCF-7 cells was used as a negative control (c). (B) The glial origin of GBM and RG2 was demonstrated with GFAP immunostaining in vivo.

FIG. 5 depicts the increased delivery of macromolecules. (A) MS enhances delivery of Her-2 MAb and Neo polyclonal antibody to intracranial GBM-xenograft and RG2 tumor models when infused (i.c.) in combination compared to erbB-2 antibody infusion alone. Abundant Her-2 MAb binding was observed in the tumor center (TC) and tumor periphery (TP) suggesting that infiltrating tumor cells over-express Her 2 receptors. Further, enhanced delivery of Neu was observed in TC when co-infused with MS (e, f), while a low amount of Neu was delivered (d) when infused alone. (B) The ability of MS to increase delivery of Adv-GFP across the BTB was studied in nude rats with intracranial GBM-xenografts. Abundant GFP expression was seen predominantly in the TC and a low extent in the TP (g, h, i). GBM cells are shown expressing GFAP (h), and GFP was co-localized with GFAP suggesting that GFP was predominantly expressed on tumor cells (i). On the other hand, in vehicle and Adv-GFP infused rat, hardly any GFP expression was detected on tumor cells, but was observed trapped in blood vessels (j), possibly due to the inability of Adv-GFP to cross the BTB. Further, GFP did not co-localize with GFAP on tumor cells indicating that Adv-GFP failed to infect tumor cells (1).

FIG. 6. K_{ATP} channels. (A) K_{ATP} channel distribution in membranes prepared from rat brain tissue (1), RG2 tissue (2), RBBC (3), RG2 cells (4), co-culture of RG2 and RBBC (5), human brain tissue (6), GBM tissue (7), HBMVEC (8), GBM cells (9), and co-culture of HBMVEC and GMB cells (10). The membranes were incubated with [³H]-Glibenclamide binding. The [³H]-Glibenclamide binding (cpon/mg protein) is significantly greater in co-cultures (***P<0.001) compared to normal and tumor cells. Tumor cells and tumor tissue also exhibit significantly (**P<0.01) greater [³H]-Glibenclamide binding compared to endothelial cells and normal brain tissues, respectively. (B) RT-PCR analysis of Kᵢᵣ6.2 subunit-lane 1: HBMVEC; lane 2: primary GBM cells: lane 3: GBM cells co-cultured with HBMVEC; lane 4: RG2 cells co-cultured with RBEC isolated from neonatal rat brain, lane 5: RBEC, and lane 6: rat glioma (RG2) cells. Also shown are the intensities of a β-actin band in the same RT-PCR to ascertain mRNA-loading variance. (C) Immunoblot analysis of SDS-PAGE samples (20 μg protein/lane reveal differential expression of K_{ATP} channel protein immunoreactive with an anti-peptide antibody specific for Kᵢᵣ6.2 subunit-lane a: GBM cells; lane b: HBMVBC; lane c: GBM cells co-cultured with HBMVEC: lane d: RBEC; lane e: RG2 cells; and lane f: RG2 cells co-cultured with RBEC. Also shown are the intensities of a 43 kDa β-actin band in the same immunoblot to ascertain protein-loading variance. (D) Changes in relative fluorescence intensity (RFL) during a 60-sec period in response to addition of MB and Gib at 20 and 40 sec respectively HBMVEC (3) and GBM (4) cells. In contrasts, no change in RFU was observed in HBMVEC and GBM cells without the addition of 1 μM MS and 2 μM Gib (1, 2). (E) K_{ATP} channel activity in HBMVEVC co-cultured with GBM cells (4) in response to MS was greater compared with the activity of GBM alone (3). Control experiments were preformed with RBMVEC alone (1) and GBM+HBMVEC (2) without addition of MS and Gib. The decrease in fluorescence intensity corresponding to membrane potential changes is plotted on the Y-axis as RFU. Addition of Gib reversed the membrane potential to resting values. Note that the MS-induced K_{ATP} channel activity is greater in co-cultures than in endothelial or tumor cells alone.

FIG. 7. Immunocytochemistry—(A) Confocal microscopic immunolocalization of K_{ATP} channels (green) and vWF (red) in human brain microvascular endothelial cells (HBMVEC) and GBM cells (a,b). (B) Colocalization of K_{ATP} channel in GBM tissue section. Representative image (20×) of a GBM tissue section showing number of capillaries immunostained with vWF (B-a), abundant expression K_{ATP} channels (green) in tumor microvessel as well as in tumor cells (B-b), and these K_{ATP} channels co-localize (B-c) with vWF on capillary endothelial cells (yellow). (C) Similarly, co-localization of K_{ATP} channels in RG2 tumor capillary endothelial cells was also observed. Control experiments were performed with secondary antibody but primary antibody was added.

FIG. 8. Mechanism of increased transport. (A) Induction of vesicular transport in RG2 tumor capillary endothelium and tumor cells in vivo. (1) In the vehicle-infused rat group, brain tumor microvessel endothelial cells show few vesicles (arrows). (2) MS infusion caused an increased formation of vesicles (arrows) by luminal membrane invaginations. These vesicles, with an average diameter of 80-90 mm, dock and fuse with the basal membrane. (3) Few vesicles are seen in vehicle-infused rat tumor cells. (4) MS, however, significantly increased the number of pinocytotic vesicles (arrows) in tumor cells. Values are mean±SD (N=rats, N=5 capillaries/rat) BM: basal membrane; EC: endothelial cell; MS: minoxidil sulfate; L: luminal; pinocytotic vesicles (arrows). (B) MS-induced accelerated formation of transendothelial pinocytotic vesicles in tumor capillary endothelium without affecting endothelial tight junctions (C). BG: basal ganglia, PBS: phosphate buffer saline, MS: minoxidil sulfate. Number of vesicles in RG2 tumor was significantly different from vehicle-treated group (**P<0.01) in tumor capillaries. Cleft indices (%) in RG2 tumor capillaries are significantly (**P<0.01) different from either vehicle or MS-treated normal brain capillaries.

FIG. 9. Survival study. (A) Kaplan-Meier survival curves show that RG2 tumor-bearing rats treated with carboplatin (CPN) and MS survived significantly (^{4**}P<0.001, N=30) longer than vehicle-treated rats. Rats treated with carboplatin alone also survived significantly (**P<0.01, N=26) longer than the untreated group. In a randomized study, three groups of rats were treated i.c. (once a day for three consecutive days) via an exteriorized catheter 7 days after tumor implantation. (B) Representative coronal sections obtained from rats from each group stained with hemotoxilin and eosin. In an untreated group, the brain sections of rats that died on day 25 had huge tumors (9.37±2 mm²) compared to rats that received either carboplatin (5.30±1.9 mm²) alone or MS with carboplatic (1.30±1.2 mm²). Notice a necrotic tumor area in a representative rat brain section following MS and carboplatin infusions. This suggests increased carboplatin delivery and, therefore, enhanced cytotoxicity of the drug.

FIG. 10. Immunofluorescent colocalization of K_{ATP} channels in human normal cerebellum and microvessels using K_{ATP} channel and Factor VIII antibodies. Confocal microscopic analysis does not show any co-expression of K_{ATP} channels with Factor VIII in microvessels. The K_{ATP} channels and Factor VIII are labeled, respectively, with FITC- and TRITC-conjugated secondary antibodies. Images were scanned at 20×. Arrow indicates a normal brain microvessel. Non-capillary endothelial cells show staining for K_{ATP} channel expression.

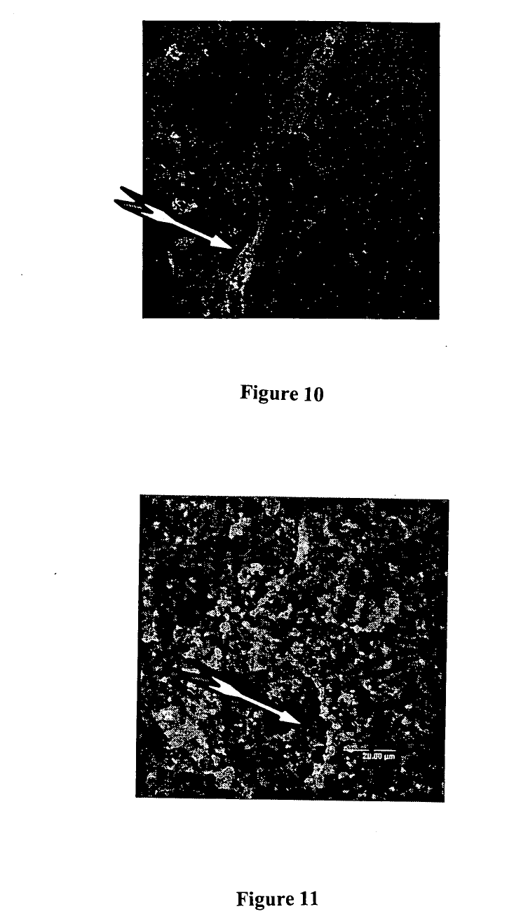

FIG. 11. A representative image (20×) of a GBM section showing expression of K_{ATP} channels (green) in tumor microvessels as well as in tumor cells. These channels appear to colocalize with Factor VIII in endothelial cells (yellow). Arrow indicates a brain tumor microvessel showing co-localization of K_{ATP} channels with Factor VIII. Tumor cells surrounding the microvessel also show intense staining for K_{ATP} channel expression.

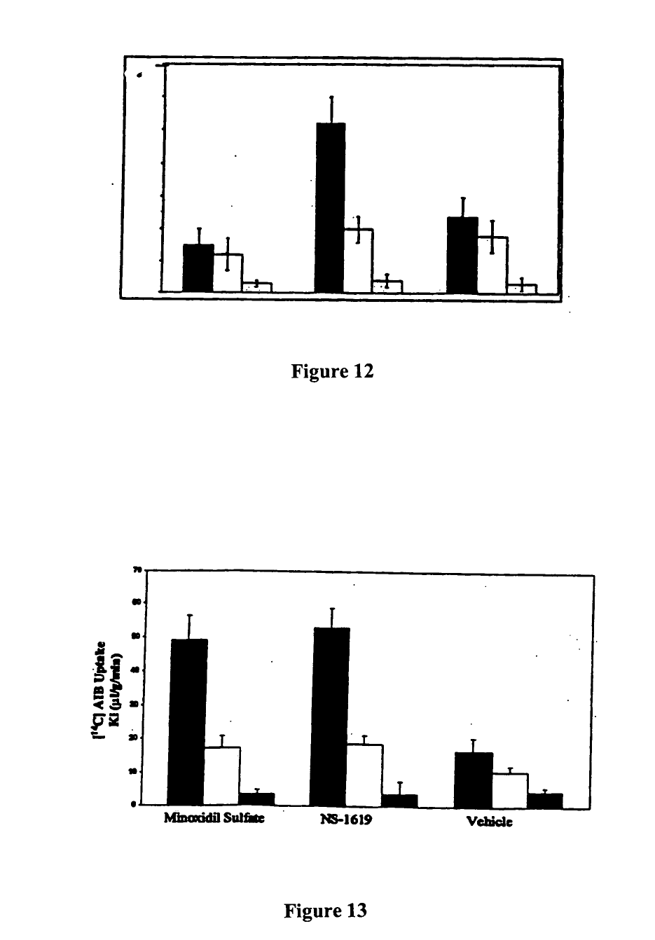

FIG. 12. Quantitative increases in BTB permeability is shown: the mean Ki for [¹⁴C]-AIB significantly increased after i.c. infusion of MS (30 μg/kg/min for 15 min, N=4) as compared to PBS controls (N=4) and was significantly attenuated by co-administration of a K_{ATP} channel antagonist, glibenclamide (N=4). Data are presented as mean±S.D. ^{***}P<0.001 versus PBS group, ^{**}P<0.01 versus agonist-treated group.

FIG. 13. Quantitative increases in BTB permeability is shown: the mean Ki for [¹⁴C]-AIB significantly increased after i.v. infusion of MS (60 μg/kg/min for 15 min, N=4) as compared to PBS controls (N=4) and significantly attenuated by co-administration of K_{ATP} channel antagonist, glibenclamide (N=4). Data are presented as mean±S.D. ^{***}P<0.001 versus PBS group, ^{**}P<0.01 versus agonist-treated group.

FIG. 14. Increased delivery of macromolecules. (A) MS enhanced delivery of Her-2 MAb to intracranial GBM-xenograft model when infused (i.c.) in combination compared to MAb infusion alone. Abundant Her-2 MAb binding was observed in the tumor center (TC) and tumor periphery (TP) suggesting that infiltrating tumor cells over express Her 2 receptors. (B) The ability of MS to increase delivery of Adv-GFP across the BTB was studied in nude rats with intracranial GBM-xenograft. Abundant GFP expression was seen predominately in the TC and to a small extent in the TP (g). GBM cells are shown expressing GFAP (h), and GFP was co-localized with GFAP suggesting that GFP was predominantly expressed on tumor cells (i). In vehicle and Ad-GFP infused rat, however, hardly any GFP expression was detected on tumor cells, but was observed trapped in blood vessels (j), possibly due to the inability of Adv-GFP to cross the BTB. Further, GFP did not co-localize with GFAP on tumor cells indicating that Adv-GFP did not infect tumor cells.

FIG. 15. Survival Study. Kaplan-Meier analysis showed that nude mice harboring intracranial human glial tumors survived longer when treated i.v. (three consecutive doses for three days) with a combination of MD, tremedor and Herceptin than vehicle-only groups (FIG. 6). The mean survival in the MS, temedor and Herceptin group was 67.2±11.8 days; P<0.001 vs. vehicle (20.93±8.9 days), MS (27.09±8.7 days), Herceptin (24.91±8.3 days) and temedor (25.5±6.3 days) alone treatment groups).

FIG. 16. Bradykinin increases BTB permeability and enhances delivery of [¹⁴C]-dextrose (70 KD) to brain tumor. Color-enhanced autoradiographs of coronal brain sections show little [¹⁴C]-dextrose uptake into RG2 tumor in PBS-treated, but significant uptake of [¹⁴C]-dextrose in BK-treated rats. The scale at left shows pseudocolor intensities of tissue-calibrated [¹⁴C] standards from 0-300 nCi/g specific activities for comparison.

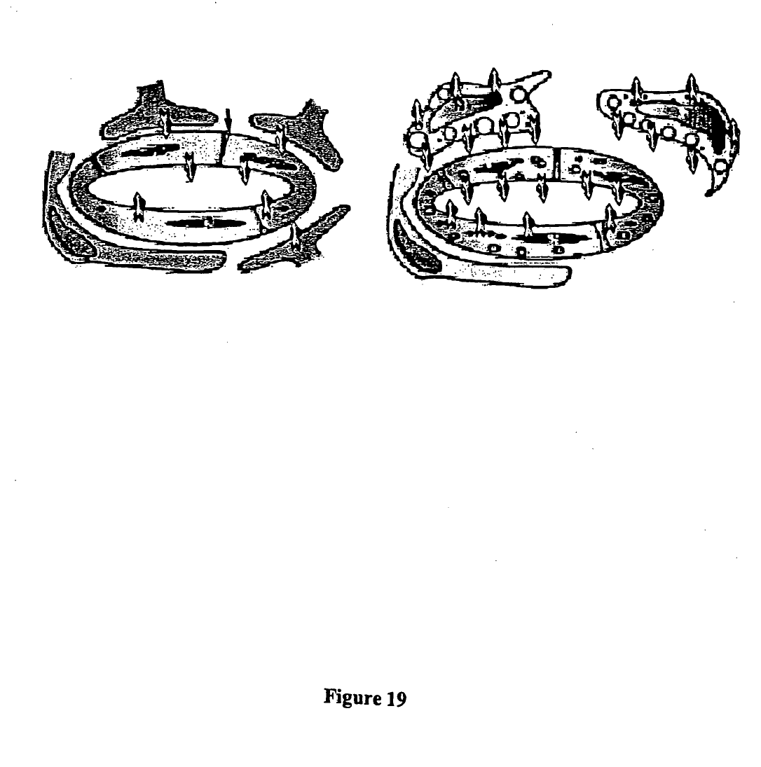

FIG. 17. Images obtained from patents with malignant gliomas. Upper row: Base line axial PET images. Center row: Axial PET images obtained after infusion of RMP-7. Lower Row: Axial T1-weighted MR images showing co-registration of baseline MR and RMP-7 PET images. Black et al. 1997, J Neurosurg.

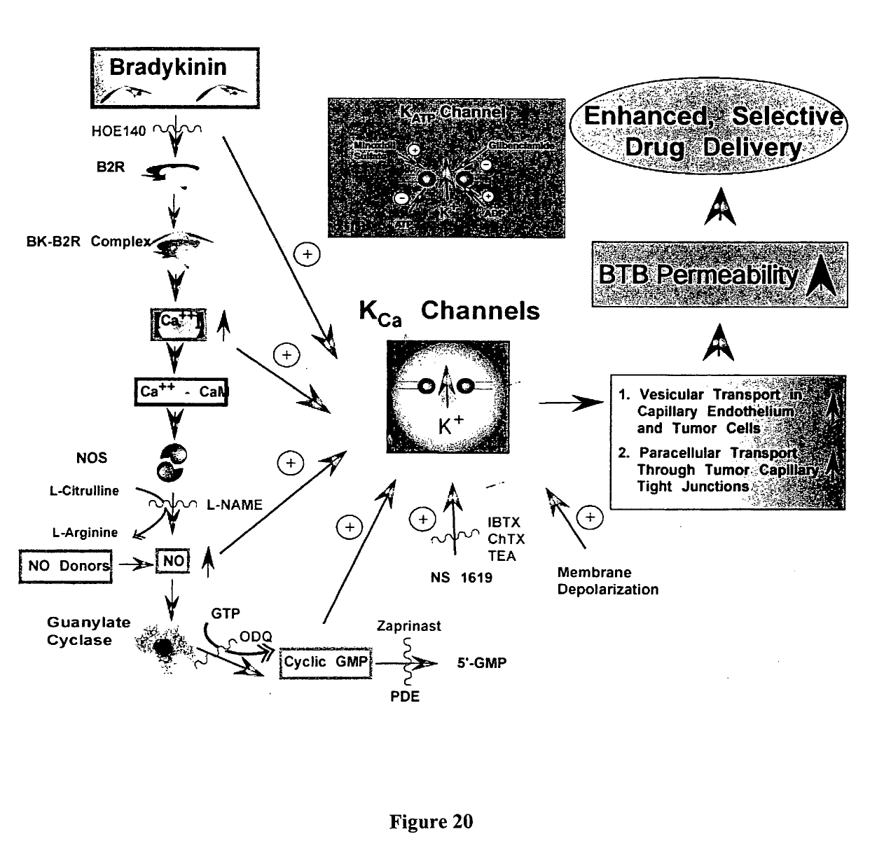

FIG. 18. Immunoco-localization of B2 receptors (FITC-green) and endothelial cell marker, Factor VIII (TRITC-red). Normal rat brain and rat brain tumor capillary endothelium lack B2 receptor expression, although, tumor (RG2, C6 and 9L) cells over-express B2 receptors compared to normal brain cells. A differential expression of B2 receptors that correlates with the degree of BTB permeability response to BK infusion. Liu et al. 2001, Neurological Res.

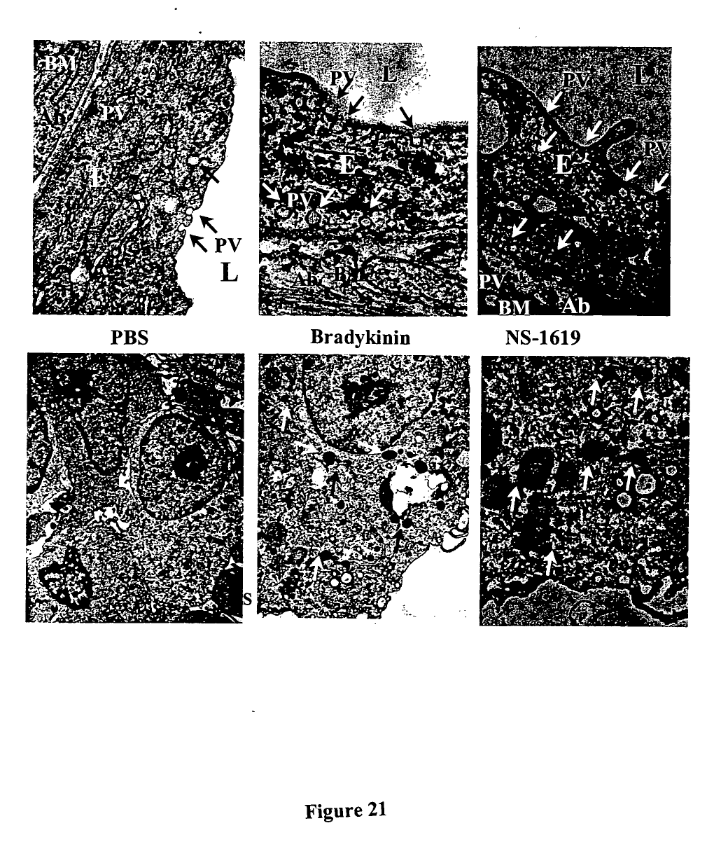

FIG. 19. Schematic representation of differences between the BBB and BTB with regard to their morphology and biochemical response to K_{Ca} and K_{ATP} channel agonists. BTB capillaries and surrounding tumor cells overexpress K_{Ca} and K_{ATP} channels. Therefore, they may readily respond to activation by K_{Ca} and K_{ATP} channel agonists resulting in enhanced formation of transport vesicles, which appear to transport molecules to tumor tissue across the BTB. In contrast, K_{Ca} and K_{ATP} channels are barely detected in normal brain microvessel endothelial cells and may not readily respond to K_{Ca} and K_{ATP}. channel agonists.

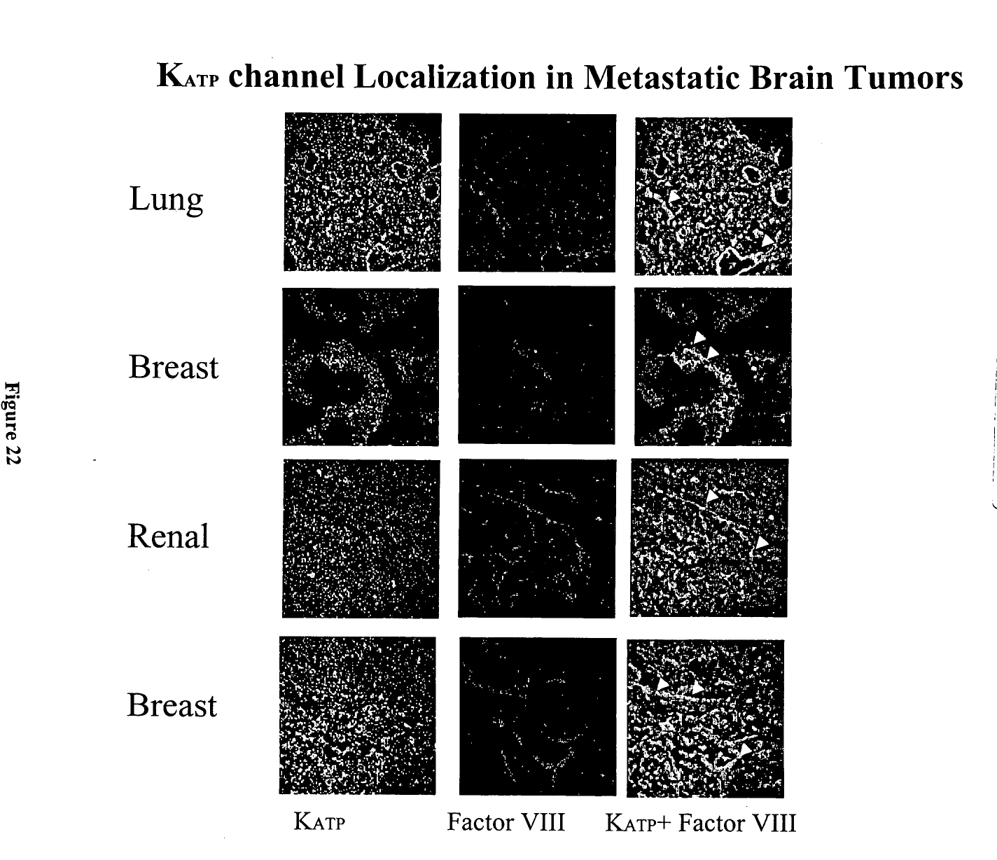

FIG. 20. The biochemical and cellular pathways for BTB permeability regulation for enhanced drug delivery to brain tumors. The main figure shows regulation of BTB permeability via direct (NS-1619) and indirect (BK, NO) activation of K_{Ca}. Cover, Ningaraj N S, Rao M, Hashizume K, Asotra K, Black K L. “Regulation of blood-brain tumor barrier permeability by calcium-activated potassium channels” Journal of Pharmacology and Experimental Therapeutics 2002 301:838-851. The inset shows regulation of BTB permeability by a direct K_{ATP} activator, minoxidil sulfate. The inhibitors used include NOS inhibitor L-NAME, guanylate cyclase inhibitor ODQ, phosphodiesterase inhibitor zaprinast, K_{Ca} channel inhibitor IBTX and K_{ATP} channel antagonist glibenclamide.

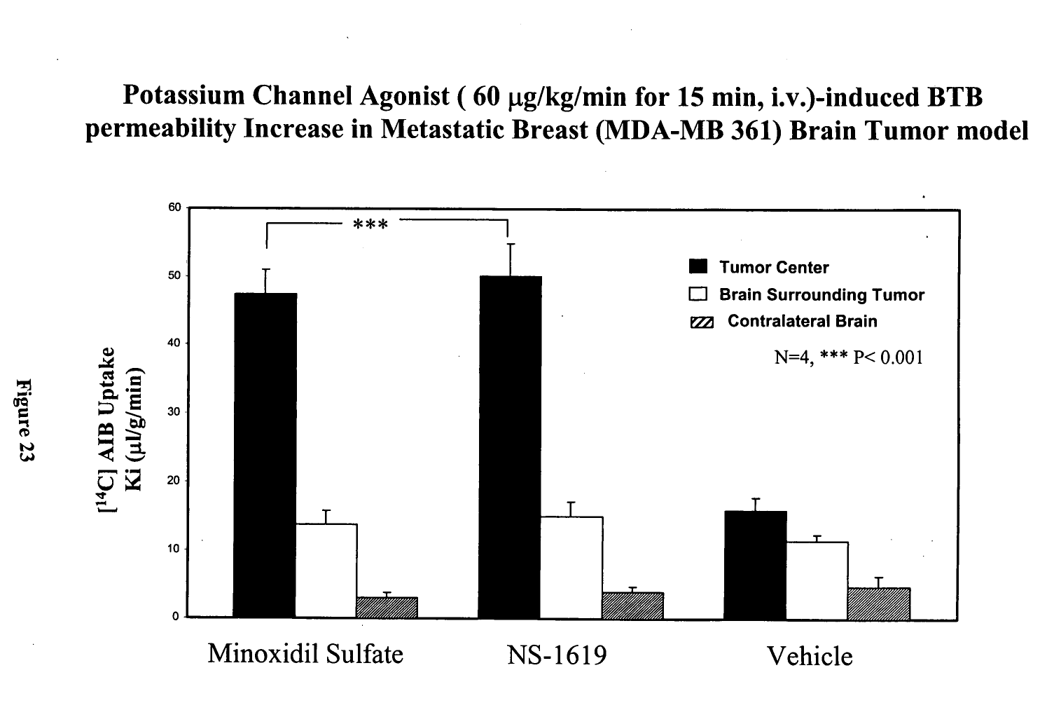

FIG. 21. Induction of vesicular transport in tumor capillary endothelium and tumor cells in vivo.

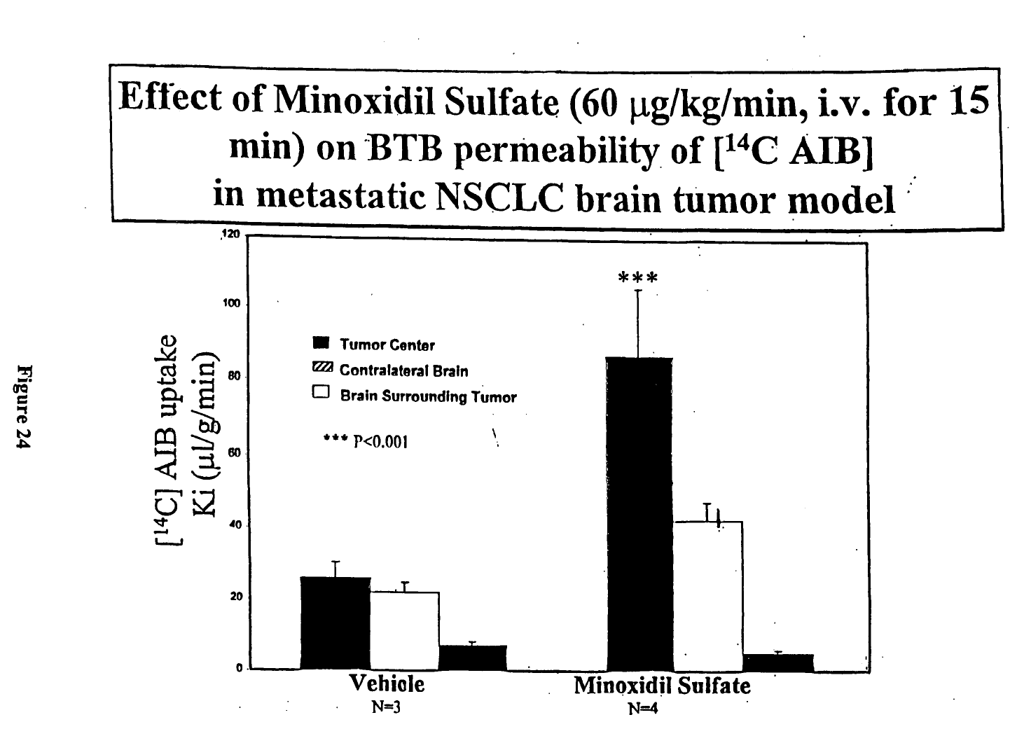

Top Row: In PBS-infused rat, a brain tumor microvessel endothelial cell shows very few vesicles (arrows). BK infusion caused an increased formation of vesicles (arrows) by luminal membrane invaginations> These vesicles with average diameter of 75-80 nm dock and fuse with the basal membrane. NS-1619 induces rapid formation of pinocytotic vesicles (arrows) in tumor capillary endothelial cytoplasm. Bottom Row: No HRP-laden vesicles are seen in a tumor cell of a vehicle-treated rat. A number of electron-dense, HRP-laden vesicles (arrows) are seen in tumor cells following intracarotid infusion of BK or NS-1619, and intravenous injection of HRP in rats with RG2 tumors. Abbreviations: Ab, abluminal; BM, basal membrane; E, endothelial cytoplasm; L, luminal; PV, pinocytic vesicles (arrows).

FIG. 22. Overexpression of KATP channels in metastatic brain tumors. Metastasized brain cancer cells originating from lung, breast and kidney all have high levels of K_{ATP} channel expression (left panels, green stain). Moreover, brain microcapillary endothelium adjacent to the tumor also has high levels of K_{ATP} channel expression (right panels, yellow cells).

FIG. 23. Potassium channel agonists enhance uptake of compounds selectively to metastatic breast brain tumors. Data obtained using a metastatic breast brain tumor model. When rats were co-injected intravenously with [¹⁴C]AIB and minoxidil sulfate, the levels of [¹⁴C]AIB in tissue taken from the brain tumor is approximately three times as great as when they are injected with saline and [¹⁴C]AIB. The level of [¹⁴C]AIB surrounding the tumor and in the contralateral brain does not differ significantly between the vehicle treated and minoxidil sulfate treated rats.

FIG. 24. Potassium channel agonists enhance uptake of compounds selectively to metastatic lung brain tumors. Data obtained using a metastatic lung brain tumor model. Intravenous co-injection of minoxidil sulfate increased the uptake of [¹⁴C]AIB specifically to the tumor vs. the brain surrounding the tumor or contralateral brain approximately three-fold.

### DETAILED DESCRIPTION OF THE INVENTION

The present invention provides several embodiments for improved methods to selectively increase the permeability of the blood brain barrier to administer therapeutic, prophylactic and diagnostic agents. K_{Ca} and K_{ATP} are upregulated in the capillaries that serve these abnormal brain regions (e.g., brain tumor capillaries), and therefore constitute effective targets for selective permeability modulation of the blood-brain barrier.

Activation of K_{Ca} and K_{ATP} channels by pharmacologic agents capable of opening these potassium channels can sustain enhanced and selective drug delivery to abnormal brain regions, including tumors and areas affected by stroke or ischemia. Pharmacologic agents capable of opening/activating the K_{Ca} and K_{ATP} channels include both direct agonists (i.e., agents that bind directly to the K_{Ca} or K_{ATP} channel, thereby causing them to open) and indirect activators (i.e., agents that interact directly with upstream regulatory elements, which then cause K_{Ca} or K_{ATP} channels to open, either directly or indirectly).

In one embodiment, the invention is the selective increase in permeability of the blood-brain barrier that includes administering one or more direct agonists of a K_{Ca} channel (e.g., NS-1619) or a K_{ATP} channel (e.g., minoxidil sulfate or MS). In a particular embodiment, the invention is the selective increase in permeability of the blood-brain tumor barrier by administration of one or more direct agonists of K_{Ca} or K_{ATP}.

In one embodiment, the invention involves administering one or more indirect activators of a K_{Ca} channel (e.g., activators of soluble guanylyl cyclase) or a K_{ATP} channel (e.g., activators of adenylyl cyclase).

In a particular embodiment, the invention is the selective increase in the permeability of the blood brain barrier that includes administering one or more NO-independent activators of soluble guanylyl cyclase other than YC-1 (e.g., BAY 41-8543).

In a particular embodiment, the invention is the selective increase in the permeability of the blood brain barrier that includes administering one or more activators of adenylyl cyclase (e.g., forskolin).

In a further embodiment, the invention is the selective increase in permeability of the blood-brain barrier that includes administering one or more direct agonists or indirect activators of a K_{ATP} channel with one or more direct agonists or indirect activators of a K_{Ca} channel. It has been observed that the maximum effect of activating the potassium channel is achieved at a concentration wherein the agonist occupies all of the binding sites of the potassium channels. Thus, it was believed that only a certain amount of a potassium channel agonist could be administered to effectuate increased vascular permeability prior to saturating the potassium channels, and that there was thus a ceiling on the amount of selective increase of permeability based on the number of channels. However, it has now been discovered that the K_{Ca} and K_{ATP} channels act independently on the blood-brain barrier, so that when an agonist of K_{ATP} channel is administered in combination or alternation with an agonist of a K_{Ca} channel, the maximum permeability increases over what was previously considered to be an upper limit of achievement of permeability.

In another embodiment, the invention is the selective increase in the permeability of the blood-brain barrier that includes administering one or more direct agonists of a K_{Ca} channel (e.g., NS-1619) in combination with one or more indirect agonists of a K_{Ca} channel (e.g., activator of soluble guanylyl cyclase).

In yet another embodiment, the invention is the selective increase in the permeability of the blood brain barrier that includes administering one or more direct agonists of a K_{ATP} channel (e.g., minoxidil sulfate) in combination with one or more indirect agonists of a K_{ATP} channel (e.g., activator of adenylyl cyclase).

In another particular embodiment of the invention, pharmaceutical compositions, methods and kits are provided for the diagnosis, treatment, and/or prevention of a disease characterized by abnormal brain tissue a direct agonist or indirect activator of an K_{ATP} potassium channel, such as minoxidil sulfate, is administered in combination or alteration with an direct agonist or indirect activator of a K_{Ca} channel, such as NS-1619, in combination/alternation with one or more diagnostic, therapeutic, and/or prophylactic agents.

In an alternative embodiment of the invention, pharmaceutical compositions, methods and kits are provided for the diagnosis, treatment, and/or prevention of a disease characterized by abnormal brain tissue, and in particular abnormally proliferating cells (i.e. tumor cells), using one or more agonist(s) or activators of an K_{ATP} channel, such as minoxidil sulfate, with one or more agonist(s) or activators of a K_{Ca} channel, such as NS-1619, in combination/alternation with one or more diagnostic, therapeutic, and/or prophylactic agent optionally further in combination/alternation with one or more of the compounds selected from the group consisting of leukotriene (LTC₄), bradykinin (BK), a BK analog, an agonist of a bradykinin receptor, a modulator of δ-glutamyl transpeptidase, a modulator of nitric oxide synthase, a modulator of guanylate cyclase, a modulator of phosphodiesterases, or a modulator of cGMP-dependent protein kinase.

In a further embodiment of the invention, pharmaceutical compositions, methods and kits are provided for the diagnosis, treatment, and/or prevention of a disease characterized by abnormal brain tissue, wherein direct agonists of a K_{Ca} channel is administered together with a indirect activator of a K_{Ca} channel in combination/alternation with one or more diagnostic, therapeutic, and/or prophylactic agents.

In a further embodiment of the invention, pharmaceutical compositions, methods and kits are provided for the diagnosis, treatment, and/or prevention of a disease characterized by abnormal brain tissue, wherein an direct agonists of a K_{Ca} channel is administered in combination/alternation with one or more diagnostic, therapeutic, and/or prophylactic agents optionally further in combination/alternation with one or more of the compounds selected from the group consisting of leukotriene (LTC₄), bradykinin (BK), a BK analog, an agonist of a bradykinin receptor, a modulator of δ-glutamyl transpeptidase, a modulator of nitric oxide synthase, a modulator of guanylate cyclase, a modulator of phosphodiesterases, or a modulator of cGMP-dependent protein kinase.

In a further embodiment of the invention, pharmaceutical compositions, methods and kits are provided for the diagnosis, treatment, and/or prevention of a disease characterized by abnormal brain tissue, wherein direct agonists of a K_{ATP} channel is administered together with a indirect activator of a K_{ATP} channel in combination/alternation with one or more diagnostic, therapeutic, and/or prophylactic agents.

This drug delivery system of the present invention is independent of B2 receptors and therefore circumvents the disadvantages of bradykinin, including variable and refractory BTB permeability responses. The biochemical modulation strategy may improve the delivery of anti-neoplastic agents, including humanized monoclonal antibodies and therapeutic viral vectors selectively to brain tumors and neuropharmaceuticals to diseased brain regions while leaving normal brain unaffected and, thereby, significantly increase the survival rate for patients stricken with debilitating neurological diseases and tumors.

While not wishing to be limited by any particular mechanism of action, the combination/alternation of the agonist of an ATP-sensitive potassium channel, such as minoxidil sulfate, with an agonist of a calcium-activated potassium channel, such as NS-1619, of the present invention is believed to induce accelerated formation of transport vesicles in abnormal brain capillary endothelium and cells. These results provide evidence that vesicular transport is largely responsible for enhanced delivery of drugs across abnormal brain capillaries to abnormal brain tissue.

The methods of the present invention can be used to selectively deliver therapeutic compounds or diagnostics to abnormal brain regions, in particular, abnormally proliferating brain regions (i.e., brain tumors). For example, the delivery of the chemotherapeutic, carboplatin, to brain tumors can be increased using a K_{ATP} channel agonist (minoxidil sulfate or MS), resulting in enhanced survival in rats with intracranial tumors. Furthermore, MS-induced BTB permeability modulation allows delivery of macromolecules, such as dextrin, Her-2 Mob and GFP-Adv, selectively to abnormal brain tissue, such as tumor cells. For example, delivery to brain tumors can be increased using MS resulting in enhanced survival in mice with intracranial gill tumors. Furthermore, MS-induced BTB permeability modulation allows delivery of macromolecules, Her-2 Mob and GFP-Adv, selectively to brain tumor. Selective and enhanced delivery of small and macro molecules across abnormal brain tissue microvessels following K_{ATP} channel activation in conjunction with K_{Ca} channel activation can be exploited to increase permeability and enhance drug delivery to the abnormal brain tissue, for example to a brain tumor. A method for improving targeted delivery of anti-neoplastic agents to brain tumors and neuropharmaceuticals to diseased brain regions while leaving normal brain unaffected is disclosed.

In one embodiment of the invention, the disease characterized by abnormal brain tissue is an abnormal cellular proliferation, and in particular a neoplastic disease or malignancy, such as a cancer or a tumor. Non-limiting examples of neoplastic diseases or malignancies treatable with the composition of the present invention in combination and/or alternation with an antiproliferative agent include: glioma, glioblastoma, glioblastoma multiforme (GBM), astrocytoma, ependymoma, medulloblastoma, oligodendroma; meningioma, pituitary adenoma, neuroblastoma, craniopharyngioma. In one embodiment of the invention, the disease is a metastatic brain tumor. Non-limiting examples of metastatic brain tumors include metastatic breast and metastatic lung brain tumors.

In another embodiment of the invention, the disease characterized by abnormal brain tissue is a migraine, convulsion, bacterial infection, viral infection (e.g., HIV infection), schizophrenia, Parkinson's, Alzheimer's, hypoxia, cerebral ischemia, cerebral palsy, cerebrovascular disease such as stroke (e.g. embolic stroke), dyspnea, or encephalopathy. In yet another embodiment of the present invention, the disease characterized by abnormal brain tissue is due to physical or biochemical injury, such as trauma.

Additional K_{Ca} and K_{ATP} channel openers, including both direct agonists and indirect activators, are also described for use alone or in combination to selectively increase the blood brain barrier for the treatment, diagnosis or prevention of diseases, as described more fully herein. In a particular embodiment of the present invention, the indirect activator of K_{Ca} is an agent responsible for increasing in vivo levels of cGMP, such as an NO-independent activator soluble guanylyl cyclase. In another embodiment, the indirect activator of K_{ATP} activator is an agent responsible for increasing in vivo levels of cAMP, such as an activator of adenylyl cyclase.

In yet another embodiment, K_{Ca} and K_{ATP} channel openers as described herein are used alone or in combination to selectively increase the uptake of chemotherapeutic or diagnostic agents in regions of the body other than the brain. Examples are in tumors or cancers found in any body region, including but not limited to breast, prostate, lung, liver, kidney, colon, skin, head or neck or mouth.

I. Therapeutic Indications

The present invention is useful for the treatment of a disease or disorder characterized by abnormal brain tissue. Abnormal brain regions may include, for example, regions of brain tissue characterized by abnormal cell proliferation (e.g., malignant brain tumors), as well as regions of brain tissue physiologically affected by physical or biochemical injury, such as degenerative brain disease (e.g., Alzheimer's disease, Parkinson's disease), stroke, brain ischemia, infection or trauma.

In one embodiment of the present invention, the abnormal brain region is characterized by abnormal cell proliferation, and in particular, particular a neoplastic disease or malignancy, such as a cancer or a tumor.

In one embodiment of the invention, the abnormal brain region is a malignant brain tumor. Among malignant brain tumors for which the inventive methods are effective are gliomas, which include any malignant glial tumor, i.e., a tumor derived from a transformed glial cell. About half of all primary brain tumors are gliomas. A glial cell is a cell that has one or more glial-specific features, associated with a glial cell type, including a morphological, physiological and/or immunological feature specific to a glial cell (e.g. astrocyte or oligodendrocyte), for example, expression of the astroglial marker fibrillary acidic protein (GFAP) or the oligodendroglial marker O4. Non-limiting examples of neoplastic diseases or malignancies treatable with the composition of the present invention in combination and/or alternation with an antiproliferative agent include gliomas, glioblastomas, glioblastoma multiforme (GBM; i.e., astrocytoma grade IV), oligodendroglioma, primitive neuroectodermal tumor, low, mid and high grade astrocytoma (i.e., astrocytoma grade II, anaplastic astrocytoma grade III, astrocytoma with oligodendrogliomal component), ependymoma (e.g., myxopapillary ependymoma papillary ependymoma, subependymoma, anaplastic ependymoma), oligodendroglioma, medulloblastoma, meningioma (i.e., atypical meningioma, malignant meningioma), pituitary tumors (i.e., pituitary adenoma), neuroblastoma, and craniopharyngioma.

Other brain tumors that can be treated according to the present invention include, for example, acoustic neuroma (e.g., Neurilemmoma, Schwannoma, Neurinoma), chordoma, chordoma, CNS lymphoma, cysts, dermoid cysts, gangliocytoma, ganglioglioma, and hemangioblastoma.

In a particular embodiment of the present invention, the abnormal brain tissue is a secondary or metastatic brain tumor (i.e., tumors that have spread to the brain from another part of the body). Non-limiting examples of metastatic brain tumors treatable with the composition of the present invention include cancers originating in breast, lung, kidney, colon, prostate, and skin (malignant melanoma).

In another embodiment of the invention, the disease characterized by a region of abnormal brain tissue is a migraine, convulsions, an infection, metal illness (e.g., schizophrenia, depression), hypoxia, cerebral ischemia, cerebral palsy, degenerative brain disease, cerebrovascular disease, dyspnea, or encephalopathy. In yet another embodiment of the present invention, the disease characterized by abnormal brain tissue is due to physical or biochemical injury, such as trauma.

In one embodiment, the disease characterized by abnormal brain tissue is a migraine or headache. Migraines include, for example, migraine with aura, migraine without aura, masilar artery migraine, carotidynia, headache-free migraine, ophthalmoplegic migraine, and status migraine.

In another embodiment, the disease characterized by abnormal brain tissue is a convulsive disease or disorder. The term convulsion (i.e., seizure) refers to a sudden change in behavior due to an excessive electrical activity in the brain. Causes include, for example, epilepsy, head injury, infection or stroke. Types of epilepsy include, for example, general epilepsy, generalized cryptogenic or symptomatic epilepsies, generalized symptomatic epilepsies of nonspecific etiology, focal or partial epilepsy, temporal lobe epilepsies and frontal lobe epilepsies.

In one embodiment, the disease or disorder characterized by abnormal brain tissue is a cerebrovascular disease. Cerbrovascular disease includes diseases in which neurological symptoms and signs result from disorders or diseases of the blood vessels (e.g., congenital anomalies and atherosclerosis). These include, for example, ischemic syndromes and hemorrhagic syndromes. Ischemic syndromes are disorders caused by insufficient cerebral circulation, and including for example, transient ischemic attacks (TIAs) and ischemic stroke. Hemorrhagic syndromes involve by bleeding into brain tissue, including the epidural, subdural, or subarachnoid space, or a combination of these sites. Intracerebral hemorrhages can occur almost anywhere in the brain, including for example, near the basal ganglia, internal capsule, thalamus, cerebellum, or brain stem. Head trauma is the most common cause of subarachnoid hemorrhage. In a particular embodiment of the invention, the abnormal brain region is a region of brain tissue physiologically affected by stroke.

In another embodiment, the disease or disorder characterized by abnormal brain tissue is a neurodegenerative disease. Neurodegenerative diseases are disorders characterized by progressive nervous system dysfunction in which neurons in particular structures or regions of the brain deteriorate or die over time. Representative, non-limiting degenerative brain diseases include Alzheimer's, cerebellar atrophies, triplet repeat diseases (e.g., Huntington's disease), Parkinson's disease, Niemann-Pick Type C Disease (NP-C), prior disorders (e.g., Creutzfeldt Jakob Disease), olivopontocerebellar degeneration, motor neuron disease, cerebellar degenerations, Amyotrophic Lateral Sclerosis (i.e., Lou Gehrig's Disease), dementia (e.g., dementia with lewy bodies), as well as diseases involving neurological autoimmune disease (e.g., multiple sclerosis). For a review of neurodegenerative diseases, see Williams A. BMJ (2002) 324:1465-1466;

In another embodiment, the disease or disorder characterized by abnormal brain tissue is a brain infection. Infections of the brain may be caused by, for example, a bacteria, virus or virus-like agent. Infections include both acute and chronic conditions. Bacterial infections include, for example, Streptococcus pneumonia, Streptococcus pyogenes, Staphylococcus aureu, Staphylococcus epidermidis, Enterobacteriacea, Propionibacterium, Pseudomonoas aeruginosa, Neisseria meningitis, Haemophilus influenzae and Listeria moncytogenes. Acute neurological syndromes associated with viral infection include, for example, acute viral encephalitis, flaccid paralysis, aspectic meningitis, and post infectious encephalomyelitis. Acute viral encephalitis may be caused by for example, herpes simplex virus, cytomegalovirus, varicella, rabies or an arbovirus. Common viral agents of asceptic meningitis include, for example, enteroviruses, mumps virus and lymphocytic choriomeningitis virus. Post infectious encephalomyelitis is a complication of infection with measles, mumps, rubella and primary varicella-zoster virus infection, for example. Gillain Barre syndrome is also an acute neurological syndrome associated with viral infection.

Chronic neurological diseases attributable to viral infection include, subacute sclerosing pan encephalitis (caused by persistent measles infection), progressive multifocal leuco-encephalopathy (caused by members of the papovavirus family) spongiform encephalopathies (prion diseases) (e.g., Creutzfeldt-Jakob disease (CJD), Gerstmann-Streussler Syndrome), and retroviral diseases (e.g., HIV-1 and HIV-2) characterized by paralysis, wasting, and ataxia.

Neurological disorders which can be treated according to the present invention include metabolic disorders, including, for example, Abetalipoproteinemi, Central pontine myelinolysis, Galactosemia, Gaucher, Homocystinuria, Kernicterus, Leigh's Disease, Lesch-Nyhan Syndrome, Menkes' Syndrome, Niemann-Pick Type C disease, Reye's Syndrome, Korsakoffs disease, Tay-Sach's disease.

Other neurological disorders which can be treated according to the present invention include, for example, Batten Disease, Canavan disease, Charcot-Marie-Tooth disorder (CMT), dystonia, Neurofibromatosis (NF), Tuberous sclerosis complex (TSC), Aicardi Syndrome; Akinetic Mutism; Amblyopia; Bardet-Biedl Syndrome; cerebral abscess; cerebral edema; Corticobasal Degeneration; Familial Mediterranean Fever; Glycogen Storage Disease Type II; Hallervorden-Spatz Syndrome; intracranial hypertension; intracranial hypotension; Joubert Syndrome; Kluver-Bucy Syndrome; Laurence-Moon Syndrome; Lowe Syndrome; Machado-Joseph; Miller Fisher Syndrome; Moyamoya; olivopontocerebellar atrophy; phenylketonuria; Schizencephaly; transient global amnesia; and Zellweger Syndrome.

In yet another embodiment, the present invention is useful for increasing the uptake of a therapeutic agent or diagnostic agent in regions of the body other than the brain. In one embodiment, the invention is useful to delivering a medicant to a malignant tumor located in an area of the body other than the brain. Representative, non-limiting tumors or cancers include breast cancer, skin cancer, bone cancer, prostate cancer, liver cancer, lung cancer, brain cancer, cancer of the larynx, gallbladder, pancreas, rectum, parathyroid, thyroid, adrenal, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, leukemias, lymphomas, malignant melanomas, epidermoid carcinomas, and other carcinomas and sarcomas.

II. Diagnostic, Therapeutic and/or Prophylactic Agents

The methods of delivering a medicament to an abnormal brain region and/or to a malignant tumor in a mammalian subject are effective in selectively delivering any medicament across the microvascularature of an abnormal brain region and/or malignant tumor. The medicament may be in one embodiment a drug, i.e., a chemotherapeutic agent. Example of chemotherapeutic agents including therapeutic cytotoxic agents (e.g., cisplatin, carboplatin, methotrexate, 5-fluorouracil, amphotericin), naked DNA expression vectors, therapeutic proteins, therapeutic oligonucleotides or nucleotide analogs, interferons, cytokines, or cytokine agonists or antagonists, adrenergic agents, anticonvulsants, anti-trauma agents, or any neuropharmaceutical agent used to treat or prevent an injury or disorder of the brain. Chemotherapeutic agents also include ischemia-protective drugs such as N-methyl-D-aspartate (NMDA) receptor antagonists; antimicrobial agents, such as antibiotics; immunotoxins, immunosuppressants, boron compounds, monoclonal antibodies and specific antigen-binding antibody fragments (e.g., Fab, Fab′, F(ab′)₂, or F(v) fragments), and cytokines, such as interferons, interleukins (e.g., interleukin [IL]-2), tumor necrosis factor (TNF)-α, or transforming growth factors (e.g., TGF-β).

The medicant also includes anticancer chemotherapeutic agents. Typically, anticancer chemotherapeutic agents are cytotoxic agents, such as 5-fluorouracil, cisplatin, carboplatin, methotrexate, daunorubicin, doxorubicin, vincristine, vinblastine, or a cytotoxic akylating agent, such as, but not limited to, busulfan, chlorambucil, cyclophosphamide, melphalan, or ethylesulfonic acid.

Medicaments also include any therapeutic viral particle, for example an adenovirus-derived or herpes simplex virus (HSV)-derived viral vector for delivering genetic material to a cellular target in vivo. Medicaments also include diagnostic agents, such as imaging or contrast agents, for example, radioactively labeled substances (e.g., [⁹⁹Tc]-glucoheptonate), gallium-labeled imaging agents (e.g., gallium-EDTA), ferrous magnetic, fluorescent, luminescent, or iodinated contrast agents. Where suitable, any of the afore-mentioned medicaments having anticancer activity can also be used in practicing the method of selectively delivering a medicament to a malignant tumor or the method of treating a malignant tumor in a human subject.

In one embodiment of the present invention, the medicament can be a molecular substance having a molecular weight between about 50 Daltons and about 250 kD. Or it can be a particle, such as a viral particle, having a diameter between about 50 to 250 nanometers.

The amount of medicament that is employed is within a conventional dose range for each medicament, however by practicing the inventive method, the increased transvascular permeability afforded can provide a greater selective therapeutic effect per dose or permit a lower effective dose to be used, if desired, for example to lessen systemic toxic effects from anti-cancer medication in a particular subject.

Antiproliferative Agents

As used herein, an antiproliferative agent is a compound that decreases the hyperproliferation of cells. Proliferative disorders are currently treated by a variety of classes of compounds includes alkylating agents, antimetabolites, natural products, enzymes, biological response modifiers, miscellaneous agents, hormones and antagonists. Any of the antiproliferative agents listed below, or any other such agent known or discovered to exhibit an antiproliferative effect can be more effectively delivered with one or more direct agonist(s) or indirect activators of the K_{Ca} or K_{ATP} channel, or both, in accordance with this invention.

Representative adjuncts include levamisole, gallium nitrate, granisetron, sargramostim strontium-89 chloride, filgrastim, pilocarpine, dexrazoxane, and ondansetron. Physicians' Desk Reference, 50th Edition, 1996.

Representative androgen inhibitors include flutamide and leuprolide acetate. Physicians' Desk Reference, 50th Edition, 1996.

Representative antibiotic derivatives include doxorubicin, bleomycin sulfate, daunorubicin, dactinomycin, and idarubicin.

Representative antiestrogens include tamoxifen citrate and analogs thereof. Physicians' Desk Reference, 50th Edition, 1996. Additional antiestrogens include nonsteroidal antiestrogens such as toremifene, droloxifene and roloxifene. Magarian et al., Current Medicinal Chemistry, 1994, Vol. 1, No. 1.

Representative antimetabolites include fluorouracil, fludarabine phosphate, floxuridine, interferon alfa-2b recombinant, methotrexate sodium, plicamycin, mercaptopurine, and thioguanine. Physicians' Desk Reference, 50th Edition, 1996.

Representative cytotoxic agents include doxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci. Physicians' Desk Reference, 50th Edition, 1996.

Representative hormones include medroxyprogesterone acetate, estradiol, megestrol acetate, octreotide acetate, diethylstilbestrol diphosphate, testolactone, and goserelin acetate. Physicians' Desk Reference, 50th Edition, 1996.

Representative immunodilators include aldesleukin. Physicians' Desk Reference, 50th Edition, 1996.

Representative nitrogen mustard derivatives include melphalan, chlorambucil, mechlorethamine, and thiotepa. Physicians' Desk Reference, 50th Edition, 1996.

Representative steroids include betamethasone sodium phosphate and betamethasone acetate. Physicians' Desk Reference, 50th Edition, 1996.

Specifically, the chemotherapeutic agent can be an antineoplastic agent.

Specifically, the antineoplastic agent can be a cytotoxic agent.

Specifically, the cytotoxic agent can be paclitaxel or doxorubicin.

Additional suitable chemotherapeutic agents include alkylating agents, antimitotic agents, plant alkaloids, biologicals, topoisomerase I inhibitors, topoisomerase II inhibitors, and synthetics. AntiCancer Agents by Mechanism, http://www.dtp.nci.nih.gov/docs/cancer/searches/standard_mechanism_list.html, Apr. 12, 1999; Approved Anti-Cancer Agents, http://www.ctep.info.nih.gov/handbook/HandBookText/fda_agen.htm, pages 1-7, Jun. 18, 1999; MCMP 611 Chemotherapeutic Drugs to Know, http//www.vet.purdue.edu/depts/bms/courses/mcmp611/chrx/drg2no61.html, Jun. 24, 1999; and Chemotherapy, http://www.vetmed.lsu.edu/oncology/Chemotherapy.htm, Apr. 12, 1999.

Representative alkylating agents include asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864. AntiCancer Agents by Mechanism, http://dtp.nci.nih.gov/docs/cancer/searches/standard_mechanism_list.html, Apr. 12, 1999.

Representative antimitotic agents include allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate. AntiCancer Agents by Mechanism, http://dtp.nci.nih.gov/docs/cancer/searches/standard_mechanism_list.html, Apr. 12, 1999.

Representative plant alkaloids include actinomycin D, bleomycin, L-asparaginase, idarubicin, vinblastine sulfate, vincristine sulfate, mitramycin, mitomycin, daunorubicin, VP-16-213, VM-26, navelbine and taxotere. Approved Anti-Cancer Agents, http://ctep.info.nih.gov/handbook/HandBook Text/fda_agent.htm, Jun. 18, 1999.

Representative biologicals include alpha interferon, BCG, G-CSF, GM-CSF, and interleukin-2. Approved Anti-Cancer Agents, http://ctep.info.nih.gov/handbook/HandBookText/fda_agent.htm, Jun. 18, 1999.

Representative topoisomerase I inhibitors include camptothecin, camptothecin derivatives, and morpholinodoxorubicin. AntiCancer Agents by Mechanism, http://dtp.nci.nih.gov/docs/cancer/searches/standard_mechanism_list.html, Apr. 12, 1999.

Representative topoisomerase II inhibitors include mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16. AntiCancer Agents by Mechanism, http://dtp.nci.nih.gov/docs/cancer/searches/standard_mechanism_list.html, Apr. 12, 1999.

Representative synthetics include hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium. Approved Anti-Cancer Agents, http://ctep.info.nih.gov/handbook/HandBookText/fda_agen.htm, Jun. 18, 1999.

Representative antibodies include Monoclonal antibodies directed to proliferating cells such as Rituximab (anti-CD20) for B-cell tumors and herceptin.

Antibiotics

Any of the antibiotics listed below, or any other such agent known or discovered to exhibit a diagnostic and/or therapeutic effect can be more effectively delivered with one or more direct agonist(s) or indirect activators of the K_{Ca} and/or K_{ATP} channel in accordance with this invention.

Cell wall synthesis inhibitors, such as beta lactam antibiotics, generally inhibit some step in the synthesis of bacterial peptidoglycan. Penicillin is generally effective against non-resistant streptococcus, gonococcus and staphylococcus. Amoxycillin and Ampicillin have broadened spectra against Gram-negative bacteria. Cephalosporins are generally used as penicillin substitutes, against Gram-negative bacteria and in surgical prophylaxis. Monobactams are generally useful for the treatment of allergic individuals.

Cell membrane inhibitors disorganize the structure or inhibit the function of bacterial membranes. Polymyxin, produced by Bacillus polymyxis, is a cell membrane inhibitor that is effective mainly against Gram-negative bacteria and is usually limited to topical usage.

Protein synthesis inhibitors include the tetracyclines, chloramphenicol, the macrolides (e.g. erythromycin) and the aminoglycosides (e.g. streptomycin). Aminoglycosides have been used against a wide variety of bacterial infections caused by Gram-positive and Gram-negative bacteria. Streptomycin has been used extensively as a primary drag in the treatment of tuberculosis. Gentamicinis active against many strains of Gram-positive and Gram-negative bacteria, including some strains of Pseudomonas aeruginosa. Kanamycin is active at low concentrations against many Gram-positive bacteria, including penicillin-resistant staphylococci.

The tetracyclines are protein synthesis inhibitors that consist of eight related antibiotics that are all natural products of Streptomyces, although some can now be produced semisynthetically. Tetracycline, chlortetracycline and doxycycline are the best known. The tetracyclines are broad-spectrum antibiotics with a wide range of activity against both Gram-positive and Gram-negative bacteria. Tetracyclines have some important uses, such as in the treatment of Lyme disease.

Chloramphenicol is a protein synthesis inhibitor that has a broad spectrum of activity but it exerts a bacteriostatic effect. It is effective against intracellular parasites such as the rickettsiae. It is infrequently used in human medicine except in life-threatening situations (e.g. typhoid fever). Macrolide antibiotics, such as erythromycin, are protein synthesis inhibitors that are active against most Gram-positive bacteria.

Some antibiotics affect the synthesis of DNA or RNA or can bind to DNA or RNA so that their messages cannot be read. For example, nalidixic acid is a synthetic quinoloid antibiotic that is active mainly against Gram-negative bacteria. The main use of nalidixic acid is in treatment of lower urinary tract infections (LUTI). In addition, the rifamycins has greater bactericidal effect against the bacteria that causes tuberculosis than other anti-tuberculosis drugs and is also useful for treatment of tuberculosis meningitis and meningitis caused by Neisseria meningitidis.

Finally, competitive inhibitors are generally synthetic antibiotics that are growth factor analogs. Growth factor analogs are structurally similar to bacterial growth factors, but do not fulfill their metabolic functions in cells. For example, sulfonamides have been extremely useful in the treatment of uncomplicated UTI caused by E. coli and in the treatment of meningococcal, meningitis.

Suitable antibiotic agents are disclosed, e.g. in Physician's Desk 30 Reference (PDR), Medical Economics Company (Montvale, N.J.), (53^{rd} Ed.), 1999; Mayo Medical Center Formulary, Unabridged Version, Mayo Clinic (Rochester, Minn.), January 1998; Merck Index An Encyclopedia of Chemicals, Drugs and Biologicals, (11^{th} Ed.), Merck & Co., Inc. (Rahway, N.J.), 1989; University of Wisconsin Antimicrobial Use Guide http://www.medsch.wisc.edu/clinsci/5amcg/amcg.html; Introduction on the Use of the Antibiotics Guideline, of Specific Antibiotic Classes, Thomas Jefferson University, http://jeffline.tju.edu/CWIS/OAC/antibiotics_guide/intro.html; and references cited therein.

Suitable antibiotics include, e.g. aminoglycosides, β-lactam antibiotics, cephalosporius, macrolides, miscellaneous antibiotics, penicillins, tetracyclines, antifungals, antimalarial agents, antituberculosis agents, antivirals, leprostatics, miscellaneous anti-infectives, quinolones, sulfonamides, urinary anti-infectives, nasal antibiotics, opthalmic antibiotics, opthalmic antivirals, opthalmicquinalones, opthalmic sulfonamides, skin and mucous membrane antibiotics, skin and mucous membrane antifungals, skin and mucous membrane antivirals, skin and mucous membrane miscellaneous anti-infectives, skin and mucous membranescabicides and pedulicides, skin and mucous membrane antineoplasts, nitrofurans and oxazolidinones. Physician's Desk Reference (PDR), Medical Economics Company (Montvale, N.J.), (53^{rd} Ed.), 1999 and Mayo Medical Center Formulary: Unabridged Version, Mayo Clinic (Rochester, Minn.), January 1998.

Aminoglycosides include, for example, Amikacin (amikacin sulfate); Craramyein (gentamicin sulfate); Nebcin (tobramycin sulfate); Netromycin (netilmicin sulfate); Streptomycin Sulfate; and TOBI (tobramycin).

β-Lactam antibiotics include, for example, Azactam (aztreonam); Cefotan (cefotetan); Lorabid (loracarbef); Mefoxin (cefoxitin); Merrem (meropenem); and Primaxin (imipenem and cilastatin for injectable suspension).

Cephalosporins include, for example, Ancef (cefazolin); Ceclor (cefaclor); Cedax (ceffibuten); Cefizox (ceffizoxime sodium); Cefobid (cefoperazone sodium); Ceftin (cefuroxime axetil); Cefzil (cefprozil); Ceptaz (ceftazidime); Claforan (cefotaxime); Duricef (cefadroxil monohydrate); Fortaz (ceftazidime); Keflex (cephalexin); Keftab (cephalexin HCl); Kefurox (cefuroxime); Kefzol (cefazolin); Mandol (cefamandole nafate); Maxipime (cefepime HCl); Monocid (cefonicidsodium); Omnicef (cefdinir); Rocephin (ceftriaxone); Suprax (cefixime); Tazicef (ceftazidime); Tazidime (ceftazidime); Vantin (cefpodoxime proxetil); and Zinacef5(cefuroxime).

Macrolides include, for example, Biaxin (clarithromycin); Dynabac (dirithromycin); E.E.S. 200 (Erythromycin Ethylsuccinate); E.E.S. 400 (Erythromycin Ethylsuccinate); EryPed 200 (Erythromycin Ethylsuccinate); EryPed 400 (Erythromycin Ethylsuccinate); Ery-Tab (Erythromycin delayed-release tablets); Erythrocin Stearate (Erythromycin stearate); Ilosone (erythromycinestolate); PCE Dispertab (erythromycin particles in tablets); Pediazole (erythromycin ethylsuccinate and sulfisoxazole acetyl for oral suspension); Tao (troleandomycin); Zithromax (azithromycin); and Erythromycin.

Miscellaneous antibiotics include, for example, Cleocin HCl (clindamycin hydrochloride); Cleotin Phosphate (elindamycin phosphate); Coly-Mycin M (colistimethate sodium); and Vancocin HCl (vancomycin hydrochloride).

Penicillins include, for example, Amoxil (amoxicillin); Augmentin (amoxicillin/clavulanate potassium); Bicillin C-R 900/300 (Penicillin G benzathine and Penicillin G procaine suspension); Bicillin C-R (Penicillin G benzathine and Penicillin G procaine suspension); Bicillin L-A (Penicillin G benzathine suspension); Geoeillin (carbencillin indanyl sodium); Mezlin (sterile mezlocillinsodium); Omnipen (ampicillin); Pen-Vee K (penicillin V potassium); Pfizerpen (penicillin G potassium); Pipracil (piperacillin sodium); Speetrobid (bacampicillin-HCl); Ticar (tiearcillin disodium); Timentin (ticarcillin disodium and clavulanate potassium); Unasyn (ampicillin sodium/sulbactam sodium); Zosyn (piperacillin sodium and tazobactam sodium); and Dicloxacillin Sodium.

Tetracyclines include, for example, Achromycin V (tetracycline HCl); Declomycin (demeclo-cycline HCl); Dynacin (minocylcine HCl); Minocin (minocycline hydrochloride); Monodox (Doxycycline monohydrate capsules); Terramycin (oxytetracyline); Vectrin (minocycline hydrochloride); Vibramycin Calcium (doxycycline sodium); Vibramycin Hyclate (doxycycline hyclate); Vibramycin Monohydrate (doxycycline monohydrate); Vibra-Tabs (doxycycline-hydrate); Declomycin (demeclocycline HCl); Vibramycin (doxycycline); Dynacin (Minocyline HCl); Terramycin (oxytetracycline HCl); Achromycin V capsules5 (tetracycline HCl); Linco-mycins; and Cleotin HCl (clindamycin HCl).

Antifungals include, for example, Abelcet (amphotericin B lipid complex); AmBisome (amphotericin B); Amphotec (amphotericin B cholesterol sulfatecomplex); Ancobon (flucytosine); Diflucan (fluconazole); Fulvicin P/Gamma (ultramicrosize griseofulvin); Fulvicin P/G 165 and 330 (ultramicrosize griseofulvin); Grifulvin V (griseofulvin); Gals-PEG (gxiseofulvin ultramicrosize); Lamisil (terbinafine hydrochloride); Nizoral (ketoconazole); Amphotericin B; Lotrimin (clotrimazole); Dapsone tablets (dapsone); Diflucan (fluconazole); Monistat-Derm cream (miconazole); Mycostalin Crc .am (nystatin); and Sporanox (itraconazole).

Antimalarial agents include, for example, Aralen hydrochloride (chloroquine HCl); Aralen phosphate (chloroquine phosphate); Dataprim (pyrimethamine); Ladam (mefloquine HCl); and Plaquenil (hydroxychloroqnine sulfate).

Antituberculosis agents include, for example, Capastat sulfate (capreomycinsulfate); Myambutol (ethambutol hydrochloride); Mycobutin (rifabutin capsules); Nydrazid (isoniazid injection); Paser (aminosalicylic acid); Prifiin (rifapentine); Pyrazinamide tablets (pyrazinamide); Rifadin (rifampin capsules); Rifadin IV (rifampin for injection); Rifamate (rifampin and isoniazid); Rifater (rifampin, isoniazid and pyrazinamide); Seromycin (cycloserine capsules); Streptomycin-Sulfate; Tice BCG (BCG vaccine); Cycloserine (seromycin capsules); Urised (Methenamine); and Trecator-SC (ethionamide tablets).

Antivirals include, for example, Alferon N (interferon alfa-n3); Crixivan (indinavir sulfate); Cytovene (ganciclovir); Cytovene-IV (ganciclovir sodium); Epivir (lamivudine); Famvir (famciclovir); Flumadine (rimantadine HCl); Foscavir (foscamet sodium); Hivid (zalcitabine); Intron A (interferon alfa-2b); Invirase (saquinavir mesylate); Norvir (ritonavir); Rebetron combination therapy, which contains Rebetrol (ribavirin) and Intron A (inteferon alfa-2b); Rescriptor (delavirdine mesylate); Retrovir (ziduvudine); Retrovir IV (ziduvudine); Symmetrel (amantadine HCl); Synagis (palivizumab); Valtrex (valacyclovir HCl); Videx (didanosine); Viracept (nelfinavir mesylate); Viramune (nevirapine); Virazole (ribavirin); Vistide (cidofovir); Zerit (stavudine (d4T)); Symmetrel Syrup(amantadine HCl); Combivir Tablets (lamiduvine); and Zovirax (acyclovir).

Leprostatics include, for example, Dapsone Tablets (dapsone).

Miscellaneous anti-infectives include, for example, Daraprim (pyrimethamine); Flagyl 375 (metronidazole); Flagyl ER Tablets (metronidazole); Flagyl I.V. (metronidazole); Furoxone (furazolidone); Mepron (atovaquone); and Neutrexin (tfimetrexate glucuronate).

Quinolones include, for example, Cipro (ciprofloxacin HCl); Floxin (ofloxacin); Levaquin (levofloxacin); Mazaquin (lomefioxacin HCl); Noroxin (norfloxacin); Penetrex (enoxacin); Raxar (grepafloxacin HCl); Trovan (trovafioxacin mesylate); and Zagam (sparfloxacin).

Sulfonamides include, for example, Bactrim. (trimethoprim and sulfamethoxazole); Bactrim DS (Irimethoprim and sulfamethoxazole double strength); Pediazole (erythromycin ethylsuccinate and sulfisoxazole acetyl); Septra (trimethoprim and sulfamethoxazole); Septra DS (trimethoprim and sulfamethoxazole); Co-Trimoxazole, Sulfadiazine, Battrim I.V. Infusion (sulfamethoxazole); Sulfapyridine and Pediazole (erythromycin ethylsuccinate and sulfisoxazole acetyl).

Nitrofurans include, for example, Furadantin Oral Suspension (nitrofurantoin).

Oxazolidinones include, for example, Zyvox (linezolid).

It is appreciated that those skilled in the art understand that the antibiotic useful in the present invention is the biologically active compound present in any of the antibiotic formulations disclosed above. For example, Azactam (aztreonam) is typically available as an injectable solution. The antibiotic agent, however, is (z)-2-[[[(2-amino-4-thiazolyl)-[[(2S,-3S)-2-methyl-4-oxo-1-sulfo-3-azetidinyl]carbamoyl]methylene]amino]-oxy]-2-methyl-propionic acid. Physician's Desk Reference (PDR), Medical Economics Company (Montvale, N.J.), (53^{rd} Ed.), pp. 820-823, 1999.

Amikacin is commercially available from Elkins-Sinn and is D-streptamine, O-3-amino-3-deoxy-α-D-glucopyranosyl-(1→6)-O-6-deoxy-α-z-D-gluco-pyranosyl-(1→4)]-N′-(4-amino-2 hydroxy-1-oxobutyl)-2-deoxy-,1 (S)—, sulfate (1:2) (salt).

Garamycin (gentamicin sulfate) is commercially available from Schering.

Nebcin (tobramycin sulfate) is commercially available from Lilly and is 0-3-amino-3-deoxy-α-D-glucopyranosyl-(1→4)-O-[2,6-diamino-2,3-6-trideoxy-α-D-ribo-hexopyranosyl-(1→6)]-2-deoxy-L-streptamine, sulfate (2:5) (salt).

Netromycin (netilmicin sulfate) is commercially available from Schering and is O-3-deoxy-4-C-methyl-3-(methylamino)-β-L-ara-binopyranosyl-(1→4)-O-[2,6-diamino-2,3,4,6-tetradeoxy-α-D-glycero-hex-4-enopyransyl-(1→6)-2-deoxy-N³-ethyl-L-streptamine sulfate (2:5) salt.

Streptomycin Sulfate is commercially available from Pfizer and is D-Streptamine, (1→4)-N,N′-bis(aminoiminomethyl)-O-2-deoxy-2-(methylamino)-α-L-glucopyranosyl-(1→2)-O-5-deoxy-3-C-formyl-L-α-lyxo-furanosyl sulfate (2:3) salt.

TOBI (tobramycin) is commercially available from Pathogenesis Corporation and is O-3-amino-3-deoxy-α-D-glucopyranosyl-(1→4)-O-[2,6-diamino-2,3,6-trideoxy-a-D-ribo-hexopyranosyl-(1-6)]-2-deoxy-L-streptamine.

Azactam (aztreonam) is commercially available from Bristol-Myers Squibb and is (Z)-2-[[[(2-amino-4-thiazolyl)[[(2S,-3S)-2-methyl-4-oxo-1-sulfo-3-azetidinyl]-carbamoyl]methylene]amino]oxy]-2-methylpropionic acid.

Cefotan (cefotetan) is commercially available from Zeneca and is [6R-(6a,7a)]-7-[[[4-(2-amino-1-carboxy-2-oxoethylidene)-1,3-dithietan-2-yl]carbonyl]-amino]-7-methoxy-3-[[(1-methyl-H-tetrazol-5-yl)thio]methyl]-8-oxo-5-thia-1-azabicyclo-[4.2.0]-oct-2-ene-2-carboxylic acid disodium salt.

Lorabid (loracarbef) is commercially available from Lilly and is (6R,7S)-7-[R-2-amino-2-phenylacetamido]-3-chloro-8-oxo-1-azabicyclo-[4.2.0]-oct-2-ene-2-carboxylic acid, monohydrate.

Mefoxin (cefoxitin) is commercially available from Merck and is sodium (6R,7S)-3-(hydroxymethyl)-7-methoxy-8-oxo-7-[2-(2-thienyl)acetamido]-5-thia-1-azabicylo-[4.2.0]-oct-2-ene-2-carboxylate carbamate (ester).

Merrem (meropenem) is commercially available from Zeneca and is (4R,5S,6S)-3-[(3S,5 S)-5-(Dimethylcarbamoyl)-3-pyrrolidinyl]thiol]-6-[(1R)-1-hydroxyethyl]-4-methyl-7-oxo-1-azabicyclo-[3.2.0]-hept-2-ene-2-carboxylic acid trihydrate.

Primaxin (imipenem and cilastatin for injectable suspension) is commercially available from Merck and is (1) imipenem is N-formimidoylthienamycin monohydrate, chemical name is [5R-[5α,6α(R*)]]-6-(1-hydroxyethyl)-3-[[2-[(iminomethyl)amino]-ethyl]-thio]-7-oxo-1-azabicylco-[3.2.0]-hept-2-ene-2-carboxylic acid monohydrate, cilastatin sodium is [R-[R*,S*,-(Z)]]-7-[(2-amino-2-carboxyethyl)thio]-2-[[(2,2-dimethyl cyclopropyl)carbonyl]amino]-2-heptenoic acid, monosodium salt.

Ancef (cefazolin) is commercially available from SmithKline Beecham and is 3-{[(5-methyl-1,3,4-thiadiazol-2-yl)thiomethyl)]}-8-oxo-7-[2-(1H-30-tetrazol-1-yl)-acetamido]-5-thia-1-azabicyclo[4.2.0]oct-2-ene-2-carboxylic acid.

Ceclor (cefaclor) is commercially available from Lilly and is 3-chloro-7-D-(2-phenylglycinamido)-3-cephem-4-carboxylic acid monohydrate;

Cedax (ceflibuten) is commercially available from Schering and is (+)-(6R,7R)-7-[(Z)-2-(2-(2-amino-4-thiazoly)-4-carboxycrotonamido]-8-oxo-5-thia-1-azabicyclo-[4.2.0]-oct-2-ene-2-carboxylic acid, dihydrate.

Cefizox (ceftizoxime sodium) is commercially available from Fujisawa and is sodium salt of [6R-[6α 7β(Z)]]-7 [[2,3, dihydro-2-imino-4-thiazolyl) (methoxy amino) acetyl]amino]-8-oxo-5-thia-1-azabicyclo[4.2.0]oct-2-ene-2-carboxyolic acid.

Cefobid (cefoperazone sodium) is commercially available from Pfizer and is sodium (6R,7R)-7-[R-2-(4-ethyl-2,3-dioxo-1-piperazine-carboxamido)-2-(p-hydroxy-phenyl)-acetamido)-3-[[(1-methyl-1H-tetrazol-5-yl)thio]methyl]-8-oxo-5-thia-1-azabicyclo[4.2.0]oct-2-ene-2-carboxylate.

Ceftin (cefuroxime axetil) is commercially available from Glaxo Wellcome and is (R,S)-1-hydroxyethyl(6R,7R)-7-[2-(2-furyl)glyoxylamido]-3-(hydroxyethyl)-(8-oxo-5-thia-1-aza-bicyclo[4.2.0]oct-2-ene-2-carboxylate, 7² (Z)-O-methyl-oxime), 1-acetate 3-carbamate.

Cefzil (cefprozil) is commercially available from Bristol-Myers Squibb and is (6R,7R)-7-[R-2-amino-2-(p-hydroxyphenyl)acetamido]-8-oxo-3-propenyl-5-thia-1-azabicyclo[4.2.0]oct-2-ene-2-carboxylic acid monohydrate.

Ceptaz (ceftazidime) is commercially available from Glaxo Wellcome and is [6R-[6α 713(Z)]]-1-[[7-[[(2-amino-4-thiazolyl)[(1-carboxy-1-methylethoxy)imine]acetyl]-amino]-2-carboxy-8-oxo-5-thia-1-azabicyclo[4.2.0]oct-2-en-3-yl]methyl]hydroxide, inner salt.

Claforan (cefotaxime) is commercially available from Hoescht Marion Roussel and is 7-[2-(2-amino-4-thiazolyl)glyoxylamido]-3-(hydroxymethyl)-8-oxo-5-thia-1-azabicyclo-[4.2.0]-oct-2-ene-2-carboxylate 7² (Z)-(O-methyloxime), acetate (ester).

Duricef (cefadroxil monohydrate) is commercially available from Bristol-Myers Squibb and is [6R-[6α,7β(R*)]]-7-[[amino (4-hydroxyphenyl)acetyl]amino]-3-methyl-8-oxo-5-Thia-1-azabicyclo[4.2.0]oct-2-ene-2-carboxylic acid, monohydrate.

Fortaz (ceftazidime) is commercially available from Glaxo Wellcome and is [6R-[6α,7β(Z)]]-1-[[7-[[(2-amino-4-thiazolyl)[1-carboxy-1-methylethoxy)imino]acetyl]-amino]-2-carboxy-8-oxo-5-thia-1-azabicyclo[4.2.0]oct-2-en-3-yl]methyl]-hydroxide, inner salt.

Keflex (cephalexin) is commercially available from Dista and is 7-(D-α-Amino-α-phenyl acetamido)-3-methyl-3-cephem-4-carboxylic acid monohydrate.

Keftab (cephalexin HCl) is commercially available from Dura and is 7-(D-2-Amino-2-phenylacetamido)-3-methyl-3-cephem-4-carboxylic acid hydrochloride mono-hydrate.

Kefurox (cefuroxime) is commercially available from Lilly and is the sodium salt of (6R.7R)3-carbamoyloxymethyl-7-[Z-2-methoxyimino-2-(fur-2-yl)acetamido]ceph-3-em-4-carboxylate.

Kefzol (cefazolin) is commercially available from Lilly and is the sodium salt of 3-{[(5-methyl-1,3,4-thiadiazol-2-yl)thio]methyl}-8-oxo-7-[2-(1H-tetrazol-1-yl)acetamido]-5-thia-1-aza-bicyclo[4.2.0]oct-2-ene-2-caboxylic acid.

Mandol (cefamandole narate) is commercially available from Lilly and is [6R-[6α-7β(R*)]]-7-[[(formyloxy)phenylacetyl]amino]-3-[[(1-methyl-1H-tetrazol-5-yl)thio]-methyl]-8-oxo-5-thia-1-azabicyclo[4.2.0]oct-2-ene-2-carboxylic acid, mono-sodium salt.

Maxipime (cefepime HCl) is commercially available from Bristol-Myers Squibb and is 1-[[6R,7R)-7-[2-amino-4-thiazolyl)-glyoxylamido]-2-carboxy-8-oxo-5-thia-1-aza-bicyclo-[4.2.0]-oct-2-en-3-yl]methyl]-1-methylpyrrolidinium chloride, 7²-(Z)-(O-methyl-oxime), monohydrochloride, monohydrate.

Monocid (cefonicid sodium) is commercially available from SmithKline Beecham and is [6R-[6α,7β(R*)]]-[(hydroxyphenyl-acetyl)-amino]-8-oxo-3-[[1-(sulfo-methyl)-1H-tetrazol-5-yl] 30 thio-methyl]-5-Thia-1-azabicyclo[4.2.0]oct-2-ene-2-carboxylic acid, disodium salt.

Omnicef (cefdinir) is commercially available from Parke Davis and is [6R-[6α,7β(Z)]]-7-[[(2-amino-4-thiazolyl)(hydroxyimino)acetyl]amino]-3-ethenyl-8-oxo-5-thia-1-azabicyclo[4.2.0]-oct-2-ene-2-carboxylic acid.

Rocephin (ceftriaxone) is commercially available from Roche Laboratories and is (6R,7R)-7-[2-(2-Amino-4-thiazolyl)glyoxylamido]-8-oxo-3-[[(1,2,5,6-tetrahydro-2-methyl-5,6-di-oxo-as-triazin-3-yl)thio]methyl]-5-thia-1-azabicyclo-[4.2.0]-oct-2-ene-2-carboxylic acid, 7²-(Z)-O-methyloxime), disodium salt, sesquaterhydrate.

Suprax (ceftixime) is commercially available from Lederle Laboratories and is (6R,7R)-7-[2-(2-amino-4-thiazolyl)glyoxylamido]-8-oxo-3-vinyl-5-thia-1-azabicyclo-[4.2.0]-oct-2-ene-2-carboxylic acid, 7²-(Z)-[O-(carboxymethyl)oxime]trihydrate.

Tazicef (ceftazidime) is commercially available from SmithKline Beecham and is a pyridinium, [6R,[6α,7β(Z)]]-1-[[7-[[2-amino-4-thiazolyl)[(1-carboxy-1-methylethoxy)-imino]acetyl]amino]-2-carboxy-8-oxo-5-thia-1-azabicyclo-(4.2.0)-oct-2-en-3-yl]methyl]-hydroxide, inner salt.

Tazidime (cefiazidime) is commercially available from Lilly and is pentahydrate of Pyridinium, 1-[[7-[[2-amino-4-thiazolyl)[(1-carboxy-1-methylethoxy)imino]acetyl]-amino]-2-carboxy-8-oxo-5-thia-1-azabicyclo(4.2.0)oct-2-en-3-yl]methyl]hydroxide, inner salt, [6R,[6α,7β(Z)]].

Vantin (cefpodoxime proxetil) is commercially available from Pharmacia & Upjohn and is (RS)-I-(isoproproxycarbonyloxy)ethyl-(+)-(6R,7R)-7-[2-(2-amino-4-thiazolyl)-2-{(Z)-methoxyimino}acetamido]-3-methoxymethyl-8-oxo-5-thia-1-azabicyclo[4.2.0]oct-2-ene-2-carboxylate.

Zinacef (cefuroxime) is commercially available from Glaxo Wellcome and is (6R,7R)-3-carbamoyloxymethyl-7-[Z-2-methoxy-imino-2-fur-2-yl)-acetamido]-ceph-3-em-4-carboxylate sodium salt.

Biaxin (clarithromycin) is commercially available from Abbott and is 6-O-methyl-erythromycin.

Dynabac (dirithromycin) is commercially available from Sanofi and is (9S)-9-Deox-11-deoxy-9,11-[imino [(1R)-2-(2-methoxyethoxy)-ethylidene]oxy]erythromycin.

E.E.S. 200 (Erythromycin Ethylsuccinate) is commercially available from Abbott and is erythromycin 2′-(ethylsuccinate).

E.E.S. 400 (Erythromycin Ethylsuccinate) is commercially available from Abbott and is erythromycin 2′-(ethylsuccinate).

Ery-Ped 200 (Erythromycin Ethylsuccinate) is commercially available from Abbott and is erythromycin 2′-(ethylsuccinate).

EryPed 400 (Erythromycin Ethylsuccinate) is commercially available from Abbott and is erythromycin 2′-(ethylsuccinate).

Ery-Tab (Erythromycin delayed-release tablets) is commercially available from Abbott and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13-hexamethyl-6-[[3,4,6-trideoxy-3-(dimethylamino)-13-β-xylo-hexopyranosyl]oxy]oxacyclotetra-decane-2,10-dione. Erythrocin Stearate (Erythromycin stearate) is commercially available from Abbott and is the stearate salt of (3R*,4S*,5S*,6R*,7R*,9R*,11R*,12R*,13S*,14R*)-4-[(2,6-dideoxy-3-C-methyl-3-O-methyl-α-L-ribo-hexopyranosy)oxy]-14-ethyl-7,12,13-trihydroxy-3,5,7,9,11,13-hexamethyl-6-[[3,4,6-trideoxy-3-(dimethylamino)-β-D-xylo-hexopyranosyl]oxy]oxa-cyclotetradecane-2,10-dione.

Ilosone (erythromycin estolate) is commercially available from Dista and is erythromycin 2′-propionate, dodecyl sulfate.

PCE Dispertab (erythromycin particles in tablets) is commercially available from Abbott and is (3R*,4S*,5S*,6R*,7R*,9R*,11R*,12R*,13S*,14R*)-4-[(2,6-dideoxy-3-C-methyl-3-O-methyl-ot-L-ribo-hexopyranosyl)oxy]-14-ethyl-7,12,13-trihydroxy-3,5,7,9,11,13-hexa-methyl-6-[[3,4,6-trideoxy-3-(dimethylamino)-β-D-xylo-hexopyranosyl]oxy]oxa-cyclotetradecane-2,10-dione.

Pediazole (erythromycin ethylsuccinate and sulfisoxazole acetyl for oral suspension) is commercially available from Ross Products and is 2′-ethylsuccinyl ester of erythromycin (erythromycin ethylsuccinate) and N-(3,4-dimethyl-5-isoxazolyl)-N-sulfanilylacetamide (sulfisoxazole acetyl).

Tao (troleandomycin) is commercially available from Pfizer and is the synthetically derived acetylated ester of oleandomycin.

Zithromax (azithromycin) is commercially available from Pfizer and is (2R,3S,4R,5R,8R,10R,11R,12S,13S,14R)-13-[(2,6-dideoxy-3-C-methyl-3-O-methyl-a-L-ribo-hexo-pyranosyl)-oxy]-2-ethyl-3,4,10-trihydroxy-3,5,6,8,10,12,14-heptamethyl-11-[[3,4,6-trideoxy-3-(dimethyl-amino)-β-D-xylo-hexopyranosyl]oxy]-1-oxa-6-aza-cyclopenta-decan-15-one.

Erythromycin, which is (3R*,4S*,5S*,6R*,7R*,9R*,11R*,12R*,13S*,14R*)-4-[(2,6-di-deoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-O-β-D-xylo-hexopyranosyl]oxy]oxa-cyclotetradecane-2,10-dione.

Cleocin HCl (clindamycin hydrochloride) is commercially available from Pharmacia & Upjohn and is the hydrated hydrochloride salt of clindamycin, asemisynthetic antibiotic produced by a 7(S)-chloro-substitution of the (7R) hydroxyl group of lincomycin.

Cleocin Phosphate (clindamycin phosphate) is commercially available from Pharmacia & Upjohn and is L-threo-α-D-galacto-Octopyranoside, (2S-trans)-methyl-7-chloro-6,7,8-trideoxy-6-[[(1-methyl-4-propyl-2-pyrrolidinyl)carbonyl]amino]-1-thio-2-(dihydrogen phosphate).

Coly-Mycin M (colistimethate sodium) is commercially available from Monarch.

Vancocin HCl (vancomycin hydrochloride) is commercially available from Lilly.

Amoxil (amoxicillin) is commercially available from SmithKline Beecham and is (2S,5R,6R)-6-[R-(−)-2-amino-2-(p-hydroxyphenyl)acetamido]-3,3-dimethyl-7-oxo-4-thia-1-aza-bicyclo-[3.2.0]-heptane-2-carboxylic acid trihydrate.

Augmentin (amoxicillin/clavulanate potassium) is commercially available from SmithKline Beecham and is the trihydrate of (2S,5R,6R)-6-[R-(−)-2-amino-2-(p-hydroxyphenyl)acetamido]-3,3-dimethyl-7-oxo-4-thia-1-azabicyc-[3.2.0]-heptane-2-carboxylic acid (amoxicillin) and potassium (Z)-(2R,5R)-3-(2-hydroxyethylidene)-7-oxo-4-oxa-1-aza-bicyclo-[3.2.0]-heptane-2-carboxylate (clavulanate potassium).

Bicillin C-R 900/300 (Penicillin G benzathine and Penicillin G procaine suspension) is commercially available from Wyeth-Ayerst and is (2S,5R,6R)-3,3-Dimethyl-7-oxo-6-(2-phenyl-acetamido)-4-thia-1-azabicyclo-[3.2.0]-heptane-2-carboxylic acid compound with N,N′-dibenzyl-ethylenediamine (2:1), tetrahydrate (Penicillin G benzathine) and (2S,5R,6R)-3,3-Dimethyl-7-oxo-6-(2-phenylacetamido)₄-thia-1-azabicyclo-[3.2.0]-heptane-2-carboxylic acid compound with 2-(diethyl-amino)ethyl p-amino benzoate compound (1:1) monohydrate (Penicillin G procaine).

Bicillin C-R (Penicillin G benzathine and Penicillin G procaine suspension) is commercially available from Wyeth-Ayerst and is (2S,5R,6R)-3,3-Dimethyl-7-oxo-6-(2-phenyl-acetamido)-4-thia-1-azabicyclo-[3.2.0]-heptane-2-carboxylic acid compound with N,N′-dibenzyl-ethylenediamine (2:1), tetrahydrate (Penicillin G benzathine) and (2S,5R,6R) 3,3-Dimethyl-7-oxo-6-(2-phenylacetamido)₄-thia-1-azabicyclo-[3.2.0]-heptane-2-carboxylic acid compound with 2-(diethylamino)ethyl p-amino benzoate compound (1:1) monohydrate(Penicillin G procaine).

Bicillin L-A (Penicillin G benzathine suspension) is commercially available from Wyeth-Ayerst and is (2S,5R,6R)-3,3-Dimethyl-7-oxo-6-(2-phenylacetamido)-4-thia-1-azabicyclo-[3.2.0]-heptane-2-carboxylic acid compound with N,N′-dibenzylethylene-di-amine (2:1), tetrahydrate.

Geocillin (carbencillin indanyl sodium) is commercially available from Pfizer and is 1-(5-Indanyl)-N-(2-carboxy-3-3-dimethyl-7-oxo-4-thia-1-azabicyclo-[3.2.0]-hept-6-yl)-2-phenyl-malonamate monosodium salt.

Mezlin (sterile mezlocillin sodium) is commercially available from Bayer and is the monohydrate sodium salt of 6-{D-213[(methyl-sulfonyl)-2-oxo-imidazolidine-1-carboxamido]-2-phenylacetamido}penicillanic acid.

Omnipen (ampicillin) is commercially available from Wyeth-Ayerst and is (2S,5R,6R)-6-[R-2-Amino-2-phenylacetamido]-3,3-dimethyl-7-oxo-4-thia-1-aza-bicyclo-[3.2.0]-heptane-2-carboxylic acid.

Pen-Vee K (penicillin V potassium) is commercially available from Wyeth-Ayerst and is the potassium salt of the phenoxymethyl analog of penicillin G.

Pfizerpen (penicillin G potassium) is commercially available from Pfizer and is monopotassium 3,3-dimethyl-7-oxo-6-(2-phenylacetamido)-4-thia-1-azabicyclo-(3.2.0)-heptane-2-carboxylate.

Pipracil (piperacillin sodium) is commercially available from Lederle and is the monosodium salt of [2S-[2α,5α,6β(S*)]]-6-[[[[(4-ethyl-2-3-dioxo-1-piperazinyl)-carbonyl]-amino]phenylacetyl]amino]-3,3-di-methyl-7-oxo-4-thia-1-azabicyclo-[3.2.0]-heptane-2-carboxylic acid, monosodium salt.

Spectrobid (bacampicillin HCl) is commercially available from Pfizer and is 1′-ethoxy-carbonyloxyethyl-6-(D-α aminophenylacetamide)penicillate hydrochloride.

Ticar (ticarcillin disodium) is commercially available from SmithKline Beecham and is the disodium salt of N-(2-carboxy-3,3-dimethyl-7-oxo-4-thia-1-azabicyclo-[3.2.0]-hept-6-yl)-3-thiophenemalonamic acid.

Timentin (ticarcillin disodium and clavulanate potassium) is commercially available from SmithKline Beecham and is N-(2-carboxy-3,3-dimethyl-7-oxo-4-thia-1-azabicyclo-[3.2.0]-hept-6-yl)-3-thiophenemalonamic acid disodium salt (ticarcillin disodium) and potassium (Z)-(2R,5R)-3-(2-hydroxyethylidene)-7-oxo-4-oxa-1-aza-bicyclo-[3.2.0]-heptane-2-carboxylate (clavulanate potassium).

Unasyn (ampicillin sodium/sulbactam sodium) is commercially available from Pfizer and is monosodium (2S,5R,6R)-6-[R-2-Amino-2-phenyl acetamido]-3,3-dimethyl-7-oxo-4-thia-1-aza-bicyclo-[3.2.0]-heptane-2-carboxylate (amipicillin sodium) and sodium penicillate sulfone; sodium (2S,5R)-3,3-dimethyl-7-oxo-4-thia-1-azabicyclo-[3.2.0]-heptane-2-carboxylate-4,4-di-oxide (sulbactam sodium).

Zosyn (piperacillin sodium and tazobactam sodium) is commercially available from Lederle and is sodium (25,5R,6R)-6[R-2-(4-ethyl-2,3-dioxo-1-piperazine-carboxamido)-2-phenyl-acetamido]-3,3-dimethyl-7-oxo-4-Thia-1-azabicylco-[3.2.0]-heptane-2-carboxylate (piperacillin) and sodium (2S,3S,5R)-3-methyl-7-oxo-3-(1H-1,2,3-triazol-1-ylmethyl)-4-thia-1-azabicyclo-[3.2.0]-heptane-2-carboxylate-4,4-dioxide (tazobactam).

Dicloxacillin Sodium is monosodium (2S,5R,6R)-6-(3-(2,6-dichlorophenyl)5-methyl-4-isoxazolecarboxamido]-3,3-dimethyl-7-OXO-4-thia-1-azabicyclo-[3.2.0]-heptane-2-carboxylate monohydrate.

Achromycin V (tetracycline HCl) is commercially available from Lederic and is the monohydrochloride of [4S-(4α,4aα,5aα,6β,12aα,)]-4-(dimethylamino)-1,4,4a,5,5a,6,11,12a-octa-hydro-3,6,10,12,12a-pentahydroxy-6-methyl-1,11-dioxo-2-naphthacenecarboxamide.

Declomycin (demeclocycline HCl) is commercially available from Lederle Laboratories and is 7-chloro-4-dimethylamino-1,4,4,4a,5,5a,6,11,12a-octahydro-3,6,10,12,12a-pentahydroxy-1,11-dioxo-2-naphthaeenecarboxamide monohydro-chloride.

Dynacin (minocylcine HCl) is commercially available from Medicis and is [4S-(4α,4aα,5aα,12aα)]-4,7-bis(dimethylamino)-1,4,4a,5,5a,6,11,12a-octahydro-3,10,12,12a-tetrahydroxy-1,11-dioxo-2-napthacene carboxamide monochloride.

Minocin (minocycline hydrochloride) is commercially available from Lederle Laboratories and is [4S-(4a,4aα,5aα,12aα)]-4,7-bis(dimethylamino)-1,4,4a,5,5a,6,11,12a-octa-hydro-3,10,12,12a-tetrahydroxy-1,11-dioxo-2-napthacene carboxamide mono-chloride.

Monodox (Doxycycline monohydrate capsules) is commercially available from Oclassen and is α-6-deoxy-5-oxytetracycline.

Terramycin (oxytetracyline) is commercially available from Pfizer.

Vectrin (minocycline hydrochloride) is commercially available from Warner Chilcott Professional Products and is the monochloride of [4S-(4α,4aα,5aα,12ax)]-4,7-bis-(di-methylamino)-1,4,4a,5,5a,6,11,12a-octahydro-3,10,12,12a-tetrahydroxy-1,11-dioxo-2-napthacenecarboxamide monochloride.

Vibramycin Calcium (doxycycline sodium) is commercially available from Pfizer and is the monohydrate of 4-(Dimethylamino)-1,4,4a,5,Sa,6,11,12a-octahydro-3,5,10,12,12a-pentahydroxy-6-methyl-1,11-dioxo-2-napthacene-carboxamide.

Vibramycin Hyclate (doxycycline hyclate) is commercially available from Pfizer and is α-6-deoxy-5-oxytetracycline.

Vibramycin Monohydrate (doxycycline monohydrate) is commercially available from Pfizer and is 4-(Dimethylamino)-1,4,4a,5,5a,6,11,12a-octahydro-3,5,10,12,12a-pentahydroxy-6-methyl-1,11-dioxo-2-napthacene-carboxamide monohydrate.

Vibra-Tabs (doxycycline hydrate) is commercially available from Pfizer and is a-6-deoxy-5-oxytetracycline.

Vibramycin (doxycycline) is commercially available from Pfizer and is 4-(Dimethylamino)-1,4,4a,5,5a,6,11,12a-octahydro-3,5,10,12,12a-pentahydroxy-6-methyl-1,11-di-oxo-2-napthacene-carboxamide monohydrate.

Lincomycins is monosodium (2S,5R,6R),6-(3-(2,6-dichlorophenyl)5-methyl-4-isoxazole-carboxamido]-3,3-dimethyl-7-oxo-4-thia-1-azabicyclo-[3.2.0]-heptane-2-carboxylate monohydrate.

Cleocin HCl (clindamycin HCl) is commercially available from Pharmacia & Upjohn and is the monohydrochloride of methyl 7-chloro-6,7,8-trideoxy-6-(1-methyl-trans-4-propyl-L-2-pyrrolidinecarboxamido)-1-thio-L-threo-α-D-galacto-octopyranoside.

Abelcet (amphotericin B lipid complex) is commercially available from Libosome Company, Inc. and is [1R-(1R*,3S*,5R*,6R*,9R*,11R*,15S*,16R*,17R*,18S*,19E,21E,23E,25E,27E,29E,31E,33R*,35S*,36R*,37S*)]-33-[(3-amino-3,6-dideoxy-β-D-manno-pyranosyl)oxy]-1,3,5,6,9,11,17,37-octahydroxy-15,16,18-trimethyl-13-oxo-14,39-dioxabi-cyclo-[33.3.1]-nonatriaconta-19,21,23,25,27,29,31-heptaene-36-carboxylic acid.

AmBisome (amphotericin B) is commercially available from Fujisawa Healthcare and is [1R-(1R*,3S*,5R*,6R*,9R*,11R*,15S*,16R*,17R*,18S*,19E,21E,23E,25E,27E,29E,31E,33R*,35S*,36R*,37S*)]-33-[(3-amino-3,6-dideoxy-β-D-manno-pyranosyl)-oxy]-1,3,5,6,9,11,17,37-octahydroxy-15,16,18-trimethyl-13-oxo-14,39-dioxa-bicyclo-[33.3.1]-nona-triaconta-19,21,23,25,27,29,31-heptaene-36-carboxylic acid.

Amphotee (amphotericin B cholesterol sulfate complex) is commercially available from Sequus Pharmaceuticals, Inc. and is [1R-(1R*,3S*,5R*,6R*,9R*,11R*,15S*,16R*,17R*,18S*,19E,21E,23E,25E,27E,29E,31E,33R*,35S*,36R*,37S*)]-33-[(3-amino-3,6-di-deoxy-β-D-manno-pyranosyl)-oxy]-1,3,5,6,9,11,17,37-oetahydroxy-15,16,18-trimethyl-13-oxo-14,39-dioxabicyclo-[33.3.1]-nonatriaconta-19,21,23,25,27,29,31-heptaene-36-carboxylic acid.

Ancobon (flucytosine) is commercially available from ICN Pharmaceuticals and is 5-fluorocytosine.

Diflucan (fluconazole) is commercially available from Pfizer Inc. and is 2,4-difluoro-α-α′-bis(1H-1,2,4-triazol-1-ylmethyl)benzyl alcohol.

Fulvicin P/G (ultramicrosize griseofulvin) is commercially available from Schering.

Fulvicin P/G 165 and 330 (ultramicrosize griseofulvin) is commercially available from Schering.

Grifulvin V (griseofulvin) is commercially available from Ortho Dermatological.

Gris-PEG (griseofulvin ultramicrosize) is commercially available from Allergan.

Lamisil (terbinafine hydrochloride) is commercially available from Novartis and is (E)-N-(6,6-dimethyl-2-hepten-4-ynyl)-N-methyl-1-naphthalenemethanamine hydrochloride.

Nizoral (ketoconazole) is commercially available from Janssen and is cis-1-acetyl-4-[4-[[2-(2,4-di-chlorophenyl)-2-(1H-imidazol-1-ylmethyl)-1,3-dioxolan-4-yl]methoxy]-phenyl]piperazine.

Amphotericin B is [1R-(1R*,3S*,5R*,6R*,9R*,11R*,15S*,16R*,17R*,18S*,19E,21E,23E,25E,27E,29E,31E,33R*,35S*,36R*,37S*)]-33-[(3-amino-3,6-dideoxy-β-D-mannopyranosyl)-oxy]-1,3,5,6,9,11,17,37-octahydroxy-15,16,18otrimethyl-13-oxo-14,39-dioxabicyclo-[33.3.1]-nonatriaconta-19,21,23,25,27,29,31-heptaene-36-carboxylic acid.

Lotrimin (clotrimazole) is commercially available from Schering and is 1-(O-chloro-α,α-diphenylbenzyl)imidazole.

Dapsone tablets (dapsone) is commercially available from Jacobus and is 4,4′-diaminodi-phenylsulfone (DDS).

Diflucan (fluconazole) is commercially available from Pfizer and is 2,4-difiuoro-α-α′-bis(1H-1,2,4-triazol-1-ylmethyl)benzyl alcohol.

Monistat-Derm cream (miconazole) is commercially available from Ortho Dermatological and is 1-[2,4-dichloro-β-{(2,4-dichlorobenzyl)oxy}phenethyl]imidazole mononitrate.

Mycostatin Cream (nystatin) is commercially available from Westwood-Squibb.

Sporanox (itraconazole) is commercially available from Janssen Pharmaceutical and is (±)-1-[(R*)-sec-butyl]4-[p-[[2R*,4S*)-2-(2,4-dichlorophenyl)-2-(1H-1,2,4-triazol-1-yl-methyl)-1,3-dioxolan-4-yl]methoxy]phenyl]-1-piperazinyl]phenyl]-Δ²-1,2,4-triazolin-5-one mixture with (±)-1-[(R*)-sec-butyl]-4-[p-[[2R*,4S*)-2-(2,4-dichlorophenyl)-2-(1H-1,2,4-triazol-1-ylmethyl)-1,3-dioxolan-4-yl]methoxy]phenyl]-1-piperazinyl]phenyl]-Δ²-1,2,4-triazolin-5-one or (±)-1-[(RS)-sec-butyl]-4-[p-[4-[p-[[(2R,4S)-2-(2,4-dichlorophenyl)-2-(1H-1,2,3,4-triazol-1-ylmethy)-1,3-dioxolan-4-yl]methoxy]phenyl]-1-piperazinyl]phenyl]-Δ²-1,2,4-triazolin-5-one.

Aralen hydrochloride (chloroquine) is commercially available from Sanofi Pharmaceuticals and is the dihydrochloride of 7-(chloro-4-[[4-diethylamino)-1-methylbutyl]-amino]quinoline.

Aralen phosphate (chloroquine phosphate) is commercially available from Sanofi Pharmaceuticals and is 7-(chloro-4-[[4-diethylamino)-1-methylbutyl]amino]-quinoline phosphate (1:2).

Daraprim (pyrimethamine) is commercially available from Glaxo Wellcome and is 5-(4-chlorophenyl)-6-ethyl-2,4-pyrimidinediamine.

Lariam (mefloquine HCl) is commercially available from Roche Laboratories and is (R*,S*)-(±)-α-2-piperidinyl-2,8-bis(trifluoromethyl)-4-quinoline methanol hydro-chloride.

Plaquenil (hydroxychloroquine sulfate) is commercially available from Sanofi Pharmaceuticals and is 2-[[4-[7-chloro-4-quinolyl)amino]pentyl]ethylamino]ethanol sulfate (1:1).

Capastat sulfate (capreomyein sulfate) is commercially available from Dura Pharmaceuticals.

Myambutol (ethambutol hydrochloride) is commercially available from Lederle Laboratories.

Mycobutin (rifabutin capsules) is commercially available from Pharmacia & Upjohn and is 1′,4-didehydro-1-deoxy-1,4-dihydro-5′-(2-methylpropyl)-1-oxorifamycin XIV or (9S,12E,14S,15R,16S,17R,18R,19R,20S,21S,22E,24Z)-6,16,18,20-tetrahydroxy-1-1′-isobutyl-14-methoxy-7,9,15,17,19,21,25-heptamethyl-spiro[9,4-(epoxypentadeca-1,11,13-tri-enimino)-2H-furo-2′,3′:7,8-naphth-[1,2-d]-imidazole-2,4′-piperidine]-5,10,26-(3H,9H)-trione-16-acetate.

Nydrazid (isoniazid injection) is commercially available from Apothecon.

Paser (aminosalicylic acid) is commercially available from Jacobus and is 4-amino-2-hydroxybenzoic acid.

Priftin (rifapentine) is commercially available from Hoechst Marion Roussel and is rifamycin 3-[[(4-cyclo-pentyl-1-piperazinyl)imino]methyl] or 3[N-(4-cyclopentyl-1-piperazinyl)-formimyidoyl]-2,7-(epoxypentadeca[1,11,13]trienimino)naphtha[2,1-b]-furan-1,11(2H)-dione-21-acetate.

Pyrazinamide tablets (pyrazinamide) is commercially available from Lederle Laboratories and is the pyrazine analogue of nicotinamide.

Rifadin (rifampin capsules) is commercially available from Hoechst Marion Roussel and is 3-[[(4-methyl-1-piperazinyl)imino]methyl]rifamycin or 5,6,9,17,19,21-hexahydroxy-23-methoxy-2,4,12,16,20,22-heptamethyl-8-[N-methyl-1-piperazinyl)-formimidoyl]-2,7-(epoxypentadeca-[1,11,13]-trienimino)naptho[2,1-b]furan-1,1′-(2H)-dione 21-acetate.

Rifadin IV (rifampin for injection) is commercially available from Hoechst Marion Roussel and is 3-[[3-(4-methyl-1-piperazinyl) formimidoyl]-2,7-(epoxypenta-deca[1,11,13]-trienimino)naphtho[2,1-b]furan-1,11-(2H)-dione-21-acetate.

Rifamate (rifampin and isoniazid) is commercially available from Hoechst Marion Roussel and is 3-(4-methyl-1-piperazinyliminomethyl) rifamycin SV (rifampin) and hydrazide of isonicotinic acid (isoniazid).

Rifater (rifampin, isoniazid and pyrazinamide) is commercially available from Hoechst Marion Roussel and is 3-(4-methyl-1-piperazinyliminomethyl) rifamycin SV (rifampin), hydrazide of isonicotinic acid (isoniazid) and pyrazine analogue of nicotinamide (pyrazinamide).

Seromycin (cycloserine capsules) is commercially available from Dura Pharmaceuticals and is R-4-amino-3-isoxazolidinone.

Streptomycin Sulfate is commercially available from Pfizer and is O-2-deoxy-2-(methylamino)-α-L-glucopyranosyl-(1→2)-O-5-deoxy-3-C-formyl-α-L-lyxofuranosyl-(1→4)-N-N′-bis(aminoiminomethyl)-, sulfate (2:3) salt.

Tice BCG (BCG vaccine) is commercially available from Organon and is attenuated live Mycobacterium bonis strains Bacillus of Calmette and Guerin.

Cycloserine (seromycin capsules) is commercially available from Dura Pharmaceuticals and is R-4-amino-3-isoxazolidinone.

Nydrazid (Isoniazid) is commercially available from Apothecon and is the hydrazide of isonicotinic acid.

Urised (Methenamine) is commercially available from Poly Medica.

Trecator-SC (ethionamide tablets) is commercially available from Wyeth-Ayerst and is 2-ethylthioisoniocotinamide.

Alferon N (interferon alfa-n3) is commercially available from Interferon Sciences and is interferon alfa-n3 (human leukocyte derived).

Crixivan (indinavir sulfate) is commercially available from Merck & Co., Inc. and is [1(1S,2R),5(S)]-2,3,5-trideoxy-N-(2,3-dihydro-2-hydroxy-1H-inden-1-yl)-5-[2-[[1,1-dimethylethyl)amino]carbonyl]-4-(3-pyridinyl-methyl)-1-piperazinyl]-2-(phenylmethyl)-D-erythropentonamide sulfate (1:1).

Cytovene (ganciclovir) is commercially available from Roche and is 9-[[2-hydroxy-1-(hydroxymethyl)ethoxy]methyl]guanine.

Cytovene-IV (ganciclovir sodium) is commercially available from Roche and is 9-[[2-hydroxy-1-(hydroxymethyl)ethoxy]methyl]guanine.

Epivir (lamivudine) is commercially available from Glaxo Wellcome and is (2R,cis)-4-amino-1-(2-hydroxymethyl-1,3-oxathiolan-5-yl)-1H)-pyrimidin-2-one.

Famvir (famciclovir) is commercially available from SmithKline Beecham and is 2-[2-(2-amino-9H-purin-9-yl) ethyl]-1,3-propanediol diacetate.

Flumadine (rimantadine HCl) is commercially available from Forest and is alpha-methyltricyclo-[3.3.1.1/3.7]decane-1-methanamine hydrochloride.

Foscavir (foscarnet sodium) is commercially available from Astra and is phosphonoformic acid, trisodium salt.

Hivid (zalcitabine) is commercially available from Roche and is 4-amino-1-β-D-2′,3′-dideoxyribofuranosyl-2-(1H)-pyrimidone or 2′, 3′, dideoxyribofuranosyl-2-(1H)-pyrimidone or 2′,3′-dideoxycytidine.

Intron A (interferon alfa-2b) is commercially available from Schering.

Invirase (saquinavir mesylate) is commercially available from Roche Labs and is N-tert-butyl-decahydro-2-[2R-hydroxy-4-phenyl-3(S)-[[N-(2-quinolylcarbonyl)-L-asparaginyl]amino]butyl-(4aS,8aS)-isoquinoline-3(S)-carboxamide methanesulfonate.

Norvir (ritonavir) is commercially available from Abbott and is [5S-(5R*,8R*,10R*,11R*)]-10-Hydroxy-2-methyl-5-(1-methylethyl)-1-[2-(1-methylethyl)-4-thiazolyl]-3,6-di-oxo-8,11-bis(phenyl-methyl)-2,4,7,12-tetraazatridencan-B-oic acid, 5-thiazolylmethyl ester.

Rebetron combination therapy, which contains Rebetrol (ribavirin which is 1-β-D-ribofuranosyl-1H-1,2,4-triazole-3-carboxamide) and Intron A (inteferon alfa-2b), is commercially available from Schering.

Rescriptor (delavirdine mesylate) is commercially available from Pharmacia & Upjohn and is piperazine, 1-[3-[(1-methylethyl)amino]-2-pyridinyl]-4-[[5-(methyl-sulfonyl)-amino]-1H-indol-2-yl]carbonyl], monomethanesulfonate.

Retrovir (ziduvudine) is commercially available from Glaxo Wellcome and is 3′-azido-3′-deoxythymidine.

Retrovir IV (ziduvudine) is commercially available from Glaxo-Wellcome and is 3′-azido-3′-deoxythymidine.

Symmetrel (amantadine hydrochloride) is commercially available from MedImmune Inc. and is humanized monoclonal antibody (IgG1ₓ).

Valtrex (valacyclovir HCl) is commercially available from Glaxo Wellcome and is L-valine, 2-[(2-amino-1,6-dihydro-6-oxo-9H-purin-9-yl)methoxy]ethyl ester, monohydrochloride.

Videx (didanosine) is commercially available from Bristol-Myers Squibb Oncology/Immunology and is 2′,3′-di-deoxyinosine.

Viracept (nelfinavir mesylate) is commercially available from Agouron and is [3S-[2(2S*,3S*),3a,4ap,8ap]]-N-(1,1-dimethylethyl)decahydro-2-[2-hydroxy-3-[3-hydroxy-2-methyl-benzoyl)amino]4-(phenylthio)butyl]-3-isoquinolinecarboxcamide monomethanesulfonate (salt).

Viramune (nevirapine) is commercially available from Roxane and is 11-cyclopropyl-5,11-dihydro-4-methyl-6H-dipyrido[3,2-b:2′,3′-][1,4]diazepin-6-one.

Virazole (ribavirin) is commercially available from ICN and is 1-beta-D-ribofuranosyl-1H-1,2,4-triazole-3-carboxamide.

Vistide (cidofovir) is commercially available from Gilead Sciences and is 1-[(S)-3-hydroxy-2-(phosphonomethoxy)propyl]cytosine dihydrate (HPMPC).

Zerit (stavudine (d4T)) is commercially available from Bristol-Myers Squibb Oncology/Immunology and is 2′,3′-didehydro-3′deoxythymidine.

Symmetrel Syrup (amantadine HCl) is commercially available from Endo Labs and is 1-adamantanamine hydrochloride.

Combivir Tablets (lamiduvine) is commercially available from Glaxo Wellcome and is 2′,3′-didehydro-3′-deoxythymidine.

Zovirax (acyclovir) is commercially available from Glaxo Wellcome and is 2-amino-1,9-dehydro-9-[(2-hydroxyethyoxy)methyl]-6H-purin-6-one.

Dapsone Tablets (dapsone) is commercially available from Jacobus and is 4,4′-diaminodiphenylsulfone (DDS).

Daraprim (pyrimethamine) is commercially available from Glaxo Wellcome and is 5-(4-chlorophenyl)-6-ethyl-2,4-pyrimidinediamine.

Flagyl 375 (metronidazole) is commercially available from Searle and is 2-Methyl-5-nitro-imidazole-1-ethanol.

Flagyl ER Tablets (metronidazole) is commercially available from Searle and is 2-Methyl-5-nitro-imidazole-1-ethanol.

Flagyl I.V. (metronidazole) is commercially available from SCS and is 2-Methyl-5-nitro-imidazole-1-ethanol.

Furoxone (furazolidone) is commercially available from Roberts and is 3-(5-nitrofurfuryliden-amino)-2-oxazolidinone.

Mepron (atovaquone) is commercially available from Glaxo Wellcome and is trans-2-[4-(4-chlorophenyl)cyclohexyl]-3-hydroxy-1,4-naphthalenedione.

Neutrexin (trimetrexate glucuronate) is commercially available from U.S. Bioscience and is 2,4-diamino-5-methyl-6-[(3,4,5-trimethoxyanilino)methyl]quinazoline mono-D-glucuronate.

Cipro (ciprofloxacin HCl) is commercially available from Bayer and is the monohydrochloride monohydrate salt of 1-cyclopropyl-6-fluoro-1,4-dihydro-4-oxo-7-1(1-piperazinyl)-3-quinolinecarboxylic acid.

Floxin (ofloxacin) is commercially available from Ortho-McNeil Pharmaceutical and is (±)-9-fluoro-2,3-dihydro-3-methyl-10-(4-methyl-1-piperazinyl)-7-oxo-7H-pyrido-[1,3,3-de]-1,4-benzoxazine-6-carboxylic acid.

Levaquin (levofloxacin) is commercially available from Ortho-McNeil Pharmaceutical) and is (−)-(S)-9-fluoro-2,3-dihydro-3-methyl-10-(4-methyl-1-piperazinyl)-7-oxo-7H-pyrido-[1,2,3-de]-1,4-benzoxazine-6-carboxylic acid hemi-hydrate.

Mazaquin (lomefloxacin HCl) is commercially available from Unimed and is monohydrochloride salt of (±)-1-ethyl-6,8-difluoro-1,4-dihydro-7-(3-methyl-1-piperazinyl)-4-oxo-3-quinolinecarboxylic acid.

Noroxin (norfloxacin) is commercially available from Merck and is 1-ethyl-6-fluoro-1,4-dihydro-4-oxo-7-(1-piperazinyl)-3-quinolinecarboxylic acid.

Penetrex (enoxacin) is commercially available from Rhone-Poulenc Rorer and is 1-ethyl-6-fluoro-1,4-dihydro-4-oxo-7-(1-piperazinyl)-1,8-naphthyridine-3-carboxylic acid sesquihydrate.

Raxar (grepafloxacin HCl) is commercially available from Glaxo Wellcome and is (±)-1-cyclopropyl-6-fluoro-1,4-dihydro-5-methyl-7-(3-methyl-1-piperazinyl)-4-oxo-3-quinolinecarboxylic acid monochloride sesquihydrate.

Trovan (trovafloxacin mesylate) is commercially available from Pfizer and is (1α, 5α,6α)-7-(6-amino-3-azabicyclo[3.1.0]hex-3-yl)-1-(2,4-difluorophenyl)-6-fluoro-1,4-di-hydro-4-oxo-1,8-naphthyridine-3-carboxylic acid, monomethanesulfonate.

Zagam (sparfloxacin) is commercially available from Rhône-Poulenc Rorer and is 5-amino-1-cyclopropyl-7-cis-3,5-dimethyl-1-piperazinyl)-6,8-difluoro-1,4,dihydro-4-oxo-3-quinolinecarboxylic acid.

Bactrim (trimethoprim and sulfamethoxazole) is commercially available from Roche Labs and is 2,4-diamino-5-(3,4,5-trimethoxybenzyl) pyrimidine (trimethoprim) and N¹-(5-methyl-3-isoxazolyl)sulfanilamide (sulfamethoxazole).

Bactrim DS (trimethoprim and sulfamethoxazole double strength) is commercially available from Roche Labs and is 2,4-diamino-5-(3,4,5-trimethoxybenzyl)pyrimidine (trimethoprim) and N¹-(5-methyl-3-isoxazolyl)sulfanilamide (sulfamethoxazole).

Pediazole (erythromycin ethylsuccinate and sulfisaxazole acetyl) is commercially available from Ross and is erythromycin 2′-(ethylsuccinate) and N′-acetylsulfisoxazole (sulfisoxizole is N-(3,4-dimethyl-5-isoxazolyl)-N-sulfanilyl acetamide.

Septra (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfa-methoxazole).

Septra DS (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfa-methoxazole).

Co-trimoxazole is a combined chemotherapeutic agent consisting of trimethoprim (T) and the sulphonamide sulphamethoxazole (S); their ratio is 1:5. It is bactericidal by virtue of a sequential blockade of the folic acid synthesis in microorganisms. The antimicrobial spectrum of co-trimoxazole includes many Gram-positive and Gram-negative aerobes, Chlamydias, nocardias, protozoa (pneumocystis carinii), etc. In addition to its use for pneumocystis, co-trimoxazole mainly has practical importance against Gram-positive aerobes (urinary tract infections), pneumococci and haemophilus influenza (respiratory tract infections and otitis). http://www.infomed.org/100drugs/ctrifram.html.

Bactrim I.V. Infusion (sulfamethoxazole) is commercially available from Roche Labs.

Pediazole (erythromycin ethylsuccinate and sulfisoxazole acetyl) is commercially available from Ross and is erythromycin 2′-(ethyl succinate) and N′ acetyl sulfisoxazole (sulfisoxizole is N-(3,4-Dimethyl-5-isoxazolyl)-N-sulfanilyl acetamide.

Furadantin (nitrofurantoin) is commercially available from Dura and is 1-[[(5-nitro-2-furanyl)methylene]amino]-2,4-imidazolidinedione.

Macrobid (nitrofurantoin monohydrate macrocrystals) is commercially available from Procter & Gamble Pharmaceuticals and is 1-[[[5-nitro2-furanyl]methylene]amino]-2-4-imidazolidinedione monohydrate.

Macrodantin (nitrofurantoin macrocrystals) is commercially available from Procter & Gamble Pharmaceuticals and is 1-[[[5-nitro-2-furanyl]methylene]amino]-24-imidazolidine-dione.

Monurol Sachet (fosfomycin tromethamine) is commercially available from Forest and is (1R,2S)-(1,2-epoxypropyl) phosphonic acid, compound with 2-amino-2-(hydroxymethyl)-1,3-propanediol (1:1).

NegGram Caplets (nalidixic acid) is commercially available from Sanofi and is 1-ethyl-1,4-dihydro-7-methyl-4-oxo-1,8-naphthyridine-3-carboxylic acid.

Septra (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfa-methoxazole).

Septra DS (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfa-methoxazole).

Urised (a combination of the antiseptics methenamine, methylene blue, phenyl salicylate, benzoic acid and parasympatholytics (atropine sulfate)hyoscyamine) is commercially available from Poly Medica.

Urobiotic-250 Capsules (oxytetracycline HCl, sulfamethizole and phenazopyridine HCl) is commercially available from Pfizer.

Uroqid Acid No. 2 Tablets (methenamine mandelate) is commercially available from Beach.

Bactroban (mupirocin) is commercially available from SmithKline Beecham and is (αE,2S,3R,4R,5S)-5-[(2S,3S,4S,5S)-2,3-Epoxy-5-hydroxy-4-methylhexyl]tetrahydro-3,4-dihydroxy-β-methyl-2H-pyran-2-crotonic acid, ester with 9-hydroxynonanoic acid, calcium salt (2:1), dihydrate.

Chloromycetin opthalmic (chloramphenical) is commercially available from Monarch and is (1) 2,2-dichloro-N-[2-hydroxy-1-(hydroxymethyl)-2-(4-nitrophenyl) ethyl]acetamide and (2) D-threo-(−)-2,2-Dichloro-N-[β-hydroxy-α-(hydroxymethyl)-p-nitrophenethyl]acetamide.

Cortisporin (neomycin and polymyxin β sulfates and hydrocortisone acetate cream) is commercially available from Monarch and is 21-(acetyloxy)-11β,17-dihydroxypregn-4-ene-3,20-dione.

Ilotycin (erythromycin opthalmic ointment) is commercially available from Dista and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13-hexa-methyl-6-[[3,4,6-tri-deoxy-3-(dimethylamino)-β-D-xylo-hexopyranosyl]oxy]oxa-cyclotetradecane-2,10-dione.

NeoDecadron (neomycin sulfate-dexamethasone sodium phosphate) is commercially available from Merck and is 9-fluoro-11β,17-dihydroxy-16α-methyl-21-(phosphonooxy)pregna-1,4-diene-3,20-dione disodium salt.

Polytrim (trimethoprim and polythyxin β sulfate opthalmic solution) is commercially available from Allergan and is 2,4-diamino-5-(3,4,5-trimethoxylbenzl)pyrimidine (trimethoprim) and the sulfate salt of polymyxin B₁ and B₂ (polythyxin β sulfate).

Terra-Cortril (oxytetracycline HCl and hydrocortisone acetate) is commercially available from Pfizer.

TobraDex (tobramycin and dexamethasone opthalmic suspension and ointment) is commercially available from Alcon and is O-3-Amino-3-deoxy-a-D-glucopyranosyl-(1→4)-αo-[2,6-diamino-2,3,6-trideoxy-a-D-ribo-hexopyranosyl-[(1→6)]-2-deoxy-L-streptamine. Dexa-methasone: Chemical Name: 9-Fluro-11b,17,21-trihydroxy-16a-methylpregna-1,4-diene-3,20-dione.

Vira-A opthalmic ointment, 3% (vidarabine) is commercially available from Monarch and is 9-β-D-arabinofuranosyl-9H-purin-6-amine monohydrate.

Chibroxin (norfloxacin opthalmic solution) is commercially available from Merck and is 1-ethyl-6-fluoro-1,4-dihydro-4-oxo-7-(1-piperazinyl)-3-quinoline-carboxylic acid.

Ciloxan opthalmic solution, (Ciprofloxacin HCl) is commercially available from Alcon and is the monohydro chloride monohydrate salt of 1-cyclopropyl-6-fluoro-1,4-dihydro-4-oxo-7-(1-piperazinyl)-3-quinoline-carboxylic acid.

Ocuflox opthalmic solution (ofloxacin) is commercially available from Allergan and is (±)-9-Fluoro-2,3-dihydro-3-methyl-10-(4-methyl-1-piperazinyl)-7-oxo-7-H-pyrido[1,2,3-de]-1,4-benzoxazine-6-carboxylic acid.

Blephamide opthalmic ointment (sulfacetamide sodium and prednisolone acetate) is commercially available from Allergan and is N-sulfanilyl-acetamide monosodium salt monohydrate (sulfacetamide sodium) and 11β,17,21-trihydroxypreyna-1,4-diene-3,20-dione-21-acetate (prednisolone acetate).

Blephamide opthalmic suspension (sulfacetamide sodium and prednisolone acetate) is commercially available from Allergan and is N-sulfanilyl-acetamide monosodium salt monohydrate (sulfacetamide sodium) and 11β,17,21-trihydroxypreyna-1,4-diene-3,20-dione-21-acetate (prednisolone acetate).

A/T/S (erythromycin) is commercially available from Hoescht Marion Roussel and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13-hexa-methyl-6-[[3,4,6-tri-deoxy-3-(dimethylamino)-β-D-xylo-hexopyranosyl]oxy]oxa-cyclotetradecane-2,10-dione.

Bactroban (mupirocin) is commercially available from SKB and is (αE,2S,3R,4R,5S)-5-[(2S,3S,4S,5S)-2,3-epoxy-5-hydroxy-4-methylhexyl]tetrahydro-3,4-dihydroxy-β-methyl-2H-pyran-2-crotonic acid, ester with 9-hydroxynonanoic acid, calcium salt (2:1), dihydrate.

Benzamycin (erythromycin-benzoyl peroxide topical gel) is commercially available from Dermik and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13-hexamethyl-6-[[3,4,6-trideoxy-3-(dimethylamino)-β-D-xylo-hexopyranosyl]-oxy]oxacyclotetra-decane-2,10-dione (erythromycin).

Betadine (povidone-iodine) is commercially available from Purdue Frederick.

Cleocin T (clindamycin phosphate topical solution) is commercially available from Pharmacia & Upjohn and is methyl-7-chloro-6,7,8-trideoxy-6-[[(1-methyl-4-propyl-2-pyrrolidiny)-carbonyl]amino]-1-thio-(2S-trans)-L-threo-I-D-galacto-octopyranoside-2-(dihydrogen phosphate).

Clindets (clindamycin phosphate pledgets) is commercially available from Stiefel and is methyl-7-chloro-6,7,8-trideoxy-6-(1-methyl-trans-4-propyl-L-2-pyrrolidine-carboxamido)-1-thio-L-threo-α-D-galacto-octopyranoside-2-dihydrogen phosphate.

Emgel (erythromycin) is commercially available from Glaxo Wellcome and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13-hexa-methyl-6-[[3,4,6-tri-deoxy-3-(dimethyl-amino)-β-D-xylo-hexopyranosyl]oxy]oxacyclotetra-decane-2,10-dione.

Erycette (erythromycin topical solution) is commercially available from Ortho Dermatological and is (3R*,4S*,5S*,6R*,7R*,9R*,11R*,12R*,13S*,14R*)-4-[(2,6-di-deoxy-3-C-methyl-3-O-methyl-α-L-ribo-hexopyranosyl)oxy]-14-ethyl-7,12,13-trihydroxy-3,5,7,9,11,13-hexamethyl-6-[[3,4,6-trideoxy-3-(dimethyl-amino)-β-D-xylo-hexopyranosyl]-oxy]oxacyclotetra-decane-2,10-dione.

Klaron (sodium sulfacetamide lotion) is commercially available from Dermik.

Mycostatin (nystatin cream) is commercially available from Westwood-Squibb.

Theramycin Z (erythromycin topical solution) is commercially available from Medicis and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-β-D-xylo-hexopyranosyl]-oxy]oxacyclotetradecane-2,10-dione.

T-Stat (erythromycin) is commercially available from Westwood-Squibb and is (3R*,4S*,5 S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13-hexa-methyl-6-[[3,4,6-tri-deoxy-3-(dimethylamino)-β-D-xylo-hexopyranosyl]oxy]oxa-cyclotetradecane-2,10-dione.

Exelderm (sulconazole nitrate) is commercially available from Westwood-Squibb and is (±)-1-[2,4-dichloro-β-[(p-chlorobenzyl)-thio]-phenethyl]imidazole mononitrate.

Fungizone (amphotericin B oral suspension) is commercially available from Bristol-Myers Squibb and is [1R-(1R*,3S*,5R*,6R*,9R*,11R*,15S*,16R*,17R*,18S*,19E,21E,23E,25E,27E,29E,31E,33R*,35S*,36R*,37S*)]-33-[(3-Amino-3,6-dideoxy-β-D-manno-pyranosyl)-oxy]-1,3,5,6,9,11,17,37-octahydroxy-15,16,18-trimethyl-13-oxo-14,39-dioxabi-cyclo-[33.3.1]-nonatriaconta-19,21,23,25,27,29,31-heptaena-36-carboxylic acid.

Lamisil (terbinafine hydrochloride cream) is commercially available from Novartis and is the hydrochloride of (E)-N-(6,6-dimethyl-2-hepten-4-ynyl)-N-methyl-1-naphthalene-methanamine.

Loprox (ciclopiroxolamine) is commercially available from Hoescht Marion Roussel and is 6-cyclohexy-1-hydroxy-4-methyl-2(1H)-pyridone, 2-amino-ethanol salt.

Lotrimin (clotrimazole) is commercially available from Schering and is 1-(O-Chloro-α,α-diphenylbenzyl)imidazole.

Lotrisone (clotrimazole and betamethasone diproprionate) is commercially available from Schering and is 1-(O-Chloro-α,α-diphenyl benzyl)imidazole (clotrimazole) and 9-fluoro-11β,17,21-trihroxy-16β-methylpregna-1,4-diene-3,20-dione-17,21-diproprionate (betamethasone diproprionate).

Mentax (butenafine HCl) is commercially available from Penederm and is N-4-tert-butylbenzyl-N-methyl-1-naphthalenemethylamine hydrochloride.

Monistat-Derm (miconazole nitrate) is commercially available from Ortho Dermatological and is 1-[2,4-dichloro-β-{(2,4-dichlorobenzyl)oxy)}phenethyl]imidazole mononitrate.

Mycelex (clotrimazole) is commercially available from Alza and is [1-(O-chloro-α,α-di-phenylbenzyl)imidazole.

Mycostatin (nystatin) is commercially available from Westwood-Squibb.

Naftin (naftifine HCl) is commercially available from Allergan and is (E)-N-cinnamyl-N-methyl-1-naphthalene-methylamine hydrochloride.

Nizoral (ketoconazole) is commercially available from Janssen and is cis-1-acetyl-4[4-[[2-(2,4-dichorophenyl)-2-(1H-imidazol-1-ylmethyl)-1,3-dioxolan-4-yl]methoxy]phenyl]-piperazine.

Nystop (nystatin) is commercially available from Paddock.

Oxistat (oxiconazole nitrate) is commercially available from Glaxo Wellcome and is 2′,4′-dichloro-2-imidazole-1-ylacetophenone-(Z)-[O-(2,4-dichlorobenzyl)oxime mononitrate.

Selsun Rx (2.5% selenium sulfide lotion) is commercially available from Ross.

Spectazole (econazole nitrate) is commercially available from Ortho Dermatological and is 1-[2-{(4-chorophenyl)methoxy}-2-(2,4-dichlorophenyl)-ethyl]-1H-imidazole mono-nitrate.

Denavir (penciclovir cream) is commercially available from SmithKline Beecham and is 9-[4-hydroxy-3-(hydroxymethyl) butyl]guanine.

Zovirax (acyclovir) is commercially available from Glaxo-Wellcome and is 2-amino-1,9-dihydro-9-(2-hydroxyethoxy)methyl-6H-purin-6-one.

Benzashave (benzoyl peroxide) is commercially available from Medicis.

Betadine (povidone-iodine) is commercially available from Purdue Frederick.

Betasept (chlorhexidine gluconate) is commercially available from Purdue Frederick.

Cetaphil (soap substitute) is commercially available from Galaderma.

Clorpactin WCS-90 (sodium oxychlorosene) is commercially available from Guardiam Laboratories.

Dapsone Tablets (dapsone) is commercially available from Jacobus and is 4,4′-diamino-diphenylsulfone (DDS).

Desquam-E (benzoyl peroxide) is commercially available from Westwood-Squibb.

Desquam-X (benzoyl peroxide) is commercially available from Westwood-Squibb.

Hibiclens (chlorhexidine gluconate) is commercially available from Zeneca.

Hibistat (chlorhexidine gluconate) is commercially available from Zeneca.

Impregon (tetrachlorosalicylanilide 2%) is commercially available from Fleming.

MetroCream (metronidazole) is commercially available from Galaderma and is 2-methyl-5-nitro-1H-imidazole-1-ethanol.

MetroGel (metronidazole) is commercially available from Galaderma and is 2-methyl-5-nitro-1H-imidazole-1-ethanol.

Noritate (metronidazole) is commercially available from Dermik and is 2-methyl-5-nitro-1H-imidazole-1-ethanol.

pHisoHex (hexachlorophene detergent cleanser) is commercially available from Sanofi and is 2,2′-methylene-bis[3,4,6-trichlorophenol].

Sulfacet-R (sodium sulfacetamide 10% and sulfur 5%) is commercially available from Dermik.

Sulfamylon (matenide acetate) is commercially available from Bertek and is α-amino-p-toluenesulfonamide monoacetate.

Triaz (benzoyl peroxide) is commercially available from Medicis.

Vanoxide-HC (benzoyl peroxide hydrocortisone) is commercially available from Dermik and is 11β,17,21-trihydroxypregn-4-ene-3,20-dione (hydrocortisone).

Acticin (permethrin) is commercially available from Penederm and is (±)-3-phenoxy-benzyl-3-(2,2-dichlorovinyl)-2,2-dimethylcyclopropanecarboxylate.

Elimite (permethrin) is commercially available from Allergan and is (±)-3-phenoxy-benzyl-3-(2,2-dichlorovinyl)-2,2-dimethylcyclopropanecarboxylate.

Eurax (crotamiton) is commercially available from Westwood-Squibb and is N-ethyl-N-(o-methylphenyl)-2-butenamide.

Lindane Lotion USP 1% (lindane) is commercially available from Alpharma.

Efudex (fluorouracil) is commercially available from ICN and is 5-flouro-2,4-(1H,3H)-pyrimidinedione.

Fluoroplex (fluorouracil) is commercially available from Allergan and is 5-flouro-2,4-(1H,3H)-pyrimidinedione.

Furadantin Oral Suspension (nitrofurantoin) is commercially available from Dura and is 1-[[5-nitro-2-furanyl)methylene]amino]-2,4-imidazolidine dione.

Zyvox (linezolid) is commercially available from Pharmacia & Upjohn.

Mood-Stabilizing Agents

The therapeutic and/or prophylactic agent can be a drug or other agent used to treat and/or prevent psychological disorders, for example, schizophrenia, clinical depression or drug-induced psychosis. Any of the mood stabilizing agents listed below, or any other such agent known or discovered to exhibit a therapeutic or diagnostic effect, can be more effectively delivered with one or more direct agonists or indirect activators of the K_{Ca} and/or K_{ATP} channel.

Antipsychotics including, for example, chlorpromazine (Thorazine), fluphenazine (Permitil), trifluoperazine, triflupromazine (Vespirin), thioridazine (Mellaril), thiothixene (Navane), haloperidol (Haldol), risperidone (Risperdal), clozapine (Clozaril), olanzepine (Zyprexa), raclopride, remoxipride, perphenazine, flupentixol-cis, S-sulpiride, molindone (Moban), prochlorperazine (Compazine), loxapine (Loxitane), and mesoridazine (Serentil).

Antidepressants including, for example, tricyclic antidepressants (TCAs), monoamine oxidase inhibitors (MAOIs), serotonin reuptake inhibitors (SSRIs) and atypical antidepressants. Further non-limiting examples include maprotiline (Ludiomil), desipramine (Norpramin, Pertofrane), nortripyline (Pamelor), protriptyline (Vivactil), amoxapine, doxepin (Adapin, Sinequan), bupropion (Wellbutrin), mirtazapine (Remeron), nefazodone (Serzone), trazodone, trimipramine (Surmontil), sertraline (Zoloft), paroxetine (Paxil), fluoxetine (Prozac), venlafaxine (Effexor), lithium, amitriptyline (Elavil), imipramine (Tofranil), nortriptyline, pheneizine (Nardil), tranylcypromine (Parnate), nefazodone, trazodone (Desyrel), and amoxapine (Asendin).

Anti-anxiety medications including, for example, benzodiazepines, lorazepam (Ativan), buspirone (Buspar), prazepam (Centrax), clonazepam (Klonopin), chlordiazepoxide (Librium), oxazepam (Serax), clorazepate (Tranxene), diazepam (Valium), alprazolam (Xanax) and halazepam.

Anticonvulsants

The therapeutic and/or prophylactic agent can be a drug or other agent used to treat and/or prevent a disorder characterized by convulsions or seizures, such as epilepsy. Any of the anti-convulsants listed below, or any other such agent known or discovered to exhibit a therapeutic or diagnostic effect, can be more effectively delivered with one or more direct agonists or indirect activators of the K_{Ca} and/or K_{ATP} channel.

Anti-convulsant medications including, for example, valproic acid (Depakene, Depakote), phenyloin (Dilantin), carbamazepine (Tegretol), ethosuximide (Zarontin), methsuximide (Celontin), acetazolamide (Diamox), felbamate (Felbatol), clonazepam (Klonopin), lamotrigine (Lamictal), phenobaritol (Luminal, etc.), mephobarbital (Mebaral), mephenyloin (Mesantoin), phensuximide (Milontin), primidone (Mysoline), gabapentin (Neurontin), paramethadione (Paradione), ethetoin (Peganone), phenacemide (Phenurone), topiramate (Topamax), chlorazepate (Tranxene), trimethadione (Tridione), lorazepam (Ativan) and diazepam (Valium).

Anti-Neurodegenerative Agents

The therapeutic and/or prophylactic agent can be a drug or other agent used to treat and/or prevent a neurodegenerative disorder, such as Parkinson's disease. Any of the anti-neurodegenerative agents listed below, or any other such agent known or discovered to exhibit a therapeutic or diagnostic effect, can be more effectively delivered with one or more direct agonists or indirect activators of the K_{Ca} and/or K_{ATP} channel.

Anti-neurodegenerative agents include, for example, anticholinergics, dopamine precursors (e.g., L-dopa (Sinemet, carbidopa)), COMT inhibitors, dopamine receptor agonists, MAO-B inhibitors, bromocriptine (Parlodel), pergolide (Permax), benztropine (Cogentin), amantadine (Symmetrel), trihexyphenidyl (Artane) and deprenyl (Eldepryl, selegiline), Huperzine A, acetylcholinesterase (AChE) inhibitors, N-methyl-D-aspartate (NMDA) receptor antagonists (e.g., Namenda (Memantine)), and cholinesterase inhibitors (e.g., Aricept (donepezil), Reminyl (Galantamine), Exelon (rivastigmine), Cognex (Tacrine).

Anti-Stroke Agents

The therapeutic and/or prophylactic agent can be a drug or other agent used to treat and/or prevent a stroke. Any of the anti-stroke agents listed below, or any other such agent known or discovered to exhibit a therapeutic or diagnostic effect, can be more effectively delivered with one or more direct agonists or indirect activators of the K_{Ca} and/or K_{ATP} channel.

Anti-stroke agents include, for example, thrombolytics (e.g., Tissue plasminogen activator (tPA); Prourokinase (r-proUK)), antiplatalet agents (e.g., aspirin, abciximab (Reopro), ozagrel); anticoagulants (e.g., warfarin, heparin, heparinoids); Platelet aggregation inhibitors (e.g, Dipyridamole); neuroprotectants (e.g., calcium channel antagonists, potassium channel openers, glutamate antagonists, antiadhesion molecules, N-methyl-D-aspartate (NMDA) receptor antagonists and modulators, alpha-amino-3-hydroxy-5-methyl-4-isoxazol propionic acid (AMPA) receptor antagonists, membrane stabilizers, growth factors, and glycine site antagonists); Thienopyridines (e.g., ticlopidine, clopidogrel); angiotensin-converting enzyme inhibitors; HMG-coASE reductase inhibitors; alpha-adrenergic-blocking agents; beta-adrenergic receptor antagonists; antifibrinolytics (e.g., tranexamic acid; Cyklokapron); calcium channel blockers (e.g, nimodipine); steroids (e.g., fludrocortisone acetate, hydrocortisone); lipid peroxidation inhibitors (e.g., tirilazad mesylate), anionic channel blockers (e.g., Nizofenone); and Cerebril.

Adrenergic Agents

Any of the anti-stroke agents listed below, or any other such agent known or discovered to exhibit a therapeutic or diagnostic effect, can be more effectively delivered with one or more direct agonists or indirect activators of the K_{Ca} and/or K_{ATP} channel.

Adrenergic agents which can be used in the present invention include, for example, chatecholamines, alpha adrenergic agents, beta adrenergic agents. Representative, non-limiting examples of adrenergic agents include isoetharine, midodrine hcl, amphetamine/dextroamphetamine, methamphetamine and d-amphetamine sulfate.

Non-limiting examples of U.S. patents disclosing adrenergic agents include U.S. Pat. Nos. 5,091,528, 5,091,528, 4,835,157, 5,708,015, 5,594,027, 5,580,892, 5,576,332, 5,510,376, 5,482,961, 5,334,601, 5,202,347, 5,135,926, 5,116,867, 5,091,528, 5,017,618, 4,835,157, 4,829,086, 4,579,867, 4,568,679, 4,469,690, 4,395,559, 4,381,309, 4,363,808, 4,343,800, 4,329,289, 4,314,943, 4,311,708, 4,304,721, 4,296,117, 4,285,873, 4,281,189, 4,278,608, 4,247,710, 4,145,550, 4,145,425, 4,139,535, 4,082,843, 4,011,321, 4,001,421, 3,982,010, 3,940,407, 3,852,468, and 3,832,470.

Cytokines and Therapeutic Proteins

The therapeutic and/or prophylactic agent can be a cytokine or therapeutic protein. Any of the cytokines or therapeutic proteins listed below, or any other such agent known or discovered to exhibit a therapeutic or diagnostic effect, can be more effectively delivered with one or more direct agonists or indirect activators of the K_{Ca} and/or K_{ATP} channel.

Representative, non-limiting examples cytokines include interferons (INFs), interleukins, peptide growth factors, colony stimulating factors, growth inhibitory and differentiation factors. Interferons include, for example, INF-α (Intron A (interferon alpha-2b), Roferon-A (interferon alpha-2a), Infergen (interferon alfacon-1), PEG-Intron A (pegylated interferon alpha-2), Pegasys (pegylated interferon alpha-2a); INF-β (Avonex (interferon beta-1a), Betaseron/Betaferon (interferon beta-1b), Rebif (interferon beta-1a), Rebif (interferon beta 1a), Avonex (beta interferon); INF-γ (Cirelli R et al. Clin Immunother (1996)5 (Suppl 1): p. 22-30). Interleukins include, for example, Proleukin (interleukin-2), Imunace (interleukin-2), Interleukin 2, Interleukin-2 fusion proteins, Nuvance (interleukin-4 receptor), Interleukin-8 and Interleukin-12. Colony stimulating factors include, for example, eupogen (filgrastim), Neumega (oprelvekin) and SD-01 (granulocyte colony stimulating factor).

Representative, non-limiting, examples of therapeutic proteins include, for example, erythropoietins (e.g., Procrit/EPO/Eprex (epoetin alpha), Epogin (epoetin beta), NeoRecormon Espo (epoetin alpha), Novel erythropiesis stimulating protein (NESP), Epogin/Epoch, Dynepo); Insulins (e.g., Novolin (insulin), Humulin (insulin), Humalog, Human insulin, Iletin (insulin), Lantus (insulin), NovoRapid (insulin aspart); plasminogen activators (e.g., Activase (alteplase), Actilyse (alteplase), Abbokinase (urokinase), Rapilysin/Retavase (reteplase), Streptase (streptokinase), Solinase (pamiteplase), Lanoteplase, TNKase (teneteplase); growth hormones (e.g., Genotropin/Nutropin (recombinant somatropin), Humatrope (recombinant somatropin), Norditropin (recombinant somatropin), Serostim (rHGH), Saizen (recombinant somatropin), Geref (sermorelin acetate).

Monoclonal antibodies include, for example, ReoPro (abciximab), Rituxan/Mabthera (rituxumab), Herceptin (trastuzumab), Remicade (infliximab), Orthoclone OKT3 (muromonab-CD3), Zenapax (daclizumab), Simulect (basiliximab), Bexxar (iodine131 tositumomab conjugate), Segard (afeliomomab), ReoPro (abciximab), ReoPro/Fragmin combination olizumab (rhuMAb-E25), Zevalin (ibritumomab tiuexetan), BEC2 (mitumomab).

Immunotoxins and Immunosuppressants

The therapeutic and/or prophylactic agent can be an immunotoxin or immunosuppressant. Any of the immunotoxins or immunosuppressants listed below, or any other such agent known or discovered to exhibit a therapeutic or diagnostic effect, can be more effectively delivered with one or more direct agonists or indirect activators of the K_{Ca} and/or K_{ATP} channel.

Representative, non-limiting examples of immunotoxins include SS1-PE38 (NeoPharm); LMB-1, 7, 2, 9; SGN-10 (BR96sFv-PE40) (Seattle Genetics); hIL13-PE38QQR (ligand-targeted toxin) (NeoPharm); Anti-Tac(Fv)-PE38; and Immunotoxin BL22; For a review of immunotoxin therapy for malignant brain tumors, see Rustamzadeh E J Neurooncol. (2003) 64(1-2):101-16).

Representative, non-limiting examples of immunosuppressants include Azathioprine (Imuran), Cyclophosphamide (Cytoxan), Cyclosporine (Sandimmune), Mycophenolate (MFM or CellCept) and Mercaptopurine (6-MP).

Gene Therapy

The therapeutic and/or prophylactic agent delivered in combination and/or alternation with the direct agonist(s) or indirect activators of K_{Ca} or K_{ATP} of the present invention can be an agent useful in gene therapy, i.e. DNA expression vectors, therapeutic oligonucleotides, viral particle, vector, etc.

Eukaryotic cells that may be transduced with vectors (infectious viral particles or plasmids) containing a gene therapeutic, but are not limited to, primary cells, such as primary nucleated blood cells, such as leukocytes, granulocytes, monocytes, macrophages, lymphocytes (including T-lymphocytes and B-lymphocytes), totipotent stem cells, and tumor infiltrating lymphocytes (TIL cells); bone marrow cells; endothelial cells; epithelial cells; keratinocytes; stem cells; hepatocytes, including hepatocyte precursor cells; hepatocytes, including hepatocyte precursor cells; fibroblasts; mesenchymal cells; mesothelial cells; parenchymal cells, or other cells of tumor derivation.

Optionally, the vector can also contain genes that enhance the therapeutic effects of the cell. The cells can be expanded in number before or after transduction with the vector containing the desired genes. Thus, the procedure is performed in such a manner that upon injection into the patient, the transformed cells will produce the relevant entity in the patient's body, preferably at the site of the diseased tissue itself.

The gene of the present invention carried by the transduced cells specifically comprises any sequence that directly or indirectly enhances the therapeutic effects of the cells. The gene carried by the transduced cells can also include sequences that allow the transduced cells to exert a therapeutic effect that it would not ordinarily have, such as a gene encoding a clotting factor useful in the treatment of hemophilia. The gene can encode one or more products having therapeutic effects. Examples of suitable genes include those that encode cytokines such as TNF, GMCSF, interleukins (interleukins 1-18), interferons (alpha, beta, gamma-interferons), T-cell receptor proteins and Fc receptors for antigen-binding domains of antibodies, such as immunoglobulins. Additional examples of suitable genes include genes that modify cells to “target” to a site in the body to which the cells would not ordinarily target, thereby making possible the use of the cell's therapeutic properties at that site. For example, blood cells such as TIL cells can be modified, for example, by introducing a Fab portion of a monoclonal antibody into the cells, thereby enabling the cells to recognize a chosen antigen. Likewise, blood cells having therapeutic properties can be used to target, for example, a tumor, that the blood cells would not normally target. Other genes useful in cancer therapy can be used to encode chemotactic factors that cause an inflammatory response at a specific site, thereby having a therapeutic effect. Other examples of suitable genes include genes encoding soluble CD4 that is used in the treatment of AIDS and genes encoding alpha-1-antitrypsin, which is useful in the treatment of emphysema caused by alpha-1-antitrypsin deficiency.

In general, a gene cannot be directly inserted into a cell. It must be delivered to the cell using a carrier known as a “vector.” The most common types of vectors used in gene therapy are viruses. Scientists use viruses because they have a unique ability to attach to or enter a cell's DNA. Viruses used as vectors in gene therapy are genetically disabled; they are unable to reproduce themselves, though they can replicate coordinately with the cellular DNA. Many gene therapy clinical trials rely on mouse retroviruses to deliver the desired gene. Other viruses used as vectors include adenoviruses, adeno-associated viruses, poxviruses and the herpes virus.

For example, cells from the patient are removed and grown in the laboratory. The cells are exposed to the virus that is carrying the desired gene. The virus enters the cells, and the desired gene becomes part of the cells' DNA. The cells grow in the laboratory and are then returned to the patient. This type of gene therapy is called ex vivo, which means “outside the body.” The gene is transferred into the patient's cells while the cells are outside the patient's body. In other studies, vectors (viral, bacterial) or liposomes (fatty particles) are used to deliver the desired gene to cells in the patient's body. This form of gene therapy is called in vivo, because the gene is transferred to cells inside the patient's body.

When these gene delivery vectors are used to carry genes into the body, they might alter more than the intended cells. Another danger is that the new gene might be inserted in the wrong location in the DNA, possibly causing cancer or other damage. In addition, when using in vivo gene delivery systems, there is a chance that the DNA could be introduced into reproductive cells, producing inheritable changes.

Other concerns include the possibility that transferred genes could be “overexpressed,” producing so much of a protein as to be harmful; that a pathogen vector could cause inflammation or an immune reaction; and in the case where a virus is used as the vector, it could be transmitted from the patient to other individuals or into the environment.

There are many vectors known in the art. Any known vector can be used in the present invention. In a preferred embodiment of the present invention, the vector can target a specific cell type for specific gene delivery.

(i) Adenoviral Vectors

Any of the adenoviral vectors can be used to transfect cells and/or cell lines with EBV-TK. Adenoviruses are non-enveloped viruses containing a linear double stranded DNA genome. While there are over 40 serotype strains of adenovirus, most of which cause benign respiratory tract infections in humans, subgroup C serotypes 2 or 5 are predominantly used as vectors. The life cycle does not normally involve integration into the host genome, rather they replicate as episomal elements in the nucleus of the host cell and consequently there is no risk of insertional mutagenesis. The wild type adenovirus genome is approximately 35 kb of which up to 30 kb can be replaced with foreign DNA (Smith A. E. (1995) Viral vectors in gene therapy. Annual Review of Microbiology 49: 807-838; Verma I. M. & Somia N. (1997) Gene therapy-promises, problems and prospects. Nature 389: 239-242). There are four early transcriptional units (E1, E2, E3 and E4) that have regulatory functions, and a late transcript, which codes for structural proteins. Progenitor vectors have either the E1 or E3 gene inactivated, with the missing gene being supplied in trans either by a helper virus, plasmid or integrated into a helper cell genome (human fetal kidney cells, line 293; Graham F. L., Smiley J., Russell W. L., Naim R. (1997) Characterization of a human cell line transformation by DNA from adenovirus 5. Gen. Virol. 36: 59-72). Second generation vectors additionally use an E2a temperature sensitive mutant (Engelhardt J. F., Litsky L., Wilson, J. M. (1994) Prolonged gene expression in cotton rat lung with recombinant adenoviruses defective in E2a. Human Gene Therapy 5: 1217-1229) or an E4 deletion (Armentano D., Zabner J., Sacks C., Sookdeo C. C., Smith M. P., St. George J. A., Wadsworth S. C., Smith A. E., Gregory R. J. (1997) Effect of the E4 region on the persistence of transgene expression from adenovirus vectors. J. Virol. 71: 2408-2416). The most recent “gutless” vectors contain only the inverted terminal repeats (ITRs) and a packaging sequence around the transgene, all the necessary viral genes being provided in trans by a helper virus (Chen H., Mack L. M., Kelly R., Ontell M., Kochanek S., Clemens P. R. (1997) Persistence in muscle of an adenoviral vector that lacks all viral genes. Proc. Natl. Acad. Sci. USA 94: 1645-1650).

Adenoviral vestors are very efficient at transducing target cells in vitro and vivo, and can be produced at high titres (>10¹¹/mL). With the exception of Geddes et al. (Geddes B. J., Harding T. C., Lightman S. L., Uney J. B. (1997) Long term gene therapy in the CNS: Reversal of hypothalamic diabetes insipidus in the Brattleboro rat by using an adenovirus expressing arginine vasopressin. Nature Medicine 3: 1402-1404), who showed prolonged transgene expression in rat brains using an E1 deletion vector, transgene expression in vivo from progenitor vectors is typically transient (Verma I. M. & Somia N. (1997) Gene therapy—promises, problems and prospects. Nature 389: 239-242). Following intravenous injection, 90% of the administered vector is degraded in the liver by a non-immune mediated mechanism (Worgall S., Wolff G., Falck-Pedersen E., Crystal R. G. (1997) Innate immune mechanisms dominate elimination of adenoviral vectors following in vivo administration. Human Gene Therapy 8: 37-44). Thereafter, an MHC class I restricted immune response occurs, using CD8+ CTLs to eliminate virus infected cells and CD4+ cells to secrete IFN-alpha which results in anti-adenoviral antibody (Yang Y., Wilson J. M. (1995) Clearance of adenovirus-infected hepatocytes by MHC class I restricted CD4+ CTLs in vivo. J. Immunol. 155: 2564-2569). Alteration of the adenoviral vector can remove some CTL epitopes, however the epitopes recognized differ with the host MHC haplotype (Sparer T. E., Wynn S. G., Clark D. J., Kaplan J. M., Cardoza L. M., Wadsworth S. C., Smith A. E., Gooding L. R. (1997) Generation of cytotoxic T lymphocytes against immunorecessive epitopes after multiple immunizations with adenovirus vectors is dependent on haplotype. J. Virol. 71: 2277-2284; Jooss K., Ertl H. C. J., Wilson J. M. (1998) Cytotoxic T-lymphocyte target proteins and their histocompatibility complex class I restriction in response to adenovirus vectors delivered to mouse liver. J. Virol. 72: 2945-2954). The remaining vectors, in those cells that are not destroyed, have their promoter inactivated (Armentano D., Zabner J., Sacks C., Sookdeo C. C., Smith M. P., St. George J. A., Wadsworth S. C., Smith A. E., Gregory R. J. (1997) Effect of the E4 region on the persistence of transgene expression from adenovirus vectors. J. Virol. 71: 2408-2416) and persisting antibody prevents subsequent administration of the vector.

Approaches to avoid the immune response involving transient immunosuppressive therapies have been successful in prolonging transgene expression and achieving secondary gene transfer (Jooss K., Yang Y., Wilson J. M. (1996) Cyclophosphamide diminishes inflammation and prolongs expression following delivery of adenoviral vectors to mouse liver and lung. Human Gene Therapy 7: 1555-1566; Kay M. A., Meuse L., Gown A. M., Linsley P., Hollenbaugh D., Aruffo A., Ochs H. D., Wilson C. B. (1997) Transient immunomodulation with anti-CD40 ligand and CTLA41g enhances presistence and secondary adenovirus-mediated gene transfer into mouse liver. Proc. Natl. Acad. Sci. USA 94: 4686-4691). A less interventionist method has been to induce oral tolerance by feeding the host UV inactivated vector (Kagami H., Atkinson J. C., Michalek S. M., Handelman B., Yu S., Baum B. J., O'Connell B. (1998) Repetitive adenovirus administration to the parotid gland: role of immunological barriers and induction of oral tolerance. Human Gene Therapy 9: 305-313). However, it is desirable to manipulate the vector rather than the host. Although only replication-deficient vectors are used, viral proteins are expressed at a very low level, which are presented to the immune system. The development of vectors containing fewer genes, culminating in the “gutless” vectors which contain no viral coding sequences, has resulted in prolonged in vivo transgene expression in liver tissue (Schiedner G., Morral N., Parks R. J., Wu Y., Koopmans S. C., Langston C., Graham F. L., Beaudet A. L., Kochanek S. (1998) Genomic DNA transfer with a high-capacity adenovirus vector results in improved in vivo gene expression and decreased toxicity. Nature Genetics 18: 180-183). The initial delivery of large amounts of DNA packaged within adenovirus proteins, the majority of which will be degraded and presented to the immune system may still cause problems for clinical trials. Moreover the human population is heterogeneous with respect to MHC haplotype and a proportion of the population will have been already exposed to the adenoviral strain (Gahry-Sdard H., Molinier-Frenkel V., Le Boulaire C., Saulnier P., Opolon P., Lengange R., Gautier E., Le Cesne A., Zitvogel L., Venet A., Schatz C., Courtney M., Le Chevalier T., Tursz T., Guillet J., Farace F. (1997) Phase I trial of recombinant adenovirus gene transfer in lung cancer. J. Clin. Invest. 100: 2218-2226).

Until recently, the mechanism by which the adenovirus targeted the host cell was poorly understood. Tissue specific expression was therefore only possible by using cellular promoter/enhancers, such as the myosin light chain 1 promoter (Shi Q., Wang Y., Worton R. (1997) Modulation of the specificity and activity of a cellular promoter in an adenoviral vector. Human Gene Therapy 8: 403-410) or the smooth muscle cell SM22a promoter (Kim S., Lin H., Barr E., Chu L., Leiden J. M., Parmacek M. S. (1997) Transcriptional targeting of replication-defective adenovirus transgene expression to smooth muscle cells in vivo. J. Clin. Invest. 100: 1006-1014), or by direct delivery to a local area (Rome J. J., Shayani V., Newman K. D., Farrell S., Lee S. W., Virmani R., Dicheck D. A. (1994) Adenoviral vector mediated gene transfer into sheep arteries using a double-balloon catheter. Human Gene Therapy 5: 1249-1258). Uptake of the adenovirus particle has been shown to be a two stage process involving an initial interaction of a fiber coat protein in the adenovirus with a cellular receptor or receptors, which include the MHC class I molecule (Hong S. S., Karayan L., Tournier J., Curiel D. T., Boulanger P. A. (1997) Adenovirus type 5 fiber knob binds to MHC class I a2 domain at the surface of human epithelial and B lymphoblastoid cells. EMBO J. 16: 2294-2306) and the Coxsackie virus-adenovirus receptor (Bergelson J. M., Cunningham J. A., Droguett G., Kurt-Jones A. E., Krithivas A., Hong J. S., Horwitz M. S., Crowell R. L., Finberg R. W. (1997) Isolation of a common receptor for Coxsackie virus B viruses and adenoviruses 2 and 5. Science 275: 1320-1323). The penton base protein of the adenovirus particle then binds to the integrin family of cell surface heterodimers (Wickham T. J., Mathias P., Cheresh D. A., Nemerow G. R. (1993) Integrins avb3 and avb5 promote adenovirus internalization but not virus attachment. Cell 73: 309-319) allowing internalization via receptor mediated endocytosis. Most cells express primary receptors for the adenovirus fiber coat protein, however internalization is more selective (Harris J. D. & Lemoine N. R. (1996) Strategies for targeted gene therapy. Trends in Genetics 12: 400-404). Methods of increasing viral uptake include stimulating the target cells to express an appropriate integrin (Davison E., Diaz R. M., Hart I. R., Santis G., Marshall J. F. (1997) Integrin a5b1-mediated adenovirus infection is enhanced by the integrin-activating antibody TS2/16. Journal of Virology 71: 6204-6207) and conjugating an antibody with specificity for the target cell type to the adenovirus (Wickham T. J., Lee G. M, Titus J. A., Titus J. A., Sconocchia G., Bakacs T., Kovesdi I., Segal D. M. (1997b). Targeted adenovirus-mediated gene delivery to T cells via CD3. J. Virol. 71: 7663-7669; Goldman C. K., Rogers B. E., Douglas J. T., Sonsowski B. A., Ying W., Siegal G. P., Baird A., Campain J. A., Curiel D. T. (1997) Targeted gene delivery to Kaposi's sarcoma cells via the fibroblast growth factor receptor. Cancer Res. 57: 1447-1451). The use of antibodies though increases the production difficulties of the vector and the potential risk of activating the complement system. By incorporating receptor binding motifs into the fiber coat protein (Wickham T. J., Tzeng E., Shears I I, L. L., Roelvink P. W., Li Y., Lee G. M., Brough D. E., Lizonova A., Kovesdi I. (1997a) Increased in vitro and in vivo gene transfer by adenovirus vectors containing chimeric fiber proteins. J. Virol. 71: 8221-8229) were able to redirect the virus to bind the integrin expressed by damaged endothelial or smooth muscle cells, or heparin sulfate receptors, which are expressed by many cell types.

(ii) Adeno-Associated Viral Vectors

Any of the adeno-associated viral vectors can be used to transfect cells and/or cell lines with EBV-TK or KHSV-TK. Adeno-associated viruses (AAV) are non-pathogenic human parvoviruses, dependant on a helper virus, usually adenovirus, to proliferate. They are capable of infecting both dividing and non dividing cells, and in the absence of a helper virus integrate at a specific point of the human host genome (19q13→qter) at a high frequency (Kotin R. M., Siniscalco M., Samulski R. J., Zhu X. D., Hunter L., Laughlin C. A., McLaughlin S., Muzyczka N., Rocchi M., Berns K. I. (1990) Site-specific integration by adeno-associated virus. Proc. Natl. Acad. Sci. USA 87: 2211-2215). The wild type genome is a single stranded DNA molecule, consisting of two genes; rep, coding for proteins which control viral replication, structural gene expression and integration into the host genome, and cap, which codes for capsid structural proteins. At either end of the genome is a 145 bp terminal repeat (TR), containing a promoter.

When used as a vector, the rep and cap genes are replaced by the transgene and its associated regulatory sequences. The total length of the insert cannot greatly exceed 4.7 kb, the length of the wild type genome (Smith A. E. (1995) Viral vectors in gene therapy. Ann. Rev. Microbiol. 49: 807-838). Production of the recombinant vector requires that rep and cap are provided in trans, along with helper virus gene products (E1a, E1b, E2a, E4 and VA RNA from the adenovirus genome). The conventional method is to cotransfect two plasmids, one for the vector and another for rep and cap, into 293 cells infected with adenovirus (Samulski R. J., Chang L., Shenk T. (1989) Helper free stocks of recombinant adeno-associated viruses: normal integration does not require viral gene expression. J. Virol. 63: 3822-3828). This method, however, is cumbersome, low yielding (<10⁴ particles/ml) and prone to contamination with adenovirus and wild type AAV. One of the reasons for the low yield is the inhibitory effect of the rep gene product on adenovirus replication (Vincent K. A, Piraino S. T., Wadsworth S. C. (1997) Analysis of recombinant adeno-associated virus packaging and requirements for rep and cap gene products. J. Virol. 71: 1897-1905). More recent protocols remove all adenoviral structural genes and use rep resistant plasmids (Xiao X., Li J., Samulski R. J. (1998) Production of high-titer recombinant adeno-associated virus vectors in the absence of helper adenovirus. J. Virol. 72: 2224-2232) or conjugate a rep expression plasmid to the mature virus prior to infection (Fisher K. J., Kelley W. M, Burda J. F., Wilson J. M. (1996) A novel adenovirus-adeno-associated virus hybrid vector that displays efficient rescue and delivery of the AAV genome. Human Gene Therapy 7: 2079-2087).

In the absence of rep, the AAV vector will only integrate at random, as a single provirus or head to tail concatamers, once the terminal repeats have been slightly degraded (Rutledge E. A. & Russell D. W. (1997) Adeno-associated virus vector integration junctions. J. Virol. 71: 8429-8436). Interest in AAV vectors has been due to their integration into the host genome allowing prolonged transgene expression. Gene transfer into vascular epithelial cells (Maeda Y., Ikeda U., Ogasawara Y., Urabe M., Takizawa T., Saito T., Colosi P., Kurtzman G., Shimada K., Ozawa, K. (1997) Gene transfer into vascular cells using adeno-associated virus (AAV) vectors. Cardiovascular Res. 35: 514-521), striated muscle (Fisher K. J., Jooss K., Alston J., Yang Y., Haecker S. E., High K., Pathak R., Raper S. E., Wilson J. M. (1997) Recombinant adeno-associated virus for muscle directed gene delivery. Nature Medicine 3: 306-316; Herzog R. W., Hagstrom J. N., Kung S., Tai S. J., Wilson J. M., Fisher K. J., High K. A. (1997) Stable gene transfer and expression of human blood coagulation factor IX after intramuscular injection of recombinant adeno-associated virus. Proc. Natl. Acad. Sci. USA 94: 5804-5809) and hepatic cells (Snyder R. O., Miao C. H., Patijn G. A., Spratt S. K., Danos O., Nagy D., Gown A. M., Winter B., Meuse L., Cohen L. K., Thompson A. R., Kay, M. A. (1997) Persistent and therapeutic concentrations of human factor IX in mice after hepatic gene transfer of recombinant AAV vectors. Nature Genetics 16: 270-275) has been reported, with prolonged expression when the transgene is not derived from a different species. Neutralizing antibody to the AAV capsid may be detectable, but does not prevent readministration of the vector or shut down promoter activity. It is possibly due to the simplicity of the viral capsid, that the immune response is so muted. As AAV antibodies will be present in the human population this will require further investigation. There has been no attempt to target particular cell types other than by localized vector delivery.

In particular, the adeno-associated vectors disclosed in U.S. Pat. No. 5,693,531, which is hereby incorporated by reference, can be used, including AAVp5neo; pSV-β-galactosidase; TRF169; LZ11; pSP72; pSP72nLacZ; pAdRSV4; pAdRSVnLacZ; AAVmLac; SV40; pBluescriptSK; pSV40 ori AAV1; and pKMT11.

(iii) Retroviral Vectors

Any of the retroviral vectors can be used to transfect cells and/or cell lines with EBV-TK. Retroviruses are a class of enveloped viruses containing a single stranded RNA molecule as the genome. Following infection, the viral genome is reverse transcribed into double stranded DNA, which integrates into the host genome and is expressed as proteins. The viral genome is approximately 10 kb, containing at least three genes: gag (encoding core proteins), pol (encoding reverse transcriptase) and env (encoding the viral envelope protein). At each end of the genome are long terminal repeats (LTRs) which include promoter/enhancer regions and sequences involved with integration. In addition there are sequences required for packaging the viral DNA (psi) and RNA splice sites in the env gene. Some retroviruses contain proto-oncogenes, which when mutated can cause cancers; however, in the production of vectors these are removed. Retroviruses can also transform cells by integrating near a cellular proto-oncogene and driving inappropriate expression from the LTR, or by disrupting a tumor suppresser gene. Such as event, termed insertional mutagenesis, though extremely rare, could still occur when retroviruses are used as vectors.

Retroviral vectors are most frequently based upon the Moloney murine leukemia virus (Mo-MLV), which is an amphotrophic virus, capable of infecting both mouse cells, enabling vector development in mouse models, and human cells, enabling human treatment. The viral genes (gag, pol and env) are replaced with the transgene of interest and expressed from plasmids in the packaging cell line. Because the non-essential genes lack the packaging sequence (psi) they are not included in the virion particle. To prevent recombination resulting in replication-competent retroviruses, all regions of homology with the vector backbone should be removed and non-essential genes should be expressed in at least two transcriptional units (Markowitz D., Goff S., Bank A. (1988) A safe packaging line for gene transfer: separating viral genes on two different plasmids. J. Virol. 62: 1120-1124). Even so, replication-competent retroviruses do arise at a low frequency.

The essential regions include the 5′- and 3′-LTRs, and the packaging sequence lying downstream of the 5′-LTR. Transgene expression can either be driven by the promoter/enhancer region in the 5′-LTR, or by alternative viral (e.g., cytomegalovirus, Rous sarcoma virus) or cellular (e.g., beta actin, tyrosine) promoters. Mutational analysis has shown that up to the entire gag coding sequence and the immediate upstream region can be removed without effecting viral packaging or transgene expression (Kim S. H., Yu S. S., Park J. S, Robbins P. D, An C. S., Kim S. (1998) Construction of retroviral vectors with improved safety, gene expression, and versatility. J. Virol. 72: 994-1004). However the exact positioning of the transgene start codon and small alterations of the 5′-LTR influence transgene expression (Rivire I., Brose K., Mulligan R. C. (1995) Effects of retroviral vector design on expression of human adenosine deaminase in murine bone marrow transplant recipients engrafted with genetically modified cells. Proc. Natl. Acad. Sci. USA 92: 6733-6737). To aid identification of transformed cells selectable markers, such as neomycin and beta-galactosidase, can be included and transgene expression can be improved by the addition of internal ribosome-binding sites (Saleh M. (1997) A retroviral vector that allows co-expression of two genes and the versatility of alternate selection markers. Human Gene Therapy 8: 979-983). The available carrying capacity for retroviral vectors is approximately 7.5 kb (Verma I. M. & Somia N. (1997) Gene therapy-promises, problems and prospects. Nature 389: 239-242), which is too small for some genes, even if the cDNA is used.

The retroviral envelope interacts with a specific cellular protein to determine the target cell range. Altering the env gene or its product has proved a successful means of manipulating the cell range. Approaches have included direct modifications of the binding site between the envelope protein and the cellular receptor, however these approaches tend to interfere with subsequent internalization of the viral particle (Harris J. D. & Lemoine N. R. (1996) Strategies for targeted gene therapy. Trends in Genetics 12: 400-404). By replacing a portion of the env gene with 150 codons from the erythropoietin protein (EPO), Kasahara et al. (Kasahara N., Dozy A. M., Kan Y. W. (1994) Tissue-specific targeting of retroviral ligand-receptor interactions. Science 266: 1374-1376) were able to target EPO receptor bearing cells with high affinity. Coupling an antibody to the viral particle with affinity for a second cell specific antibody via a streptavodin bridge, improves viral uptake, but internalization tends to lead to viral degradation (Roux P., Jeanteur P., Piechaczyk M. (1989) A versatile and potentially general approach to the targeting of specific cell types by means of major histocompatibility complex class I and class II antigens by mouse ecotropic murine leukemia virus-derived viruses. Proc. Natl. Acad. Sci. USA 86: 9079-9083)., Neda et al. (Neda H., Wu C. H., Wu G. Y. (1991) Chemical modification of an ecotropic murine leukemia virus results in redirection of its target cell specificity. J. Biol. Chem. 266: 14143-14146) treated viral particles with lactose, which resulted in uptake by cells, principally hepatocytes, expressing asialoglycoprotein receptors. Subsequently, there was efficient viral transgene expression, possibly due to acidification of the endosome, allowing fusion of the viral envelope with the endosome membrane.

Viruses differ with respect to their tropisms; therefore, by replacing the env gene with that of another virus, the host range can be extended, in a technique known as pseudotyping. Vesicular stomatitis virus G protein has been included in Mo-MLV derived vectors (Burns J. C., Matsubara T., Lozinski G., Yee J., Freidmann T., Washabaugh C. H., Tsonis P. A. (1994) Pantropic retroviral vector-mediated gene transfer, integration, and expression in cultured newt limb cells. Dev. Biol. 165: 285-289), which are also more stable when purified by ultracentrifugation. Recently, Qing et al. (Qing K., Bachelot T., Mukherjee P., Wang X., Peng L., Yoder M. C., Leboulch P., Srivastava A. (1997) Adeno-associated virus type 2-mediated transfer of ecotropic retrovirus receptor cDNA allows ecotropic retroviral transduction of established and primary human cells. J. Virol. 71: 5663-5667) improved transduction into numerous cell lines by first treating the recipient cells with an adeno-associated vector (discussed below) expressing the cellular receptor for retroviral envelope protein.

A requirement for retroviral integration and expression of viral genes is that the target cells should be dividing. This limits gene therapy to proliferating cells in vivo or ex vivo, whereby cells are removed from the body, treated to stimulate replication and then transduced with the retroviral vector, before being returned to the patient. When treating cancers in vivo, tumor cells are preferentially targeted (Roth J. A., Nguyen D., Lawrence D. D., Kemp B. L., Carrasco C. H., Ferson D. Z., Hong W. K., Komaki R., Lee J. J., Nesbitt J. C., Pisters K. M. W., Putnam J. B., Schea R., Shin D. M., Walsh G. L., Dolormente M. M., Han C. I., Martin F. D., Yen N., Xu K., Stephens L. C., McDonnell T. J., Mukhopadhyay T., Cai D. (1996). Retrovirus mediated wild-type p53 gene transfer to tumors of patients with lung cancer. Nature Medicine 2: 985-991; Tait D. L., Obermiller P. S., Redlin-Frazier S., Jensen R. A., Welcsh P., Dann J., King M, Johnson D. H., Holt J. T. (1997). A phase I trial of retroviral BRCA1sv gene therapy in ovarian cancer. Clin. Cancer Res. 3: 1959-1968). However, ex vivo cells can be more efficiently transduced, because of exposure to higher virus titers and growth factors (Glimm H., Kiem H. P., Darovsky B., Storb R., Wolf J., Diehl V., Mertelsmann R., Kalle C. V. (1997). Efficient gene transfer in primitive CD34+/CD38lo human bone marrow cells reselected after long term exposure to GALV-pseudotyped retroviral vector. Human Gene Therapy 8: 2079-2086). Furthermore ex vivo-treated tumor cells will associate with the tumor mass and can direct tumoricidal effects (Oldfield E. H. & Ram Z. (1995) Intrathecal gene therapy for the treatment of leptomeningeal carcinomatosis. Human Gene Therapy 6: 55-85; Abdel-Wahab Z., Weltz C., Hester D., Pickett N., Vervaert C., Barber J. R., Jolly D., Seigler H. F. (1997) A phase I clinical trial of immunotherapy with interferon-gamma gene-modified autologous melanoma cells. Cancer 80: 401-412).

Though transgene expression is usually adequate in vitro and initially in vivo, prolonged expression is difficult to attain. Retroviruses are inactivated by c1 complement protein and an anti-alpha galactosyl epitope antibody, both present in human sera (Rother P. R., William L. F., Springhorn J. P., Birks W. C., Sandrin M. S., Squinto S. P., Rollins S. A. (1995) A novel mechanism of retrovirus inactivation in human serum mediated by anti-a-galactosyl natural antibody. J. Exp. Med. 182: 1345-1355; Rollins S. A., Birks C. W., Setter E., Squinto S, P., Rother R. P. (1996) Retroviral vector producer cell killing in human serum is mediated by natural antibody and complement: strategies for evading the humoral immune response. Human Gene Therapy 7: 619-626). Transgene expression is also reduced by inflammatory interferons, specifically IFN-alpha and IFN-gamma acting on viral LTRs (Ghazizadeh S., Carroll J. M., Taichman L. B. (1997) Repression of retrovirus-mediated transgene expression by interferons: implications for gene therapy. J. Virol. 71: 9163-9169). As the retroviral genome integrates into the host genome, it is most likely that the viral LTR promoters are inactivated; therefore, one approach has been to use promoters for host cell genes, such as tyrosine (Diaz R. M., Eisen T., Hart I. R., Vile R. G. (1998) Exchange of viral promoter/enhancer elements with regulatory sequences generated targeted hybrid long terminal repeat vectors for gene therapy of melanoma. J. Virol. 72: 789-795). Clearly this is an area where continued research is needed.

Lentiviruses are a subclass of retroviruses that are able to infect both proliferating and non-proliferating cells. They are considerably more complicated than simple retroviruses, containing an additional six proteins, tat, rev, vpr, vpu, nef and vif. Current packaging cell lines have separate plasmids for a pseudotype env gene, a transgene construct, and a packaging construct supplying the structural and regulatory genes in trans (Naldini L., Blmer U., Gallay P., Ory D., Mulligan R., Gage F. H., Verma I. M., Trono D. (1996) In vivo gene delivery and stable transduction of non-dividing cells by a lentiviral vector. Science 272: 263-267). Early results using marker genes have been promising, showing prolonged in vivo expression in muscle, liver and neuronal tissue (Blmer U., Naldini L., Kafri T., Trono D., Verma I. M., Gage F. H. (1997) Highly efficient and sustained gene transfer in adult neurons with a lentivirus vector. J. Virol. 71: 6641-6649; Miyoshi H., Takahashi M., Gage F. H., Verma I. M. (1997) Stable and efficient gene transfer into the retina using an HIV-based lentiviral vector. Proc. Natl. Acad. Sci. USA 94: 10319-10323; Kafri T., Blmer U., Peterson D. A., Gage F. H., Verma I. M. (1997) Sustained expression of genes delivered into liver and muscle by lentiviral vectors. Nature Genetics 17: 314-317). Interestingly the transgenes are driven by an internally engineered cytomegalovirus promoter, which unlike the situation in MoMLV vectors, is not inactivated. This may be due to the limited inflammatory response to vector injection, which was equal in magnitude to the saline control (Blmer U., Naldini L., Kafri T., Trono D., Verma I. M., Gage F. H. (1997) Highly efficient and sustained gene transfer in adult neurons with a lentivirus vector. J. Virol. 71: 6641-6649).

The lentiviral vectors used are derived from the human immunodeficiency virus (HIV) and are being evaluated for safety, with a view to removing some of the non-essential regulatory genes. Mutants of vpr and vif are able to infect neurons with reduced efficiency, but not muscle or liver cells (Blmer U., Naldini L., Kafri T., Trono D., Verma I. M., Gage F. H. (1997) Highly efficient and sustained gene transfer in adult neurons with a lentivirus vector. J. Virol. 71: 6641-6649; Kafri T., Blmer U., Peterson D. A., Gage F. H., Verma I. M. (1997) Sustained expression of genes delivered into liver and muscle by lentiviral vectors. Nature Genetics 17: 314-317).

In a particular embodiment, the retroviral vectors pLXIN, pSIR, pLXSH, pLNCX, pLAPSN, pFB and pFB-Neo are used.

(iv) Herpes Simplex Viral Vectors

Any of the herpes simplex viral vectors can be used to transfect cells and/or cell lines with EBV-TK. Herpes simplex virus type 1 (HSV-1) is a human neurotropic virus; consequently interest has largely focused on using HSV-1 as a vector for gene transfer to the nervous system. Wild-type HSV-1 virus is able to infect neurons and either proceed into a lytic life cycle or persist as an intranuclear episome in a latent state. Latently infected neurons function normally and are not rejected by the immune system. Though the latent virus is transcriptionally almost silent, it does possess neuron-specific promoters that are capable of functioning during latency. Antibodies to HSV-1 are common in the human population; however complications due to herpes infection, such as encephalitis, are very rare.

The viral genome is a linear, double-stranded DNA molecule of 152 kb. There are two unique regions, long and short (termed UL and US) which are linked in either orientation by internal repeat sequences (IRL and IRS). At the non-linker end of the unique regions are terminal repeats (TRL and TRS). There are up to 81 genes (Marconi P., Krisky D., Oligino T., Poliani P. L., Ramakrishnan R., Goins W. F., Fink D. A., Glorioso J. C. (1996) Replication-defective herpes simplex virus vectors for gene transfer in vivo. Proc. Natl. Acad. Sci. USA 93: 11319-11320), of which about half are not essential for growth in cell culture. Once these non-essential genes have been deleted, 40-50 kb of foreign DNA can be accommodated within the virus (Glorioso J. C., DeLuca N. A., Fink D. J. (1995) Development and application of herpes simplex virus vectors for human gene therapy. Annual Review of Microbiology 49: 675-710). Three main classes of HSV-1 genes have been identified: the immediate-early (IE or alpha) genes, early (E or beta) genes, and late (L or gamma) genes.

Following infection in susceptible cells, lytic replication is regulated by a temporally coordinated sequence of gene transcription. Vmw65 (a tegument structural protein) activates the immediate early genes (IP0, ICP4, ICP22, ICP27 and ICP477) that are transactivating factors allowing the production of early genes. The early genes encode proteins for nucleotide metabolism and DNA replication. Late genes are activated by the early genes and encode structural proteins. The entire cycle takes less than 10 h and invariably results in cell death.

The molecular events leading to the establishment of latency have not been fully determined. Gene expression during latency is driven by the latency associated transcripts (LATs) located in the IRL region of the genome. Two LATs (2.0 and 1.5 kb) are transcribed in the opposite direction to the IE gene ICP0. LATs have a role in HSV-1 reactivation from latency (Steiner I., Spivack J. G., Lirette R. P., Brown S. M., MacLean A. R., Subak-Sharpe J. H., Fraser N. W. (1989) Herpes simplex virus type 1 latency associated transcripts are evidently not essential for latent infection. EMBO Journal 8: 505-511) and the establishment of latency (Sawtell N. M. & Thompson R. L. (1992) Herpes simplex virus type 1 latency-associated transcription unit promotes anatomical site-dependant establishment and reactivation from latency. J. Virol. 66: 2157-2169). Two latency active promoters that drive expression of the LATs have been identified (Marconi P., Krisky D., Oligino T., Poliani P. L., Ramakrishnan R., Goins W. F., Fink D. A., Glorioso J. C. (1996) Replication-defective herpes simplex virus vectors for gene transfer in vivo. Proc. Natl. Acad. Sci. USA 93: 11319-11320) and may prove useful for vector transgene expression.

Two basic approaches have been used for production of HSV-1 vectors, namely amplicons and recombinant HSV-1 viruses. Amplicons are bacterially produced plasmids containing col E1 ori (an Escherichia coli origin of replication), OriS (the HSV-1 origin of replication), HSV-1 packaging sequence, the transgene under control of an immediate-early promoter and a selectable marker (Federoff H. J., Geschwind M. D., Geller A. I., Kessler J. A. (1992) Expression of nerve factor in vivo from a defective herpes simplex virus 1 vector prevents effects of axotomy on sympathetic ganglia. Proc. Natl. Acad. Sci. USA 89: 1636-1640). The amplicon is transfected into a cell line containing a helper virus (a temperature sensitive mutant), which provides all the missing structural and regulatory genes in trans. Both the helper- and amplicon-containing viral particles are delivered to the recipient. More recent amplicons include an Epstein-Barr virus-derived sequence for plasmid episomal maintenance (Wang S. & Vos J. (1996) A hybrid herpesvirus infectious vector based on Epstein-Barr virus and herpes simplex virus type 1 for gene transfer into human cells in vitro and in vivo. J. Virol. 70: 8422-8430).

Recombinant viruses are made replication-deficient by deletion of one of the immediate-early genes (e.g., ICP4), which is provided in trans. Though they are less pathogenic and can direct transgene expression in brain tissue, they are toxic to neurons in culture (Marconi P., Krisky D., Oligino T., Poliani P. L., Ramakrishnan R., Goins W. F., Fink D. A., Glorioso J. C. (1996) Replication-defective herpes simplex virus vectors for gene transfer in vivo. Proc. Natl. Acad. Sci. USA 93: 11319-11320). Deletion of a number of immediate-early genes substantially reduces cytotoxicity and also allows expression from promoters that would be silenced in the wild-type latent virus. These promoters may be of use in directing long term gene expression.

Replication-conditional mutants are only able to replicate in certain cell lines. Permissive cell lines are all proliferating and supply a cellular enzyme to complement for a viral deficiency. Mutants include thymidine kinase (During M. J., Naegele J. R., O'Malley K. L., Geller A. I. (1994) Long-term behavioral recovery in Parkinsonian rats by an HSV vector expressing tyrosine hydroxylase. Science 266: 1399-1403), ribonuclease reductase (Kramm C. M., Chase M., Herrlinger U., Jacobs A., Pechan P. A., Rainov N. G., Sena-esteves M., Aghi M., Barnett F. H., Chiocca E. A., Breakefield X. O. (1997) Therapeutic efficiency and safety of a second-generation replication-conditional HSV1 vector for brain tumor gene therapy. Human Gene Therapy 8: 2057-2068), UTPase, or the neurovirulence factor g34.5 (Kesari S., Randazzo B. P., Valyi-Nagy T., Huang Q. S., Brown S. M., MacLean A. R., Lee V. M., Trojanowski J. Q., Fraser N. W. (1995) Therapy of experimental human brain tumors using a neuroattenuated herpes simplex virus mutant. Lab. Invest. 73: 636-648). These mutants are particularly useful for the treatment of cancers, killing neoplastic cells, which proliferate faster than other cell types (Andreansky S. S., He B., Gillespie G. Y., Soroceanu L., Market J., Chou J., Roizman B., Whitley R. J. (1996) The application of genetically engineered herpes simplex viruses to the treatment of experimental brain tumors. Proc. Natl. Acad. Sci. USA 93: 11313-11318; Andreansky S. S., Sorcoceanu L., Flotte E. R., Chou J., Markert J. M., Gillespie G. Y., Roizman B., Whitley R. J. (1997) Evaluation of genetically engineered herpes simplex virus as oncolytic agents for human malignant brain tumors. Cancer Res. 57: 1502-1509).

A number of neurological diseases could be amenable to gene therapy by HSV-1 vectors (Kennedy P. G. E. (1997) Potential uses of herpes simplex virus (HSV) vectors for gene therapy of neurological disorders. Brain 120: 1245-1259). Though most attention has focused on cancers, there has been some success in Parkinson's disease by expressing tyrosine hydroxylase in striatal cells (Geller A. I., During M. J., Oh J. Y., Freese F., O'Malley K. (1995) An HSV-1 vector expressing tyrosine hydroxylase causes production and release of L-DOPA from cultured rat striatal cells. J. Neurochem. 64: 487-496; During M. J., Naegele J. R., O'Malley K. L., Geller A. I. (1994) Long-term behavioral recovery in Parkinsonian rats by an HSV vector expressing tyrosine hydroxylase. Science 266: 1399-1403), thus replacing the supply of L-dopa. Federoffet al. (Federoff H. J., Geschwind M. D., Geller A. I., Kessler J. A. (1992) Expression of nerve factor in vivo from a defective herpes simplex virus 1 vector prevents effects of axotomy on sympathetic ganglia. Proc. Natl. Acad. Sci. USA 89: 1636-1640) induced nerve repair following axotomy of the superior cervical ganglion, by injection of a vector expressing nerve growth factor. This resulted in restored levels of tyrosine hydroxylase.

However, Wood et al. (Wood M. J. A., Byrnes A. P., Pfaff D. W., Rabkin S. D., Charlton H. M. (1994) Inflammatory effects of gene-transfer into the CNS with defective HSV-1 vectors. Gene Therapy 1: 283-291) observed strong inflammatory responses to HSV-1 amplicon vectors, both at the primary site of the injection and at secondary sites supplied by nerve fibers originating from area of the injection. In addition up to 20% of experimental animals may die shortly after injection with HSV-1 vector (Kucharczuk J. C., Randazzo B., Chang M. Y., Amin K. M., Elshami A. A., Sterman D. H., Rizk N. P., Molnar-Kimber K. L., Brown S. M., MacLean A. R., Litzky L. A., Fraser N. W., Albelda S. M., Kaiser L. R. (1997) Use of a replication-restricted herpes virus to treat experimental human malignant mesothelioma. Cancer Research 57: 466-471), though the-reason is unknown. A viral protein, ICP47, has been identified, which reduces viral antigen presentation and may be employed in future HSV-1 vectors to reduce cytotoxicity (York I. A., Roop C., Andrews D. W., Riddell R., Graham F. L., Johnson D. C. (1994) A cytosolic herpes simplex virus protein inhibits antigen presentation to CD8+ T lymphocytes. Cell 77: 525-535).

Because of its tropism for neuronal tissue issues of cellular targeting have been largely overlooked. However, wild type HSV-I can infect and lyse other non-neuronal cell types, such as skin cells (Al-Saadi S. A., Clements G. B., Subak-Sharpe J. H. (1983) Viral genes modify herpes simplex virus latency both in mouse footpad and sensory ganglia. J. Gen. Virol. 64: 1175-1179), and it would be advantageous to target a specific subset of neurons. As HSV-1 will travel down the length of nerve efficient cellular targeting would improve its safety profile when used as a vector. Indeed a replication-restricted HSV-1 vector has been used to treat human malignant mesothelioma (Kucharczuk J. C., Randazzo B., Chang M. Y., Amin K. M., Elshami A. A., Sterman D. H., Rizk N. P., Molnar-Kimber K. L., Brown S. M., MacLean A. R., Litzky L. A., Fraser N. W., Albelda S. M., Kaiser L. R. (1997) Use of a replication-restricted herpes virus to treat experimental human malignant mesothelioma. Cancer Res. 57: 466-471).

(v) Pox Virus Vectors

Pox virus vectors can also be used (e.g., Yaba-like disease virus: an alternative replicating poxvirus vector for cancer gene therapy. Hu Y, Lee J, McCart J A, Xu H, Moss B, Alexander H R, Bartlett D L J Virol 2001 75:10300-8).

(vi) Non-Viral Vectors

Viral vectors all induce some degree of immunological response and may have other safety risks, such as insertional mutagenesis and direct toxicity. Furthermore, large-scale production may be difficult to achieve. Therefore, in some embodiments of the invention, non-viral methods of gene transfer are used, which may require only a small number of proteins, have a virtually infinite capacity, have no infectious or mutagenic capability, and large-scale production is possible using pharmaceutical techniques. There are at least three methods of non-viral DNA transfer, including naked DNA, liposomes and molecular conjugates.

Naked DNA (in the form of a plasmid) can be directly injected into muscle cells (Wolff J. A., Malone R. W., Williams P., Chong W., Acsadi G., Jani A., Felgner P. L. (1990) Direct gene transfer into mouse muscle in vivo. Science 247: 1465-1468) or attached to gold particles that are bombarded into the tissue (Cheng L., Ziegelhoffer P. R., Yang N. S. (1993) In vivo promoter activity and transgene expression in mammalian somatic tissues evaluated by using particle bombardment. Proc. Natl. Acad. Sci. USA 90: 4455-4459). Though not very efficient, this can result in prolonged, low-level expression in vivo. The simplicity of this method and sustained expression has led to the development of DNA vaccines. Compared to conventional attenuated and protein-based vaccines, they are unaffected by pre-existing immunity, such as that due to maternal antibodies, relatively cheap, and can deliver several pathogen antigens on a single plasmid (Manickan E., Karem K. L., Rouse B. T. (1997) DNA vaccines—a modern gimmick or a boon to vaccinology. Critical Reviews in Immunology 17: 139-154). DNA vaccines are being developed for those pathogens where there is no existing vaccine, such as HIV (Lekutis C., Shiver J. W., Liu M. A., Letvin L. N. (1997) HIV1 env DNA vaccine administered to rhesus monkeys elicits MHC class II-restricted CD4+ T helper cells that secrete IFN-gamma and TNF-alpha. J. Immunol. 158: 4471-4477), or where the current vaccine is not fully effective, such as influenza (Macklin M. D., McCabe D., McGregor M. W., Neumann V., Meyer T., Callan R., Hinshaw V. S., Swain W. S. (1998) Immunization of pigs with a particle mediated vaccine to influenza A virus protects against challenge with homologous virus. J. Virol. 72: 1491-1496). By using a highly conserved gene Ulmer et al. (Ulmer J. B., Donnelly J. J., Parker S. E., Rhodes G. H., Felgner P. L., Dwarki J. J., Gromkowski S. H., Deck R., DeWitt C. M., Friedman A., Hawe L. A., Laender K. R., Martinz D., Perry H. C., Shiver J., Montgomery D. L., Liu M. A. (1993) Heterologous protection against influenza by injection of DNA encoding a viral protein. Science 254: 1745-1749) were able to immunize mice against two serologically distinct influenza virus strains. In most cases, however, DNA vaccines have not been shown to be better than the existing vaccines (Macklin M. D., McCabe D., McGregor M. W., Neumann V., Meyer T., Callan R., Hinshaw V. S., Swain W. S. (1998) Immunization of pigs with a particle-mediated vaccine to influenza A virus protects against challenge with homologous virus. J. Virol. 72: 1491-1496). The actual type of immune response can be controlled by cotransformation of a gene encoding an appropriate cytokine (Xiang Z. & Ertl H. C. (1995) Manipulation of the immune response to a plasmid-encoded viral antigen by co-inoculation with plasmids expressing cytokines. Immunity 2: 129-135) and this method may prove useful in redirecting inappropriate immune responses (Manickan E., Karem K. L., Rouse B. T. (1997) DNA vaccines—a modern gimmick or a boon to vaccinology. Critical Reviews in Immunology 17: 139-154). Other uses for naked DNA include cancer immunopotentiation (discussed below; see also Corr M., Tighe H., Lee D., Dudler J., Trieu M., Brinson D. C., Carson D. A. (1997) Costimulation provided by DNA immunization enhances antitumor immunity. J. Lab. Invest. 159: 4999-5004), repair of pancreatic insulin function (Goldfine I. D., German M. S., Tseng H., Wang J., Bolaffi J. L., Chen J., Olson D. C., Rothman S. S. (1997). The endocrine secretion of human insulin and growth hormone by exocrine glands of the gastrointestinal tract. Nature Biotech. 15: 1378-1382), and stimulation of collateral blood vessel development (Takeshita S., Tsurumi Y., Couffinahl T., Asahara T., Bauters C., Symes J., Ferrara N., Isner J. M. (1996) Gene transfer of naked DNA encoding for three isoforms of vascular endothelial growth factor endothelial growth factor stimulates collateral development in vivo. Lab. Invest. 75: 487-501). Expression of the gene product in muscle tissue can be improved by the coadministration of collagenase, papaverine and ischemic conditions (Budker V., Zhang G., Danko I., Williams P., Wolff J. (1998) The efficient expression of intravascularly delivered DNA in rat muscle. Gene Therapy 5: 272-276). The use of a muscle specific promoter (Skarli M., Kiri A., Vrbova G., Lee C. A., Goldspink G. (1998) Myosin regulatory elements as vectors for gene transfer by intramuscular injection. Gene Therapy 5: 514-520) and other intragene regulatory sequences, such as the poly A and transcription termination sequence (Hartikka J., Sawdey M., Conefert-Jensen F., Margalith M., Barnhardt K., Nolasco M., Vahlsing H. L., Meek J., Marquet M., Hobart P., Norman J., Manthorpe M. (1996) An improved plasmid DNA expression vector for direct injection into skeletal muscle. Human Gene Therapy 7: 1205-1217) will also improve transgene expression.

Liposomes are lipid bilayers entrapping a fraction of aqueous fluid. DNA will spontaneously associate to the external surface of cationic liposomes by virtue of its charge, and these liposomes will interact with the cell membrane (Felgner J. H., Kumar R., Sridhar C. N., Wheeler C. J., Tasi Y. J., Border R., Ramsey P., Martin M., Felgner P. L. (1994) Enhanced gene delivery system and mechanism studies with a novel series of cationic lipid formulations. J. Biol. Chem. 269: 2550-2561). In vitro up to 90% of certain cell lines may be transfected. By including a small amount of an anionic lipid in an otherwise cationic liposome, the DNA can be incorporated into the internal surface of the liposome, protecting it from enzymatic degradation (Alio S. F. (1997) Long term expression of the human alpha-1-antitrypsin gene in mice employing anionic and cationic liposome vector. Biochem. Pharmacol. 54: 9-13). To facilitate uptake into the cell as endosomes, targeting proteins have been included in liposomes, such as an anti-MHC antibody (Wang C. & Huang L. (1987) pH-sensitive immunoliposomes mediate target-cell-specific delivery and controlled expression of a foreign gene in mouse. Proc. Natl. Acad. Sci. USA 84: 7851-7855), transferrin (Stavridis J. C., Deliconstantinos G., Psallidopoulos M. C., Armenakas N. S., Hadjiminas D. J., Hadjiminas J. (1986) Construction of transferrin-coated liposomes for in vivo transport of exogenous DNA to bone marrow erythroblasts in rabbits. Experimental Cell Research 164: 568-572), and the Sendai virus or its F protein (Dzau J. V., Mann M. J, Morishita R., Kaneda Y. (1996). Fusigenic viral liposome for gene therapy in cardiovascular disease. Proc. Natl. Acad. Sci. USA 93: 11421-11425). The Sendai virus additionally allows the plasmid DNA to escape from the endosome into the cytoplasm, thus avoiding degradation. The inclusion of a DNA-binding protein (e.g., 28 kDa high mobility group 1 protein) enhances transcription by bringing the plasmid into the nucleus (Dzau J. V., Mann M. J, Morishita R., Kaneda Y. (1996). Fusigenic viral liposome for gene therapy in cardiovascular disease. Proc. Natl. Acad. Sci. USA 93: 11421-11425). Further proposed improvements include incorporating the Epstein-Barr virus Ori p and EBNA1 genes in the plasmid (as described above for HSV-1 amplicons) to maintain the plasmid as an episomal element (Alio S. F. (1997) Long term expression of the human alpha-1-antitrypsin gene in mice employing anionic and cationic liposome vector. Biochem. Pharmacol. 54: 9-13).

Molecular conjugates consist of protein or synthetic ligands to which a DNA binding agent has been attached. Delivery to the cell can be improved by using similar techniques to those for liposomes. Targeting proteins include asialoglycoprotein (Wagner E., Cotten M., Foisner R., Birnstiel M. L. (1991) Transferrin-polycation-DNA complexes: the effect of polycations on the structure of the complex and DNA delivery to cells. Proc. Natl. Acad. Sci. USA 88: 4255-4259), transferrin (Wu C. H., Wilson J. M., Wu. G. Y. (1989) Targeting genes: delivery and persistent expression of a foreign gene driven by mammalian regulatory elements in vivo. J. Biol. Chem. 264: 16985-16987), polymeric IgA (Ferkol T., Kaetzel C. S., Davis P. B. (1993) Gene transfer into respiratory epithelial cells by targeting the polymeric immunoglobulin receptor. J. Clin. Invest. 92: 2394-2400) and adenovirus (Madon J. & Blum H. E. (1996) Receptor mediated delivery of hepatitis B virus DNA and antisense oligodeoxynucleotides to avian liver cells. Hepatology 24: 474-481). Transgene expression tends to be transient and is limited by endosome/lysosomal degradation.

Detectable Radionuclides

Any of the radionuclide listed below, or any other such agent known or discovered to exhibit a diagnostic and/or therapeutic effect can be more effectively delivered with one or more agonist(s) in accordance with this invention. As used herein, a “detectable radionuclide” is any suitable radionuclide (i.e., radioisotope) capable of being detected in a diagnostic procedure in vivo or in vitro, which may or may not have therapeutic and/or prophylactic effects. Suitable detectable radionuclide include metallic radionuclide (i.e., metallic radioisotopes) and non-metallic radionuclide (i.e., non-metallic radioisotopes).

Suitable metallic radionuclide (i.e., metallic radioisotopes or metallic paramagnetic ions) include Antimony-124, Antimony-125, Arsenic-74, Barium-103, Barium-140, Beryllium-7, Bismuth-206, Bismuth-207, Cadmium-109, Cadmium-115m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-67, Erbium-169, Europium-152, Gallium-64, Gallium-68, Gadolinium-153, Gadolinium-157 Gold-195, Gold-199, Hafnium-175, Hafnium-175-181, Holmium-166, Indium-110, Indium-111, Iridium-192, Iron-55, Iron-59, Krypton-85, Lead-210, Manganese-54, Mercury-197, Mercury-203, Molybdenum-99, Neodymium-147, Neptunium-237, Nickel-63, Niobium-95, Osmium-185+191, Palladium-103, Platinum-195m, Praseodymium-143, Promethium-147, Protactinium-233, Radium-226, Rhenium-186, Rhenium-188, Rubidium-86, Ruthenium-103, Ruthenium-106, Scandium-44, Scandium-46, Selenium-75, Silver-110m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65, and Zirconium-95.

The compounds of the invention can also comprise one or more (e.g., 1, 2, 3, or 4) non-metallic radionuclide which can be administered in combination and/or alternation with the agonist(s) of the present invention. Specifically, the non-metallic radionuclide can be a non-metallic paramagnetic atom (e.g., Fluorine-19); or a non-metallic positron emitting radionuclide (e.g., Carbon-11, Fluorine-18, Iodine-123, or Bromine-76). Fluorine-18 is a suitable non-metallic radionuclide for use the compounds of the present invention in part because there is typically little or no background noise associated with the diagnostic use of fluorine in the body of a mammal (e.g., human). Preferably, the detectable radionuclide is a non-metallic radionuclide, e.g., Carbon-11, Fluorine-18, Bromine-76, Iodine-123, Iodine-124.

Chelating Group

Chelating groups can be used to stabilize the radionuclide of the present invention. Any suitable chelating group can be employed. Suitable chelating groups include those disclosed in U.S. Pat. No. 5,739,313. In one embodiment, the chelating group can be NTA, HEDTA, DCTA, RP414, MDP, DOTATOC, CDTA, HYNIC, EDTA, DTPA, TETA, DOTA, DOTMP, DCTA, 15N4, 9N3, 12N3, or MAG3 (or another suitable polyamine acid cheater), which are described herein below, or a propionate cheater (e.g. EDMT). In a preferred embodiment, the chelating group is DTPA.

DTPA is diethylenetriaminepentaacetic acid; TETA is 1,4,8,11-tetraaza-cyclo-tetradecane-N,N′,N″,N′″-tetraacetic acid; DOTA is 1,4,7,10-tetraaza-cyclododecane-N,N′,N″,N′″-tetraacetic acid; 15N4 is 1,4,8,12-tetraazacyclo-pentadecane-N,N′,N″,N′″-tetra-acetic acid; 9N3 is 1,4,7-triazacyclononane-N,N′,N″-triacetic acid; 12N3 is 1,5,9-triazacyclo-dodecane-N,N′,N″-triacetic acid; MAG3 is (N-(N-(N-((benzoylthio)acetyl)glycyl)glycyl)glycine); and DCTA is a cyclohexane-based metal chelator of the formula 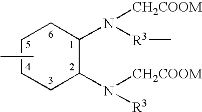
wherein R³ may by (C₁-C₄)alkyl or CH₂CO₂—, which may be attached through positions 4 or 5, or through the group R³ and which carries from 1 to 4 detectable metal or nonmetal cations (M), monovalent cations, or the alkaline earth metals. Thus, with metals of oxidation state +1, each individual cyclohexane-based molecule may carry up to 4 metal cations (where both R³ groups are CH₂COOM). As is more likely, with higher oxidation states, the number of metals will decrease to 2 or even 1 per cyclohexane skeleton. This formula is not intended to limit the molecule to any specific stereochemistry.

NTA, HEDTA, and DCTA are disclosed in Poster Sessions, Proceedings of the 46th Annual Meeting, J. Nuc. Med., p. 316, No. 1386. RP414 is disclosed in Scientific Papers, Proceedings of the 46th Annual Meeting, J. Nuc. Med., p. 123, No. 499. MDP is disclosed in Scientific Papers, Proceedings of the 46th Annual Meeting, J. Nuc. Med., p. 102, No. 413. DOTATOC is disclosed in Scientific Papers, Proceedings of the 46th Annual Meeting, J. Nuc. Med., p. 102, No. 414 and Scientific Papers, Proceedings of the 46th Annual Meeting, J. Nuc. Med., p. 103, No. 415. CDTA is disclosed in Poster Sessions, Proceedings of the 46th Annual Meeting, J. Nuc. Med., p. 318, No. 1396. HYNIC is disclosed in Poster Sessions, Proceedings of the 46th Annual Meeting, J. Nuc. Med., p. 319, No. 1398.

Bifunctional chelators (i.e., chelating groups) based on macrocyclic ligands in which conjugation is via an activated arm attached to the carbon backbone of the ligand can also be employed as a chelating group, as described by M. Moi et al., J. Amer. Chem., Soc., 49, 2639 (1989) (2-p-nitrobenzyl-1,4,7,10-tetraazacyclododecane-N,N′,N″,N′″-tetraacetic acid); S. V. Deshpande et al., J. Nucl. Med., 31, 473 (1990); G. Kuser et al., Bioconj. Chem., 1, 345 (1990); C. J. Broan et al., J. C. S. Chem. Comm., 23, 1739 (1990); and C. J. Anderson et al., J. Nucl. Med. 36, 850 (1995) (6-bromoacetamido-benzyl-1,4,8,11-tetraazacyclotetadecane-N,N′,N″,N′″-tetraacetic acid (BAT)).

In addition, the diagnostic chelator or diagnostic chelating groups can be any of the chelating groups disclosed in Scientific Papers, Proceedings of the 46th Annual Meeting, J. Nuc. Med., Wednesday, Jun. 9, 1999, p. 124, No. 500.

Specifically, the chelating group can be any one of the carbonyl complexes disclosed in Waibel et al., Nature Biotechnology, 897-901, Vol. 17, September 1999; or Sattelberger et al., Nature Biotechnology, 849-850, Vol. 17, September 1999.

Specifically, the detectable chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising a metallic radionuclide. More specifically, the detectable chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising Technetium-99m.

Specifically, the detectable chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising a metallic radionuclide. More specifically, the detectable chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising Rhenium-186 or Rhenium-188.

III. Potassium Channels

Ion channels, including potassium channels, are found in all mammalian cells and are involved in the modulation of various physiological processes and normal cellular ionic homeostasis. Potassium channels are the most common, and are found in both excitable and nonexcitable cells. They are known to be involved in cellular signaling and processes regulating diverse physiological events, including: neurotransmitter release, smooth muscle contraction, heart rate, insulin secretion, neuronal excitability, epithelial electrolyte transport, and cell volume regulation. More than 50 genes encoding various K⁺ channels have been cloned (Shieh C, Coghlan M, Sullivan, J and Murali Gopalakrishnan, M. Pharmacologic Reviews 2000 52(4), 557-594).

Potassium channels are typically classified according to their biophysical and pharmacological characteristics. The four main types of potassium channels include: inverse rectifier potassium channels (Kᵢᵣ); voltage-gated potassium channels (K_{V}); calcium-activated potassium channels (Ca²⁺-activated K⁺ channel; K_{Ca}); and ATP-sensitive potassium channels (K_{ATP}) (Nelson M T and Quayle, J M. “Physiological roles and properties of potassium channels in arterial smooth muscle” Am. J. Physiol. 1995, 268(4 Pt 1), C799-822). Subclasses of these channels are further classified according to amino acid sequence and functional properties.

Calcium-Activated Potassium Channels. The K_{Ca} channel is ubiquitously distributed in tissues, including brain capillaries, and plays an important role in diverse physiological processes. K_{Ca} channels are classified into three groups, including large conductance (BK), intermediate conductance (IK), and (3) small conductance (SK) channels, each with a distinct pharmacology. SK channels are abundant in the nervous system, where they mediate slow after-hyperpolarizations that modulate excitability. IK channels are found in red and white blood cells, colon, lung, pancreas, and other tissues. BK channels are nearly ubiquitous.

Large conductance, calcium-activated potassium channels (BK, maxi-K or BK_{Ca}) are present in many excitable cells, including neurons, cardiac cells, and types of smooth muscles. BK_{Ca} have diverse physiologic properties and have a tissue specific distribution. In neurons, BK_{Ca} channels are functionally co-localized with calcium channels, shape action potential wave forms and modulate neurotransmitter release. BK_{Ca} channels regulate constriction in arteries, uterine contraction, and filtration rate in the kidney. BK channels are activated by highly synergistic manner by depolarization and elevation in intracellular calcium concentrations (micromolar), providing a link between the calcium signaling pathways and the metabolic state of the cell with the electrical state of cells (Piskorowski R, Aldrich R “Calcium activation of BK(Ca) potassium channels lacking the calcium bowl and RCK domains” Nature 2002 420(6915):499-502; Brayden, J E. “Potassium channels in vascular smooth muscle” Clin Exp Pharmacol Physiol. 1996 23(12):1069-76. When activated, the efflux of K⁺ out of the cell hyperpolarizes the membrane potential, shutting down voltage-dependent Ca²⁺ and Na⁺ channels. In smooth muscle cells, this hyperpolorization produces relaxation or vasodilation.

The structure of the BK_{Ca} channel differs among tissues. In certain tissues, it appears that this channel is composed simply of four identical a subunits surrounding a central pore (Rothberg, B. S., and Magleby, K. L. 1999 “Gating kinetics of single large-conductance Ca²⁺-activated K⁺ channels in high Ca²⁺ suggest a two-tiered allosteric gating mechanism” J. Gen. Physiol. 114:93-124. In other tissues, including smooth muscle, the BK_{Ca} channel is composed of a pore-forming a subunit and a smaller β subunit. Over the past few years, it has become clear that the tissue-specific expression of auxiliary beta subunits provide an important mechanism by which BK_{Ca} activity is regulated. The beta1 subunit, for example, is found predominately in smooth muscle and dramatically increases the channels affinity for calcium versus BK_{Ca} channels in other tissues. Tanaka, Y., Meera, P., Song, M., Knaus, H. G., and Toro, L. “Molecular constituents of maxi KCa channels in human coronary smooth muscle: predominant alpha+beta subunit complexes” J. Physiol. 1997 502:545-557 BK_{Ca} expressed in the brain is characterized by an extremely sensitive α-subunit.

Regulation of the K_{Ca} Channel

K_{Ca} channels are regulated by several distinct mechanisms, including phosphorylation/dephosphorylation events, and by protein protein interactions.

In smooth muscle, the regulation of blood vessel tone is well-known to involve the regulation of the K_{Ca} channel by a NO-sGC-cGMP signaling pathway (Bang, L. et al. “Nitroglycerin-mediated vasorelaxation is modulated by endothelial calcium-activated potassium channels” Cardiovasc. Res. 1999 43(3):772-778; Berg T, Koteng O, “Signaling pathways in bradykinin- and nitric oxide-induced hypotension in the normotensive rat; role of K-channels” Br. J. Pharmacol. 1997 121:(6), 1113-1120; Bolotina, V M. et al. “Nitric oxide directly activates calcium-dependent potassium channels in vascular smooth muscle” Nature 1994 368:(6474), 850-853; Robertson B E et al. “cGMP-dependent protein kinase activates Ca-activated K channels in cerebral artery smooth muscle cells” Am. J. Physiol. 1993 265(1 Pt 1): C299-303; Sobey C G et al. “Mechanisms of bradykinin-induced cerebral vasodilatation in rats. Evidence that reactive oxygen species activate K⁺ channels” Stroke 1997 28(11):P2290-2294, discussion 2295; Sobey C G and Faraci F M. “Effect of nitric oxide and potassium channel agonists and inhibitors on basilar artery diameter” Am. J. Physiol. 1997 272(1 Pt 2): H256-262; Hardy P. et al. “A major role for prostacyclin in nitric oxide-induced ocular vasorelaxation in the piglet” Circ. Res. 1998 83(7): 721-29; Bychkov R. et al. “Calcium-activated potassium channels and nitrate-induced vasodilatation in human coronary arteries” J. Pharmacol. Exp. Ther. 1998 285(1): 293-298; Armstead W M. “Contribution of K_{Ca} channel activation to hypoxic cerebrovasodilation does not involve NO” Brain Res. 1998 799(1): 44-48; Joo F. et al. “Regulation of the macromolecular transport in the brain microvessels: the role of cyclic GMP” Brain Res. 1983 278(1-2), 165-174; Faraci F M. et al. “Role of soluble guanylate cyclase in dilator responses of the cerebral microcirculation” Brain Res. 1999 821(2): 368-373). Glial tumors and ischemic tissue are more immunopositive for nNOS and eNOS relative to normal brain. (Cai, Z. et al. “Prenatal hypoxia-ischemia alters expression and activity of nitric oxide synthase in young rat brain and causes learning deficits” Brain Res. Bull. 1999 49(5): 359-365).

In the smooth muscle K_{Ca} signaling pathway, cyclic GMP (cGMP or guanosine 3′,5′-cyclic monophosphate) is responsible for the activation of several effector proteins, including cGMP dependent protein kinases, which phosphorylate the K_{Ca} channel (Robertson B E. et al. “cGMP-dependent protein kinase activates Ca-activated K channels in cerebral artery smooth muscle cells” Am. J. Physiol. 1993 265(Cell Physiol. 34):C299-C303; Fukao M. et al. “Cyclic GMP-dependent protein kinase activates cloned BK_{Ca}, channels expressed in mammalian cells by direct phosphorylation at serine 1072” J. Biol. Chem. 1999 274(16):10927-10935; Becker, E M et al. “The vasodilator-stimulated phosphoprotein (VASP): target of YC-1 and nitric oxide effects in human and rat platelets” J. Cardiovasc. Pharmacol. 2000 35(3): 390-397; Lohse M J. et al. “Pharmacology of NO:cGMP signal transduction” Naunyn-Schmiedebergs Arch. Pharmacol. 1998 358(1): 111-112; Smolenski A. et al. “Functional analysis of cGMP-dependent protein kinases I and H as mediators of NO/cGMP effects” Naunyn-Schmiedebergs Arch. Pharmacol. 1998 358:134-139; He P. et al. “cGMP modulates basal and activated microvessel permeability independently of [Ca²⁺]ᵢ” Am. J. Physiol. 1998 274(6 Pt 2):H1865-1874; Holschermann H. et al. “Dual role of cGMP in modulation of macromolecule permeability of aortic endothelial cells” Am. J. Physiol. 1997 272(1 Pt 2): H91-98). cGMP-dependent protein kinases (PKG or cGK) are known to directly or indirectly activate K_{Ca} channels through enzymatic ATP dependent phosphorylation. (Robertson, B E. et al. “cGMP-dependent protein kinase activates Ca-activated K channels in cerebral artery smooth muscle cells” Am. J. Physiol. 1993 265(Cell Physiol. 34): C299-C303; Fukao M. et al. “Cyclic GMP-dependent protein kinase activates cloned BK_{Ca}, channels expressed in mammalian cells by direct phosphorylation at serine 1072” J. Biol. Chem. 1999 274(16):10927-10935; Becker, EM et al. “The vasodilator-stimulated phosphoprotein (VASP): target of YC-1 and nitric oxide effects in human and rat platelets” J. Cardiovasc. Pharmacol. 2000 35(3): 390-397).

Production of cGMP from gTP is itself catalyzed by soluble guanylyl cyclase (sGC). Soluble guanylyl cyclase is a heterodimeric protein consisting of α and β subunits, the composition of which differs among isotypes characteristic of different tissues. (Lucas K. et al., “Guanylyl cyclases and signaling by cyclic GMP” Pharmacological Reviews 2000 52(3):375-414). Each subunit is itself divided into three functional domains: home-binding, dimidiation and catalytic. The home-binding domain is located at the N terminus of each submit. Changes in the heme moiety are considered to be a major mechanism of physiological activation of sCG.

Soluble guanylyl cyclase is itself regulated by a diverse variety of ligands. (Friebe A and Koesling D. “Regulation of nitric oxide-sensitive guanylyl cyclase” Circ Res. 2003 93(2): 96-105). Nitric oxide (NO) is one such regulator, and binds directly to the heme domain of soluble guanylyl cyclase to increase the vMax of sCG 100-200-fold (Bellamy T C, Wood J, Garthwaite J. “On the activation of soluble guanylyl cyclase by nitric oxide” Proc Natl Acad Sci USA. 2002 99(1): 507-10; Humbert, P, Niroomand, F, Fischer, G, Mayer, B, Koesling, D, Hinsch, K D, Gausepohl, H, Frank, R, Schultz, G & Bohme, E. “Purification of soluble guanylyl cyclase from bovine lung by a new immunoaffinity chromatographic method” Eur. J. Biochem. 1990 190:273-278; Stone J R and Marletta M A. “Spectral and kinetic studies on the activation of soluble guanylyl cyclase by nitric oxide” Biochemistry 1996 35:1093-10; Patel, A. I. and Diamond, J. “Activation of guanosine 3′,5′-cyclic monophosphate (cGMP)-dependent protein kinase in rabbit aorta by nitroglycerin and sodium nitroprusside” J. Pharmacol. Exp. Ther. 1997, 283(2), 885-893). Carbon monoxide (CO) also activates sGC in vascular smooth muscle, but to a lesser extent than NO (Utz J, Ullrich V. “Carbon monoxide relaxes ileal smooth muscle through activation of guanylate cyclase” Biochem Pharmacol. 1991 41(8): 1195-201; Stone J R and Marletta M A. “Soluble guanylate cyclase from bovine lung: activation with nitric oxide and carbon monoxide and spectral characterization of the ferrous and ferric states” Biochemistry. 1994 33(18): 5636-40). The effects of CO can be potentiated, however, to produce levels of sCG activation similar to NO (Friebe A, Koesling D. “Mechanism of YC-1-induced activation of soluble guanylyl cyclase” Mol Pharmacol. 1998 53(1): 123-7; Friebe A, Schultz G and Koesling D “Sensitizing soluble guanylyl cyclase to become a highly CO-sensitive enzyme” EMBO J. 1996 15: 6863-6868.

There is also evidence that nitric oxide can activate K_{Ca} to produce vasorelaxation by both cGMP-dependent and cGMP-independent mechanisms (Chen, C H et al. “Nitric oxide activates Ca²⁺-activated K⁺ channels in cultured bovine adrenal chromaffin cells” Neurosci. Lett. 1998 248(2):127-129; Vaali K. et al. “Relaxing effects of NO donors on guinea pig trachea in vitro are mediated by calcium-sensitive potassium channels” J. Pharmacol. Exp. Ther. 1998 286(1): 110-114; Sobey C G and Faraci F M. “Inhibitory effect of 4-aminopyridine on responses of the basilar artery to nitric oxide” Br. J. Pharmacol. 1999 126(6):1437-1443; Kurtz A. et al. “Mode of nitric oxide action on the renal vasculature” Acta Physiol. Scand. 2000 168(1): 41-45).

K_{ATP} Channels. K_{ATP} channels were first described in cardiac ventricular myocytes (Noma A. “ATP-regulated K+ Channels in cardiac muscle” Nature 1983 305:147-148). Once termed “K_{ATP} regulated” channels, they are now termed “K_{ATP}-sensitive” channels and known to be widely distributed in a variety of tissues and cell types. (Yokoshiki H, Sunagawa M, Seki T, and Sperelakis N “ATP-sensitive K⁺ channels in pancreatic, cardiac, and vascular smooth muscle cells” Am J Physiol Cell Physiol 274:25-37). K_{ATP} channels have been identified in cerebral blood vessels (Kitazono, T., Fraci, F., Taguchi, H. and Heistad, D. “Role of potassium channels in cerebral blood vessels” Stroke 1995, 26:1713-1723) and in brain microvascular endothelial cells (Janigro, D., West, G. A., Gordon, E. L., and Winn, H. R., “K_{ATP}-sensitive K+ channels in rat aorta and brain microvascular endothelial cells” Am J Physiol. 1993, 812-821).

The structure of the plasma membrane K_{ATP} channel is well-known (Mannhold R. “K_{ATP} channel openers: structure activity relationships and therapeutic potential” Medicinal Research Reviews 2004 24(2): 213-266; Grover G J and Garlid K D. “ATP-sensitive potassium channels: a review of their cardioprotective pharmacology” J Mol Cell Cardiol 2000 32: 677-695). K_{ATP} channels are structurally unique because they require two unrelated subunits to generate functional channels including a (1) a member of the Kir inward rectifier potassium channel family, and (2) a sulfonylurea receptor (SUR), a member of the ATP-binding cassette transporter family. (Seino S. “ATP-sensitive potassium channels: a model of heteromultimeric potassium channel/receptor assemblies” Annu Rev Physiol. 1999 61: 337-62). K_{ATP} channels are also found on the mitochondria, although the detailed structure of the mito K_{ATP} is less well understood that their plasma membrane counterparts (Szewczyk A, Marban E. “Mitochondria: a new target for K+ channel openers?” Trends Pharmacol Sci 1999 20:157-161).

After some controversy, the Kir subunit is now considered to be the location of the ATP-binding site (Mannhold R. “KATP channel openers: structure-activity relationships and therapeutic potential” Medical Research Reviews 2004 24(2) 213-266, see Table I. page 117). SUR is considered the regulatory subunit of K_{ATP}, providing the channel with sensitivity to the stimulatory effects of MGADP and K⁺ channel openers or agonists, and to the inhibitory effects of sulphonylureas (Trapp, S. and Ashcroft, F. M. “A metabolic sensor in action: News from the ATP-sensitive K⁺-channel” News in Physiological Sciences 1997 12: 255-263; Tucker, S. J., Gribble, F. M., Proks, P., Trapp, S., Ryder, T. J., Haug, T., Reimann, F. and Ashcroft, F. M. “Molecular determinants of K_{ATP} channel inhibition by ATP. EMBO Journal 1998 17: 3290-3296; Inagaki N, Gonoi T, Clement J P, Wang C Z, Aguilar-Bryan L, Bryan J, Seino S. “A family of sulfonylurea receptors determines the pharmacological properties of ATP-sensitive K+ channel” Neuron. 1996 16(5): 1011-7). Nucleotide-binding folds (NBFs) of the SUR are though to play an important role in regulation by ADP and pharmacologic agonists, while the Kir channel subunit possesses the ATP-sensing mechanism (Yokoshiki H, Sunagawa M, Seki T, Sperelakis N. “ATP-sensitive K+ channels in pancreatic, cardiac, and vascular smooth muscle cells” Am J Physiol. 1998 274(1 Pt 1): C25-37; Schwanstecher M, Sieverding C, Dorschner H, Gross I, Aguilar-Bryan L, Schwanstecher C, Bryan J. “Potassium channel openers require ATP to bind to and act through sulfonylurea receptors” EMBO J. 1998 17(19): 5529-35).

The isoforms of Kir and SUR subunits can combine to form K_{ATP} channels with different properties (i.e., pharmacological and nucleotide sensitivity) (Mannhold R. “KATP channel openers: structure-activity relationships and therapeutic potential” Medical Research Reviews 2004 24(2) 213-266, see Table I. page 117). Pancreatic, cardiac, and vascular smooth muscle K_{ATP} channels have different subtypes of SUR. (Matsuo M, Tanabe K, Kioka N, Amachi T, Ueda K “Different binding properties and affinities for ATP and ADP among sulfonylurea receptor subtypes, SUR1, SUR2A, and SUR2B” J. Biol. Chem. 2000 275(37):28757-63). A SUR1/KIR6.2 combination makes up the neuronal/pancreatic beta-cell channel, whereas SUR2A/KIR6.2 and SUR2B/KIR6.1 (or KIR6.2) combination make up the cardiac and the vascular-smooth-muscle-type K_{ATP} channels, respectively (Schwanstecher M, Sieverding C, Dorschner H, Gross I, Aguilar-Bryan L, Schwanstecher C, Bryan J. “Potassium channel openers require ATP to bind to and act through sulfonylurea receptors” EMBO J. 1998 17(19): 5529-35). Smooth muscle K_{ATP} channels typically involve a SUR2B and Kir6.2 combination (Isomoto S, Kondo C, Yamada S, Matsumoto S, Higachiguchi O, Horio Y, Matsuzawz Y, Kurachi Y. “A novel sulfonylurea receptor forms with BIR (Kir6.2) a smooth muscle type ATP-sensitive K+ channel” J Biol Chem 1996 271: 24321-24324; Chutkow W A, Simon M C, Le Beau M M, Burant C F. “Cloning, tissue expression, and chromosomal localization of SUR2, the putative drug-binding subunit of cardiac, skeletal muscle, and vascular KATP channels” Diabetes 1996 45: 1439-1445). K_{ATP} channels expressed in cerebral vessels are heterodimers of sulfonylurea receptors (SUR) and pore forming inwardly rectifying potassium channel subunits (Kᵢᵣ6.x) with a (SUR-Kᵢᵣ6.x)4. (Chan K W, Zhang H, Logothetis D E “N-terminal transmembrane domain of the SUR controls trafficking and gating of Kir6 channel subunits” EMBO J. 2003 22(15):3833-43).

K_{ATP} channels are involved in diverse cellular functions including hormone secretion (i.e., insulin from pancreatic beta cells, growth hormone and prolactin from adenohypophysis cells), cardiac action potential duration, and neurotransmitter release. K_{ATP} channels are thought to play an important role in the regulation of vascular tone and relaxation, including cerebral vascular tone, in both normal and disease states. (Brayden, J. E. “Functional roles of K_{ATP} channels in vascular smooth muscle” Clin. Exp. Pharmacol. Physiol. 2002, 29:312-316). As a result, ATP-sensitive potassium channels are therapeutic targets for several diseases, including angina, hypertension, and diabetes ((Yokoshiki H, Sunagawa M, Seki T, Sperelakis N. “ATP-sensitive K+ channels in pancreatic, cardiac, and vascular smooth muscle cells” Am J Physiol. 1998 274(1 Pt 1): C25-37;

Regulation of the K_{ATP} Channel

Among the endogenous modulators, it is well known that native K_{ATP} channels are inhibited by ATP and stimulated by nucleotide diphosphates such as ADP (Yokoshiki H, Sunagawa M, Seki T, Sperelakis N. “ATP-sensitive K+ channels in pancreatic, cardiac, and vascular smooth muscle cells” Am J Physiol. 1998 274(1 Pt 1): C25-37) The ratio between ATP/ADP is though to modulate channel activity In addition, other endogenous modulators are said to include adenosine, prostacyclin, B-adrenoreceptor agonists and calcitonin gene-related peptide (Grover, G J and Garlid K D. ATP-sensitive potassium channels: a review of their cardioprotective pharmacology. J Mol Cell Cardiol 2000 32: 677-695).

Activation of K_{ATP} in vascular smooth muscle by various endogenous vasodilators (i.e., adenosine, calcitonin gene-related peptide (CGRP), prostaglandin I2) has been shown to involve an adenylyl cyclase-cAMP-protein kinase A pathway. (Brayden, J E. “Functional roles of K_{ATP} channels in vascular smooth muscle” Clin. Exp. Pharmacol. Physiol. 2002 29(4): 312-6.; Mutafova-Yambolieva N, Keef, K “Adenosine-induced hyperpolarization in guinea pig coronary artery involves A2b receptors and K_{ATP} channels” Am J Physiol Heart Circ Physiol 1997 273:H2687-2695; Kleppisch T, Nelson M T. “Adenosine activates ATP-sensitive potassium channels in arterial myocytes via A2 receptors and cAMP-dependent protein kinase” Proc Natl Acad Sci USA. 1995 92(26):12441-5; Quayle J M, Bonev A D, Brayden J E, Nelson M T., “Calcitonin gene-related peptide activated ATP-sensitive K+ currents in rabbit arterial smooth muscle via protein kinase” A. J Physiol. 1994 475(1):9-13; Wellman G C, Quayle J M, Standen N B, “ATP-sensitive K+ channel activation by calcitonin gene-related peptide and protein kinase A in pig coronary arterial smooth muscle” J Physiol. 1998 507(1):117-29; Standen N B, Quayle J M. K+ channel modulation in arterial smooth muscle Acta Physiol Scand. 1998 164(4): 549-57). Protein kinase-catalyzed phosphorylation is an important mechanism by which the activity the K_{ATP} channel can be controlled. (Light P E, Bladen C, Winkfein R J, Walsh M P, French R J “Molecular basis of protein kinase C-induced activation of ATP-sensitive potassium channels” Proc Natl Acad Sci USA. 2000 97(16):9058-63; Light, P. E. “Regulation of ATP-sensitive potassium channels by phosphorylation” Biochim. Biophys. Acta 1996 1286, 65-73).

The adenylyl cyclase-cAMP signaling pathway has been the subject of considerable study (Reithmann C, Gierschik P, Jakobs K H, “Stimulation and inhibition of adenylyl cyclase” Symp Soc Exp Biol. 1990 44:207-24; Jakobs K H, Minuth M, Bauer S, Grandt R, Greiner C, Zubin P., “Dual regulation of adenylate cyclase. A signal transduction mechanism of membrane receptors” Basic Res Cardiol. 1986 81(1):1-9). Adenylate cyclase catalyzes the formation of cAMP from ATP. Cyclic AMP functions as a second messenger to activate cAMP-dependent protein kinase A, which is then phosphorylates various ubiquitous and cell-specific proteins.

Adenylyl cyclases are intrinsic plasma membrane proteins that contain a duplicated module consisting of a hexahelical transmembrane region followed by a roughly 40-kDa cytosolic domain, which cytosolic domain contains highly conserved and homologous sequences which contribute to the active site of the enzyme (Sunahara, R K, Dessauer C W and Gilman, A G. “Complexity and diversity of mammalian adenylyl cyclases” Annu. Rev. Pharm. Toxicol. 1996 36: 461-480; Tang, W.-J., and Gilman, A. G. “Construction of a soluble adenylyl cyclase activated by Gs alpha and forskolin” Science 1995 268: 1769-1772; Chen J, Iyengar, R, “Inhibition of cloned andenylyl cyclases by mutant Gi-α and specific suppression of type 2 adenylyl cyclase inhibition by phorbol ester treatment” J Biol Chem 1993 268:12253-12256).

Adenylyl cyclases are structurally closely related to soluble guanylyl cyclase (Sunahara R K, Beuve A, Tesmer J J, Sprang S R, Garbers D L, Gilman A G. “Exchange of substrate and inhibitor specificities between adenylyl and guanylyl cyclases” J Biol Chem. 1998 273(26):16332-8; Friebe A, Russwurm M, Mergia E, Koesling D. “A point-mutated guanylyl cyclase with features of the YC-1-stimulated enzyme: implications for the YC-1 binding site?” Biochemistry. 1999 38(46):15253-7; Beuve A. “Conversion of a guanylyl cyclase to an adenylyl cyclase” Methods. 1999 19(4): 545-50). Multiple isoforms of adenylyl cyclase have been located in the brain. (Mons N, Yoshimura H, Ikeda H, Hoffman P L, Tabakoff B, “Immunological assessment of the distribution of type VII adenylyl cyclase in brain” Brain Res. 1998 788(1-2):251-6; Guillou J L, Rose G M, Cooper D M. “Differential Activation of Adenylyl Cyclases by Spatial and Procedural Learning” J Neuroscience 1999 19(14):6183-6190).

Adenylyl cyclase activity is regulated by multiple effectors, including G proteins and protein kinases (PKA, PKC and calmodulin kinase). At least five of the known isoforms are regulated by calcium, such that there is coordination between cAMP and calcium cellular signaling (Cooper D N, Mons N, Karpen J W, “Adenylyl cyclcase and the interaction between calcium and cAMP signaling” Nature 1995 374(6521):421-4). Forskolin is a direct adenylyl cyclase activator (Seamon K B, Daly J W, “Forskolin: a unique diterpene activator of cyclic AMP-generating systems” J Cyclic Nucleotide Res. 1981 7(4):201-24. The role of the adenylyl cyclase-cAMP pathway in the modulation of the K_{ATP} channel during smooth muscle relaxation is the subject of continued study.

Activation of K_{ATP} is associated with various pathological states including hypoxia and ischemia (Kitazono, T., Faraci, F. M., Tuguchi, H., and Heistad, D. D., “Role of potassium channels in cerebral blood vessels” Stroke 1995, 26, 1713-1723).

IV. Potassium Channel Agonists and Activators

The potassium channel modulator is an agonist or activator of either a calcium-activated potassium channel (K_{Ca}) of any conductance level, whether of large, intermediate, or small conductance, and/or of ATP-sensitive potassium channel (K_{ATP}). Cook et al., Potassium Channels: Structure, Classification, Function and Therapeutic Potential ed. N. S. Cook, Ellis Horwood, Chichester 1990 pp. 181-255. These pharmacologic agents cause the potassium channel to open, either directly or indirectly, thereby hyperpolarizing the cell membrane.

Potassium channel agonists, also known as potassium channel openers, are a structurally diverse group of compounds that share the common ability to open the K+ channel by binding directly thereto(Lawson, K. “Potassium channel openers as therapeutic weapons in ion channel disease: Kidney Int. 2000 57(3):838-45; Edwards, G, Weston, A H “Pharmacology of the potassium channel openers” Cardiovasc Drugs Ther. 1995 2:185-93; Edwards, G and Weston A H “Structure-activity relationships of K+channel openers” Trends Pharmacolog Sci 1990 11:417-422). These agonists may be specific to a particular type of potassium channel (i.e., K_{Ca} or K_{ATP}).

Potassium channel agonists are well known in the art and have been synthesized and used therapeutically in the treatment of any number of diseases, including hypertension and coronary artery disease (Lawson, K. “Potassium channel activation: a potential therapeutic approach?” Pharmacol. Ther. 1996, 70:39-63; Robertson, D W and Steinberg M I. “Potassium channel modulators: scientific applications and therapeutic promise” Journal of Medicinal Chemistry 1990, 33:1529-1541). In recent years, several well-known clinical compounds have been found to acts as potassium channel agonists, including minoxidil (a well-known hypertensive and hair loss treatment) and diazoxide (Andersson K E. Clinical pharmacology of potassium channel openers” Pharmacol Toxicol. 1992 April; 70(4): 244-54; Quast U. “Potassium channel openers: pharmacological and clinical aspects” Fundam Clin Pharmacol. 1992 6(7): 279-93. The number of compounds known as potassium openers or agonists continues to grow. Empirically discovered potassium channel agonists serve as templates for the design of new agents (Lawson, K. “Potassium channel openers as a potential therapeutic weapon in ion channel diseases” Kidney Int. 2000 57(3):838-45; Mannhold R. “K_{ATP} channel openers: structure activity relationships and therapeutic potential” Medicinal Research Reviews 2004 24(2): 213-266; Grover G J and Garlid K D. “ATP-sensitive potassium channels: a review of their cardioprotective pharmacology” J Mol Cell Cardiol 2000 32: 677-695). Insight into the structure and function of channel proteins promises to spur rational design and development of novel potassium channel agonists (Kaczorowski G J, Garcia M L, “Pharmacology of voltage-gated and calcium-activated potassium channels” Curr Opin Chem Biol. 1999, 3(4):448-58).

One area of scientific and commercial interest is development of tissue selective potassium channel agonists (Mannhold R. “K_{ATP} channel openers: structure activity relationships and therapeutic potential” Medicinal Research Reviews 2004 24(2): 213-266; Grover G J and Garlid K D. “ATP-sensitive potassium channels: a review of their cardioprotective pharmacology” J Mol Cell Cardiol 2000 32: 677-695). Many known potassium channel agonists are vasodilators, and it may be useful in some circumstances to avoid side effects (e.g., hypotension) associated systemic delivery while retaining useful therapeutic properties. Potassium channel agonists have reportedly been developed which are devoid of vasodilatory effects, but retain desirable cardioprotective activity (Atwal K, Grover G, Ahmed S, Ferrara F, Harper T, Kim K, Sleph P, Dzwonczyk S, Russell A, Moreland S. “Cardioselective anti-ischemic ATP-sensitive potassium channel openers” J Med Chem 1993 36: 3971-3974). New insights into structure activity relationships (SAR) permits further enhancement of tissue selectivity (Mannhold R. “K_{ATP} channel openers: structure activity relationships and therapeutic potential” Medicinal Research Reviews 2004 24(2): 213-266).

The potassium channel agonist of the present invention is not the vasodilator bradykinin (Arg-Pro-Pro-Gly-Phe-Ser-Pro-Phe-Arg), or a polypeptide bradykinin analog, such as receptor mediated permeabilizer [(RMP)-7] or A7 (e.g., Kozarich et al., U.S. Pat. No. 5,268,164 and PCT Application No. WO 92/18529). Other analogs of bradykinin include related peptide structures which exhibit the same properties as bradykinin but have modified amino acids or peptide extensions on either terminal end of the peptide. Examples of bradykinin analogs include [Phe⁸(CH₂NH)Arg⁹]-bradykinin, N-acetyl [Phe⁸(CH₂NH)Arg⁹] bradykinin and desArg9-bradykinin.

K_{Ca} Agonists and Activators

In addition to endogenous modulators, a number of pharmacologic agents are known to open the K_{Ca} channel, including various synthetic small molecules and naturally derived products (Lawson, K. “Potassium channel openers as potential therapeutic weapons in ion channel disease” Kidney Int. 2000 57(3):838-45). Many of these compounds are direct agonists, meaning they bind directly to the K_{Ca} channel thereby causing it to open, while other compounds are indirectly activators, exert this effect on the channel as the indirect result of their activity on an upstream regulatory element.

Many compounds have been reported to be specific K_{Ca} channel openers, (Lawson, K. “Potassium channel openers as potential therapeutic weapons in ion channel disease” Kidney Int. 2000 57(3):838-45); Coghlan M J, Carroll W A and Gopalakrishnan M. “Recent developments in the biology and medicinal chemistry of potassium channel modulators: update from a decade of progress” J Med Chem 2001 44: 1627-1653; Starrett J E, Dworetzky S I and Gibkoff V K “Modulators of large-conductance calcium-activated potassium (BK) channels as potential therapeutic targets” Current Pharmaceut Design 1996 2: 413-428; Imaizumi Y, Sakamoto K, Yamada A, Hotta A, Ohya S, Muraki K, Uchiyama M, Ohwada T. “Molecular Basis of Pimarane Compounds as Novel Activators of Large-Conductance Ca²⁺-Activated K⁺ Channel α-Subunit” Molecular Pharmacology 2002 62(4): 836-846; Koh D S et al, Neuroscience Letters 1994 165: 167-170); Singh S B, Goetz M A, Zink D L, Dombrowski A W, Polishook J D, Garcia M L, Schmalhofer W, McManus O B and Kaczorowski G J. “Maxikdiol: a novel dihydroxyisoprimane as an agonist of maxi-K channels. J Chem Soc Perkin Trans 1994 1: 3349-3352; Lee S H, Hensens O D, Helms G L, Liesch J M, Zink D J, Giacobbe R A, Bills G F, Stevens-Miles S, Garcia M L, Schmalhofer W A, et al. “L-735,334, a novel sesquiterpenoid potassium channel-agonist from Trichoderma virens” J Nat Prod 1995 58: 1922-1828).

Many known K_{Ca} channel openers are benzimidazolone derivatives, including NS-4 and NS-1619, biarylureas (NS-1608), arylpyrrole (NS-8), and indole-3-carboxylic acid esters (CGS-7181 and CGS-7184). Others are substituted oxinodoles (e.g., U.S. Pat. No. 5,563,383 to Hewasam). A non-limiting list of known K_{Ca} channel openers is provided herein. NS004 and NS1619, have served as the basis for design of several novel and heterogeneous synthetic K_{Ca} openers. (Lawson, K. “Potassium channel openers as potential therapeutic weapons in ion channel disease” Kidney Int. 2000 57(3):838-45). The discovery of numerous variants of the alpha and beta subunits of the BK_{Ca} channel gives potential to target specific tissues with selective openers.

Indirect activators of the K_{Ca} channel include endogenous and pharmacologic agents that increase the amount of NO or cGMP or cGMP-dependent protein kinases (e.g., PKA) in vivo, and thereby indirectly activate the K_{Ca} channel via the NO-sGC-cGMP signaling pathway. Activators of nitric oxide synthase provide one example, as do activators of soluble guanylyl cyclase. Nitric oxide (NO) is a known activator of soluble guanylyl cyclase and an indirect K_{Ca} channel activator (Bellamy T C, Wood J, Garthwaite J. “On the activation of soluble guanylyl cyclase by nitric oxide” Proc Natl Acad Sci USA. 2002 99(1): 507-10).

A number of nitric oxide-independent activators of soluble guanlyly cyclase are also known, both endogenous and pharmacological (Behrends S. “Drugs that activate specific nitric oxide sensitive guanylyl cyclase isoforms independent of nitric oxide release” Current Medicinal Chemistry 2003 10: 291-301). Carbon monoxide (CO), for example, is an endogenous NO-independent activator of soluble guanylyl cyclase (Schultz G and Koesling D “Sensitizing soluble guanylyl cyclase to become a highly CO-sensitive enzyme” EMBO J 1996 15: 6863-6868). Porphyrins and metalloporphyrins, such as Protoporphyrin IX, are known NO-independent activators of sCG (Ignarro L J, Wood K S and Wolin M S. “Activation of purified soluble guanylate cyclase by protoporphyrin IX” Proc Natl Acad Sci USA 1982 79: 2870-2873; Ignarro L J, Wood K S and Wolin M S. “Regulation of purified soluble guanylate cyclase by porphyrins and metalloporphyrins: a unifying concept” Adv Cyclic Nucleotide Protein Phosphorylation Res 1984 17: 267-274). YC-1 [3-(5′-hydroxymethyl-2′furyl)-1-benzyl indazole] is a synthetic benzylindazole compound that is known to activate soluble guanylyl cyclase by direct binding to the enzyme (Denninger J W, Schelvis J P, Brandish P E, Zhao Y, Babcok G T, Marletta M A. “Interaction of soluble guanylate cyclase with YC-1: kinetic and resonance Raman studies” Biochemistry 2000 39: 4191-4198; Friebe A, Koesling D. “Mechanism of YC-1-induced activation of soluble guanylyl cyclase” Mol Pharmacol. 1998 53(1):123-7). In the presence of YC-1, CO produces more than a 100-fold increase in soluble guanylyl cyclase activity, a level equivalent to the activation of sCG by NO (Friebe A, Schultz G and Koesling D. “Sensitizing soluble guanylyl cyclase to become a highly CO-sensitive enzyme” EMBO J 1996 15: 6863-6868). Potentiation of protoporphyrin IX is also pronounced in the presence of YC-1 (Friebe A, Koesling D. “Mechanism of YC-1-induced activation of soluble guanylyl cyclase” Mol Pharmacol. 1998 53(1):123-7). YC-1 is thought to increases the affinity for heme ligands of these activators by reduction of dissociation rates (Russwurm M, Mergia E, Mullershausen F, Koesling D. “Inhibition of deactivation of NO-sensitive guanylyl cyclase accounts for the sensitizing effect of YC-1” J Biol Chem. 2002 277(28):24883-8).

Recently, a new class of NO-independent compounds has been discovered that activate soluble guanylyl cyclase independent of nitric oxide, including the YC-related compounds BAY 41-2272 and BAY 41-8543 and the chemically unrelated BAY 58-2667 (Behrends S. “Drugs that activate specific nitric oxide sensitive guanylyl cyclase isoforms independent of nitric oxide release” Current Medicinal Chemistry 2003 10: 291-301; Stasch J P, Becker E M, Alonso-Alija C, Apeler H, Dembowsky K, Feurer A, Gerzer R, Minuth T, Perzborn E, Pleiss U, Schroder H, Schroeder W, Stahl E, Steinke W, Straub A, Schramm M. “NO-independent regulatory site on soluble guanylate cyclase” Nature. 2001 8:410(6825):212-5). These guanylate cyclase stimulators act through novel regulatory sites (“NO-independent regulatory site of direct sGC stimulators like YC-1 and BAY 41-2272” BMC Pharmacol. 2001 1(1):13; Stasch J P, Schmidt P, Alonso-Alija C, Apeler H, Dembowsky K, Haerter M, Heil M, Minuth T, Perzborn E, Pleiss U, Schramm M, Schroeder W, Schroder H, Stahl E, Steinke W, Wunder F. “NO- and haem-independent activation of soluble guanylyl cyclase: molecular basis and cardiovascular implications of a new pharmacological principle” Br J Pharmacol. 2002 136(5):773-83; Straub A, Stasch J P, Alonso-Alija C, Benet-Buchholz J, Ducke B, Feurer A, Furstner C. “NO-independent stimulators of soluble guanylate cyclase” Bioorg Med Chem Lett. 2001 11(6):781-4). NO-independent activators of sCG have been proposed for the treatment of hypertension, angina and erectile dysfunction. These drugs have also been proposed for use in combination with NO-releasing drugs to make such therapies more efficient.

In one embodiment of the present invention, the K_{Ca} activator is a direct agonist of the K_{CA} channel. Suitable K_{CA} agonists may include, without limitation, benzimidazolone derivatives, substituted oxinodoles, and 4-aryl hyudroxyuinolin-2-one derivatives. Representative, non-limiting examples of KCA agonists suitable for use in the present invention include:
NS-1619 (also known as 1,3-dihydro-1-[2-hydroxy-5-(trifluoromethyl)phenyl]-5-(trifluoromethyl)-2-H-benzimidazol-2-one; available from RBI, Natick, Mass.) (Olesen S P, Munch E, Moldt P, Drejer J. “Selective activation of Ca(2+)-dependent K+ channels by novel benzimidazolone” Eur J Pharmacol. 1994 251(1): 53-9; Yamamura H, Ohi Y, Muraki K, Watanabe M, Imaizumi Y. “BK channel activation by NS-1619 is partially mediated by intracellular Ca2+ release in smooth muscle cells of porcine coronary artery” Br J Pharmacol. 2001 132(4): 828-34. NS-1619 is known to be a selective opener of the large-conductance Ca(2+)-dependent K+ channel (BK channel).
NS-1608, a biarylurea (also known as N-(3-(trifluoromethyl)phenyl)-N′-(2-hydroxy-5-chlorophenyl)urea (StrobaekD, Christophersen P, Holm N R, Moldt P, Ahring P K, Johansen T E, Olesen S P. “Modulation of the Ca(2+)-dependent K+ channel, hslo, by the substituted diphenylurea NS 1608, paxilline and internal Ca2+” Neuropharmacology. 1996 35(7): 903-14.
NS004 (also known as 5-trifluoromethyl-1-(5-chloro-2-hydroxyphenyl)-1,3,-dihydro-2H-benzimidazole-2-one (McKay M C, Dworetzky S I, Meanwell N A, Olesen S P, Reinhart P H, Levitan I B, Adelman J P, Gribkoff V K. “Opening of large-conductance calcium-activated potassium channels by the substituted benzimidazolone NS004” J Neurophysiol. 1994 71(5): 1873-82; Olesen S P, Munch E, Watjen F, Drejer J. “NS 004—an activator of Ca(2+)-dependent K+ channels in cerebellar granule cells” Neuroreport. 1994 5(8): 10014. Gribkoff V K, Lum-Ragan J T, Boissard C G, Post-Munson D J, Meanwell N A, Starrett J E Jr, Kozlowski E S, Romine J L, Trojnacki J T, Mckay M C, Zhong J, Dworetzky S I. “Effects of channel modulators on cloned large-conductance calcium-activated potassium channels Mol Pharmacol. 1996 July; 50(1): 206-17)
NS-008 (an arylpyrrole also known as 2-amino-5-(2-fluorophenyl)-4-methyl-1H-pyrrole-3-carbonitrile and CGS-7181 and CGS-7184, both indole-3-carboxylic acid esters (Sanchez M, McManus O B. “Paxilline inhibition of the alpha-subunit of the high-conductance calcium-activated potassium channel” Neuropharmacology. 1996 35(7): 963-8).
EBIO, also known as 1-ethyl-2-benzimidazolinone; an avena pyrone extracted from avena sativa-common oats [International Patent application WO 93/08800, published May 13, 1993] (Walker S D, Dora K A, Ings N T, Crane G J, Garland C J. “Activation of endothelial cell IK(Ca) with 1-ethyl-2-benzimidazolinone evokes smooth muscle hyperpolarization in rat isolated mesenteric artery” Br J. Pharmacol. 2001 134(7): 1548-54.

Other known activators of the K_{Ca} channel suitable for use in the present invention include:
DHS-1 (or dehydrosaponin 1) McManus O B, Helms L M, Pallanck L, Ganetzky B, Swanson R, Leonard R J. “Functional role of the beta subunit of high conductance calcium-activated potassium channels” Neuron. 1995 14(3): 645-50.
Maxikdiol (Lawson, K. “Potassium channel openers as potential therapeutic weapons in ion channel disease” Kidney Int. 2000 57:838-45; McManus O B, Harris G H, Giangiacomo K M, Feigenbaum P, Reuben J P, Addy M E, Burka J F, Kaczorowski G J, Garcia M L. “An activator of calcium-dependent potassium channels isolated from a medicinal herb” Biochemistry. 1993 32(24): 6128-33).
the flavonoid phloretin, (Koh, D S et al., “Activating effect of the flavonoid phloretin on Ca(2+)-activated K+ channels in myelinated nerve fibers of Xenopus laevis [corrected]” Neuroscience Lett. 1994165:167-170).
L-735,334 (Lee S H, Hensens O D, Helms G L, Liesch J M, Zink D L, Giacobbe R A, Bills G F, Stevens-Miles S, Garcia M L, Schmalhofer W A, et al. “L-735,334, a novel sesquiterpenoid potassium channel-agonist from Trichoderma virens” J Nat Prod. 1995 58(12): 1822-8; Imaizumi Y, Sakamoto K, Yamada A, Hotta A, Ohya S, Muraki K, Uchiyama M, and Ohwada T. “Molecular basis of pimarane compounds as novel activators of large-conductance Ca²⁺-activated K⁺ channel alpha-subunit” Molecular Pharmacology 2002 62(4), 836-846).
Pimaric acid (PiMA) and related compounds (Imaizumi Y, Sakamoto K, Yamada A, Hotta A, Ohya S, Muraki K, Uchiyama M, and Ohwada T. “Molecular basis of pimarane compounds as novel activators of large-conductance Ca²⁺-activated K⁺ channel alpha-subunit” Molecular Pharmacology 2002 62(4), 836-846).
niflumic acid (Wallner M, Meera P, Ottolia M, Kacaorowski G J, Latoree R, Garcia M L, Stefani E, Toro L. “Characterization and modulation by a beta-subunit of a human maxi KCa channel cloned from myometrium” Receptors Channels 1995 3(3): 185-99).
Flufenamic acid (Gribkoff V K, Lum-Ragan J T, Boissard C G, Post-Munson D J, Meanwell N A, Starrett J E, Kozlowski E S, Romine J L, Trojnacki J T, Mckay M C, Zhong J and Dworetzky S I. “Effects of channel modulators on cloned large-conductance calcium-activated potassium channels” Molecular Pharmacology 1996 50(1): 206-217).
Nitrendipine
6-bromo-8-(methylamino)imidazo[1,2-a]pyrazine-2-carbonitrile(SCA-40) (Laurent F et al., Br. J. Pharmacol. 1993 108, 622-626).
BMS-2-4352, also known as (3S)-(+)-(5-Chloro-2-methoxyphenyl)-1,3-dihydro-3-fluoro-6-(trifluoromethyl)-2H-indole-2-one); a fluorooxindole targeted for use against stroke. Jensen B S. CNS Drug Rev 2002; MacKay Current Opinion Investig Drugs 2001
Retigabine (also known as N-(2-amino-4-(4-fluorobenzylamino)-phenyl)carbamic acid ethyl ester) a potassium channel opener developed for epilepsy; Cooper E C. Epilepsia 2001; EP 554543 (and another PCT on December 2001??)
LP-805 (also known as 8-tert-butyl-6,7-dihydropyrolo[3,2-e]-5-methylpyrazolo[1,5-a]pyrimidine-3-carbonitrile) (Inazu M, Zhang H, Daniel E E. “Properties of the LP-805-induced potassium currents in cultured bovine pulmonary artery endothelial cells” J Pharmacol Exp Ther. 1994 268(1): 403-8; A target K+ channel for the LP-805-induced hyperpolarization in smooth muscle cells of the rabbit portal vein” Naunyn Schmiedebergs Arch Pharmacol. 1993 347(3): 329-35
tamoxifen (Dick, G M, Hunter C A, and Sanders K M. “Ethylbromide Tamoxifen, a Membrane-Impermeant Antiestrogen, Activates Smooth Muscle Calcium-Activated Large-Conductance Potassium Channels from the Extracellular Side” Molecular Pharamcology 2002 61(5): 1105-1113)
17-³-estradiol (Behrens R Nolting A Reimann F, Schwarz M, Waldschutz R, Pongs O. “hKCNMB3 and hKCNMB4, cloning and characterization of two members of the large conductance calcium-activated potassium channel β submit family” FEBS Letters 2000 474:99-106; Valverde, M A, Rojas P, Amigo J, Cosmelli D, Orio P, Bahamonde M I, Mann G E, Vergara C, Latorre R. “Acute activation of Maxi-K channels (hSlo) by estradiol binding to the beta subunit” Science 1999 285(5435): 1929-31). 17-estradiol is thought to activate BKCa channels.
epoxyeicosatrienoic acids (Fukao M, Mason H S, Kenyon J L, Horowitz B and Keef K D. “Regulation of BKCa channels expressed in human embryonic kidney 293 cells by epoxyeicosatrienoic acid” Mol Pharmacol 2001 59: 16-23)
nordihydroguaiaretic acid (NDGA) (Yamamura H, Nagano N, Hirano M, Murakin K, Watanabe M, Imaizumi Y. “Activation of Ca²⁺-Dependent K⁺ Current by Nordihydroguaiaretic Acid in Porcine Coronary Arterial Smooth Muscle Cells Journal of Pharmacology and Experimental Therapeutics 1999 291(1): 140-146)
5-nitro-2-(-3-phenylpropylamino)benzoic acid (NPPB) (Gribkoff V K, Lum-Ragan J T, Boissard C G, Post-Munson D J, Meanwell N A, Starrett J E, Kozlowski E S, Romine J L, Trojnacki J T, Mckay M C, Zhong J and Dworetzky S I. “Effects of channel modulators on cloned large-conductance calcium-activated potassium channels” Molecular Pharmacology 1996 50(1): 206-217).

In one embodiment, the K_{Ca} activator is one that is disclosed in U.S. Pat. No. 5,200,422 to Olesen et al. (NeuroSearch), which is herein incorporated by reference. The benzamidazole K_{Ca} agonist is of the formula: 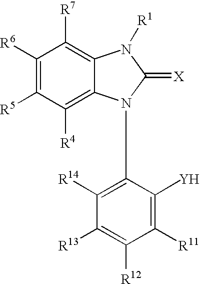
or a nontoxic pharmaceutically acceptable salt, solvate or hydrate thereof, wherein:
R¹ is hydrogen, NH₂ or C₁₋₆-alkyl which may be branched
X is O, S, NCN;
Y is O, S;
R⁴, R⁵, R⁶, and R⁷ independently of each other are hydrogen, halogen, CF₃, NO₂, NH₂, OH, C₁₋₆-alkoxy, C(═O)-phenyl or SO₂NR^{I}R^{II} wherein R^{I} and R^{II} independently are hydrogen or C₁₋₆-alkyl;
R¹¹ is hydrogen, halogen, NO₂, or SO₂NR′R″ wherein R′ and R″ independently are hydrogen or C₁₋₆-alkyl;
R¹³ is hydrogen, halogen, phenyl, CF₃, NO₂;
R¹² is hydrogen or together with R¹³ forms a C₄₋₇-carbocyclic ring which may be aromatic or partially saturated;
R¹⁴ is hydrogen or together with R¹³ forms a C₄₋₇-carbocyclic ring which may be aromatic or partially saturated.

In a sub-embodiment, the K_{Ca} agonist is 5-trifluoromethyl-2,3-dihydro-1-(5-chloro-2-hydroxyphenyl)-1H-2-oxo-benzimidazole, 5-trifluoromethyl-2,3-dihydro-1-(5-phenyl-2-hydroxyphenyl)-1H-2-oxo-benzimidazole, or 5-trifluoromethyl-6-nitro-2,3-dihydro-1-(3-nitro-5-chloro-2-hydroxyphenyl)-1H-2-oxo-benzimidazole.

U.S. Pat. No. 5,565,483 to Hewawasam et al. (Bristol-Myers Squibb), which is herein incorporated by reference, discloses various 3-substituted oxindole derivatives that open calcium-activated potassium channels that can be used with the present invention. The 3-phenyl oxindole K_{Ca} agonist is of the formula: 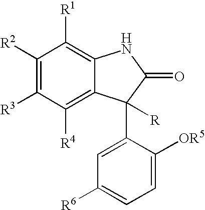
or a nontoxic pharmaceutically acceptable salt, solvate or hydrate thereof, wherein:
R is hydrogen, hydroxy or fluoro;
R¹, R², R³, and R⁴ each are independently hydrogen, C₁₋₄ alkyl, halogen, trifluoromethyl, phenyl, p-methylphenyl or p-trifluoromethylphenyl; or
R¹ and R², R² and R³, or R³ and R⁴ are joined together to form a benzo fused ring;
R⁵ is hydrogen or C₁₋₄ alkyl; and
R⁶ is chlorine or trifluoromethyl.

In another embodiment, the K_{Ca} activator is one that is disclosed in U.S. Pat. No. 5,399,587 to Garcia et al. (Merck), which is herein incorporated by reference. The sesquiterpene K_{Ca} agonist is of the formula: 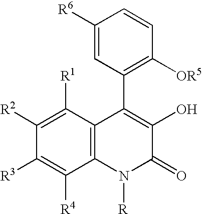
or a nontoxic pharmaceutically acceptable salt, solvate or hydrate thereof, wherein:
R is hydrogen or methyl;
R¹, R², R³, and R⁴ each are independently hydrogen, bromo, chloro or trifluoromethyl, and when R¹, R³, and R⁴ are hydrogen, R² is nitro;
R⁵ is hydrogen or methyl; and
R⁶ is bromo or chloro.

In one embodiment, the K_{Ca} activator is one that is disclosed in U.S. Pat. No. 5,399,587 to Garcia et al. (Merck), which is herein incorporated by reference. The sesquiterpene K_{Ca} agonist is of the formula: 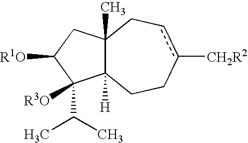
or a nontoxic pharmaceutically acceptable salt, solvate or hydrate thereof.

In one embodiment of the invention, the K_{Ca} activator is a compound that indirectly activates the K_{Ca} channel. Indirect activators of K_{Ca} include, without limitation, agents that increase the amount of nitric oxide (NO), cGMP or cGMP dependent protein kinases in vivo, and thereby indirectly activate the K_{Ca} channel via a NO-sCG-cGMP signaling pathway.

Activators of nitric oxide synthase (NOS) represent on such class of indirect activators. NOSs are a family of complex enzymes that catalyze the five-electron oxidation of L-arginine to form NO and L-citrulline. See Wang Y. Adv Pharmacol. (1995) 34:71-90). They are cytochrome P-450-like hemeproteins that depend on molecular oxygen, NADPH, flavins, and tetrahydrobiopterin. Three isoforms include ecNOS, nNOS, and iNOS. In a particular embodiment, the indirect activator of K_{Ca} is an activator of nitric oxide synthase. See Nathan C. Regulation of the Biosynthesis of Nitric Oxide J Biol Chem. (1994) 13: 269(19):13725-8.

Activators of soluble guanylyl cyclase represent another, as agents that increase the amount of cGMP and thereby indirectly activate the K_{Ca} channel (See, e.g., Koesling, D., Modulators of soluble guanylyl cyclase, Naunyn-Schmiedebergs Arch. Pharmacol. 1998 358: 123-126; Hobbs A J. “Soluble guanylate cyclase: an old therapeutic target re-visited” Br J Pharmacol. 2002 136(5):637-40; Schmidt H H. “NO., CO and .OH. Endogenous soluble guanylyl cyclase-activating factors” FEBS Lett. 1992 307(1):102-7.).

Nitric oxide is a known to activate soluble guanylyl cyclase. A preferred potassium channel activator is nitric oxide gas, which is fully permeable across biological membranes. Inhalable nitric oxide gas can be administered to the subject by mask in a controlled gas mixture as is known in the art. (Kieler-Jensen, N. et al., Inhaled nitric oxide in the evaluation of heart transplant candidates with elevated pulmonary vascular resistance, J Heart Lung Transplant. 1994 13 (3): 366-75; Rajek, A. et al., “Inhaled nitric oxide reduces pulmonary vascular resistance more than prostaglandine E(1) during heart transplantation” Anesth Analg. 2000 90 (3): 523-30; Solina, A. et al., “A comparison of inhaled nitric oxide and milrinone for the treatment of pulmonary hypertension in adult cardiac surgery patients” J Cardiothorac Vasc. Anesth. 2000 14 (1): 12-17; Fullerton, D. A. et al., “Effective control of pulmonary vascular resistance with inhaled nitric oxide after cardiac operation” J Thorac Cardiovasc Surg 1996 111 (4): 753-62, discussion 762-3). The concentration in the gas mixture of nitric oxide (NO) is preferably about 1 to 100 ppm NO, more preferably about 4 to 80 ppm NO, and most preferably about 20 to 40 ppm NO. The gas mixture also contains appropriate concentrations of oxygen and nitrogen and/or other inert gases, such as carbon dioxide, helium or argon. Optionally, gaseous anesthetics, such as nitrous oxide [(N20),] xenon, and halogenated volatile anesthetics (HVAs), e.g., halothane, sevoflurane, and isoflurane, can also be included in the gas mixture.

When administration of the potassium channel activator (and/or the medicament or chemotherapeutic agent) is by intracarotid infusion, general anesthesia is typically not required using intravenous or other delivery routes. The skilled practitioner is aware of evidence that HVAs can inhibit soluble guanylyl cyclase activity. (Masaki, E., Halogenated volatile anesthetics inhibit carbon monoxide-stimulated soluble guanylyl cyclase activity in rat brain, Acta Anaesthesiol. Scand. 2000 44 (3): 321-25; Masaki E. and Kondo I. “Methylene blue, a soluble guanylyl cyclase inhibitor, reduces the sevoflurane minimum alveolar anesthetic concentration and decreases the brain cyclic guanosine monophosphate content in rats” Anesth. Analg. 1999 89 (2): 484-88). Consequently, an HVA is not the preferred choice of inhalable anesthesia for use with a guanylyl cyclase activator in accordance with the method.

Nitric oxide donors are compounds that produce NO-related physiological activity when applied to biological systems. Thus, NO-donors can mimic an endogenous NO-related response or substitute for an endogenous NO deficiency. Compounds that have the capacity to release NO have been widely used as therapeutic agents (Ignarro, L. J., Napoli, C., Loscalzo, J. “Nitric oxide donors and cardiovascular agents modulating the bioactivity of nitric oxide: an overview” Circ Res. 2002, 90(1):21-8). Nitrovasodilators, organic nitrate and nitrite esters, including nitroglycerin, amyl nitrite, isosorbide dinitrate, isosorbide 5-mononitrate, and nicorandil have been in clinical use for decades in the treatment of cardiovascular diseases. Their vasomodulatory effects are mediated by guanylyl cyclase activation and by direct inhibition of nonspecific cation channels in vascular smooth muscle cells (VSMCs). These agents provide NO-replacement therapy. A variety of new NO donor drugs have been developed which provide advantages over commonly used NO donors, including limits on pharmacologic tolerance (Loskove J A, Frishman W H. “Nitric oxide donors in the treatment of cardiovascular and pulmonary disease” Am Heart J. 1995 129(3):604-13), spontaneous release of NO in solution (e.g., S-nitrosopenacillamine), prolonged release over period of time at predictable rates (e.g., NCX-4016), and tissue selectivity. Muscara, M. N. and Wallace, J. L. “Nitric oxide: therapeutic potential of nitric oxide donors and inhibitors” Am J Physiol Gastrointest Liver Physiol 1999, 276: G1313-G1316, 1999; Megson, I. L., “Nitric oxide donor drugs” Drugs of the Future 2000, 25: 701-715).

The skilled artisan is aware that in biological systems there are at least three redox states of NO that can be released by various NO donors [(NO⁺, NO^{o}, or NO⁻), all of which are encompassed by the terms “nitric oxide” or “NO” for purposes of the present invention. The redox state of NO makes a substantial difference to the NO donors reactivity towards other biomolecules, the profile of by-products, and the bioresponse (Feelisch, M., The use of nitric oxide donors in pharmacological studies, Naunyn-Schmiedebergs Arch. Pharmacol. 358: 113-22 [1998]). The pathways leading to enzymatic and/or nonenzymatic formation of NO differ greatly among individual compound classes, as do their chemical reactivities and kinetics of NO release. Some classes of NO donors require enzymatic catalysis, while others produce NO non-enzymatically; some NO donors require reduction, for example by thiols, and some oxidation, in order to release NO.

Preferred examples of nitric oxide donors include organic nitrate compounds which are nitric acid esters of mono-and polyhydric alcohols. Typically, these have low water solubility, and stock solutions are prepared in ethanol or dimethyl sulfoxide (DMSO). Examples are glyceryl trinitrate (GTN) or nitroglycerin (NTG), pentaerythrityl tetranitrate (PETN), isosorbide dinitrate (ISDN), and isosorbide 5-mononitrate (IS-5-N). Administration of organic nitrates can be done intravenously, intraperitoneally, intramuscularly, transdermally, or in the case of PETN, ISDN, NTG, and IS-5-N, orally.

Other preferred examples are S-nitrosothiol compounds, including S-nitroso-N-acetyl-D,L-penicillamine (SNAP), S-nitrosoglutathione (SNOG), S-nitrosoalbumin, S-nitrosocysteine. S-Nitrosothiol compounds are particularly light-sensitive, but stock solutions kept on ice and in the dark are stable for several hours, and chelators such as EDTA can be added to stock solutions to enhance stability. Administration is preferably by an intravenous or intra-arterial delivery route. NO releasers such as sodium nitroprusside (SNP) and 8-bromo-cGMP are contemplated as indirect activators of KCl according to the present invention.

Other preferred examples of nitric oxide donors include sydnonimine compounds, such as molsidomine (N-ethoxycarbonyl-3-morpholino-sydnonimine), linsidomine (SIN-1; 3-morpholino-sydnonimine or 3-morpholinylsydnoneimine or 5-amino-3-morpholinyl-1,2,3-oxadiazolium, e.g., chloride salt), and pirsidomine (CAS 936).

Stock solutions are typically prepared in DMSO or DMF, and are stable at [4° C.] to room temperature, if protected from light. Linsidomine is highly water soluble and stable in acidic solution in deoxygenated distilled water, adjusted to about pH 5, for an entire day. At physiological pH, SIN-1 undergoes rapid non-enzymatic hydrolysis to the open ring form SIN-1A, also a preferred nitric oxide donor, which is stable at pH 7.4 in the dark.

Also useful as nitric oxide donors are iron nitrosyl compounds, such as sodium nitroprusside (SNP; sodium pentacyanonitrosyl ferrate (II)). Aqueous stock solutions are preferably made freshly in deoxygenated water before use and kept in the dark; stability of stock solutions is enhanced at pH 3-5. Inclusion in the delivery buffer of a physiologically compatible thiol, such as glutathione, can enhance release of NO. SNP is administered by intravenous infusion, and the skilled practitioner is aware that long-term use is precluded by the release of five equivalents of toxic CN-per mole SNP as NO is released.

A most preferred nitric oxide donor is chosen from among the so-called NONOate compounds. The NONOates are adducts of NO with nucleophilic residues (X⁻), such as an amine or sulfite group, in which an NO dimer is bound to the nucleophilic residue via a nitrogen atom to form a functional group of the structure X[—N(O)NO]. The NONOates typically release NO at predictable rates largely unaffected by biological reactants, and NO release is thought to be by acid-catalyzed dissociation with the regeneration of X⁻ and NO. This property is particularly useful in accordance with the inventive methods of selectively delivering a medicament, because abnormal brain regions and malignant tumors can typically be relatively hypoxic and possess a relatively low ambient pH (e.g., pH 6.5-7.0), which concentrates release of NO selectively in the microvasculature of the abnormal brain region or malignant tumor.

NONOates include most preferably diethylamine-NONOate (DEA/NO; N-ethylethanamine: 1,1-diethyl-2-hydroxy-2-nitrosohydrazine (1:1) or [1-[N,N-diethylamino]diazen-1-ium-1,2-diolate). Other preferred NONOates include diethylene-triamine-NONOate (DETA/NO; 2,2′-hydroxynitrosohydrazino]bis-ethanamine), spermine-NONOate (SPER/NO; N-(4-[1-(3-aminopropyl)-2-hydroxy-2-nitroso-hydrazino]-butyl)-1,3-propanediamine), propylamino-propylamine-NONOate (PAPA/NO); 3-(2-hydroxy-2-nitroso-1-propylhydrazino)-1-propanamine or (Z)-1-[N-(3-aminopropyl)-N-(n-propyl)amino]diazen-1-ium-1,2-diolate), MAHMA-NONOate (MAHMA/NO; 6-(2-hydroxy-1-methyl-2-nitrosohydrazino)-N-methyl-1-hexanamine), dipropylenetriamine-NONOate (DPTA/NO; 3,3′-(hydroxynitrosohydrazino)bis-1-propanamine), PIPERAZI/NO, proli-NONOate (PROLI/NO; 1-([2-carboxylato]-pyrrolidin-1-yl)diazen-1-ium-1,2-diolate-methanol, e.g., disodium salt), SULFO-NONOate (SULFO/NO; hydroxydiazenesulfonic acid 1-oxide, e.g., diammonium salt), the sulfite NONOate (SULFI/NO), and Angelis salt (OXI/NO).

Almost all NONOate compounds are highly soluble in water, and aqueous stock solutions are prepared in cold deoxygenated 1 to 10 mM NaOH (preferably about pH 12) just prior to use. Alkaline stock solutions are stable for several hours if kept on ice in the dark. The characteristic UV absorbance of NONOates can be used for spectrophotometric quantification of NONOate in aqueous solutions. NONOates are preferably administered intravenously or intra-arterially.

Nitric oxide donors have different potencies (Ferraro, R. et al., Comparative effects of several nitric oxide donors on intracellular cyclic GMP levels in bovine chromaffin cells: correlation with nitric oxide production, Br. J. Pharmacol. 127 (3): 779-87 [1999]). For example, DEA/NO is among the most potent nitric oxide donors, with a half-life of about 2 to 4 minutes; less potent are PAPA/NO (t_{1/2} about 15 minutes), SPER/NO (t_{1/2} about 34-40 minutes); even less potent are DETA/NO (t_{1/2} about 20 hours) and SNAP (t % A about 33 to 41 hours, although this can be shortened in the presence of a physiological reductant such as glutathione). SNP is also a potent NO donor. (See, Ferrero et al. [1999]; Salom, J. B. et al., Relaxant effects of sodium nitroprusside and NONOates in rabbit basilary artery, Pharmacol. 57 (2): 79-87 [1998]; Salom, J. B. et al., Comparative relaxant effects of the NO donors sodium nitroprusside, DEA/NO and SPER/NO in rabbit carotid arteries, Gen. Pharmacol. 32 (1): 75-79 [1999]; Salom, J. B. et al., Relaxant effects of sodium nitroprusside and NONOates in goat middle cerebral artery delayed impairment by global ischemia-reperfusion, Nitric Oxide 3(1): 85-93 [1999]; Kimura, M. et al., Responses of human basilar and other isolated arteries to novel nitric oxide donors, J. Cardiovac. Pharmacol. 32 (5): 695-701 [1998]). Consequently, effective concentrations or doses of NONOates or other NO donors will vary over the preferred dose ranges for potassium channel activators described herein.

Stock solutions of NO donors are preferably made up freshly before use (at the appropriate pH for each particular NO donor), chilled on ice, and protected from light (e.g., by the use of darkened glass vials wrapped in aluminum foil), although organic nitrates can be stored for months to years if the vial is properly sealed. Preferably, immediately before administration to the subject, final dilutions are prepared in pharmaceutically acceptable buffer and the final pH of the NO donor-containing buffer is checked for physiological suitability, especially when strongly acidic (e.g., hydrochloride salts) or alkaline (e.g., NONOates) stock solutions are used.

The product of NO exposure time and NO concentration largely determines the quality and magnitude of the biological response to exogenously supplied NO. Short-lived NO donors, such as DEA/NO, are most preferably administered by continuous infusion rather than by bolus to avoid delivering only a short burst of NO.

Activators of soluble guanylyl cyclase also include guanylyl cyclase activating protein (CCAP). Guanylin provides on example of a peptide capable of activating guanylyl cyclase in the intestine (Currie, et al., “Guanylin: an endogenous activator of intestinal guanylate” Proc. Natl. Acad. Sci. USA 89, 947-951 (1992) Atrial Natriuretic Peptide Fragment 5-27 (i.e., Atriopeptin II, Atriopeptin-23, rat, rANF (103-125)) provides another example, and is a known activator of both intestinal and vascular smooth muscle (Currie, M. G., et al., Science 1984 223: 67).

Isoliquiritigenin represents one example of a guanylyl cyclase activating protein (Yu S M, Kuo S C. “Vasorelaxant effect of isoliquiritigenin, a novel soluble guanylate cyclase activator, in rat aorta” Br J Pharmacol. 1995 114(8):1587-94; Yamamoto, et al., “The potent anti-tumor-promoting agent isoliquiritigenin” Carcinogenesis 1991 2: 317-323);

Soluble guanylyl cyclase may also be activated in an NO-independent fashion (Behrends S. “Drugs that activate specific nitric oxide sensitive guanylyl cyclase isoforms independent of nitric oxide release” Current Medicinal Chemistry 2003 10: 291-301; A NO-independent regulatory site has been identified on soluble guanylyl cyclase (Becker E M, Alonso-Alija C, Apeler H, Gerzer R, Minuth T, Pleiss U, Schmidt P, Schramm M, Schroder H, Schroeder W, Steinke W, Straub A, Stasch J P. “NO-independent regulatory site of direct sGC stimulators like YC-1 and BAY 41-2272” BMC Pharmacol. 2001 1(1):13).

Various compounds that activate soluble guanylyl cyclase independently of NO have been described (Behrends S. “Drugs that activate specific nitric oxide sensitive guanylyl cyclase isoforms independent of nitric oxide release” Current Medicinal Chemistry 2003 10: 291-301; Hobbs A J. “Soluble guanylate cyclase: an old therapeutic target re-visited” Br J Pharmacol. 2002 136(5):637-40; Stasch J P, Becker E M, Alonso-Alija C, Apeler H, Dembowsky K, Feurer A, Gerzer R, Minuth T, Perzborn E, Pleiss U, Schroder H, Schroeder W, Stahl E, Steinke W, Straub A, Schramm M. “NO-independent regulatory site on soluble guanylate cyclase” Nature. 2001 8:410(6825):212-5); NO-independent regulatory site of direct sGC stimulators like YC-1 and BAY 41-2272” BMC Pharmacol. 2001 1(1):13; Stasch J P, Schmidt P, Alonso-Alija C, Apeler H, Dembowsky K, Haerter M, Heil M, Minuth T, Perzborn E, Pleiss U, Schramm M, Schroeder W, Schroder H, Stahl E, Steinke W, Wunder F. “NO- and haem-independent activation of soluble guanylyl cyclase: molecular basis and cardiovascular implications of a new pharmacological principle” Br J Pharmacol. 2002 136(5):773-83; Straub A, Stasch J P, Alonso-Alija C, Benet-Buchholz J, Ducke B, Feurer A, Furstner C. “NO-independent stimulators of soluble guanylate cyclase” Bioorg Med Chem Lett. 2001 11(6):7814).

Non-limiting examples of NO-independent activators of soluble guanylyl cyclase include carbon monoxide (CO), porphyrins and metalloporphyrins (e.g., Protoporphyrin IX), YC-1 and the BAY family of compounds (e.g., BAY 41-2272, BAY 41-8543, BAU 58-2667).

Carbon monoxide (CO), for example, is an NO-independent activator of soluble guanlyl cyclase (Schultz G and Koesling D “Sensitizing soluble guanylyl cyclase to become a highly CO-sensitive enzyme” EMBO J 1996 15: 6863-6868). The relative of ability of CO to activate soluble guanylyl cyclase is significantly less than NO, but can be enhanced to NO-levels through potentiation by compounds such as YC-1. In one embodiment of the present invention, CO indirectly activates the KCa channel, either alone or in combination with a potentiator such as YC-1. While toxic at high doses, low doses of CO (i.e., exposure to 250 parts per million of CO for 1 hour) have been proposed for therapeutic use in vascular and inflammatory diseases (Otterbein L E, Zuckerbraun B S, Haga M, Liu F, Song R, Usheva A, Stachulak C, Bodyak N, Smith R N, Csizmadia E, Tyagi S, Akamatsu Y, Flavell R J, Billiar T R, Tzeng E, Bach F H, Choi A M, Soares M P. “Carbon monoxide suppresses arteriosclerotic lesions associated with chronic graft rejection and with balloon injury” Nat Med. 2003 9(2):183-90; Otterbein L E, Bach F H, Alam J, Soares M, Tao Lu H, Wysk M, Davis R J, Flavell R A, Choi A M. “Carbon monoxide has anti-inflammatory effects involving the mitogen-activated protein kinase pathway” Nat Med. 2000 6(4):422-8.

Porphyrins and metalloporphyrins, such as Protoporphyrin IX, are known NO-independent activators of sCG (Ignarro L J. “Regulation of cytosolic guanylyl cyclase by porphyrins and metalloporphyrins” Adv Pharmacol. 199426:35-65; Ignarro L J, Wood K S and Wolin M S. “Activation of purified soluble guanylate cyclase by protoporphyrin IX” Proc Natl Acad Sci USA 1982 79: 2870-2873; Ignarro L J, Wood K S and Wolin M S. “Regulation of purified soluble guanylate cyclase by porphyrins and metalloporphyrins: a unifying concept” Adv Cyclic Nucleotide Protein Phosphorylation Res 1984 17: 267-274).

Protoporphyrin IX (i.e., Kammerer's porphyrin, Ooporphyrin) for example, is an NO-independent activator of soluble guanylyl cyclase (Wolin, M S, et al., J. Biol. Chem. 1982 257:13312) and to a lesser extent, hematoporphyrin IX.

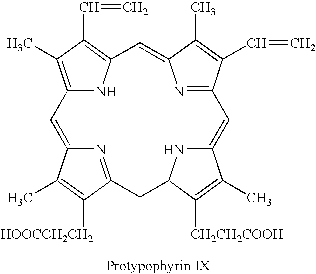

YC-1 [[3-(5′-hydroxymethyl-2′furyl)-1-benzyl indazole) is a synthetic benzylindazole compound, which has been shown to bind directly to sCG (Denninger J W, Schelvis J P, Brandish P E, Zhao Y, Babcok G T, Marietta M A. “Interaction of soluble guanylate cyclase with YC-1: kinetic and resonance Raman studies” Biochemistry 2000 39: 4191-4198;).

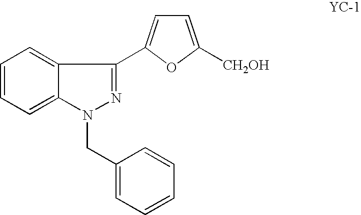

While YC-1 is known to bind directly to sCG, the exact binding site is the subject of differing reports (Denninger J W, Schelvis J P, Brandish P E, Zhao Y, Babcok G T, Marietta M A. “Interaction of soluble guanylate cyclase with YC-1: kinetic and resonance Raman studies” Biochemistry 2000 39: 4191-4198; Friebe A, Koesling D. “Mechanism of YC-1-induced activation of soluble guanylyl cyclase” Mol Pharmacol. 1998 53(1):123-7). In the presence of YC-1, CO produces more than a 100-fold increase in soluble guanylyl cyclase activity, a level equivalent to the activation of sCG by NO (Friebe A, Schultz G and Koesling D. “Sensitizing soluble guanylyl cyclase to become a highly CO-sensitive enzyme” EMBO J 1996 15: 6863-6868). Potentiation of protoporphyrin IX is also pronounced in the presence of YC-1 (Friebe A, Koesling D. “Mechanism of YC-1-induced activation of soluble guanylyl cyclase” Mol Pharmacol. 1998 53(1):123-7). YC-1 is thought to increases the affinity for heme ligands of these activators by reduction of dissociation rates (Russwurm M, Mergia E, Mullershausen F, Koesling D. “Inhibition of deactivation of NO-sensitive guanylyl cyclase accounts for the sensitizing effect of YC-1” J Biol Chem. 2002 277(28):24883-8).

Recently, a new class of NO-independent compounds has been discovered that activate soluble guanylyl cyclase independent of nitric oxide, including the YC-related compounds BAY 41-2272 and BAY 41-8543 and the chemically unrelated BAY 58-266 (Behrends S. “Drugs that activate specific nitric oxide sensitive guanylyl cyclase isoforms independent of nitric oxide release” Current Medicinal Chemistry 2003 10: 291-301; Stasch J P, Schmidt P, Alonso-Alija C, Apeler H, Dembowsky K, Haerter M, Heil M, Minuth T, Perzborn E, Pleiss U, Schramm M, Schroeder W, Schroder H, Stahl E, Steinke W, Wunder F. “NO- and haem-independent activation of soluble guanylyl cyclase: molecular basis and cardiovascular implications of a new pharmacological principle” Br J Pharmacol. 2002 136(5): 773-83; Nakane M. “Soluble guanylyl cyclase: physiological role as an NO receptor and the potential molecular target for therapeutic application” Clin Chem Lab Med. 2003 (7): 865-70.)

BAY41-2272 [3-(4-Amino-5-cyclopropylpyrimidine-2yl)-1-(2-flourobenzyl)-1H-pyrazolo[3,4-b]pyridine], a pyrazolopyridine, is a novel direct activator of soluble guanylate cyclase (sGC) that is similar is structure to YC-1 but is more specific and more potent. (Stasch J P, Becker E M, Alonso-Alija C, Apeler H, Dembowsky K, Feurer A, Gerzer R, Minuth T, Perzborn E, Pleiss U, Schroder H, Schroeder W, Stahl E, Steinke W, Straub A, Schramm M. “NO-independent regulatory site on soluble guanylate cyclase” Nature. 2001 410(6825): 212-5).

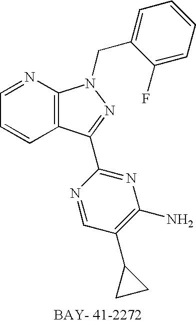

Unlike YC-1, BAY 41-2272 is highly bioavailable and can be given orally. Unlike YC-1, BAY 41-2272 is devoid of an effect on phosphodiesterases (Koglin M, Stasch J P, Behrends S. “BAY 41-2272 activates two isoforms of nitric oxide-sensitive guanylyl cyclase” Biochem Biophys Res Commun. 2002 292(4): 1057-62). BAY 41-2272 is though to be promising as an anti-hypertensive. BAY 41-2272 been suggested for use in treatment of erectile dysfunction (Kalsi J S, Rees R W, Hobbs A J, Royle M, Kell P D, Ralph D J, Moncada S, Cellek S “BAY41-2272, a novel nitric oxide independent soluble guanylate cyclase activator, relaxes human and rabbit corpus cavernosum in vitro” J Urol. 2003 169(2): 761-6; Cellek S. “The Rho-kinase inhibitor Y-27632 and the soluble guanylyl cyclase activator BAY41-2272 relax rabbit vaginal wall and clitoral corpus cavemosum” Br J Pharmacol. 2003 138(2): 287-90).

In one embodiment of the present invention, BAY 41-2272 indirectly activates the K_{Ca} channel. In a further embodiment of the present invention, BAY 41-2272 is used in combination with another K_{Ca} channel opener, including a direct agonist or an indirect activator, to activate the K_{Ca} channel.

BAY 41-8543 (also known as 2-[1-[2-fluorophenyl)methyl]-1H-pyrazolo[3,4-b]pyridin-3-yl]-5(4-morpholinyl)-4,6-pyrimidinediamine) is an analog of BAY41-2272 and directly stimulates soluble guanylyl cyclase in an NO-independent manner (Stasch J P, Dembowsky K, Perzborn E, Stahl E, Schramm M. “Cardiovascular actions of a novel NO-independent guanylyl cyclase stimulator, BAY 41-8543: in vivo studies” Br J Pharmacol. 2002 135(2):344-55. Similar to BAY 41-2272, and unlike YC-1, BAY 41-8543 has high bioavailability and lacks inhibitory action on phosphodiesterases. Similar to BAY 41-2272, BAY 41-8543 is thought to be useful in the treatment of cardiovascular disease.

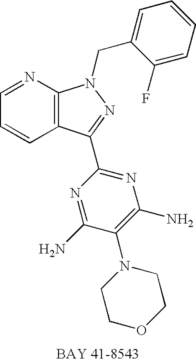

BAY 58-2667 (also known as 4-[((4-carboxybutyl)[2-[(4-phenethylbenzol) oxy]phenethyl]amino)methyl[benzoic]acid) is also a direct activator of soluble guanylyl cyclase, but it is structurally unrelated to YC-1 (Stasch J P, Schmidt P, Alonso-Alija C, Apeler H, Dembowsky K, Haerter M, Heil M, Minuth T, Perzborn E, Pleiss U, Schramm M, Schroeder W, Schroder H, Stahl E, Steinke W, Wunder F. “NO- and haem-independent activation of soluble guanylyl cyclase: molecular basis and cardiovascular implications of a new pharmacological principle” Br J Pharmacol. 2002 136(5): 773-83 In contrast to other known activators of soluble guanylyl cyclase (NO, YC-1 and BAY 41-2272), BAY 58-2267's mechanism of action is independent of the presence of a prosthetic heme group within soluble guanylyl cyclase (Schmidt P, Schramm M, Schroder H, Stasch J P. “Mechanisms of nitric oxide independent activation of soluble guanylyl cyclase” Eur J Pharmacol. 2003 468(3): 167-74. It is considered useful for the treatment of angina and hypertension.

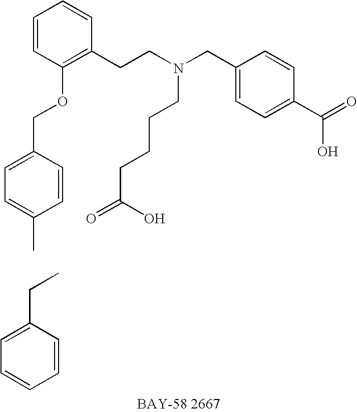

In one embodiment of the present invention, the indirect activator of K_{Ca} is an activator of soluble guanylyl cyclase. In a particular embodiment of the present invention, the indirect activator of K_{Ca} is nitric oxide. In another embodiment, the indirect activator of K_{Ca} is a guanylyl cyclase activating protein.

In a further embodiment of the present invention, the indirect activator of K_{Ca} is a NO-independent activator of soluble guanylyl cyclase other than YC-1. In a particular embodiment of the present invention, NO-independent activator of soluble guanylyl cyclase is CO. In another embodiment of the present invention, the NO-independent activator of soluble guanylyl cyclase is BAY 41-8543. In another embodiment of the present invention, the NO-independent activator of soluble guanylyl cyclase is BAY 41-2272.

According to the present invention, NO-independent activators may be used alone or in combination (e.g., YC-1 can be used to potentiate the effects of CO). According to another embodiment of the present invention, NO-independent activators can be used in combination with nitric oxide and nitric oxide donors to potentiate the effects of NO as an activator of soluble guanylyl cyclase, thereby activating the K_{Ca} channel.

Also included activators of any endogenous species of cyclic GMP or cyclic GMP-dependent protein kinase (PKG or cGK) that activates the K_{Ca} channel directly (e.g., by directly phosphorylation) or indirectly (e.g., by phosphorylating another regulatory protein that itself modulates K_{Ca} activity) (Robertson, BE et al., “cGMP dependent protein kinase activates Ca-activated K channels in cerebral artery smooth muscle cells” Am J Physiol 1993 256 (Cell physio.34) C299-2303); Fukao M. et al. “Cyclic cGMP-dependent protein kinase activates cloned BKCa channels expressed in mammalian cells by direct phosphorylation at serine 2072” J Biol Chem 1999 274(16) 10927-10935). Useful examples of PKG activators include, but are not limited to, octoroon-cyclic GMP (8Br-cGMP) and dibutyryl cyclic GMP. Included are activators of cGK I, cGK II, or other isoforms of cGMP-dependent protein kinase. (e.g., Smolenski, A. et al. [1998]).

In one embodiment of the present invention, the indirect activator of K_{Ca} is an activator of cGMP. In another embodiment, the indirect activator of K_{Ca} is an activator of cGMP-dependent protein kinase. In a particular embodiment, the indirect activator of K_{Ca} is an activator of PKG. In another particular embodiment, the indirect activator of K_{Ca} is an activator of cGK.

Phosphodiesterases (PDE) are enzymes that convert cGMP to GMP. Phosphodiesterase inhibitors are capable of increasing the amount of available cGMP and thereby indirectly activating the K_{Ca} channel. PDE5, PDE6 and PDE9 are known to be specific for cGMP. Sildenafil (Viagra) is a specific PDE5 inhibitor (Turko I V, Ballard S A, Francis S H, Corbin J D. “Inhibition of cyclic GMP-binding cyclic GMP-specific phosphodiesterase (Type 5) by sildenafil and related compounds” Mol Pharmacol. 1999 56(1):124-30). Many other examples of PDE5 specific inhibitors are known (Corbin J D and Francis S H. “Pharmacology of phosphodiesterase-5 inhibitors” Int. J. Clin. Pract. 2002 56: 453; Rotella D P. “Phosphodiesterase 5 inhibitors: current status and potential applications” Nat. Rev. Drug Discov. 2002 1:674; Gibson, A. “Phosphodiesterase 5 inhibitors and nitrergic transmission-from zaprinast to sildenafil” Eur. J. Pharmacol. 2001 411: 1; Non-limiting include tadalafil (i.e., Cialis™), vardenafil (i.e., Levitra™), MY 5445, zaprinast, quazinone, vardenafil and many others:

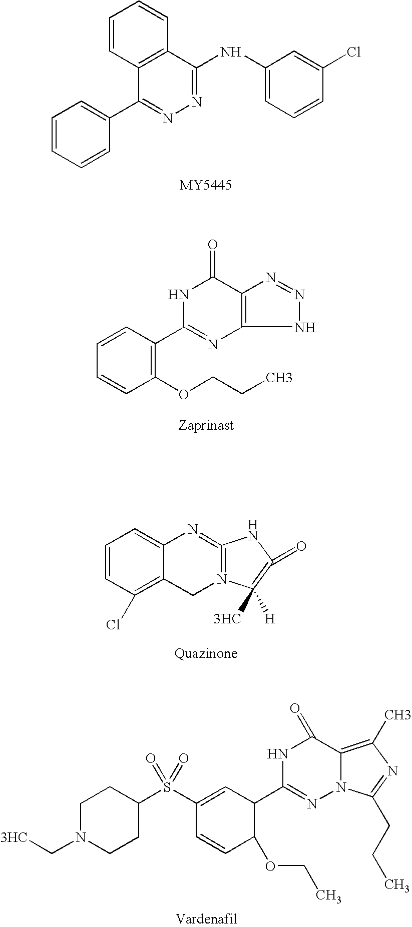

In a particular embodiment of the present invention, the indirect activator of the KCa channel is a phosphodiesterase inhibitor.

Also included among useful K_{Ca} activators is the vasodilator bradykinin (Arg-Pro-Pro-Gly-Phe-Ser-Pro-Phe-Arg), or a polypeptide bradykinin analog, such as receptor mediated permeabilizer (RMP)-7 or A7 (e.g., Kozarich et al., U.S. Pat. No. 5,268,164 and PCT Application No. WO 92/18529). Other useful analogs of bradykinin include related peptide structures which exhibit the same properties as bradykinin but have modified amino acids or peptide extensions on either terminal end of the peptide. For example, such bradykinin analogs include [Phe⁸(CH₂NH)Arg⁹]-bradykinin, N-acetyl [Phe⁸(CH₂NH)Arg⁹] bradykinin and desArg9-bradykinin.

Included among useful K_{Ca} channel activators are pharmaceutically acceptable molecular conjugates or salt forms that still have activity as K_{Ca} channel activators. Examples of pharmaceutically acceptable salts comprise anions including sulfate, carbonate, bicarbonate, nitrate, or the like. Other embodiments of pharmaceutically acceptable salts contain cations, such as sodium, potassium, magnesium, calcium, or the like. Other embodiments of useful potassium channel agonists are hydrochloride salts.

In one embodiment of the present invention, a direct agonist of K_{Ca} is used in combination or alternation with an indirect agonist of K_{Ca} to selectively deliver a therapeutic or diagnostic agent across the blood brain barrier.

K_{ATP} Agonists and Activators

Pharmacologic modulators of K_{ATP} channels are well-known and are of great therapeutic interest (Edwards G and Weston A H “The pharmacology of ATP-sensitive potassium channels” Annu. Rev. Pharmacol. Toxicol. 1993 33: 597-637;). K_{ATP} channel blocking agents, such as sulphonylureas (i.e., glibenclamide and tolbutamide) and imidazolines are used in the treatment of non-insulin dependent diabetes mellitus in order to stimulate insulin secretion. K_{ATP} channel blocking agents can also be used to cause vascular smooth muscle constriction. K_{ATP} channel openers, in particularly direct agonists such as minoxidil sulfate, have been used to treat angina and hypertension (Longman S D, Hamilton T C. “Potassium channel activator drugs: mechanism of action, pharmacological properties, and therapeutic potential” Med Res Rev. 1992 12(2): 73-148) Activation of K_{ATP} channels is thought to be involved in ischemic preconditioning, suggesting a role for K_{ATP} agonists as cardioprotective agents (i.e., as part of an intervention strategy directly after acute myocardial infarction in high-risk patients) (Markham A, Plosker G L, Goa K L. “Nicorandil. An updated review of its use in ischemic heart disease with emphasis on its cardioprotective effects” Drugs. 2000 60(4): 955-74). The cardioprotective effects of K_{ATP} openers are independent of vasodilator and cardiodepressant effects (Grover G J “Pharmacology of ATP-sensitive potassium channel (KATP) openers in models of myocardial ischemia and reperfusion” Can J Physiol Pharmacol. 1997 April; 75(4): 309-15). Numerous other potential therapeutic applications have been identified for K_{ATP} openers.

K_{ATP} channel agonists a chemically heterogeneous group of compounds including benzopyrans (e.g., cromakalim), cyanoguanidines (e.g., pinacidil), thioformamides (aprikalim), thiadiazines (e.g., diazoxide), pyridyl nitrates (nicorandil), and pyrimidine sulfates (e.g., minoxidil sulfate) (Mannhold, R. “KATP channel openers: Structure-activity relationships and therapeutic potential.” Med Res Rev. 2004 March; 24(2): 213-66; Brayden, J. E. “Functional roles of K_{ATP} channels in vascular smooth muscle” Clin. Exp. Pharacol. Physiol. 2002, 29, 312-316; U.S. Pat. No. 6,624,186 to Teuber et al. entitled “Ion channel modulating agents comprising novel benzimidazolone derivatives; U.S. Pat. No. 6,649,609 to Teuber et al, entitled “Benzimidazole derivatives and pharmaceutical compositions comprising these compounds”; U.S. Pat. No. 6,525,043 Jensen et al. entitled “Use of ion channel modulating agents”).

Second generation K_{ATP} agonists have been identified in diverse chemical classes such as cyclobutenediones (e.g., WAY-1516), dihydropyridine (DHP) related structures (e.g., ZM-244085) and entirely new chemotypes such as tertiary carbinols (e.g., ZD-6169). Many second generation agonists have been developed to provide tissue selectivity for particular compounds.

A comprehensive review of K_{ATP} agonists and an analysis of known structure-function relationships (SAR) can be found in Mannhold R. “K_{ATP} channel openers: structure activity relationships and therapeutic potential” Medicinal Research Reviews 2004 24(2): 213-266, which is incorporated herein by reference. Additional K_{ATP} channel activators are described in Bristol, et al., “Annual Reports in Medicinal Chemistry,” Vol. 29, Chap. 8, pp. 73-82, Academic Press (1991).

K_{ATP} agonists activate ATP-sensitive K⁺ channels (K_{ATP} channels) by binding directly to K_{ATP} channel (Ashcroft F M, Gribble F M. “New windows on the mechanism of action of K(ATP) channel openers” Trends Pharmacol Sci. 2000 21(11): 439-45). Specifically, the sulphonylurea receptors (SURs), the regulatory channel subunits, has been shown to be the site of binding (Schwanstecher M, Sieverding C, Dorschner H, Gross I, Aguilar-Bryan L, Schwanstecher C, Bryan J “Potassium channel openers require ATP to bind to and act through sulfonylurea receptors” EMBO J. 1998 17(19):5529-35; M. Dabrowski, F. M. Ashcroft, R. Ashfield, P. Lebrun, B. Pirotte, J. Egebjerg, J. Bondo Hansen, and P. Wahl “The novel diazoxide analog 3-isopropylamino-7-methoxy-4H-1,2,4-benzothiadiazine 1,1-dioxide Is a Selective Kir6.2/SUR1 Channel Opener” Diabetes 2002 51(6):1896-1906. The binding site for K_{ATP} agonists has been localized to the COOH-terminal transmembrane domain complex of SUR. Hambrock, A, Preisig-Müller, R, Russ, U, Piehl, A, Hanley, P, Ray, J, Daut, J, Quast U, Derst C Am J Physiol Cell Physiol 2002 283:C587-598; Uhde I, Toman A, Gross I, Schwanstecher C, Schwanstecher M “Identification of the potassium channel opener site on sulfonylurea receptors” J Biol Chem. 1999 274(40):28079-82; Bray, K. and Quast, U. “A specific binding site for K+ channel openers in rat aorta” Journal of Biological Chemistry 1992, 267:11689-11692;). Binding inhibition studies suggest that the K_{ATP} channel has not one but two binding sites for minoxidil sulfate.

Tissue-specific actions exist among the different subclasses of K_{ATP} agonists. (Yokoshiki H, Sunagawa M, Seki T, and Sperelakis N “ATP-sensitive K⁺ channels in pancreatic, cardiac, and vascular smooth muscle cells” Am J Physiol Cell Physiol 274:25-37). This tissue specificity is thought to be the result of tissue specific forms of the SUR regulatory channel subunit. (Babenko A, Gonzalez G, Bryan J, “Pharmaco-topology of Sulfonylurea Receptors” J Biol Chem 2000 275(2) 717-720).

Agents suitable for use in the presence invention include both direct agonists and indirect activators of K_{ATP}. Non-limiting examples of direct agonists of K_{ATP} include minoxidil, minoxidil sulfate, pinacidil, cromakalim and diazoxide. Non-limiting examples of indirect activators of the K_{ATP} channel include activators of adenylate cyclase such as forskolin.

Direct agonists of the K_{ATP} channel bind to and act through the sulfonylurea receptor (SUR) subunit of the K_{AT}P channel (Hambrock A, Loffler-Walz C, Kloor D, Delabar U, Horio Y, Kurachi Y, Quast U. “ATP-Sensitive K+ channel modulator binding to sulfonylurea receptors SUR2A and SUR2B: opposite effects of MgADP” Mol Pharmacol. 1999 55(5): 832-40. Schwanstecher, M., Sieverding, C., Dorschner, H., Gross, I., Aguilar-Bryan, L., Schwanstecher, C., and Bryan, J., “Potassium channel openers require ATP to bind to and act through the sulfonylurea receptors” The EMBO Journal 1998, 17:5529-5535). SURs are a member of the ATP-binding cassette superfamily and are characterized by two nucleotide binding folds (NBFs) thought to play an important role in regulation by K_{ATP} agonists (Yokoshiki H, Sunagawa M, Seki T, Sperelakis N. “ATP-sensitive K+ channels in pancreatic, cardiac, and vascular smooth muscle cells” Am J Physiol. 1998 274(1 Pt 1): C25-37). In particular, the C-terminus of SURs is thought to affects binding affinity by K_{ATP} agonists (Schwanstecher, M., Sieverding, C., Dorschner, H., Gross, I., Aguilar-Bryan, L., Schwanstecher, C., and Bryan, J., “Potassium channel openers require ATP to bind to and act through the sulfonylurea receptors” The EMBO Journal 1998, 17:5529-5535). Mutations in NBF regions have been shown to eliminate K_{ATP} agonist activation. Competition binding experiments suggest that minoxidil sulfate is unique among K_{ATP} agonists in having two potential binding sites on the KATP channel (Schwanstecher, M., Sieverding, C., Dorschner, H., Gross, I., Aguilar-Bryan, L., Schwanstecher, C., and Bryan, J., “Potassium channel openers require ATP to bind to and act through the sulfonylurea receptors” The EMBO Journal 1998, 17:5529-5535). Potencies of many K_{ATP} agonists have been shown to be higher for SUR2B (the vascular smooth muscle cell SUR) than for SUR2A, the cardiac SUR (Hambrock A, Loffler-Walz C, Kloor D, Delabar U, Horio Y, Kurachi Y, Quast U. “ATP-Sensitive K+ channel modulator binding to sulfonylurea receptors SUR2A and SUR2B: opposite effects of MgADP” Mol Pharmacol. 1999 55(5): 832-40).

Benzopyran Derivatives

Benzopyran derivatives represent one major class of ATP-sensitive K+ channel openers suitable for use in the present invention. Cromakalim [(3S-trans)-3,4-dihydro-3-hydroxy-2,2-dimethyl-4-(2-oxo-1-pyrrolidynyl)-2H-1-benzopyran-6 carbonitrile] a well-known member of this class. In a preferred embodiment of the present invention, the K_{ATP} agonist is cromakalim (±)-cromakalim, (+)-cromakalim, (−)-cromakalim. Derivatives of benzopyrans can be generally classified into 4′ benzopyran ring substituents, 3′ substituents, 2′ substituents, aromatic substitutions and transformations of the benzopyran ring. Each structural variation produces corresponding changes in properties, including potency and specificity. These relationships are well understood in the art and reviewed in detail in Mannhold, R. “KATP channel openers: structure activity relationships and therapeutic potential” Medicinal Research Reviews 2004 24(2): 213-266.

In general, most K_{ATP} agonists that are benzopyran derivatives are 4′ benzopyran ring substituents, including unbridged cyclic substituents (e.g., levcromakalim, celikalim, bimkalim, emakalim, U96501 and RO 31-6930), bridged cyclic substituents (e.g., SDZ PCO 400) and acyclic substituents (e.g., KC-399, KC 515). Other known agonists result from modifications in the 3′ position (e.g. BRL 49381), the 2′ position (e.g., JTV 506) as well as from aromatic substitutions (e.g., NIP-121, rilmakalim). The K_{ATP} agonist of the present invention is optionally a benzopyran with a substituent of the pyran moiety (e.g., YM 934, UR-8225, KOCN7) or the aromatic ring (e.g., RMJ 29009). Also included in this category is Compound 15, a cromakalim analogue (Maruyama M et al. FASEB J 1989; 3:A897).

A general formula for benzopyran derivatives suitable as K_{ATP} agonists in the present invention:

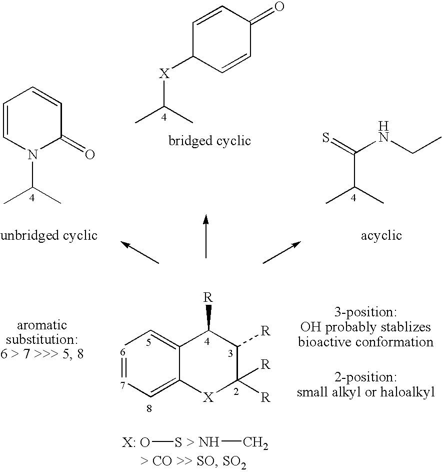

Non-limiting examples of K_{ATP} agonists considered benzopyran derivatives include:

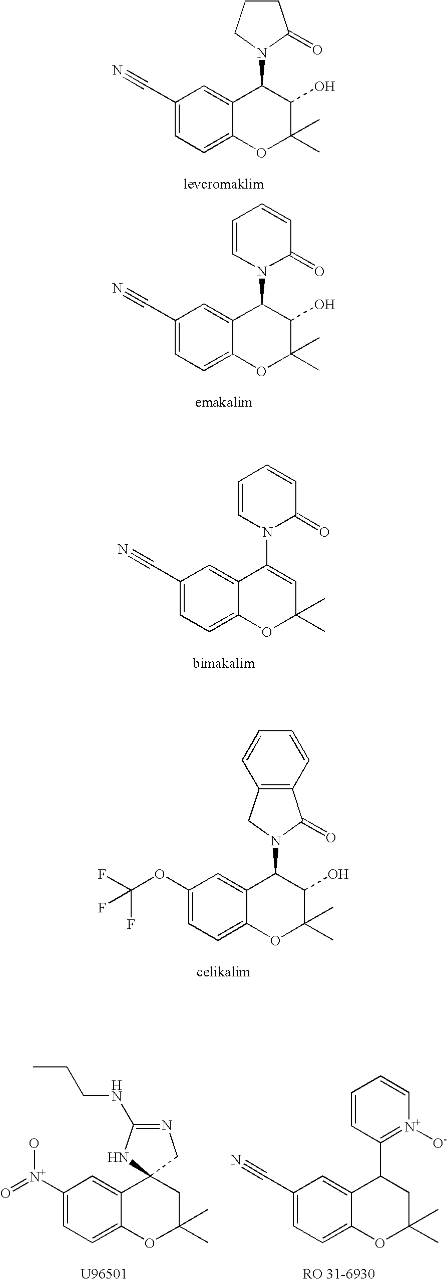

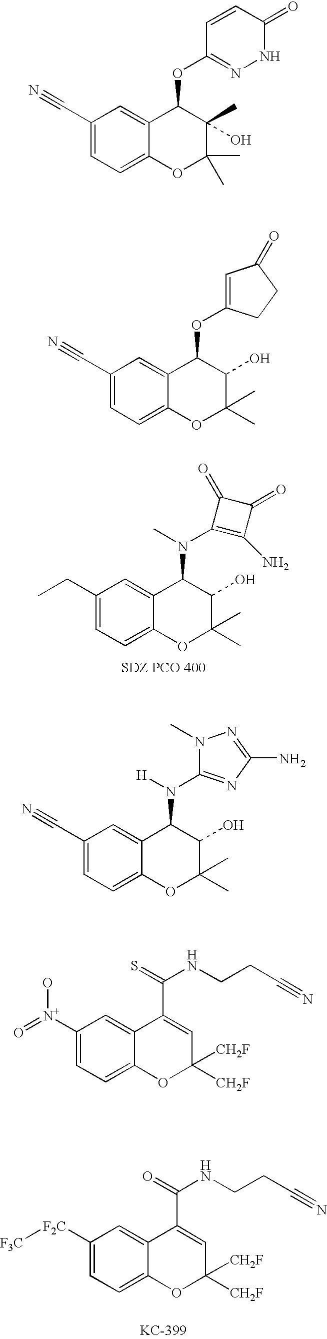

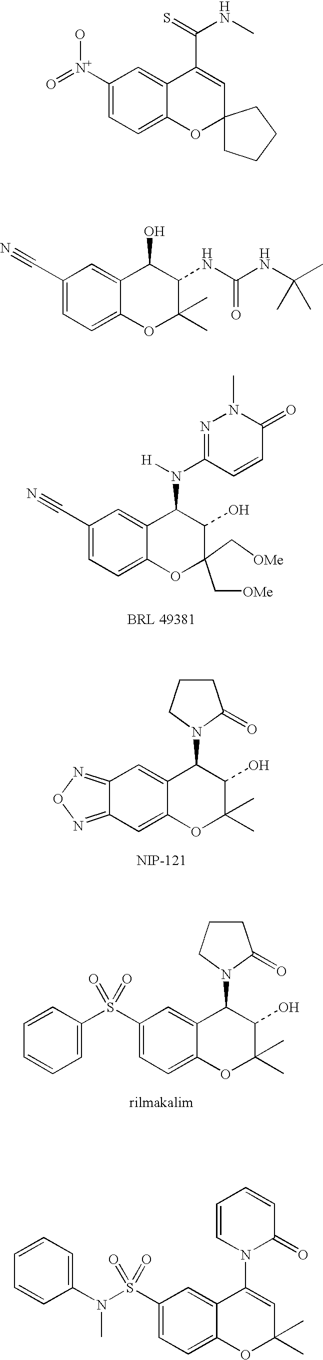

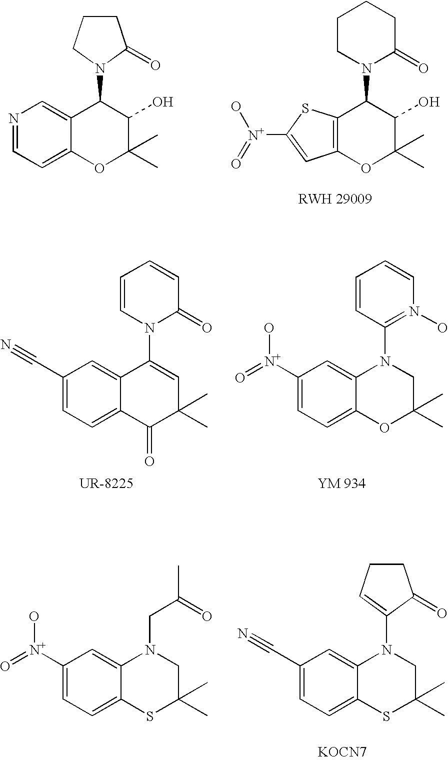

Second generation benzopyran derivates have been developed to provide more tissue selective K_{ATP} agonists. Airway selective K_{ATP} agonists considered benzopyrans or closely related diaklylnapthaleones include KC-128, BRL 55834, SDZ-217-740 and KCO912, shown below together with other representative airway selective compounds of this class:

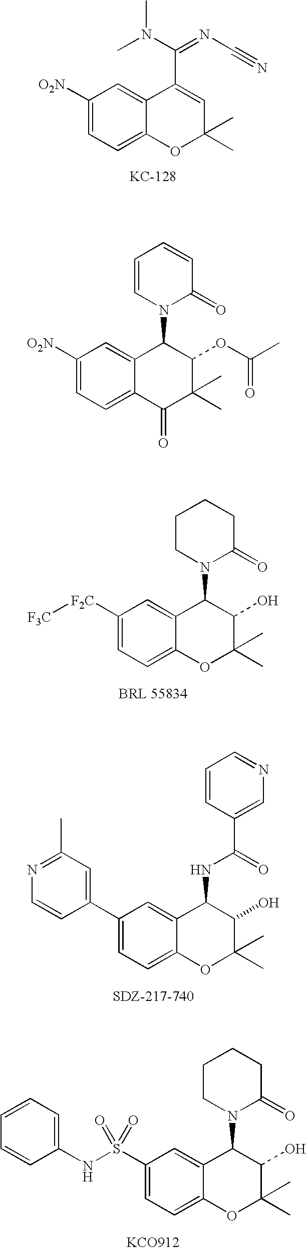

Other tissue specific benzopyran derivatives include bladder selective K_{ATP} agonists for the treatment of urinary incontinence, including the structures below:

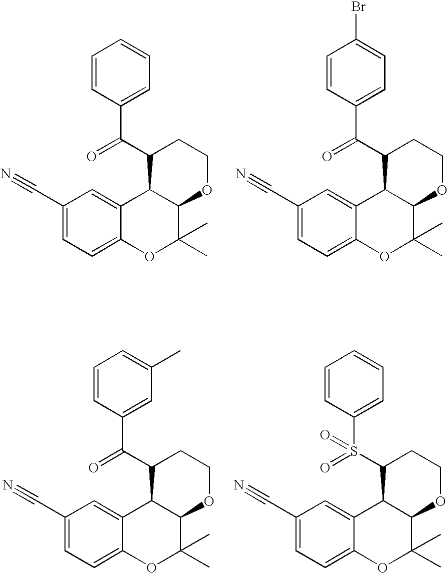

Hybrid molecules between benzopyrans and cyanoguanidines such as BMS0180448 have been developed to enhance cardioselectivity. Cardioselective K_{ATP} agonists have enhanced cardioprotective anti-ischemic properties and diminished hemodynamic effects (i.e., vasodilatation) when given systemically. Various 4-(N-aryl)-substituted benzopyrans have been developed, with BMS 191095 exhibiting the highest anti-ischemic potency and selectivity.

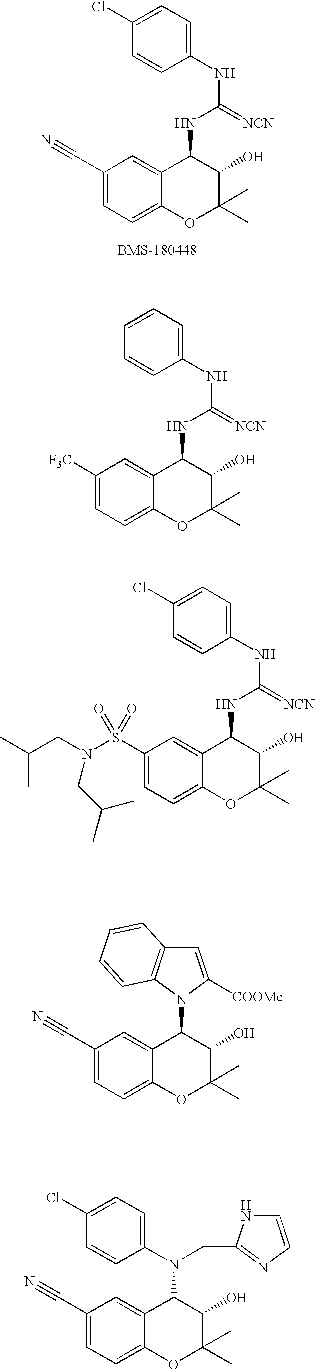

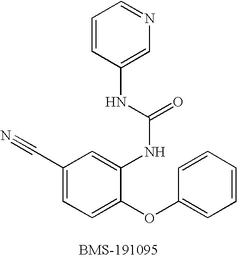

Cyanoguanidines

Pinacidil [(±)N-cyano-N′-4-pyridynyl-N″(1,2,2-trimethylopropyl)guanidine monohydrate] is representative of the cyanoguanidine class of K_{ATP} agonists considered suitable for use in the present invention. Potassium channel agonist activity resides in the R-(−) enantiomer. P1075 (N-cyano-N′-[1,1-dimethyl-[2,2,3,3-H]propyl]-N″-(3-pyridinyl)guanidine) is a pinacidil derivative that functions as a K_{ATP} agonist (Bray K M and Quast U “A specific binding site for K+ channel openers in rat aorta” J. Biol. Chem. 1992 267(17): 11689-11692). Other derivatives include P1060 and AL0670. Non-limiting examples of cyanoguanidine K_{ATP} agonists include:

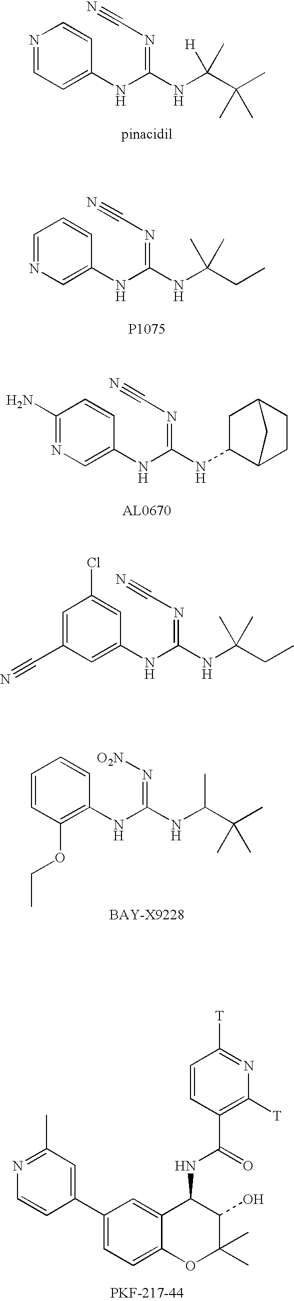

Thioformamides

Thioformamides and their analogs provide yet another class of K_{ATP} agonists useful in the present invention. Apriklim is a prototypical thioformamide KATP agonist, although it's K_{ATP} opening properties was discovered after it's antihypertensive activity was recognized. MCC-134 [(1-[4-(H-imidazol-1-yl)benzoyl]-N-methylcyclobutane-carbothioamide] is a recently developed thioformamide derivative K_{ATP} agonist (Sasaki N, Murata M, Guo Y, Jo S, Ohler A, Akao M, O'Rourke B, Xiao R, Bolli R, Marbán E. “MCC-134, a Single Pharmacophore, Opens Surface ATP-Sensitive Potassium Channels, Blocks Mitochondrial ATP-Sensitive Potassium Channels, and Suppresses Preconditioning” Circulation. 2003 107:1183). Other common derivatives include modifications of the thiamide function, the saturated heterocycle and the (hetero)cyclic aromatic group. In general, the structure-activity relationships of thioformamide K_{ATP} agonists are represented as follows:

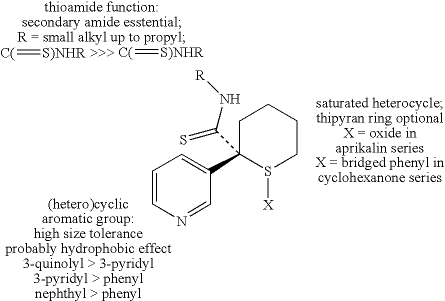

Non-limiting examples of thioformamide derivate K_{ATP} agonists include:

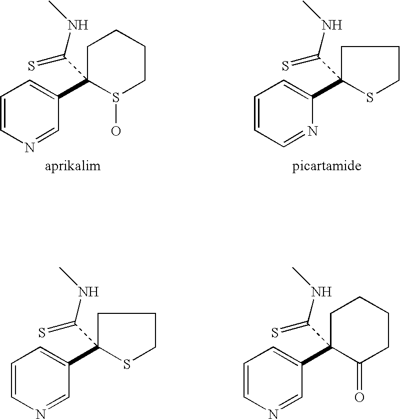

Benzo-Pyridothiadiazines

K_{ATP} agonists useful in the present invention also come from the benzo-pyridothiadiazine class. Diazoxide [7-chloro-3-methyl-2H-1,2,4-benzothiadiazine 1,1-oxide] for example is a benzothiadiazine. In one embodiment of the present invention, the K_{ATP} agonist is diazoxide. Diazoxide is though to be the only K_{ATP} agonist that binds with similar affinity to the smooth muscle SUR (SUR2B) and the pancreatic SUR (SUR1), thereby both relaxing vascular smooth muscle and inhibiting insulin secretion almost equipotently (Antoine M H, Berkenboom G, Fang Z Y, Fontaine J, Herschelz A, Lebrun P. “Mechanical and ionic responses of rat aorta to diazoxide” Eur J Pharmacol 1992 216:299-306). The SUR binding site of diazoxide is also distinct from the binding site for the benzopyrans and the cyanoguanidines (Ashcroft F M, Gribble F M, “New windows on the mechanism of action of K_{ATP} channel openers: Trends Pharmacol Sci 2000 21:439-445). Other derivates have been developed to improve potency and selectivity. One diazoxide analog K_{ATP} agonist with selectively for SUR1 is 3-isopropylamino-7-methoxy-4H-1,2,4-benzothiadiazine 1,1-dioxide (NNC 55-9216) (Dabrowski M, Ashcroft F M, Ashfield R, Lebrun P, Pirotte B, Egebjerg J, Bondo Hansen J, Wahl P. “The novel diazoxide analog 3-isopropylamino-7-methoxy-4H-1,2,4-benzothiadiazine 1,1-dioxide is a selective Kir6.2/SUR1 channel opener” Diabetes. 2002 51(6): 1896-906). Another novel benzothidiazine is 6,7-dichloro-3-isopropylamino-4H-1,2,4-benzothiadiazine 1,1-dioxide (BPDZ 154) (Cosgrove K E, Antoine M H, Lee A T, Barnes P D, de Tullio P, Clayton P, McCloy R, De Lonlay P, Nihoul-Fekete C, Robert J J, Saudubray J M, Rahier J, Lindley K J, Hussain K, Aynsley-Green A, Pirotte B, Lebrun P, Dunne M J. “BPDZ 154 activates adenosine 5′-triphosphate-sensitive potassium channels: in vitro studies using rodent insulin-secreting cells and islets isolated from patients with hyperinsulinism” J Clin Endocrinol Metab. 2002 87(11): 4860-8). New K_{ATP} agonists have been developed that represent hybrids of diazoxide and pinacidil-type potassium channel agonists. These include for example, BPDZ-44, BPDZ-79 and BPDZ-83. Non-limiting examples of benzo- and pyridothiadiazines with K_{ATP} agonist properties include:

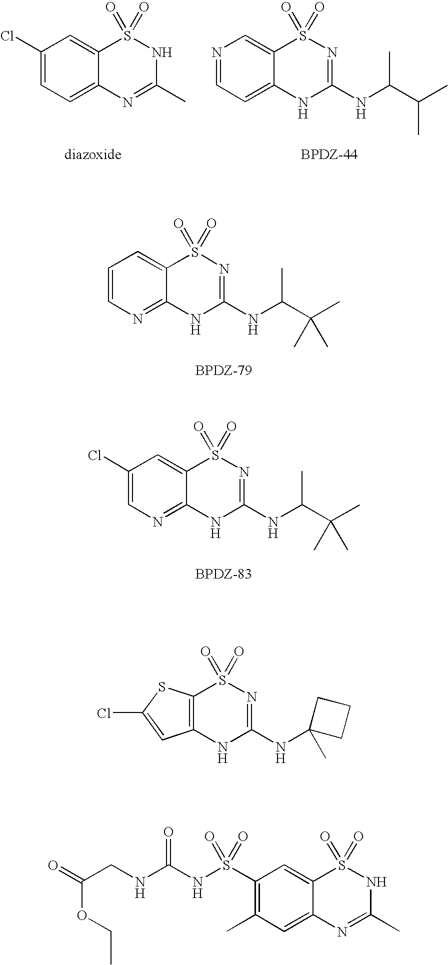

Pyridil Nitrates

Pyridyl nitrates such as nicorandil are also suitable for use as K_{ATP} agonists in the present invention. Nicorandil is unique amoung the K_{ATP} agonists described herein because it has a dual mechanism of action which also involves stimulation of guanylate cyclase (Holzman S. “Cyclic GMP as a possible mediator of coronary arterial relaxation by nicorandil (SG-75)” Cardiovasc Pharmacol 1983 5: 364-370). The pyridul nitrate analog KRN2391 (N-cyano-N′-(2-nitroxyethyl)-3-pyridinecarboximidamide monomethansulphonate) differs from nicorandil, however, in that the guanylyl cyclase activating property is eliminated, rendering the compound a pure K_{ATP} agonist. Another nicorandil derivative that functions as a K_{ATP} agonist is KRN4884 [5-amino-N-[2-(2-chlorophenyl)ethyl]-N′-cyano-3-pyridinecarboxamidine] (Shinbo A, Ono K, Iijima T. “Activation of cardiac ATP-sensitive K+ channels by KRN4884, a novel K+ channel opener” J Pharmacol Exp Ther. 1997 283(2): 770-7.

Non-limiting examples of pyridyl nitrate analogs as K_{ATP} agonists include:

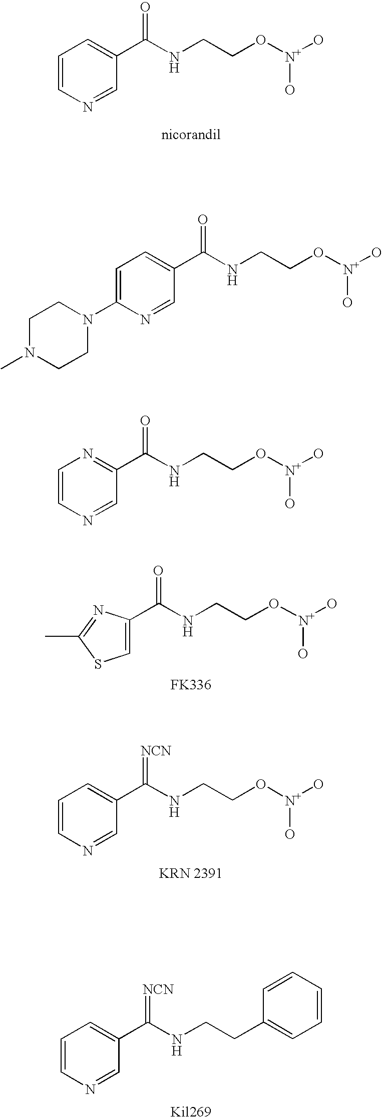

Pyrimidine Sulfates

Pyrimidine sulfates and their analogs are considered suitable for use in the present invention as K_{ATP} agonists. In a preferred embodiment of the present invention, the K_{ATP} agonist is the pyrimidine sulfate minoxidil [2,4-diamino-6-piperidino pyramidine-3-oxide] or minoxidil sulphate.

Other KATP Agonists

Other K_{ATP} agonists suitable for use in the present invention include diaminocyclobutenediones derived from cyanoguanidine potassium channel agonists. Non-limiting examples of diaminocyclobutenedione K_{ATP} agonists include:

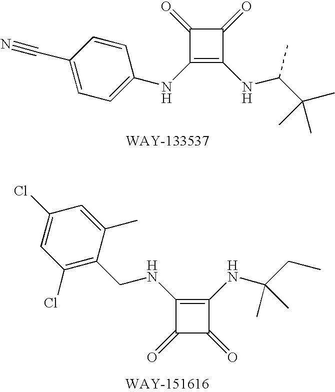

Dihydropyridines (DHPs) are a class of well-known antihypertensives. DHP-related structures have been developed as K_{ATP} agonists. These agents are considered suitable for use in the present invention and include, for example:

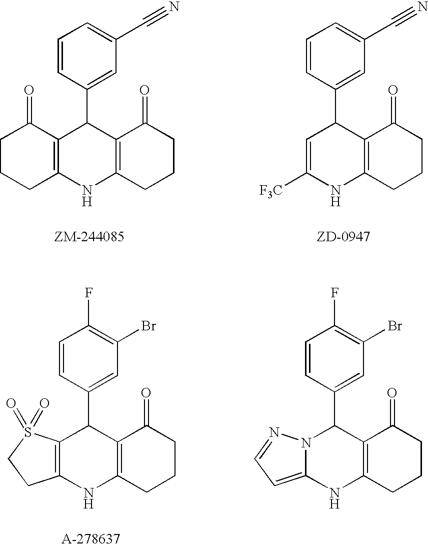

Other non-limiting examples of K_{ATP} agonists suitable for use in the present invention include:
ER001533 and E4080 (See Terzic A, Tung R, Shen W, Yamada M and Kurachi Y. “ER001533 See Cardiovascular Profile of E4080 and Its Analogue ER001533, Novel Potassium Channel Openers with Bradycardic Properties” Cardiovascular Drugs Reviews: 2002 11(2): 223-233);
A-312110 [(9R)-9-(4-fluoro-3-iodophenyl)-2,3,5,9-tetrahydro-4H-pyrano[3,4-b]thieno[2,3-e]pyridin-8(7H)-one-1,1-dioxide) (Davis-Taber R, Molinari E J, Altenbach R J, Whiteaker K L, Shieh C C, Rotert G, Buckner S A, Malysz J, Milicic I, McDermott J S, Gintant G A, Coghlan M J, Carroll W A, Scott V E, Gopalakrishnan M. “125I]A-312110, a novel high-affinity 1,4-dihydropyridine ATP-sensitive K+ channel opener: characterization and pharmacology of binding” Mol Pharmacol. 2003 64(1): 143-53);
levosimendan (Pataricza J, Krassoi I, Hohn J, Kun A, Papp J G. “Functional role of potassium channels in the vasodilating mechanism of levosimendan in porcine isolated coronary artery” Cardiovasc Drugs Ther. 2003 17(2): 115-21; Kopustinskiene D M, Pollesello P, Saris N E. “Levosimendan is a mitochondrial K(ATP) channel opener” Eur J Pharmacol 2001 428:3114));
RP 49356 (Raeburn D and Brown T J. “RP 49356 and cromakalim relax airway smooth muscle in vitro by opening a sulphonylurea-sensitive K+ channel: a comparison with nifedipine” Journal of Pharmacology and Experiemental Therapeutics 1991 256(2) 480-485);
R 44866 (Findlay, I. “Effects of pH upon the inhibition by sulphonylurea drugs of ATP-sensitive K+ channels in cardiac muscle” Journal of Pharmacology and Experimental Therapeutics 1992 262(1) 71-79).
LP-805 [8-tert-butyl-6,7-dihydropyrrolo-[3,2-e]-5-methylpyrazolo-[1,5a]-pyrimidine-3-carbonitrile] is a KATP agonist (Kishii K, Morimoto T, Nakajima N, Yamazaki K, Tsujitani M, Takayanagi I. “Effects of LP-805, a novel vasorelaxant agent, a potassium channel opener, on rat thoracic aorta. Gen Pharmacol. 1992 23(3): 347-53) but is considered to have a dual mechanism of action, including a potent releasing action on endothelium-derived relaxing factor (Nakashima M, Akata T, Kuriyama H. “Effects on the rabbit coronary artery of LP-805, a new type of releaser of endothelium-derived relaxing factor and a K+ channel opener” Circ Res. 1992 71(4): 859.
SO121
HOE-234 or [(3S,4R)-3-hydroxy-2,2-dimethyl-4-(2-oxo-1-pyrrolidinyl)-6-phenylsulfonylchroman hemihydrate] ((Terzic A, Jahangir A Kurachi Y. “HOE-234, a second generation K+ channel opener, antagonizes the ATP-dependent gating of cardiac ATP-sensitive K+ channels” Journal of Pharmacology and Experimental Therapeutics 1994 268(2): 818-825).

The structures of some exemplary K_{ATP} agonists are as set forth below:

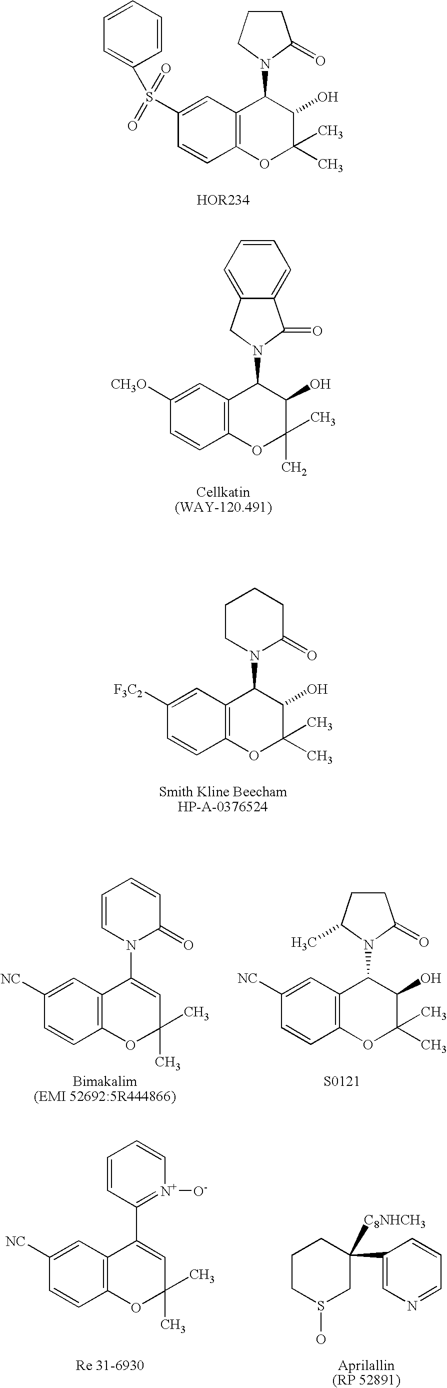

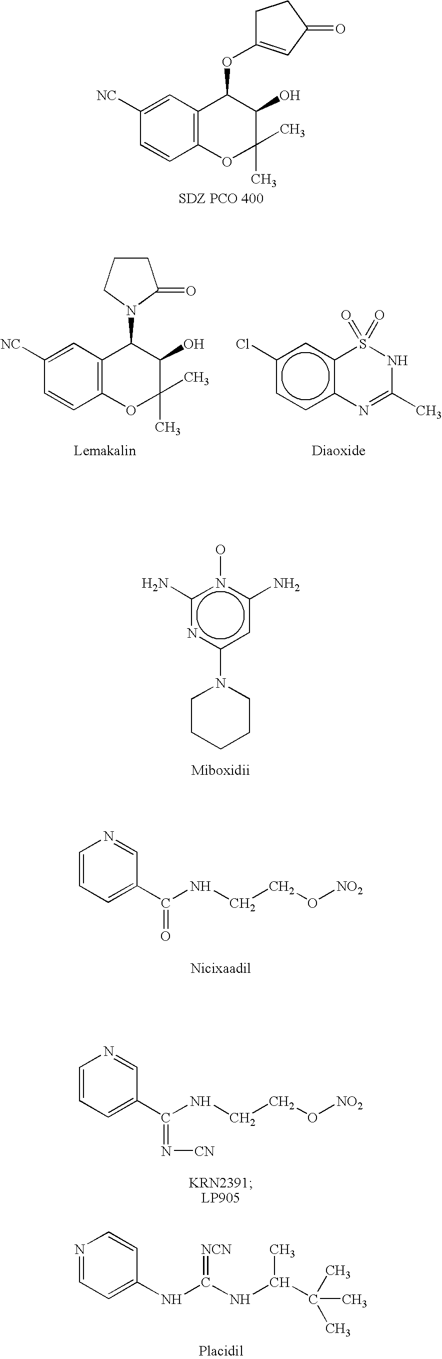

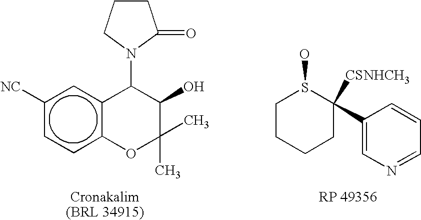

Further, several patent publications disclose assays to screen for other agonists of K_{ATP} channels. See for example WO 98/33905. Methodical progress through the use of high-throughout put screening against cloned subtypes of the K_{ATP} channel, and the application of structure-based designed, should allow for continued developed of novel KATP agonists (Mannhold R. “K_{ATP} channel openers: structure-activity relationships and therapeutic potential” Medicinal Research Reviews 2004 24(2): 213-266.

Indirect Activators of the K_{ATP} Channel

In one embodiment of the present invention, the potassium channel activators are compounds that indirectly activate the K_{ATP} channel. For example, indirect activators or agonists may include activators of regulatory elements involved in the activation of K_{ATP}, such as the adenylyl cyclase-cAMP signaling pathway. Indirect activators of the K_{ATP} channel include agents that increase the amount of cAMP or cAMP dependent protein kinases in vivo, and thereby indirectly activate the K_{ATP} channel via the adenylyl cyclase-cAMP signaling pathway.

Activators of adenylyl cyclase provides one non-limiting example of an agent that increases the amount of cAMP in vivo. Adenylyl cyclase catalyzes the formation of cAMP from ATP. Adenylyl cyclase activators and cAMP-dependent protein kinase inhibitors can be found in U.S. Patent Application No. 0020198136, incorporated here by reference. A preferred adenylyl cyclcase activators is forskolin (7 beta-acetoxy-8,13-epoxy-1 alpha, 6 beta, 9 alpha-trihydroxylabd-14-en-11-one), a diterpene from the Indian plant Coleus forskohlii.

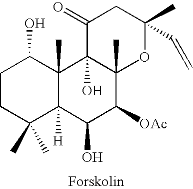

Derivatives of forskolin, including colforsin daropate hydrochloride, are also suitable for use in the present invention (Laurenza A, Khandelwal Y, De Souza N J, Rupp R H, Metzger H, Seamon K B. “Stimulation of adenylate cyclase by water-soluble analogues of forskolin” Mol Pharmacol. 1987 32(1):133-9; Seamon K B, Daly J W, Metzger H, de Souza N J, Reden J. “Structure-activity relationships for activation of adenylate cyclase by the diterpene forskolin and its derivative” J Med Chem. 1983 26(3):436-9). Forskolin derivatives have been used and suggested for use in the treatment of cardiac and respiratory disease (Kikura M, Morita K, Sato S. “Pharmacokinetics and a simulation model of colforsin daropate, new forskolin derivative inotropic vasodilator, in patients undergoing coronary artery bypass grafting” Pharmacol Res. 2004 49(3):275-81; Bristow M R, Ginsburg R, Strosberg A, Montgomery W, Minobe W. “Pharmacology and inotropic potential of forskolin in the human heart” J Clin Invest. 1984 74(1):212-23; Wajima Z, Yoshikawa T, Ogura A, Imanaga K, Shiga T, Inoue T, Ogawa R. “Intravenous colforsin daropate, a water-soluble forskolin derivative, prevents thiamylal-fentanyl-induced bronchoconstriction in humans” Crit Care Med. 2002 30(4):820-6) Other non-limiting examples of adenylyl cyclase activators according to the present invention include A02011-1 (a pyrazole derivative) (Yu S M, Cheng Z J, Kuo S C. “Antiproliferative effects of A02011-1, an adenylyl cyclase activator, in cultured vascular smooth muscle cells of rat” Br J Pharmacol. 1995 114(6): 1227-35), dexamethasone and PGE1.

Peptides or proteins are also suitable for activating adenylyl cyclase. These ACAp's include for example litorin (i.e., pituitary adenylate cyclase activating fragment 21-38; Pyr-Gln-Trp-Ala-Val-Gly-His-Phe-Met). Another known activator of adenylate cyclase is pituitary adenylate cyclase activating polypeptide-38 (i.e., PACAP-38; His-Ser-Asp-Gly-Ile-Phe-Thr-Asp-Ser-Tyr-Ser-Arg-Tyr-Arg-Lys-Gln-Met-Ala-Val-Lys-Lys-Tyr-Leu-Ala-Ala-Val-Leu-Gly-Lys-Arg-Tyr-Lys-Gln-Arg-Val-Lys-Asn-Lys-NH₂) (Kinhult, J., et al., “Pituitary adenylate cyclase-activating peptide 38 a potent endogenously produced dilator of human airways” Eur. Respir. J. 2000 15: 243-247; Yada T et al., “Pituitary adenylate cyclase-activating polypeptide is an extraordinarily potent intra-pancreatic regulator of insulin secretion from isletbcells” J. Biol. Chem. 1994 26: 1290-1293 (1994)). PACAP-27 is also known to stimulate adenylate cyclase.

In one embodiment of the present invention, the indirect activator of KATP is an activator of adenylyl cyclase. In a particular embodiment, the activator of adenylyl cylcase is forskolin or a forskolin analog. In another embodiment, the activator of adenylyl cyclase is a adenylyl cyclase activating peptide or protein.

Other suitable indirect K_{ATP} activators include agents capable of raising the level of cAMP. Prostacyclin, prostaglandin E1, and isoproterenol are all known to increase cAMP formation. Phosphodiesterase inhibition of the breakdown of cyclic AMP increases it (Levy J H. “The ideal agent for perioperative hypertension” Acta Anaesth Scand 1993; 37: 20). Phophodiesterases specific for cAMP include PDE4, PDE 7 and PDE 8 (Barnette M S. “Phosphodiesterase 4 (PDE4) inhibitors in asthma and chronic obstructive pulmonary disease (COPD)” Progress in Drug Research, 1999 Vol. 53 (E. Jucker, Ed.). PDE4 inhibitors have been targeted for the treatment of airway disorders, including chronic obstructive pulmonary disease (COPD), emphysema & bronchitis. Rolipram provides one example of a PDE 4 inhibitor and many others are known (Huang Z, Ducharme Y, MacDonald D, Robichaud A. Curr. Opin. Chem. Biol. 2001 5:432-38. Raboisson P, Lugnier C, Muller C, Reimund J M, Schultz D, Pinna G, Le Bec A, Basaran H, Desaubry L, Gaudiot F, Seloum M, Bourguignon J J. “Design, synthesis and structure-activity relationships of a series of 9-substituted adenine derivatives as selective phosphodiesterase type-4 inhibitors” Eur J Med Chem. 2003 38(2):199-214). Non-limiting examples of known selective PDE4 inhibitors include:

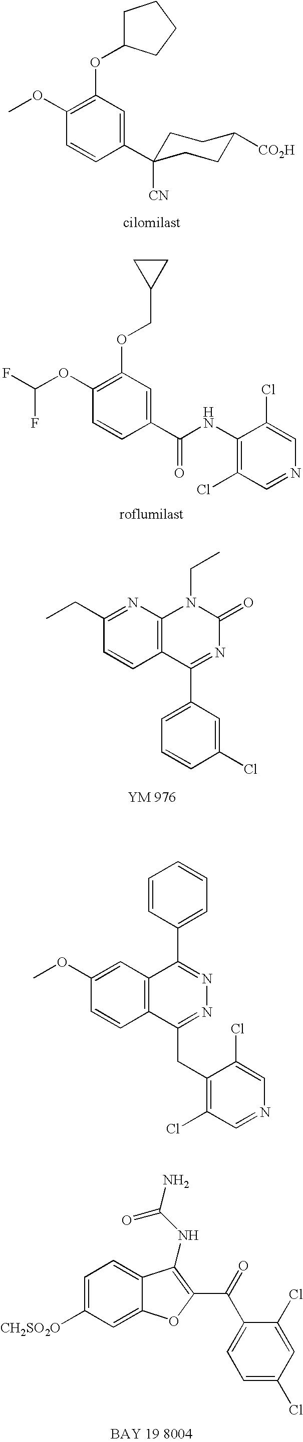

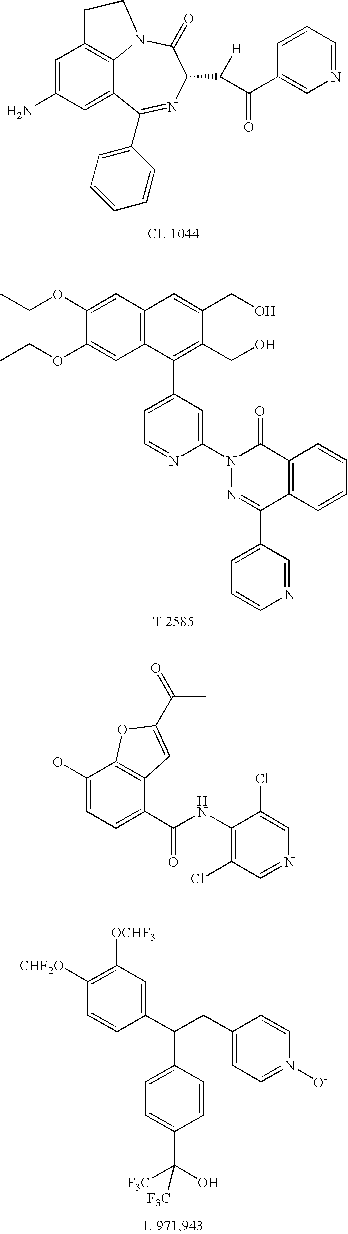

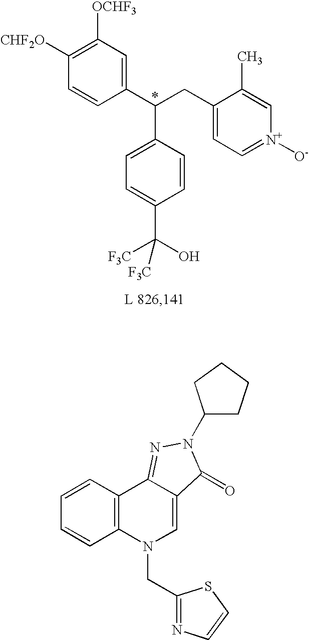

In one embodiment of the present invention, the indirect activator of KATP is an agent that increases cAMP. In a particular embodiment, the agent that increases cAMP is prostacyclin, prostaglandin E1, isoproterenol or a phosphodiesterase inhibitor.

The K_{ATP} channel can also be indirectly activated by agents that activate protein cAMP dependent protein kinases (i.e., protein kinase A). Non-limiting examples of cAMP-dependent protein kinase activators or elevating agents are found in U.S. Patent Application No. 0020198136, incorporated here by reference. U.S. Pat. No. 5,432,172 to Specter et al., entitled “Biological applications of alkaloids derived from the tunicate Eudistoma sp.”, teaches that Eudistoma alkaloids and the two synthetic pyridoacridines have effects similar to those obtained by chronic treatment with cAMP analogues or agents that elevate cAMP. These compounds include Segoline A, Segoline B, Isosegoline A, Norosegoline, Debromoshermilamine, Eilatin, 4-methylpyrido[2,3,4-kl]acridine, pyrido[2,3,4-kl]acridine, 1-acetyl-2,6-dimethylpyrido[2,3,4-kl]acridine, and derivatives.

In one embodiment of the present invention, the indirect activator of K_{ATP} is a agent that activates protein cAMP dependent protein kinases. In a particular embodiment, the indirect activator of K_{ATP} is a agent that activates protein kinase A.

V. Pharmaceutically Acceptable Salts

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids which form a physiological acceptable anion, for example, maleate, salyicylate, formate, fumarate, propionate, glycolate, lactate, pyruvate, oxalate, tannate, palmoate, alginate, tosylate, methanesulfonate, napthalenesulfonate, napthalenedisulfonate, acetate, citrate, malonate, tartarate, succinate, benzoate, ascorbate, galactouronate, polygalactouronate, glutamate, polyglutamate, α-ketoglutarate, and α-glycerophosphate. Suitable inorganic salts may also be formed, including, chloride, bromide, phosphate, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made. Other base addition salts can be formed with cations such as zinc, bismuth, barium, magnesium, aluminum, copper, cobalt, nickel, cadmium, or with an organic cation formed from N,N-dibenzylethylenedi-amine, ammonium, or ethylenediamine.

Pharmaceutically acceptable salts also may be combinations of the acid addition salts and base addition salts, for example a zinc tannate salt, or the like.

One, non-limiting example of a pharmaceutically acceptable salt is minoxidil sulfate, but other pharmaceutically acceptable salts comprise anions other than sulfate, such as chloride, carbonate, bicarbonate, nitrate, or the like.

VI. Pharmaceutical Compositions and Administration

Preferred modes of administration of the agonist(s) and diagnostic/therapeutic/prophylactic agent(s) are parenteral, intravenous, intra-synovial, intrathecal, intra-arterial, intraspinal, intrasternal, peritoneal, percutaneous, surgical implant, internal surgical paint, infusion pump, or via catheter. In one embodiment, the agent and carrier are administered in a slow release formulation such as an implant, bolus, microparticle, microsphere, nanoparticle or nanosphere. For standard information on pharmaceutical formulations, see Ansel, et al., Pharmaceutical Dosage Forms and Drug Delivery Systems, Sixth Edition, Williams & Wilkins (1995).

The agonist(s) and diagnostic/therapeutic/prophylactic agent(s) can, for example, be administered intravenously or intraperitoneally by infusion or injection. Solutions of the substance can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the substance which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, normal saline, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the substance in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Injectable solutions are particularly advantageous for local administration of the therapeutic composition. In particular, intramuscular injection can be used to deliver the therapeutic composition directly to the abnormal tissue, i.e. cancerous growth.

Useful dosages of agonist(s) and diagnostic/therapeutic/prophylactic agent(s) can be determined by comparing their in vitro activity, and in vivo activity in animal models. Methods for the extrapolation of effective dosages in mice, and other animals, to humans are known to the art; for example, see U.S. Pat. No. 4,938,949. The amount of the substance required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day. The substance is conveniently administered in unit dosage form; for example, containing 5 to 1000 mg, conveniently 10 to 750 mg, most conveniently, 50 to 500 mg of active ingredient per unit dosage form.

Ideally, the substance should be administered to achieve peak plasma concentrations of from about 0.5 to about 75 μM, preferably, about 1 to 50 μM, most preferably, about 2 to about 30 μM. This may be achieved, for example, by the intravenous injection of a 0.05 to 5% solution of the substance, optionally in saline, or orally administered as a bolus containing about 1-100 mg of the substance. Desirable blood levels may be maintained by continuous infusion to provide about 0.01-5.0 mg/kg/hr or by intermittent infusions containing about 0.4-15 mg/kg of the substance.

The substance may conveniently be presented in a single dose or as divided doses administered at appropriate intervals, for example, as two, three, four or more sub-doses per day.

The invention will now be illustrated by the following non-limiting Examples.

### EXAMPLES

Minoxidil sulfate, glibenclamide (Sigma Chemicals, St. Louis, Mo.); 1,3-dihydro-1-[2-hydroxy-5-(trifluoromethyl)phenyl]-5-(trifluoromethyl)-2H-benzimidazol-2-one (NS-1619); iberiotoxin (IBTX) (RBI chemical, Natik, Mass.); membrane potential assay kit (Molecular Devices, Sunnyvale, Calif.), and radiotracers (from NEN Co., Boston, Mass.) such as [¹⁴C] a-aminoisobutyric acid ([¹⁴C]-AIB, 57.6 mCi/mmol, MW, 103 Dalton), [¹⁴C] carboplatin (25 mCi/mmol, MW, 371 Dalton), [¹⁴C] dextran (2 mCi/mmol, MW, 70,000 Dalton), [³H]-glibenclamide (50 mCi/mmol, MW, 494 Dalton), von Willebrand factor (vWF, DAKO, CA), Neu polyclonal antibody (Santa Cruz Biotech, CA), glial fibrillary acidic protein (GFAP, Chemicon, CA), Her-2 monoclonal antibody (Zymed Labs. CA), and fluorescent labeled secondary antibodies obtained from Chemicon, Molecular Probes (OR) were used in the present study.

### Example 1

In Vivo BBB/BTB Permeability.

All animal experiments were conducted in accordance with policies set by the Institutional Animal Care and Usage Committee and NIH guidelines. A rat syngeneic tumor model was prepared using female Wistar rats and a human-tumor xenograft model in athymic nude rats weighing 180-200 g were prepared for the BBB/BTB permeability studies. Although brain tumor blood vessel development is rapid in rats compared to humans, this variable should not affect the findings or conclusions because the study analyzes the response of blood vessels, regardless of how rapidly they developed, to vasomodulators. The optimum number of tumor cells and incubation period for in vivo tumor growth was determined in separate experiments. Rat glioma (RG2) cells (1×10⁵) in 5 μl of medium with 1.2% methylcellulose were injected into the basal ganglia of Wistar rats, while nude rats were injected with glioblastoma multiforme (GBM) primary cells (5×10⁵). The coordinates were 5-mm lateral to the bregma and 4.5 mm deep to the basal ganglia. Seven days (for RG2 tumor) and 3-4 weeks (for GBM) after tumor implantation, the rats were prepared for permeability study as described by (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002; Ningaraj, N. S., Rao, M., and Black, K. L. Calcium-dependent potassium channels as a target protein ir. the modulation of blood-brain humor barrier. Drug News and Perspectives, 16:1-8, 2003). In regional permeability studies, 5 min after the start of the intracarotid (i.c.) infusion, 100 μCi/kg of [¹⁴C] AIB, [¹⁴C] dextran or [¹⁴C] carboplatin to 1 ml phosphate buffer saline (PBS) was injected as an intravenous (i.v.) bolus within a 15 second period. To determine whether the infusion of vasomodulators would affect cerebral blood flow (CBF) at the tumor area, Cerebral Laser-Doppler Flowmetry (LDF) using a Laser-Doppler (DRT4, Moore Instruments Ltd., England) equipped with a DP3 optical (1 mm diameter) probe during 15 min i.c. infusion of vasomodulators in some rats (n=3/group) was used as described in Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002; and Ningaraj, N. S., Rao, M., and Black, K. L. Calcium-dependent potassium channels as a target protein ir. the modulation of blood-brain humor barrier. Drug News and Perspectives, 16:1-8, 2003. Rats with abnormal CBF, blood gases or blood pressure were excluded from the study.

### Example 2

Ki Measurement.

The unilateral transfer constant, Ki (μl/g/min), which is an initial rate for blood-to-brain transfer of a radiotracer, was calculated as described by Ohno et al. (Ohno, K., Pettigrew, K. O., and Rapsport, S. T. Lower limits of cerebrovascular permeability to nanoelectrolytes in the conscious rat. Am. J. Physiol., 253: H299-H307, 1978). The Ki was determined for radiotracers, [¹⁴C]-AIB, [¹⁴C]-carboplatin, and [¹⁴C] dextran in the tumor core, tumor-adjacent brain tissue, and contralateral brain tissue using the quantitative autoradiographic (QAR) method as described earlier (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002; Ningaraj, N. S., Rao, M., and Black, K. L. Calcium-dependent potassium channels as a target protein ir. the modulation of blood-brain humor barrier. Drug News and Perspectives, 16:1-8, 2003). An optimum dose (10 μg/kg/min, i.c.) of BK, which was established in earlier studies (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002; Ningaraj, N. S., Rao, M., and Black, K. L. Calcium-dependent potassium channels as a target protein ir. the modulation of blood-brain humor barrier. Drug News and Perspectives, 16:1-8, 2003; Black, K. L., Cloughesy, T., Huang, S. C., Gobin, Y. P., Zbou, Y., Grous, J., Nelson, G., Faraberi, K., Hob, C. K., and Phelps, M. Intracarotid infusion of RMP-7, a bradykioin analog and transport of gallium-68 alkylenediamine tetraacetic acid into human gliomas. J. Neurosorg., 86: 603-609, 1997; Hashizume, K., and Black, K. L. Increased endothelial vesicular transport correlates with increased blood-tumor barrier permeability induced by bracykioin and leukotriene CA. J. Neuropathol. Exp. Neurol., 61-725-735, 2002; Sugita, M., and Black, K. L. Cyclic GMP-specific phosphodiesterasc inhibition and intracaretid bradykiaim intusion enhances permeability into brain tumors. Cancer Res., 58:914-923, 1998), and optimal dose (30 μg/kg/min for 15 min, i.c.) of MS was used for Ki measurements in rats (n=6/group). In a separate QAR study, varying doses of glibenclamide (0-10 μg/kg/min) were used to establish an optimal dose, which maximally attenuated the MS-induced BTB permeability increase in RG2 tumor-bearing rats. Additional experiments were performed in rats with RG2 tumor by co-infusing MS with glibenclamide (5 μg/kg/min) to investigate whether inhibition of K_{ATP} channels by glibenclamide attenuates MS-induced permeability increase. In addition, the effect of MS (with optimum dose and time established in rats (n=4/dose) harboring intracranial RG2 tumor) on BTB permeability in nude rats harboring GBM was investigated.

### Example 3

Dose Response Study.

To establish the optimal and safe dose range that would result in selective increases in BTB permeability without appreciably altering systemic blood pressure, varying doses (0-60 μg/kg/min) of MS were administered. In a separate study, Evans blue dye was administered i.v. in rats (n=4/group) with RG2 tumors to achieve a semi-qualitative measure of BTB permeability increase.

### Example 4

Time Course.

To determine whether vasomodulator-induced BTB permeability increase in RG2 tumor-bearing rats (n=4/time period) was transient or could be sustained over a long period, a separate QAR study was performed. BK (10 μg/kg/min), 30 μg/kg/min of MS or a (K_{Ca}) channel activator NS-1619, was infused (i.c.) separately for 15, 30 and 60 min periods, and Ki determined as described above.

### Example 5

Synergistic Effect of K_{Ca} and K_{ATP} Channel Agonists on BTB Permeability.

To investigate whether MS-induced BTB permeability increase was independent of (K_{Ca}) channel inhibitor iberiotoxin (IBTX) in RG2 tumor bearing rats (n=4/group). Further, to investigate whether K_{Ca} and K_{ATP} channel agonists administered simultaneously would exert a synergistic effect on BTB permeability; NS-1619 and MS (30 μg/kg/min each) were co-infused (i.c.) for 15 min and Ki measured by QAR.

### Example 6

Isolation of Brain Endothelial Cells.

In order to investigate the expression and activity of K_{ATP} channels in rat brain endothelial cells (RBEC), endothelial cells were isolated from the brains of neonatal rats using the method described earlier (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002). The homogeneity (>90-95%) of endothelial cells was verified by immunostaining with an endothelial cell marker, Factor VIII/von Willebrand factor (vWF). For potentiometric assays, RBEC cells were seeded alone and in co-culture with RG2 tumor cells in a gelatin-coated, 96 well plate to obtain a monolayer.

### Example 7

Co-Culture Experiments.

To investigate whether brain tumor cells can induce over expression of K_{ATP} channels in RBEC and human brain microvessel endothelial cells (HBMVEC), RBEC was co-cultured with RG2 cells, and HBMVEC with GMB cells. Suitable conditions for the co-culture's growth on glass cover slips in six-well tissue culture plates were standardized. Initially, about 1×10⁴ cells of RBEC and HBMVEC were co-coated with 1×10⁴ cells of RG2 and GBM, respectively, and allowed to achieve 70% confluency. RT-PCR and Western blot analyses in RBEC and HBMVEC alone or in co-culture with tumor cells were performed to study whether tumor cells induce over-expression of K_{ATP} channels at mRNA and protein levels in endothelial cells. In addition, immunocytochemical analysis of co-cultures was performed with vWF and K_{ATP} channel antibodies to support RT-PCR and Western blot data.

### Example 8

Western Blot Analysis.

To investigate the differential expression of K_{ATP} channels by the Western blot method, protein homogenates of normal brain and tumor tissues, tumor cells alone or in co-cultures were prepared by rapid homogenization in 10 volumes of lysis buffer (1% SDS, 1.0 mM sodium vanadate, 10 mM Tris pH 7.4). The extracts of normal brain and tumor tissues obtained from nude rats, which harbored intracerebral glioma for 3-4 weeks post tumor cell implantation, were used. Immunoblot analysis was performed on RG2 and GBM cells. Control protein lysates for the protein/receptor of interest were purchased from various vendors. Samples were fractionated on a 6-12% SDS-polyacrylamide gel, transferred to polyvinylidene difluoride membranes (Imobilon-P, Millipore, Bedford, Mass.) and probed with respective affinity-purified antibodies (optimum dilution was determined) for 1 h. After incubating with the primary antibody, membranes were incubated with peroxidase conjugated rabbit anti-mouse/rabbit immunogloblin (IgG) for 1 hr. The signals were detected with an enhanced chemiluminescence kit (Amersnan Pharmacia Biotech, NJ). Rabbit polyclonal primary anti Kᵢᵣ6.2 antibody was raised against the peptide sequences (EDP AEP RYR ARQ RRA RFV SKK). Anti β-actin monoclonal antibody was obtained from Santa Cruz Biotech (Santa Cruz, Calif.).

### Example 9

Immunocytochemistry (ICC).

K_{ATP} channels were co-localized in rat and human brain, and tumor sections with an anti-K_{ATP} channel antibody raised against a Kᵢᵣ6.2 subunit that was shown to be present in brain along with SUR 2A/B (Kitazono, T., Faraci, F. M., Taguchi, H., and Heistad, D. D. Role of potassium channels in cerebral blood vessels. Stroke 26:1713-1723, 1995; Melarned-Frank, M., Terzic, A., Carrasco, A. J., Nevo, E., Avivi, A., and Levy, A. P. Reciprocal regulation of expression of pre-forming K_{ATP} channel genes by hypoxia Mol. Cell Biochem, 225:145-150, 2001). ICC and confocal-LSM analysis were used to detect co-localization and differential expression of K_{ATP} channels with Kᵢᵣ6.2 antibody in normal and brain tumor microcapillary, and in human and rat tumor tissues. In ICC studies of K_{ATP} channels, HBMVEC and COS cells were positive and negative controls, respectively. All ICC experiments included either a control sample pre-incubated with a respective blocking peptide, whenever available, or control samples that did not contain primary antibodies. ICC analysis of K_{ATP} channels in athymic nude rats with intracerebral GBMs was performed as described recently (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002; Ningaraj, N. S., Rao, M., and Black, K. L. Calcium-dependent potassium channels as a target protein ir. the modulation of blood-brain humor barrier. Drug News and Perspectives, 16:1-8, 2003). To determine whether K_{ATP} channels are present on capillary endothelial and tumor cells, brain tumor sections were incubated with polyclonal Kᵢᵣ6.2 and monoclonal anti-vWF primary antibodies. The co-localization of K_{ATP} channels and vWF antibodies was accomplished with anti-rabbit IgG conjugated with a fluorescent (FITC) and anti-mouse IgG conjugated with rhodamine (TRITC). To demonstrate the expression of K_{ATP} channels in endothelial RG2 and GBM tumor cells, monolayers of RG2 and GBM cells were fixed with 4% paraformaldehyde, processed similarly and immumostained with anti-Kᵢᵣ6.2 primary antibody and anti-rabbit IgG conjugated with FITC. To demonstrate that tumor cells in-vitro and in-vivo were of glial origin, RG2 and GBM cells were immunostained with anti-rabbit polyclonal GFAP and anti-rabbit IgG conjugated with TRITC. Immunostaining of rat brain sections after i.c. delivery of Neu (human Her-2 equivalent) in RG2 bearing rats was performed with anti-rabbit Neu and secondary anti-rabbit IgG conjugated with TRITC. In contrast, immunostaining of rat brain sections after i.c. delivery of Her-2 MAb in GBM tumor bearing rat was detected using a Zeno Immunostaining kit (Molecular Probes).

### Example 10

RT-PCR Analysis.

RNA was isolated from endothelial (HBMVEC, RBEC), and tumor (RG2, GBM) cells and tissue samples, and purified using TRIzol (Life Technologies, CA), following the manufacturer's instructions. First-strand synthesis of cDNA using random hexamers was carried out in 2 μg aliquot of DNase 1-treated total RNA using Superscript II. Subsequently, 4 μl cDNA was amplified in a standard 50 μl PCR reaction (at appropriate conditions) containing 0.12 μmol/l of each primer. An aliquot (20 μl) of the RT-PCR product was analyzed on a 1% agarose gel and visualized using ethidium bromide. The locations of the primers indicated are based on the subunit sequence information obtained from GeneBank Kᵢᵣ6.2 subunit of K_{ATP} channel (human D50582), and Her-2 (human, X03363). The following primers were used for amplification of: Kir6.2 subunit of K_{ATP} channel-(5′-GTCACCGGAGCCATGCTGTCCCGC-3′ and 5′-GGGGGCCCGAGAGACCATGGCTCA-3′), and β-actin-(5′-AATCTGGCACCACACCTTCTAC-3′ and 5′-CTTCTCCTTAATGTCACGCACG-3′).

### Example 11

Confocal Microscopy.

Immunoreactive visualization of K_{ATP} channels, Her-2 and vWF was performed as described earlier (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002; Ningaraj, N. S., Rao, M., and Black, K. L. Calcium-dependent potassium channels as a target protein ir. the modulation of blood-brain humor barrier. Drug News and Perspectives, 16:1-8, 2003). In brief, confocal images were captured using a Leica (Heidelberg, Germany) TCS SP laser scanning confocal microscope (inverted) equipped with Argon (Ar: 488 nm) and Krypton (Kr: 568 nm) lasers. Fluorescent signals for K_{ATP} and Neu/Her-2 expressed on cultured rat and human endothelial cells, brain tumor cells and brain tumor-bearing rat brain sections were visualized using the 488 nm Ar laser line and fluorescent signals on vWF were visualized using the 568 nm-Kr laser line. Fluorescence signals for flour-488 or 568 were displayed individually as green and red pseudocolor projections or merged as overlay projections to visualize possible co-localization of two different antigens within specific subcellular structures.

### Example 12

[³H]-Glibenclamide Binding Study.

The densities of K_{ATP} channels in rat brain endothelial and tumor cells and in human normal brain and tumor tissue were determined by [³H]-glibenclamide binding study. To quantify K_{ATP} channels in vitro, RBEC alone or co-cultured with RG2 cells, rat brain, RG2 tumor tissue, HBMVEC alone or co-cultured with GBM, and normal human brain and GBM tissues were incubated with glibenclamide (5 μM) and [³H]-glibenclamide (5 nM) for 1 h in sodium-phosphate (Na-PB) buffer (10 mM, pH 7.4). The assay was modified from Rogers et al. (Challinor-Rogers, J. L., Kong, D. C., Iskander, M. N., and McPherson, G. A. Structure-activity relationship of [³H]-glibeociamide binding to membranes from rat cerebral cortex. J. Pharmacol. Exp. Ther., 273:778-786, 1995). Samples were washed thrice with Na-PB and cell tissue were lysed with ice-cold Na-PB, mixed with liquid scintillation fluid counted on a liquid scintillation counter. The actual binding of [³H]-glibenclamide was calculated by measuring the difference between non-specific and specific bindings in three separate experiments conducted in duplicate.

### Example 13

Membrane Potential Assay.

Functional activities of putative K_{ATP} channels in HBMVEC and GBM cells were measured by detecting changes in voltage across the cell membrane as described earlier (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002). A membrane potential assay kit (Molecular Devices, Sunnyvale, Calif.) provided a fast, simple and consistent mix-and-read procedure. In brief, HBMVEC and GBM cells obtained from cell passages 2-5 was suspended in 5 ml of growth medium. In two separate experiments, the cell density was adjusted to 1×10³ cells/well and coated in sterile, clear bottom, blank 96-well plates (Corning, Inc., Acton, Mass.), pre-coated with poly L-Lysine, and allowed to achieve a monolayer within 24 h. The monolayer cells were incubated with the membrane potential assay kit reagents for 30 min and read directly by FLEXstation. The anionic pchemiometric dye that traverses between cells and the extracellular solution in a membrane potential-dependent manner serves as an indicator of vasomodulator-induced voltage changes across the cell membrane. To determine the optimum dose that elicits detectable voltage changes across HBMVEC and GBM cell membranes, dose response studies were performed with 0-50 μM K_{ATP} channel agonist, MS, and K_{ATP} channel antagonist, glibenclamide (100 nM), using the Spectrofluorometer set to the following parameters: excitation (530 μm), emission (565 μm), and emission cut-off (550 μm) wavelengths. Additionally, the effects of the optimum dose (10 μM) of MS and glibenclamide (100 nM) on K_{ATP} channels activity in HBMVEC and GBM cells were determined.

### Example 14

GFP-Adenoviral (GFP-Adv) and crbB-2 Antibody Delivery.

GFP-Adv constructs were prepared as described by Smith et al. (Smith, G. M., Berry, R. L., Yang, J., and Tanelian, D. Electrophysiological analysis of dorsal root ganglion neurons pre- and post-coexpression of green fluorescent protein and functional 5-HHT3 recepter. J. Neurophysiol. 77:3115-3121, 1997) with slight modifications. GFP gene was first cloned into a shuttle vector, pAdTrack-CMV, and the resultant plasmid was linearized by digesting it with restriction endonuclease Pipe I, and subsequently co-transformed into E. Coli cells with an adenoviral backbone plasmid, pAdEasy-1. Recombinant plasmids were transfected into 293 cells. The presence of recombinant adenoviruses was verified by RT-PCR. GFP-Adv (1×10⁹ pfu/ml) was infused intracarotidly (i.c.) with or without MS in rats with implanted GBM. Neu rabbits polyclonal and Her-2 MAb, which are specific for rat and human erbB-2 receptors, respectively, were used. Both antibodies were dialyzed using 100 KD cut-off dialysis tubing (0.5 ml capacity, 10 μm pore size, Spectrum Labs) to remove bovine serum albumin (BSA) and other additives. MS (30 μg/kg/min) was infused (i.c.) for 15 min followed by i.c. infusion of Neu or Her-2 MAb (1 mg/kg) or GFP-Adv (1×10⁹ pfu/ml) for 45 min. After 2 h rats (n=2) were perfuse fixed with 4% paraformaldehyde transcardially, and the brain removed. Her-2 MAbs bound to Her-2 receptors, in vivo was detected by incubating rat brain cryosections as per the manufacturer's (Molecular Probes, OR) procedure with Alexa Flour-647 conjugated secondary antibody (Zecon labeling kit) that specifically binds to the IgG1 fragment of Her-2 MAb. Neu antibody binding to erbB-2 receptors in RG2 brain tumor sections was detected using anti-rabbit IgG conjugated with TRITC. To investigate whether there was Adv-GFP delivery and expression of GFP in tumor cells, another group of rats (n=2) were transcardially perfuse-fixed with 4% paraformaldehyde after 96 hr, brains removed, and cryosections were imaged for GFP's presence by confocal microscopy. Further, to demonstrate the expression of GFP on glial tumor cells, GFP was colocalized with GFAP.

### Example 15

Transmission Electron Microscopy (TEM).

Seven days after RG2 tumor cell implantation, rats (n=3/group) were infused i.c. with PBS, BK (10 μg/kg/min in 0.5% DMSO), NS-1619 (30 μg/kg/min in 0.5% ethanol) or MS (30 μg/kg/min in 0.5% DMSO) for 15 min. Rats were infused with 10 ml cold PBS, and perfuse-fixed with 250 ml of 1% glutaraldehyde in PBS (pH 7.4) through the heart. Tumor-bearing brains were removed and 1 mm³ tissue pieces encompassing tumor mass, brain surrounding the tumor and normal brain cut, and samples were processed for TEM analysis (JEOL electron microscope operating at 80 kV) as described earlier (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002).

### Example 16

Quantitative Analysis.

At least 10 profiles of capillaries from each group sectioned transversely and photographed at low magnification (X=7,200) were evaluated for their general features as described earlier (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002). Briefly, micrographs were placed on a digitizing screen and structural features measued using Scan Pro 4™, a computer-assisted, image analysis system (Jandel Scientific, Corte Madera, Calif.). Abluminal and luminal circumferences, areas of endothelial cytoplasm excluding nuclei and vacuoles, and mean thickness of endothelial cytoplasm were measured and compared with those in the control group. The mean thickness of endothelium was calculated by subtraction of the luminal radius from the abluminal radius, which was obtained from the areas encircled by the luminal and abluminal circumferences, respectively. The proportion of total vesicular area to the cytoplasmic area was expressed as a percentage in order to derive another parameter to characterize vesicular transport.

### Example 17

Survival Study.

Wistar rats with implanted intracranial tumors were used to study the effect of MS on increased carboplatin delivery and survival. RG2 cells (1×10⁵) were implanted intracranially to form a tumor (2 mm in size) in rats within a week. After a week, rats were given saline, carboplatin (5 mg/kg) or carboplatin after 15 min of MS (30 μg/kg/min for 15 min) infusion through an exteriorized catheter once a day for three consecutive days. The rats were carefully monitored for mortality and clinical signs due to brain tumor growth for 90 days or until death, whichever came first. Brains of dead, moribund or those rats that survived beyond 90 days were removed, frozen and cryosectioned for histological evaluation, in order to compare tumor volumes between treated and untreated groups. Kaplan-Meier analysis was performed to determine the statistical significance of the results.

### Example 18

Statistics.

Results are expressed as mean±SD where applicable. For all in vivo permeability studies, n≧4 rats/group were used unless otherwise stated. Unpaired two-tailed Student's t-tests were used to compare the control and treated groups. The statistical analyses of Ki, vesicle density, vesicular area, cleft index and cleft area index comparison among different groups, with or without drug treatment, were performed using ANOVA followed by either impaired parametric analysis of Student's t test or non-parametric analysis of Mann-Whitney's U test. P<0.05 was considered statistically significant. Statistical analysis of the vesicular density and proportion of cumulative vesicular area compared the effect of i.c. infusion of MS with that of PBS infusiors.

### Example 19

BBB Capillaries Differ from BTB Capillaries.

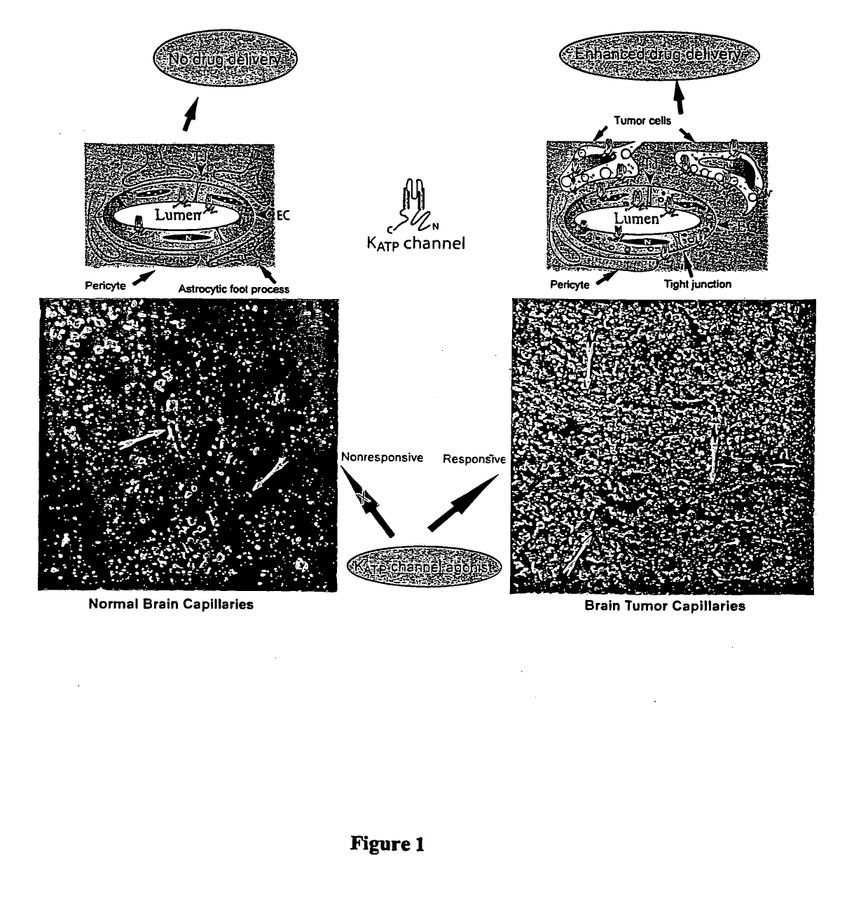

The differences in the morphology and biochemical response to K_{ATP} channel agonists between the BBB and BTB are shown in FIG. 1. BTB capillaries and surrounding tumor cells over-express K_{ATP} channels and, therefore, may readily respond to activation by K_{ATP} channel agonists. In contrast, K_{ATP} channels are hardly detected in normal brain microvessel endothelial cells, which, therefore, and may not respond to K_{ATP} channel agonists.

### Example 20

K_{ATP} Channels Mediate MS-Induced BTB Permeability Increase.

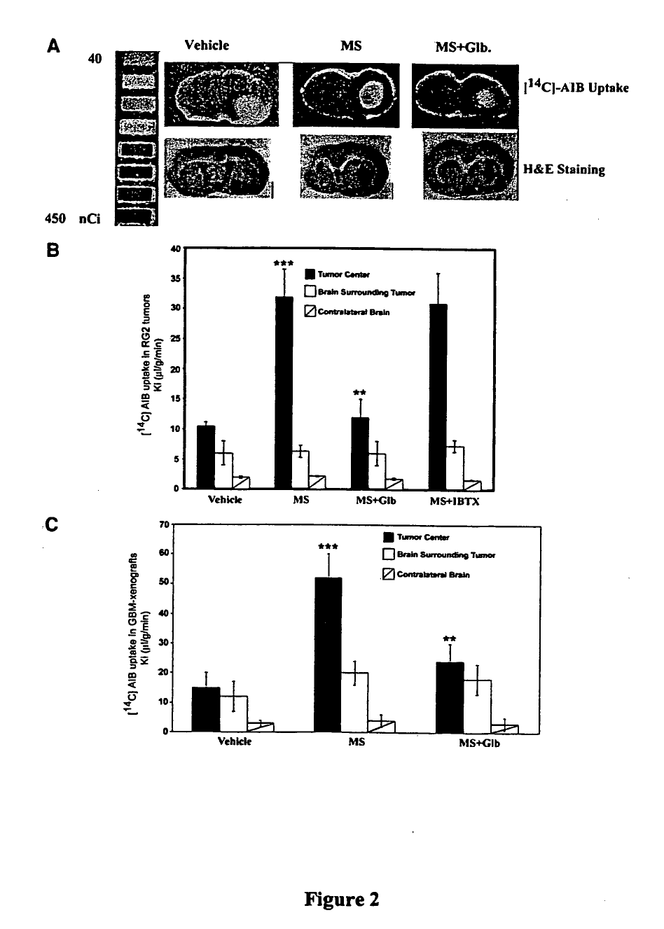

BTB permeability, Ki (μl/g/min), was measured by QAR of cryosections obtained from RG2 or GBM tumor-bearing rat brains following the injection of a [¹⁴C]-labeled tracer, 5 min after i.c. MS infusion. To determine whether K_{ATP} channels mediate MS-induced BTB permeability increase, rats with implanted intracerebral RG2 tumors received i.c. MS infusion, either alone or with glibenclamide. Ki was determined for radiotracer [¹⁴C]-AIB in the tumor. core, tumor-adjacent brain tissue, and contralateral brain tissue. Comparison of psuedocolor-enhanced autoradiographs of rat brain sections showed enhanced delivery of [¹⁴C]-AIB upon i.e. MS infusion (FIG. 2A). Glibenclamide co-infusion blocked MS-induced [¹⁴C]-AIB uptake (FIG. 2A). After i.c. infusion of MS (30 μg/kg/min) for 15 min, Ki was significantly increased in the tumor center (32±5 μl/g/min; P<0.001) compared to PBS controls (10.5±1.5 μl/g/min). MS induced increase in BTB permeability was significantly inhibited (12.2±3.0 μl/g/min, P<0.01) by co-infusion of glibenclamide at a dose of 5 μg/kg/min for 15 min (FIG. 2B). MS induced Ki increase was not blocked by IBTX (a specific K_{Ca} channel inhibitor), suggesting that MS's action is independent of K_{Ca} channels (FIG. 2B). Increases in BTB permeability obtained with a fixed dose of MS (30 μg/kg/min) was shown to be blocked by co-infusion with glibenclamide (0-25 μg/kg/min) in a dose-dependent manner. Additionally, in a GBM xenograft nude rat model, MS (30 μg/kg/min) was shown to significantly increased BTB permeability (52±8 μl/g/min; P<0.001), which was attenuated (24±6 μl/g/min; P<,0.01) when glibenclamide (5 μg/kg/min for 15 min) was co-infused with MS (FIG. 2C). In contrast to the tumor center, MS with or without glibenclamide did not significantly affect BBB permeability in normal brain surrounding tumor (BST, 2 mm area outside tumor margin) or in normal contralateral brain (FIG. 2C). Similarly, when infused alone or prior to MS infusion, glibenclamide did not affect BBB or BTB permeability (data not shown).

### Example 21

Time Course.

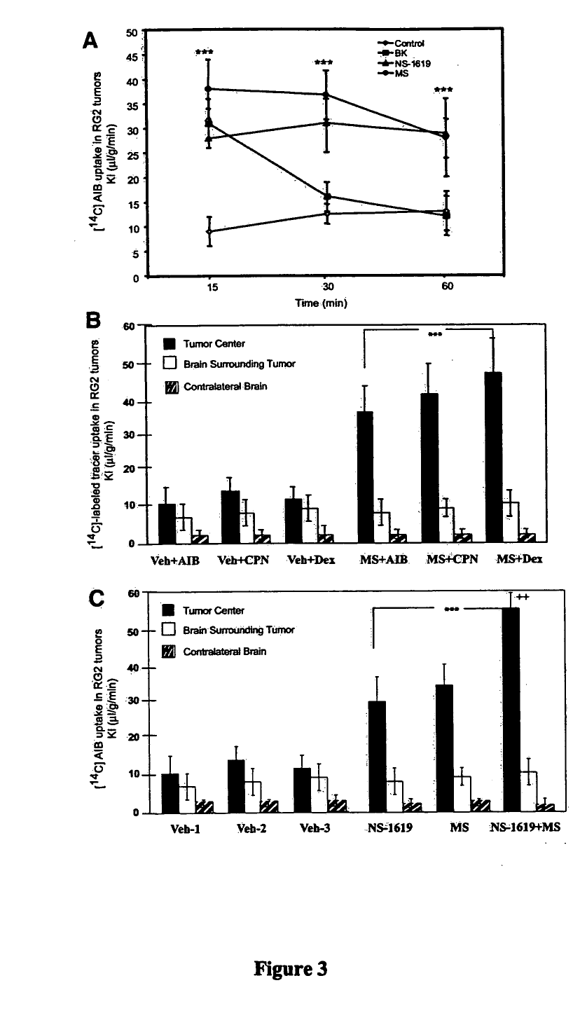

The present QAR study in rats with implanted RG2 tumor showed that i.c. infusion of MS (30 μg/kg/min) significantly (P<0.001) enhanced sustained delivery of [¹⁴C]-AIB to the tumor for 15, 30 and 60 min infusions (FIG. 3A). MS's ability to sustain BTB permeability increase up to 60 min was consistent with the data on NS-1619 in a similar model (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002). In contrast, a 30 min or 60 min infusion of BK failed to sustain the initial increase of Ki (P<0.001) attained at 15 min (FIG. 3A). For comparative purpose, a similar molar concentration of BK and NS-1619 was infused.

### Example 22

K_{ATP} Channel Activation Elicits Increased Delivery of Molecules of Varying Sizes.

Intracarotid infusion of MS (30 μg/kg/min) increased BTB permeability in implanted intracerebral RG2 tumor to various-sized radiotracers that normally fail to cross the BTB including hydrophilic compounds such as [¹⁴C]-labeled-AIB, dextran and a chemotherapeutic agent, carboplatin (CPN), that normally fail to cross the BTB. Co-fusion with MS significantly enhanced delivery of [¹⁴C]-labeled AIB, dextran and carboplatin (FIG. 3B). In contrast, [¹⁴C]-carboplatin delivery was negligible in vehicle-treated rats. These studies further suggest that K_{ATP} channel-mediated increases in BTB permeability allow delivery of drugs of varying molecular size, including AIB (MW 103), CPN (MW 361), and dextran (70 KD), suggesting that the effect is independent of molecular size (FIG. 3B).

### Example 23

Synergistic Effect of K_{Ca} and K_{ATP} Channel Agonists on BTB Permeability.

To investigate whether K_{ATP} and K_{Ca} channel agonists exert a synergistic effect on BTB permeability increase, MS and NS-1619 were co-infused i.c. for 15 min. This combination drug infusion significantly increased BTB permeability compared to the BTB permeability increase elicited when these drugs were infused alone (FIG. 3C). This result suggests that K_{ATP} channel-mediated BTB permeability increase occurs by a different pathway than the K_{Ca} channel-mediated effect.

### Example 24

ErbB-2 Antibody and GFP-Adv Delivery Across the BTB.

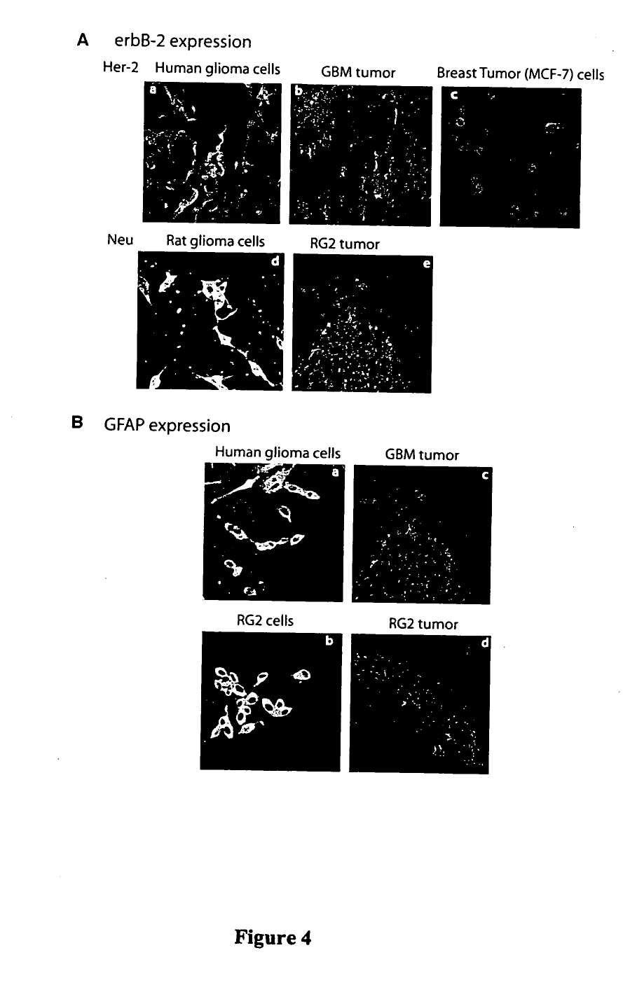

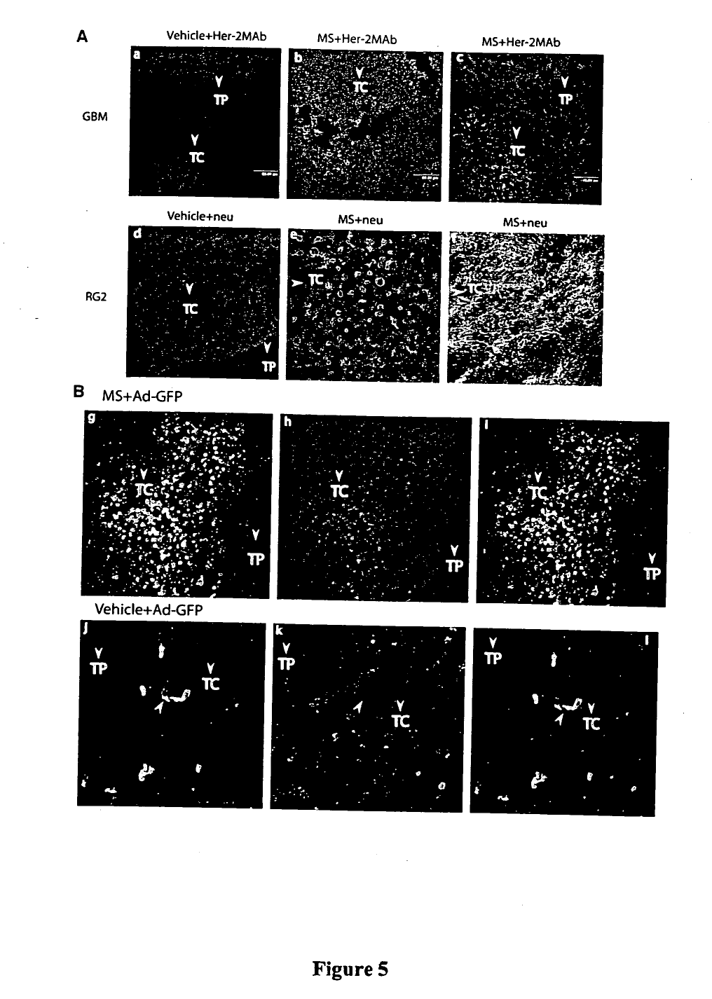

ErbB-2 expression was demonstrated in RG2 and GBM in vitro and in vivo (FIG. 4). Low Her-2 expressing MCF-7 (human breast tumor cells) were used as a negative control. Further, the glial origin of RG2 and GBM was demonstrated by GFAP expression in vitro and in vivo (FIG. 4). After studying MS's ability to increase BTB permeability to allow the delivery of various sized molecules, MS was infused to increase Neu, Her-2 MAb and GFP-Adv delivery to rat brain tumor in vivo. RG2 (Neu-positive) tumor-bearing Wistar rats and GBM (Her-2 positive) tumor bearing athymic nude rats were used. MS infusion enhanced delivery of Her-2 MAb selectively to GBM and Neu to RG2 (FIG. 5A) tumor tissues without any delivery to contralateral brain tissues (FIG. 5A). In contrast, very little Her-2 MAb and Neu was delivered to tumor tissue in vehicle-treated rats (FIG. 5A-a,d). MS also enhanced adv-GFP delivery to brain tumors. Abundant GFP expression was seen in brain tumor cells in rats co-infused with MS and adv-GFP (FIG. 5B-g) but not in the tumor periphery (TP). In addition, GFP expression was observed predominantly on the tumor cells because GFAP co-localized with GFAP (FIG. 5B-i). In contrast, adv-GFP infused alone failed to cross the BTB and infect tumor cells; hence a negligible GFP expression was detected on tumor cells. Furthermore, GFP expression was observed in the cerebral blood vessels but not in the tumor cells because the GFP did not co-localize with GFAP (FIG. 5B-1).

### Example 25

K_{ATP} Channel Mediated BTB Permeability Modulation and CBR.

I.c. infusion of low doses of K_{Ca} channel activators, such as BK and NS-1619, did not alter CBF in tumor and normal brain (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002). Using a laser-operated Doppler, i.c. administration of 30 μg/kg/min of MS was shown not to significantly affect CBF (Table 1), even though BTB permeability increased in the tumor area. Mean arterial blood pressure (MAP) in the drug-treated groups was not significantly different from the vehicle-treated group (Table 1).

TABLE 1
Physiological measurements during BTB
permeability determination

| | | | | | CBF |
|---|---|---|---|---|---|
| | | PaO₂, | PaCO₂, | MAP, | (% change |
| Groups | pH | mm Hg | mm Hg | mm Hg | vs. vehicle) |
| Vehicle - | 7.3 ± 0.02 | 87 ± 4 | 42 ± 2 | 90 ± 5 | 0 |
| MS-treated | 7.38 ± 0.01 | 90 ± 5 | 45 ± 1 | 83 − 4 | 8 ± 3 |
| (30 μg/kg/min) | | | | | |
| MS-Gib | 7.35 ± 0.02 | 88 ± 5 | 49 ± 3 | 81 ± 5 | 5 ± 2 |
| (30 + 10 | | | | | |
| μg/kg/min) | | | | | |
| MS + NS-1619 | 7.30 ± 0.02 | 92 ± 5 | 44 ± 4 | 75 ± 5 | 12 ± 2 |
| (both 30 | | | | | |
| μg/kg/min) | | | | | |

The physiological parameters were determined either before or during vasomodulator infusion. The values are mean±SD of n>4 rats, except for cerebral blood flowmetry (CBF) where three representative rats per group were used. pH is arterial pH; PaO₂ is arterial oxygen; PaCO₂ is arterial carbon dioxide; and MAP is mean arterial blood pressure. A dose of MS and NS-1619 (30 μg/kg/min) that did not appreciably alter MAP in rats was selected for BTB permeability studies by QAR.

### Example 26

Tumor Cells Increase K_{ATP} Channel Expression in Endothelial Cells.

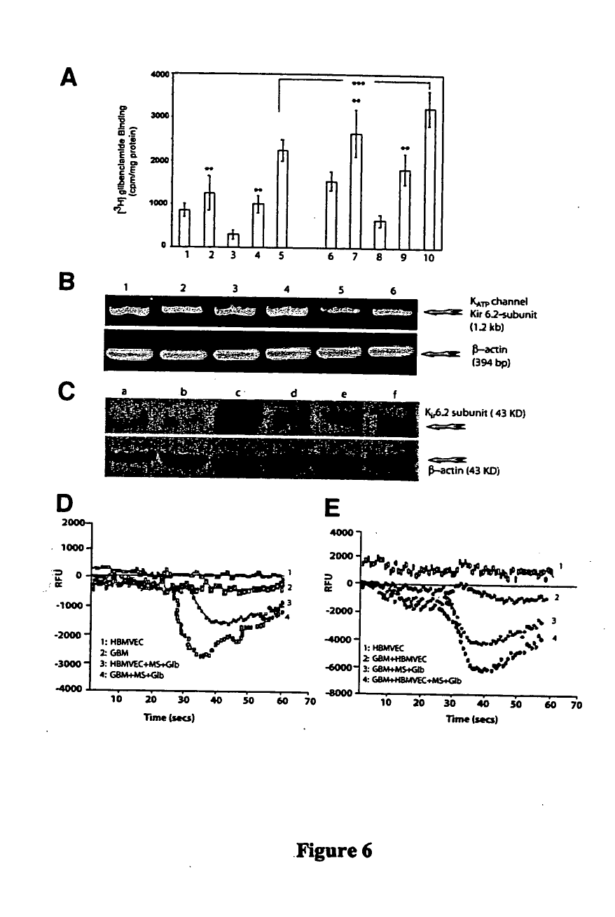

The specific binding of [³H]-glibenclamide in the membranes of tumor cells and tissues was significantly higher than in membranes prepared from normal endothelial cells and brain tissue (FIG. 6A). When co-cultured with tumor cells, endothelial cells showed increased [³H]-glibenclamide binding compared to endothelial cells and tumor cells alone (FIG. 6A), suggesting an increase in K_{ATP} channel density distribution in the co-cultured cells, possibly influenced by signals arising from the tumor cells. An increased [³H]-glibenclamide binding in GBM compared to normal brain tissue was also observed (FIG. 6A). RT-PCR analysis also showed the influence of tumor cells on K_{ATP} channel on RNA expression in endothelial cells co-cultured with RG2 and GBM primary cells (FIG. 6B). Western blot analysis (FIG. 6C) and K_{ATP} channel activity assay by potentiometry (FIG. 6C, FIG. 6D) confirmed the RT-PCR results and also indicated that tumor cells may induce over-expression of K_{ATP} channels in endothelial cells.

### Example 27

K_{ATP} Channel Activity.

The functional activity of putative K_{ATP} channels in a monolayer of HBMVEC and GBM cells was determined by measuring their membrane potential in response to MS at varying concentrations. The depolarization action of MS was highly pronounced in GBM when compared to HMBVEC (FIG. 6D). The membrane potential decreases in HBMVEC and GBM cells in response to the addition of MS, and a return to resting membrane potential with the addition of glibenclamide was measured spectrofluorometrically using potassium ion-fluorescent dye. These results demonstrate K_{ATP} channel activity in HBMVEC and GBM cells in response to MS. Further, endothelial cells, when co-cultured with tumor cells, exhibited higher activity than endothelial or tumor cells alone (FIG. 6E). This finding suggests the presence of higher K_{ATP} channel density distribution on endothelial cells that were grown with brain tumor cells than on endothelial cells alone. This observation confirmed similar results obtained by RT-PCR, Western blot and [³H]-glibenclamide binding experiments (FIG. 6A). In addition, MS caused a dose-dependent decrease in K⁺ dye-specific fluorescence intensity in RBEC, HBMVEC, RG2 and GBM cells, which was reversed by glibenclamide administration.

### Example 28

Immunolocalization of K_{ATP} Channels.

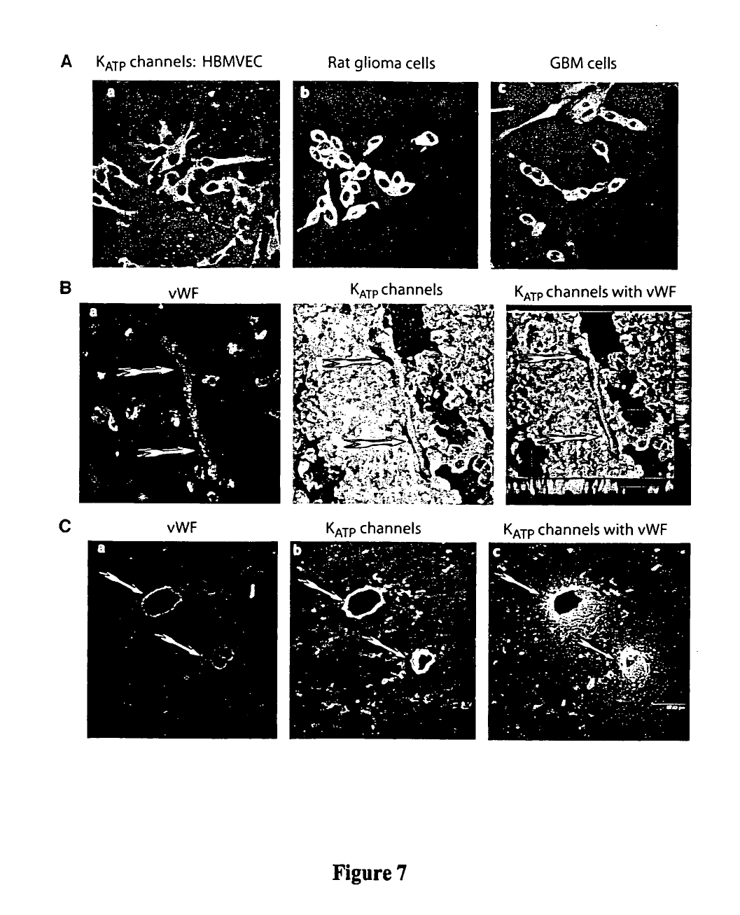

Why and where K_{ATP} channel modulators selectively induce BTB permeability without affecting BBB permeability? It was thought that such a selective effect might be due to increased expression of K_{ATP} channels in tumor capillaries and tumor cells compared with normal brain. To address this issue, the anti-Kᵢᵣ6.2 subunit (the pore forming) antibody to immunolocalize K_{ATP} channels was used in paraformaldehyde perfusion-fixed GBM and RG2 tumor bearing rat brain in sections. This analysis of rat brain or human brain tumor sessions for expression of K_{ATP} channels and endothelial cell marker, vWF, by two-color immunocytochemistry indicated that vWF-positive tumor vessels (red) were also positive for K_{ATP} channels (green). K_{ATP} channel expression on the plasma membrane of endothelial, RG2 and GBM cells was demonstrated. More intense immunostaining for K_{ATP} channels in tumor cells (FIG. 7A-b) compared to normal endothelial cells (FIG. 7A-a). Microvessels positive for both antigens are shown in yellow (FIG. 1 and FIG. 7B,C). It was determined whether K_{ATP} channels are expressed differentially and more abundantly on tumor cells than in normal brain, which might explain the MS-induced selective BTB permeability increase. The immunolocalization study of normal brain-sections showed some positive expression for K_{ATP} channels in non-capillary cells while no positive K_{ATP} channel expression was observed in normal human brain endothelial cells (FIG. 1). However, a robust expression to K_{ATP} channels in GBM cells (FIG. 7A-b), tumor capillary endothelium (FIG. 7B-b,c), and rat brain tumor capillary endothelial cells (FIG. 7C-b,c) compared to low K_{ATP} channel coloralization in normal brain capillaries (FIG. 1) was observed. These results strongly suggest that the selective BTB permeability effects of MS, and MS-induced BTB permeability increase attenuation by glibenclamide is due to the increased density distribution of K_{ATP} channels on brain tumor capillary endothelium and tumor cells compared with normal brain capillary endothelium and normal brain cells.

### Example 29

Vesicular Transport.

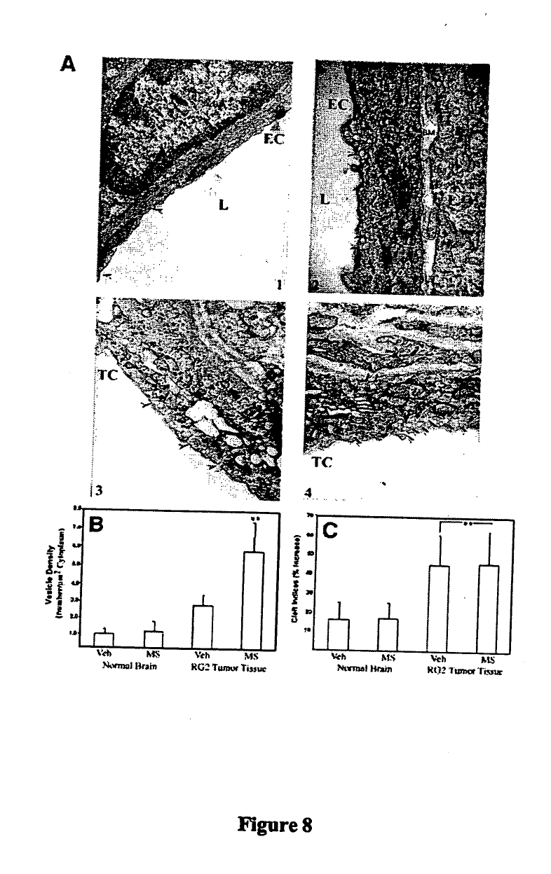

It was further investigated whether vesicular transport is largely responsible for enhanced delivery of drugs and macromolecules across the BTB. Transmission electron microscopy (TEM) was used to demonstrate that no changes occurred in the normal capillary endothelium of contralateral brain tissue following i.c. MS infusion (data not shown). Similarly, PBS infusion in tumor-bearing rats did not elicit changes in tumor capillary endothelium (FIG. 8A2), although i.c. MS infusion accelerated formation of pinocytotic vesicles by invaginations of the luminal membrane of tumor capillary endothelium (FIG. 8A4), and the alignment and movement of vesicle arrays along the luminal-abluminal axis of the capillary endothelium. These vesicles dock and fuse with basement membrane, and then appear to release their contents on the abluminal side of the endothelial membrane (FIG. 8A4). MS significantly increased vesicular density (FIG. 8B), although MS did not alter the endothelial tight junction indices in the tumor capillaries (FIG. 8C). These results demonstrate for the first time that the primary cellular mechanism for macromolecular delivery across the BTB after K_{ATP} channel activity is via increased vesicular transport and not via the paracellular route through endothelial tight junctions.

### Example 30

Survival Study.

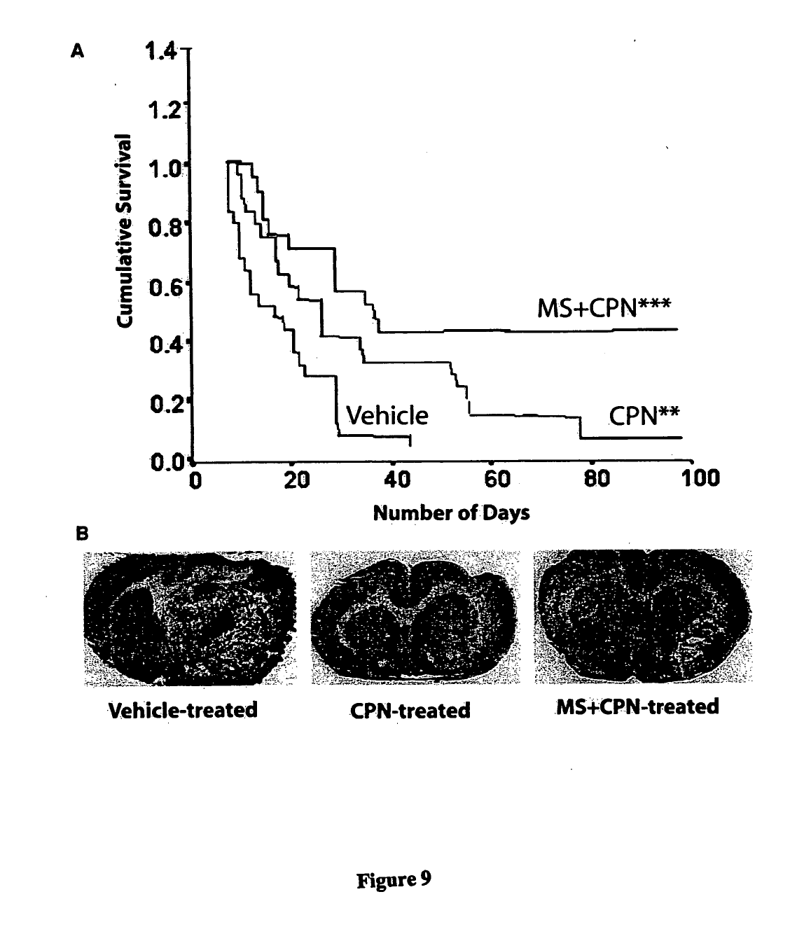

In Wistar rats harboring intracranial RG2 tumors, MS significantly enhanced [¹⁴C]-carboplatin delivery to tumor without affecting normal brain (FIG. 3B). In addition, Kaplan-Mercer analysis showed that rats with RG2 tumors survived significantly longer when treated with carboplatin and MS in combination compared to carboplatin only and vehicle only groups (FIG. 9A). The mean survival in the MS and carboplatin-treated group was 90.57±6.56 days (P<0.01 vs. carboplatin alone; P<0.001 vs. untreated group) compared to the carboplatin alone group (55.46±5.71 days) and the untreated group (29.56±2.44 days). Combination treatment (MS+carboplatin) resulted in a significant reduction in tumor size (FIG. 9B). The mean tumor size in the combined treatment (1.30±1.2 mm²) group was the smallest of any group, followed by the group treated with carboplatin only (5.30±1.2 mm²) and the vehicle-treated group (9.37±2.2 mm²) (FIG. 9A).

### Example 31

Therapy for Brain Tumors

The prognosis for patients with GBM is extremely poor, with a median survival of 9 months (Smith, G. M., Berry, R. L., Yang, J., and Tanelian, D. Electrophysiological analysis of dorsal root ganglion neurons pre- and post-coexpression of green fluorescent protein and functional 5-HHT3 recepter. J. Neurophysiol. 77:3115-3121, 1997; Fellner, S., Baner, B., Miller, D. S., Schaffrik, M., Fankbanel, M. Spruss, T., Bernhardt, G., Grueff, C., Ferber, L., Gechaidmeier, H., Buscheuer, A., and Fricker, G. Transport of paclitaxel (Taxol) across the blood brain barrier in vitro and in vivo. J. Clin. Invest., 110:1309-1318, 2002), primarily due to a paucity of effective treatment options. Even though some agents have proved effective against tumors outside the brain, impaired drug delivery across the BTB limits drug delivery to primary and metastatic brain tumors (Fellner, S., Baner, B., Miller, D. S., Schaffrik, M., Fankbanel, M. Spruss, T., Bernhardt, G., Grueff, C., Ferber, L., Gechaidmeier, H., Buscheuer, A., and Fricker, G. Transport of paclitaxel (Taxol) across the blood brain barrier in vitro and in vivo. J. Clin. Invest., 110:1309-1318, 2002). Usually, primary brain tumors are intrinsically resistant to drugs, only adding to the problem of inadequate drug delivery across the BTB. In contrast, metastatic brain tumors that spread to the brain are sensitive to anti-cancer drugs. The BTB, however, prevents the delivery of anticancer drugs in sufficient amounts to achieve any therapeutic benefit. Therefore, improved delivery of anti-cancer agents to metastatic brain tumors may greatly improve the prognosis for those afflicted with brain metastases. Breast and lung cancers are the most frequent tumors to spread to the brain and together account for 70% of brain tumor metastases. In non-small cell lung cancers (NSCLC) there is a 70% response rate in NSCLC patients to chemotinerspy using carboplatin/etoposide while the response rate for NSCLC patients with brain metastases drops to 10-30%. Similarly, with regard to Her2-positive metastatic breast cancer, 88% of breast cancer patients develop bone matastasis and 33% develop brain metastasis. In contrast, of breast cancer patients receiving trasbuzamab only 4% develop bone metastasis but 28% still develop brain metastasis.

### Example 32

BBB Capillaries Differ from BTB Capillaries

Brain tumor capillaries over-express K_{Ca} channels (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002; Ningaraj, N. S., Rao, M., and Black, K. L. Calcium-dependent potassium channels as a target protein ir. the modulation of blood-brain humor barrier. Drug News and Perspectives, 16:1-8, 2003). These findings are consistent with other studies that showed over-expression of vascular proteins (Fuchs, I. B., Loebbecke, M., Buhler, H., Stoltenburg-Didinger, G., Heine, B., Lichtenegger, W., and Schaller, G., HER2 in brain metastatses: issues of concordance, survival and treatment. J. Clin. Oncol. 20:4130-4133, 2002; 29. Folkman, J. Angiogenesis in cancer, vacular, rheumatoid and other disease. Nat. Med., 1:27-31, 1995; Pardridge, W. M., Blood-brain barrier genomics. In: W. M. Pardridge (ed.), Brain Drug Targeting-The Future of Brain Drug Development, pp 275-3000, United Kingdom: Cambridge University Press, 2001), such as angiogenic vascular endothelial growth factors (VEGF), fibronectin, and αvβ integrins in tumor capillaries. The BTB is structurally and functionally different from the BBB (Ruoslanti, E. Specialization of tumor vasculature. Nature Reviews/Cancer, 2:83-90, 2002; Hashizume, K., and Black, K. L. Increased endothelial vesicular transport correlates with increased blood-tumor barrier permeability induced by bracykioin and leukotriene CA. J. Neuropathol. Exp. Neurol., 61-725-735, 2002; Asotra, K., and Black, K. L. Blood-brain barrier as portal for drug delivery. Advances Clin. Neurosci., 10:323-339, 2000; Uchida, M., Ninganaj, N., Chen Z., Black, L., and Asotra, K. Cycllic GMP-induced blood-brain-tumor barrier permeability is not mediated by Cyclic GMP-dependent protein kinase. Am. Assoc. Neurolog. Surg., 1160, 2001). Recent studies (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002; Ningaraj, N. S., Rao, M., and Black, K. L. Calcium-dependent potassium channels as a target protein ir. the modulation of blood-brain humor barrier. Drug News and Perspectives, 16:1-8, 2003; Uchida, M., Ninganaj, N., Chen Z., Black, L., and Asotra, K. Cycllic GMP-induced blood-brain-tumor barrier permeability is not mediated by Cyclic GMP-dependent protein kinase. Am. Assoc. Neurolog. Surg., 1160, 2001), however, have shown that certain protein are specifically expressed or over-expressed in tumor capillaries. Tumor cells over-express bradykinin type 2 receptors (Liu, Y., Hashizums, K., Samoto, K. Sugita, M., Ningaraj, N., Asotra, K., and Black, K. L. Correlation between bradykinin-induced blood tumor barrier permeability and B2 receptor expression in experimental brain tumors. Neurological Res., 23:379-387, 2001), while both tumor and tumor capillary endothelial cells over-express K_{Ca} channels (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002; Ningaraj, N. S., Rao, M., and Black, K. L. Calcium-dependent potassium channels as a target protein ir. the modulation of blood-brain humor barrier. Drug News and Perspectives, 16:1-8, 2003) and protein kinase-G (Uchida, M., Ninganaj, N., Chen Z., Black, L., and Asotra, K. Cycllic GMP-induced blood-brain-tumor barrier permeability is not mediated by Cyclic GMP-dependent protein kinase. Am. Assoc. Neurolog. Surg., 1160, 2001) and K_{ATP} channels rendering them potential targets for biochemical modulation of BTB permeability. Although, the magnitude of differences in protein expression between normal and tumor capillaries is qualitative, using a series of complimentary studies it was quantitatively shown that tumor capillaries have increased K_{ATP} channel density, activity and/or responsiveness to MS.

### Example 33

Biochemical Mediation of BTB Permeability

Vasomodulators, such as BK, nitric oxide donors, soluble guanylate cyclase activators, and NS-1619, increase BTB permeability via K_{Ca} channels (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002). MS can also biochemically modulate K_{ATP} channels resulting in a significant BTB permeability increase, allowing the delivery of hydrophilic tracers, GFP-adenoviral vectors and Her-2 antibodies specifically to brain tumor tissue. Glibenclamide co-infusion with MS attenuated MS-induced BTB permeability. Glibenclamide's efflux may be a potential concern because it is a substrate for multidrug-resistance-associated proteins present on brain tumor microvessels. In the study design, however, glibenclamide efflux may not significantly affect the blockage of MS-induced BTB permeability increase because glibenclamide was co-infused with MS for only a 15 min period. Furthermore, there was a synergistic effect between K_{Ca} and K_{ATP} channel agonists, suggesting that they act independently. The absence of any significant functional alteration in CBF by K_{Ca} (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002) and K_{ATP} (Esaki, T., Itolc, Y., Shimoji, K., Cook, M., Jehle, J., and Sokoloff, L. Blockade of K(ATP) channels with glibeaclamide does not alter functional activation of cerebral blood flow in the unamethelized rat. Brain Res 945:56-63, 2002) channel agonists is consistent with the present data (Table 1). The low doses of these vasoactive agents required to achieve a significant charge in permeability allows increased drug delivery without the hypotensive effect expected with high doses of these agents. MS, when administered i.v., also increased BTB permeability in a rat brain tumor model without increasing BBB permeability. Based on these observations, an i.v. formulation of MS was developed to test whether MS enhances anti-cancer drug delivery to brain tumor in human patients.

### Example 34

K_{ATP} Channels in Cerebral Vasculature

The presence and role of K_{ATP} channels in normal cerebravascular endothelium have been described (Nelson, M. T. and Quayle, J. M. Physiological roes and properties of potassium channels in arterial smooth muscle. Am. J. Physiol., 268:C799-C822, 1995; Yokoshiki, H., Sunagawa, M., Seki, T., and Sperelakis, N. ATP-sensitive K-Channels in pancreatic, cardiac, and vascular smooth muscle cells. Am. J. Physio. 274:C25-37, 1998), but their role in BBB/BTB permeability has not been elucidated. FIG. 6A, B, FIG. 7 demonstrate abundant expression of K_{ATP} channels in tumor cells (in vitro and in vivo) in contrast to normal astrocytes and endothelial cells. Confocal images clearly revealed K_{ATP} channel over-expression on tumor and endothelial cells compared to normal brain. More importantly, the tumor capillaries (FIG. 7B, C) showed abundant expression of K_{ATP} channels as K_{ATP} channels co-localize with vWF in tumor capillary endothelial cells. Increased presence of K_{ATP} channels (FIG. 6A, B, C) and their activity in endothelial cells (FIG. 6D, E), possibly by tumor cell-induced signaling, was observed when endothelial cells were co-cultured with tumor cells. Others also reported increased K_{ATP} channels activity in pathological conditions such as hypoxia (Schoch, H. J., Fischer, S., and Warti, H. H. Hypoxia-induced vascular endothelial growth factor expression causes vascular leakage in the brain. Brain 125: 2549-2557), which might also be true in tumors, since tumors thrive in hypoxic environments. In normal brain capillaries, however, K_{ATP} channels were barely detectable even when over-expressed K_{ATP} channels were detected in tumor capillaries (FIG. 1). This unique feature of tumor capillaries offers a mechanism that can be exploited to alter tumor capillary permeability selectively without concomitant effects on normal brain.

### Example 35

Brain Tumor Cells Induce K_{ATP} Channels Over-Expression

In rat and human brain cells, the functional activity of K_{ATP} channels in normal and tumor cells alone and in co-cultures was demonstrated. MS elicited higher membrane potential changes in tumor cells than in normal cells, suggesting greater density distribution or sensitivity of K_{ATP} channels in tumor cells than in normal cells. Furthermore, in vitro [³H]-glibenclamide binding studies showed that K_{ATP} channel density is significantly higher in tumor than normal cells tissue (FIG. 6A). Therefore, K_{ATP} channels are over-expressed both on tumor cells and tumor capillary endothelial cells compared to those of normal brain, which may explain why MS selectively increases BTB permeability while leaving the BBB unaffected. In addition, because of the observed synergistic effect of K_{Ca} and K_{ATP} channel agonists on BTB permeability, K_{ATP} channels are an additional target besides K_{Ca} channels (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002; Ningaraj, N. S., Rao, M., and Black, K. L. Calcium-dependent potassium channels as a target protein ir. the modulation of blood-brain humor barrier. Drug News and Perspectives, 16:1-8, 2003) for BTB permeability modulation to enhance drug delivery to brain tumors.

### Example 36

Mechanism of Increased Transport: Pinocylotic Vesicles or via Tight Junctions

The results demonstrate that MS induces accelerated formation of transport vesicles in both brain tumor capillary endothelium and tumor cells by MS-induced activation of K_{ATP} channels (FIG. 8A). Therefore, vesicular transport is largely responsible for enhanced delivery of drugs across the BTB rather than via the opening of endothelial tight junctions. This finding is consistent with the earlier study (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002), which reported that a K_{Ca} channel agonist, NS-1619, increased the density of rat brain tumor microvessel endothelial vesicles. A slight increase in basal BTB permeability might be due to a small increase in the number of pinocytotic vesicles and the right functional cleft index (Stewart, P. A. Endothelial vesicles in the blood-brain barrier: are they related to permeability? Cell Mol. Neurobiol., 20:149-163, 2002; Liebner, S., Fischman A., Rascher, G., Duffner F., Grote, E. H., Kalbacher, H., and Wolburg, H. Claudin-1 and claudin-5 expression and tight junction morphology are altered in blood vessels of human glioblastoma multiforme. Acta Neuropathol., 100:323-331, 2000). A direct relationship between an increase in the number of brain tumor capillary endothelial vesicles and increased BTB permeability was found. Most importantly, rat brain tumor capillary endothelial cells form far more vesicles (FIG. 8B) than normal brain capillary endothelial cells without altering the endothelial tight junctions in response to vasomodulators, such as MS (FIG. 8C) or NS-1619 (Ningarsj, N. S., Rao, M., Hashizume, K., Asotra, K., and Black, K. L. Regulation of blood-brain tumor barrier permeability by calcium-activated potassium channels J. Pharmacol. Exp. Ther., 301:838-851, 2002).

### Example 37

Enhanced Survival

In an earlier studies, it was shown that carboplatin enhanced survival when rats with intracranial glioma were co-treated with NS-169 (Ningaraj, N. S., Rao, M., and Black, K. L. Calcium-dependent potassium channels as a target protein ir. the modulation of blood-brain humor barrier. Drug News and Perspectives, 16:1-8, 2003) or BK (Matsukado, K., Sugita, M. and Black, K. L. Intracarotid low dose bradykinin infusion selectively increases tumor permeability through activation of bradykinin B2 receptors in malignant gliomas. Brain Res., 792: 10-15, 1998). In the present invention, it was shown that i.c. MS infusion selectively enhanced [¹⁴C]-carboplatin delivery to tumor tissue without increasing delivery to normal brain (FIG. 3B). It was also shown that MS co-infusion with carboplatin in rats resulted in tumor regression, significantly increasing survival (FIG. 8A,B). Primary brain tumors, particularly GBM, frequently have altered genes resulting in tumor cell proliferation, as well as poor prognosis and patient survival (Newcomb, E. W., Cohen, H., Lee, S. R., Bhalla, S. K., Bioom, J., Hayes, R. L., and Miller, D. C. Survival of patients with glioblastoma multiforme is not influenced by altered expression of p16, p53, EGF4r, MDM2 or Bcl-2 genes. Brain Pathol., 8:655-667, 1998; Ljubimova, J. Y., Loksh, A., Yong, W. H. Riedinger, M. S., Miner, J. H., Sorokin, L. M. Ljubimov, A. V., and Black, K. L. Overexpression of Alpha 4 chain-containing laminins in human glial tumors identified by gene microarray analysis. Cancer Res., 61:5601-5610, 2001; Schlegel, J., Stamm, G., Brandle, K., Merdes, A., Mechtersheimer, G., Hynes, N. E. and, Kiessling, M. J. Amplification and differential expression of members of the erbB-gene family in human giioblastoma. Neurooncol., 22:201-207, 1994). In particular, Her-2, which is over-expressed in 17-20% of GBMs (Forseen, S. E., Pott, A., Koka, V., Koch, M., Fraiman, G., and Levitt, R. Identification and relationship of HER-2 neuroverexpression to short-term mortality to primary malignant brain tumors. Anticancer Res. 22:1599-602, 2002), facilitates tumor cell proliferation (Schlegel, J., Stamm, G., Brandle, K., Merdes, A., Mechtersheimer, G., Hynes, N. E. and, Kiessling, M. J. Amplification and differential expression of members of the erbB-gene family in human giioblastoma. Neurooncol., 22:201-207, 1994). Trestuzumab and 2CA, developed by Genentech Inc. are potential Her-2 receptor-based therapies for gliomas. The molecular sizes of these MAbs, however, prevent their efficient delivery across the BTB to tumor. MS-induced biochemical modulation of K_{ATP} channels enhanced delivery of macromolecules, including Her-2 MAb, selectively to tumors without increasing MAb delivery to normal brain in a GBM-xenograft model (FIG. 5A). This finding suggests that therapeutic antibodies could be efficiently and selectively directed to glioma cells in vivo. In addition to MAb therapy, gene therapy for GBM is emerging as a potential treatment strategy. Efficient and selective delivery of adenoviral vectors to tumor across the BTB, however, is difficult when viral products are administered through an intravascular route because of the difficulty of getting such vectors across the BTB. Enhanced and selective GFP-Adv delivery across the BTB following i.c. co-infusion with MS (FIG. 5B) was demonstrated. This strategy may be clinically useful to deliver therapeutic antibodies with a chemotherapeutic drug or gene product selectively to brain tumor while leaving healthy brain intact.

The Blood-Brain Tumor Barrier (BTB) hinders drug delivery to brain tumors, including glioblastoma multiforme (GBM), an especially devastating cancer. In a human-tumor xenograft rat model, minoxidil sulfate (MS), a specific agonist of K_{ATP} channels, selectively enhances drug delivery across the BTB to tumor without affecting normal tissue. Highly proliferative GBM overexpress Her-2 receptors (Her-2r) are an ideal targets for glioma therapy, The BTB, however, limits delivery of a monoclonal Her-2 antibody. In nude rats with intracerebrally implanted GBM, MS's ability to enhance survival by increasing BTB permeability to labeled Her-2 monoclonal antibody with or without MS was investigated.

### Example 38

Nude Rat and Mouse Tumor Model

GBM-xenograft rat and mouse models were developed by intracranially implanting primary GBM cells isolated from patient brain tumor tissue. The IC₅₀ of Herceptin on GBM cells in vitro by FACS analysis was established. To investigate whether MS administration by intracarotid (i.c.) and intravenous (i.v.) routes, increases BTB permeability to different tracers, and enhances delivery to brain tumor, permeability studies were performed. The effect of i.c. and i.v. MS administration on BTB permeability in a GBM-xenograft rat model is presented below.

The following data was obtained using a human brain tumor-xenograft rat model and human brain tissues. The data show that in brain tumor, K_{ATP} channels are over-expressed on tumor cells and tumor capillaries compared to normal brain cells and capillaries. Immunohistochemistry data show that K_{ATP} channels do not appear to be expressed on normal brain microvessels. There is, however, expression of K_{ATP} channels in normal brain, as would be expected. There is also expression of K_{ATP} channels in GBM tumor tissue. In tumor microvessels, there was co-localization of K_{ATP} channels with factor VIII. In general, these data shows high expression of K_{ATP} channels in GBM by immunohistochemistry. K_{ATP} channels appear also to be over-expressed on tumor microvessels with double labeling of factor VIII and K_{ATP} channels.

Immunolocalization of K_{ATP} channels and Factor VIII in human brain tumor sections is shown. To colocalize K_{ATP} channels in brain microvessels with an endothelial specific marker (Factor VIII/von Willebrand factor (vWF), K_{ATP} channel and anti-vWF antibodies were used. Tumor microvessels appear to co-express K_{ATP} channels and Factor VIII, suggesting that the expression of K_{ATP} channels occur in the tumor microvessels. Tumor cells also appear to overexpress K_{ATP} channels.

Images were captured using a Leica (Heidelberg, Germany) TCS SP laser scanning confocal microscope (inverted) equipped with Argon (Ar, 488 nm) and Krypton (Kr, 568 nm) lasers. FITC signals for K_{ATP} channels expressed on normal and metastatic brain tumor sections were visualized using the 488-nm Ar laser line. TRITC signals for factor VIII were visualized using the 568 nm-Kr laser line. Fluorescence signals for FITC and TRITC were displayed individually as green and red pseudocolor projections or merged as overlay projections to visualize possible co-localization of two different antigens within specific subcellular structures.

### Example 39

Delivery of Macromolecules to Brain Tumor

GFP-Adv constructs were prepared, and GFP-Adv (1×10⁹ pfu/ml) was infused i.c. with or without MS in rats with implanted GBM. Her-2 MAb (Zymed Labs) was dialyzed to remove bovine serum albumin (BSA) and other additives. MS (30 μg/kg/min) was infused (i.c.) for 15 min followed by infusion (i.c.) of Her-2 MAb (1 mg/ml/kg) or GFP-Adv (1×10⁹ pfu/ml) for 45 min. For the Her-2 MAb study, after 2 h the rat was perfuse-fixed with 4% paraformaldehyde transcardially, and the brain removed. Brain cryosections were incubated as per the manufacturer's (Molecular Probes, OR) procedure with Alexa Flour-647-conjugated secondary antibody (Zenon labeling kit) that specifically binds to the IgG1 fragment of Her-2 MAb. The sections were examined under a confocal microscope. To investigate whether there was GFP-Adv delivery and expression of GFP in tumor cells, another group of rats were transcardially perfuse-fixed with 4% paraformaldehyde after 48 hrs, brains removed, and cryosections imaged for GFP's presence by confocal microscopy.

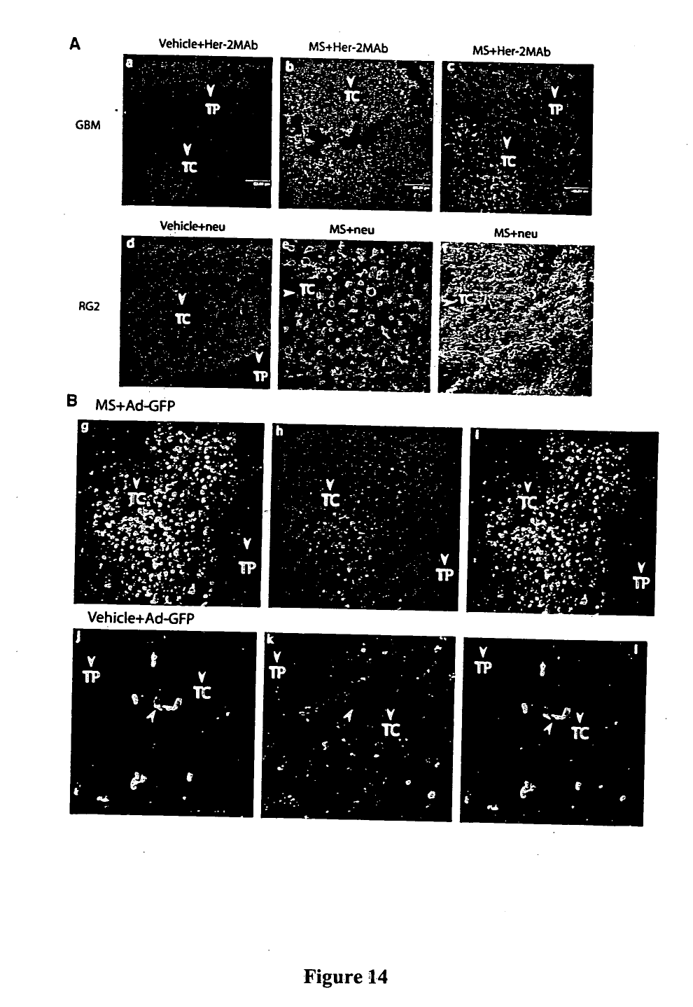

Permeability study: The ability of i.c. MS infusion on BTB permeability in a GBM-xenograft rat model was demonstrated (FIG. 12).

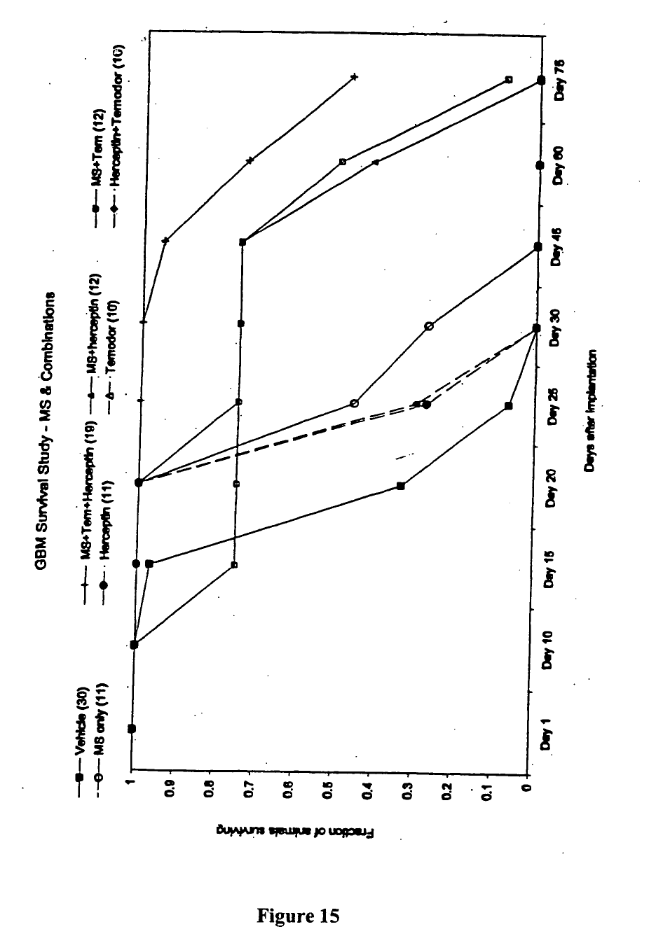

The ability of i.v. MS infusion on BTB permeability in a GBM-xenograft rat model was demonstrated (FIG. 13).

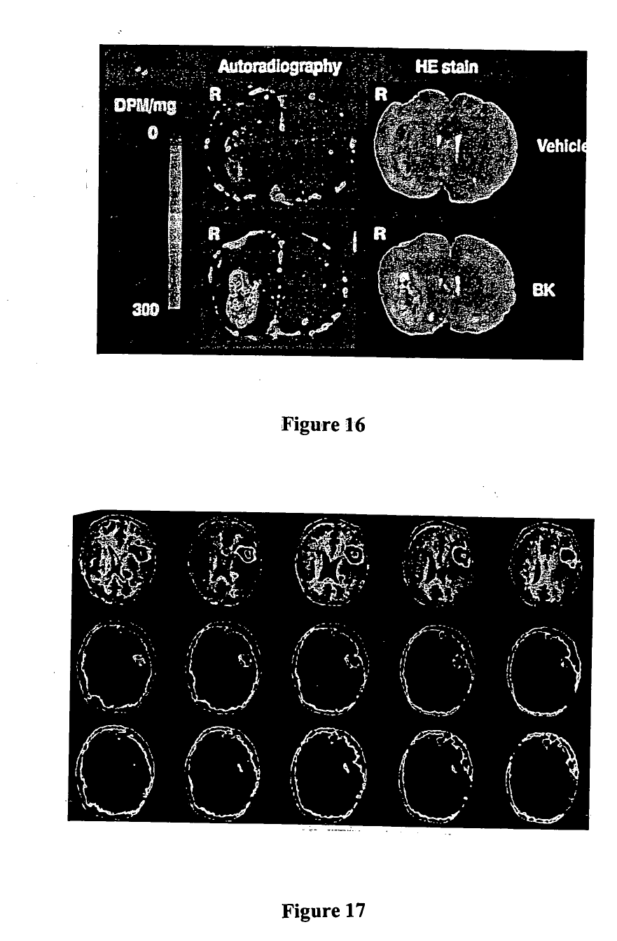

The ability of MS to enhance BTB permeability to macromolecules such as Her-MAb in a GBM-xenograft rat model was demonstrated (FIG. 14).

### Example 40

Survival Study

Nude mice were intracranially implanted with cells. The tumor was allowed to grow for 7 days before treatment was initiated. Mice were divided into groups: (1) control, (2) MS (0.9 mM), (3) Temodor (27 mM), (4) Herceptin (4 mg/kg), (5) MS+Temodor, (6) minoxidil sulfate+Herceptin, (7) herceptin+temodor, and (8) MS+Herceptin+temodor. Untreated mice were either dead or euthanized due to tumor-related complications within 30 days. Immediately after 75-days post tumor implantation, all surviving treated mice were euthanized.

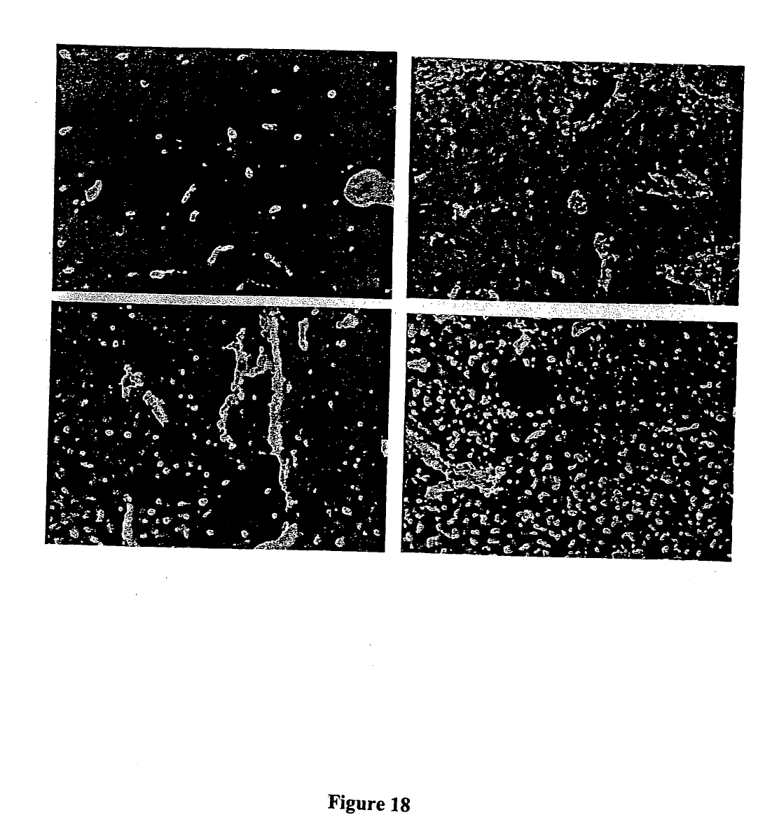

Survival study: Kaplan-Meier analysis showed that nude mice harboring intracranial human glial tumors survived longer when treated i.v. (three consecutive doses for three days) with a combination of MS, temodor and Herceptin than vehicle-only groups (FIG. 15). The mean survival in the MS, temodor and Herceptin group was 67.2±11.8 days; P<0.001 vs. vehicle (20.93±8.9 days), MS (27.09±8.7 days, Herceptin (24.91±8.3 days) and temodor (25.5±6.3 days) alone treatment groups).

### Example 40

Overexpression of KATP Channels in Metastatic Brain Tumors.

Metastasized brain cancer cells originating from lung, breast and kidney all have high levels of K_{ATP} channels (left panels, green stain). Moreover, metastasized tumors also have high levels of expression of K_{ATP} channels on the brain microcapillary endothelium adjacent to the tumor (right panels, yellow cells). See FIG. 22.

### Example 41

Potassium Channel Agonists Enhance Uptake of Compounds Selectively to Metastatic Breast Brain Tumors.

When were co-injected intravenously with [¹⁴C]AIB and minoxidil sulfate, the levels of 14 C in tissue taken from the brain tumor is approximately three times as great as when they are injected with saline and [¹⁴C]AIB. The level of [¹⁴C]AIB surrounding the tumor and in the contralateral brain does not differ significantly between the vehicle treated and minoxidil sulfate treated rats. See FIG. 23.

### Example 42

Potassium Channel Agonists Enhance Uptake of Compounds Selectively to Metastatic Lung Brain Tumors.

Intravenous co-inection of minoxidil sulfate increased the uptake of [¹⁴C]AIB specifically to the tumor vs. the brain surrounding the tumor or contralateral brain approximately three-fold. See FIG. 24.

1. A method for delivering a therapeutic, prophylactic or diagnostic agent to an abnormal brain region in a mammalian subject comprising administering one or more activators of an ATP-sensitive potassium channel in combination with one or more activators of a calcium-activated potassium channel, under conditions, and in an amount sufficient to increase the permeability to the therapeutic, prophylactic or diagnostic agent of a capillary or arteriole delivering blood to cells of the abnormal brain region; and also administering to the subject the therapeutic or diagnostic agent, so that the therapeutic, prophylactic or diagnostic agent is delivered selectively to the cells of the abnormal brain region.

2. The method of claim 1, wherein the activator of the ATP-sensitive potassium channel is a direct agonist.

3. The method of claim 2, wherein the direct agonist is minoxidil or minoxidil sulfate.

4. The method of claim 2, wherein the direct agonist is cromakalim, levcromakalim, emakalim, bimakalim, celikalim, rimakalim, pinacidil, aprikalim, picartamide, diazoxide or nicorandil.

5. The method of claim 1, wherein the activator of the ATP-sensitive potassium channel is an indirect activator.

6. The method of claim 5, wherein the indirect activator is an activator of adenylyl cyclase.

7. The method of claim 6, wherein the activator of adenylyl cyclase is forskolin.

8. The method of claim 1, wherein the activator of the calcium-activated potassium channel is a direct agonist.

9. The method of claim 8, wherein the direct agonist of the calcium-activated potassium channel is NS1619, NS-1608, NS-04, NS-08 or EBIO.

10. The method of claim 1, wherein the activator of the calcium-activated potassium channel is an indirect activator.

11. The method of claim 10, wherein the indirect activator is an activator of soluble guanylyl cyclase.

12. The method of claim 11, wherein the activator of soluble guanylyl cyclase is nitric oxide.

13. The method of claim 10, wherein the activator of soluble guanylyl cyclase is an NO-independent activator of soluble guanylyl cyclase.

14. The method of claim 13, wherein the NO-independent activator of soluble guanylyl cyclase is carbon monoxide, a porphyrin, a metallopophyrin, YC-1, BAY-2272, BAY 41-2272, BAY 41-8543.

15. The method of claim 1, wherein the abnormal brain region is a region of tumor tissue.

16. The method of claim 1, wherein the tumor tissue is malignant.

17. The method of claim 1, wherein the abnormal brain region is a region of brain tissue physiologically affected by stroke.

18. The method of claim 1, wherein the abnormal brain region is a region of brain tissue physiologically affected by a bacterial, viral or prion infection.

19. The method of claim 1, wherein the abnormal brain region is a region of brain tissue physiologically affected by neurodegeneration.

20. The method of claim 1, where in the abnormal brain region is a region of brain tissue physiologically affected by injury or trauma.

21. The method of claim 1, wherein the therapeutic agent is an anti-proliferative agent.

22. The method of claim 21, wherein the anti-profliferative agent is carboplatin or cisplatin.

23. The method of claim 1, wherein the therapeutic agent is an anti-stroke agent.

24. The method of claim 1, wherein the therapeutic agent is a mood stabilizing agent, an anti-convulsant, an anti-neurodegenerative agent, an adrenergic agent, a cytokine, a therapeutic protein, an immunotoxin, an immunosuppressant, a DNA expression vector, a viral particle or a therapeutic oligonucleotide.

25. The method of claim 1, wherein the mammal is a human.

26. The method of claim 1, wherein the therapeutic or diagnostic agent is administered to the mammalian subject substantially simultaneously with the activators.

27. The method of claim 1, wherein the activators are administered by intravenous or inter-arterial infusion or injection.

28. The method of claim 1, wherein the activators are administered by intra-carotid infusion or injection.

29. A method for delivering a therapeutic, prophylactic or diagnostic agent to an abnormal brain region in a mammalian subject comprising administering a direct agonist of an ATP-sensitive potassium channel in combination or alternation with a direct agonist of a calcium-activated potassium channel, under conditions, and in an amount sufficient to increase the permeability to the therapeutic, prophylactic or diagnostic agent of a capillary or arteriole delivering blood to cells of the abnormal brain region; and administering to the subject the therapeutic or diagnostic agent, so that the therapeutic, prophylactic or diagnostic agent is delivered selectively to the cells of the abnormal brain region.

30. The method of claim 29, wherein the direct agonist of the ATP-sensitive potassium channel is minoxidil or minoxidil sulfate.

31. The method of claim 29, wherein the direct agonist of the ATP-sensitive potassium channel is cromakalim, levcromakalim, emakalim, bimakalim, celikalim, rimakalim, pinacidil, aprikalim, picartamide, diazoxide or nicorandil.

32. The method of claim 29, wherein the direct agonist of the calcium-activated potassium channel is NS1619, NS-1608, NS-04, NS-08 or EBIO.

33. The method of claim 29, wherein the abnormal brain region is a region of benign or malignant tumor tissue.

34. The method of claim 29, wherein the abnormal brain region is a region of brain tissue physiologically affected by stroke.

35. The method of claim 29, wherein the abnormal brain region is a region of brain tissue physiologically affected by bacterial, viral or prion infection.

36. The method of claim 29, wherein the abnormal brain region is a region of brain tissue physiologically affected by neurodegeneration.

37. The method of claim 29, wherein the abnormal brain region is a region of brain tissue physiologically affected by injury or trauma.

38. The method of claim 29, wherein the medicant is an anti-proliferative agent.

39. The method of claim 38, wherein the anti-proliferative agent is carboplatin or cisplatin.

40. The method of claim 29, wherein the mammal is human.

41. The method of claim 29, wherein the therapeutic or diagnostic agent is administered to the mammalian subject substantially simultaneously with the direct agonists.

42. The method of claim 29, wherein the direct agonists are administered to the mammalian subject by intravenous or intra-arterial infusion or injection.

43. The method of claim 29, wherein the direct agonists are administered to the mammalian subject by intravenous or intra-arterial infusion or injection.

44. A method for delivering a therapeutic, prophylactic or diagnostic agent to a malignant tumor in a mammalian subject comprising administering one or more activators of an ATP-sensitive potassium channel in combination with one or more activators of a calcium-activated potassium channel, under conditions, and in an amount sufficient to increase the permeability to the therapeutic, prophylactic or diagnostic agent of a capillary or arteriole delivering blood to cells of the malignant tumor; and administering to the subject the therapeutic or diagnostic agent, so that the therapeutic, prophylactic or diagnostic agent is delivered selectively to the cells of the abnormal brain region.

45. The method of claim 44, wherein the malignant tumor is a located in the brain.

46. The method of claim 44, wherein the malignant tumor is located in the breast, bone, prostate, liver, lung, larynx, gall bladder, head, neck, stomach, kidney, skin cervix, connective tissue, adrenal gland, pancreas, spine, thorax, peritoneum, bowel, colon, rectum, or lymphatic system of the mammalian subject.

47. The method of claim 44, wherein the mammal is a human.

48. The method of claim 44, wherein the activator of the ATP-sensitive potassium channel is a direct agonist.

49. The method of claim 48, wherein the direct agonist is minoxidil or minoxidil sulfate.

50. The method of claim 48, wherein the direct agonist is cromakalim, levcromakalim, emakalim, bimakalim, celikalim, rimakalim, pinacidil, aprikalim, picartamide, diazoxide or nicorandil.

51. The method of claim of claim 44, wherein the activator of the calcium-activated potassium channel is a direct agonist.

52. The method of claim 51, wherein the direct agonist is NS1619, NS-1608, NS-04, NS-08 or EBIO.

53. The method of claim 44, wherein the activator of the ATP-sensitive potassium channel is an indirect activator.

54. The method of claim 53, wherein the indirect activator is an activator of adenylyl cyclase.

55. The method of claim 54, wherein the activator of adenylyl cyclase is forskolin.

56. The method of claim 44, wherein the activator of the calcium-activated potassium channel is an indirect activator.

57. The method of claim 56, wherein the indirect activator is an activator of soluble guanylyl cyclase.

58. The method of claim 57, wherein the activator of soluble guanylyl cyclase is nitric oxide.

59. The method of claim 57, wherein the activator of soluble guanylyl cyclase is an NO-independent soluble guanylyl cyclase activator.

60. The method of claim 59, wherein the NO-independent activator of soluble guanylyl cyclase is carbon monoxide, a porphyrin, a metallopophyrin, YC-1, BAY-2272, BAY 41-2272, BAY 41-8543.

61. The method of claim 44, wherein the medicant is an anti-proliferative agent.

62. The method of claim 61, wherein the anti-proliferative agent is carboplatin or cisplatin.

63. The method of claim 44, wherein the therapeutic or diagnostic agent is administered to the mammalian subject substantially simultaneously with the activators.

64. The method of claim 44, wherein the activators are administered to the mammalian subject by intravenous or intra-arterial infusion or injection.

65. The method of claim 44, wherein the activators are administered to the mammalian subject by intravenous or intra-arterial infusion or injection.
